SEQUENCE LISTING (149 sequences)

SEQ ID NO: 1 - DNA, 919 bp, artificial
  Sequence of the first arm of the cheA/gaCA/tolC/pglA/engXCA1/engXCA2 dsRNA used to concomitantly target cheA, gaCA, tolC, pglA, engXCA1, engXCA2 genes and regions from xylella fastidiosa
atgaacgcacttcgtgatgcgatgagccatgctgtgcttggctgggtaaagcctgatctagatgaagctttgggccaagcacgccaggagattgagttattcgttgagaaccctgtagacactagtcggatactttcttgcgctgattagatcatgactattaagatttttctgattgatgatcatactctcgtgcgtgttggcatgaagatgatcttatccaatgaattagatctcgaagtgataggggaagcggagacaggggaggcggctttaccacagatccgtgagctacgcccgaatatgcatgatccgccgctgcctcgccttggccgtgaccaccgccctatttcccgtcgtttcacaagcgacaaacctattacaagtctacgaaatggcacgcgctgctgaccctcaattaagcgcagccgaatccactcggctatacagcgttgaagtgcatgaaccttgaccgtttcctccccctagtgtttggactgcactgttttgccgcaatgggcggcactgctgatgtctttcgagtgtcacagctgtcctcatcagatctttccgagccggtatcggtgaaaacgctatggggcgaggtgcaaaagctggcctccgccatgtacggtggaacggtgggtgcacaaaatgcagtgtctgatactcactttgttgaaccactccgcggcgttaactggcgtggtttggaaacggcgcagcacctgccgcaaggtttaaatcaacgtccctggcgtgaagtccaatgtcgttttccaaacacttcggcaaactcatgttggccacgtgcctatccttatctgcggcaccagcattcagttacagcatcagtcacgggaaggtcgtcgatgacaaaggcaaccaaatccaattaaggggcgtcaactggtttggt

SEQ ID NO: 2 - DNA, 919 bp, artificial
  Sequence of the second arm of the cheA/gaCA/tolC/pglA/engXCA1/engXCA2 dsRNA used to concomitantly target cheA, gaCA, tolC, pglA/engXCA1, engXCA2 genes and regions from xylella fastidiosa
accaaaccagttgacgccccttaattggatttggttgcctttgtcatcgacgaccttcccgtgactgatgctgtaactgaatgctggtgccgcagataaggataggcacgtggccaacatgagtttgccgaagtgtttggaaaacgacattggacttcacgccagggacgttgatttaaaccttgcggcaggtgctgcgccgtttccaaaccacgccagttaacgccgcggagtggttcaacaaagtgagtatcagacactgcattttgtgcacccaccgttccaccgtacatggcggaggccagcttttgcacctcgccccatagcgttttcaccgataccggctcggaaagatctgatgaggacagctgtgacactcgaaagacatcagcagtgccgcccattgcggcaaaacagtgcagtccaaacactagggggaggaaacggtcaaggttcatgcacttcaacgctgtatagccgagtggattcggctgcgcttaattgagggtcagcagcgcgtgccatttcgtagacttgtaataggtttgtcgcttgtgaaacgacgggaaatagggcggtggtcacggccaaggcgaggcagcggcggatcatgcatattcgggcgtagctcacggatctgtggtaaagccgcctcccctgtctccgcttcccctatcacttcgagatctaattcattggataagatcatcttcatgccaacacgcacgagagtatgatcatcaatcagaaaaatcttaatagtcatgatctaatcagcgcaagaaagtatccgactagtgtctacagggttctcaacgaataactcaatctcctggcgtgcttggcccaaagcttcatctagatcaggctttacccagccaagcacagcatggctcatcgcatcacgaagtgcgttcat

SEQ ID NO: 3 - DNA, 1073 bp, artificial
  Sequence of the first arm of the cheA/GumH/GumD/ XpsE/ LesA/ HolC dsRNA used to concomitantly target cheA, GumH, GumD, Xpse, LesA, HolC genes and regions from xylella fastidiosa
atgaacgcacttcgtgatgcgatgagccatgctgtgcttggctgggtaaagcctgatctagatgaagctttgggccaagcacgccaggagattgagttattcgttgagaaccctgtagacactagtcggatactttcttgcgctgattagatcatgaaagtggtgcatgttgttcgtcaatttcatccgtcaattggtgggatggaggatgtggtcttcaatattgcgatgcagctgcatttgcatgctggaatcggtgttgatgttgtgacgttgaaccgagtatttactcagtctgatgttatgcatgcttctggcagatctgaacagtgttactgatattgcttcttcaccgcggttattgtcgaaatattctgctgccgctgatctaatacttcgtgtttttgatctaacaatggtggtgctgtctggtgtactgatttacagattgttgtttgtgcatgaaccttgaccgtttcctccccctagtgtttggactgcactgttttgccgcaatgggcggcactgctgatgtctttcgagtgtcacagctgtcctcatcagatctttccgagccggtatcggtgaaaacgctatggggcgaggtgcaaaagcgtggaaaataggaataacgacaatacgttgttgtgcgttgttggcggaagtatgaatgtaagtatggaagcatccatcgttgagtcgttgttgcagcgtcgttgcttgaaagaaagtgatctgtatcgtgcgcgtcagttgcaggccgaatccaatgatgtatggttctcaacgaaaaccggtgcttcttatcctgagttactttatttttatctgtgctctgttcccctgtgttgcagctgcttggccttcacgtgggatggtgattaacagaaaaattattggtagctatacgcagcgtcagtccaatggcacgcgccgacttctatttgattgccaaaccgcgctttcttgagaaaccgctgcaattagtctgcgaattagtccgcaaagcctacgacacccatcagcccacactgattctggcccgcgaccaggcccaggccgaagcactggat

SEQ ID NO: 4 - DNA, 1073 bp, artificial
  Sequence of the second arm of the cheA/GumH/GumD/ XpsE/ LesA/ HolC dsRNA used to concomitantly target cheA, GumH, GumD, Xpse, LesA, HolC genes and regions from xylella fastidiosa
atccagtgcttcggcctgggcctggtcgcgggccagaatcagtgtgggctgatgggtgtcgtaggctttgcggactaattcgcagactaattgcagcggtttctcaagaaagcgcggtttggcaatcaaatagaagtcggcgcgtgccattggactgacgctgcgtatagctaccaataatttttctgttaatcaccatcccacgtgaaggccaagcagctgcaacacaggggaacagagcacagataaaaataaagtaactcaggataagaagcaccggttttcgttgagaaccatacatcattggattcggcctgcaactgacgcgcacgatacagatcactttctttcaagcaacgacgctgcaacaacgactcaacgatggatgcttccatacttacattcatacttccgccaacaacgcacaacaacgtattgtcgttattcctattttccacgcttttgcacctcgccccatagcgttttcaccgataccggctcggaaagatctgatgaggacagctgtgacactcgaaagacatcagcagtgccgcccattgcggcaaaacagtgcagtccaaacactagggggaggaaacggtcaaggttcatgcacaaacaacaatctgtaaatcagtacaccagacagcaccaccattgttagatcaaaaacacgaagtattagatcagcggcagcagaatatttcgacaataaccgcggtgaagaagcaatatcagtaacactgttcagatctgccagaagcatgcataacatcagactgagtaaatactcggttcaacgtcacaacatcaacaccgattccagcatgcaaatgcagctgcatcgcaatattgaagaccacatcctccatcccaccaattgacggatgaaattgacgaacaacatgcaccactttcatgatctaatcagcgcaagaaagtatccgactagtgtctacagggttctcaacgaataactcaatctcctggcgtgcttggcccaaagcttcatctagatcaggctttacccagccaagcacagcatggctcatcgcatcacgaagtgcgttcat

SEQ ID NO: 5 - DNA, 758 bp, artificial
  Sequence of the first arm of the LesA/GumH/GumD dsRNA used to concomitantly target LesA, GumH, GumD  genes and regions from xylella fastidiosa
atgatgtatggttctcaacgaaaaccggtgcttcttatcctgagttactttatttttatctgtgctctgttcccctgtgttgcagctgcttggccttcacgtgggatggtgattaacagaaaaattattggtagctatacgcagcgtcagattgctgctctgcttactgatgagccgccatccgagcaacccaaatgcaacgttcgagtggttgaaatgacgtatacaacggttggtgtggttggtgaacgatcatgaaagtggtgcatgttgttcgtcaatttcatccgtcaattggtgggatggaggatgtggtcttcaatattgcgatgcagctgcatttgcatgctggaatcggtgttgatgttgtgacgttgaaccgagtatttactcagtctgatgttctacttccatgtactgataagtatcagggtgtgtcgatccagcgcattggttatcgtggttcttcgcgttatccattggcaccaggagtgttgagtatgtatgcatgcttctggcagatctgaacagtgttactgatattgcttcttcaccgcggttattgtcgaaatattctgctgccgctgatctaatacttcgtgtttttgatctaacaatggtggtgctgtctggtgtactgatttacagattgttgtttggtaattggatgttaacggccacatatcgtattgctattggtaacacgttattgtattcggtgatttgtttttcttttttcccgttgtatcgcagttggc

SEQ ID NO: 6 - DNA, 758 bp, artificial
  Sequence of the second arm of the LesA/GumH/GumD dsRNA used to concomitantly target LesA, GumH, GumD genes and regions from xylella fastidiosa
gccaactgcgatacaacgggaaaaaagaaaaacaaatcaccgaatacaataacgtgttaccaatagcaatacgatatgtggccgttaacatccaattaccaaacaacaatctgtaaatcagtacaccagacagcaccaccattgttagatcaaaaacacgaagtattagatcagcggcagcagaatatttcgacaataaccgcggtgaagaagcaatatcagtaacactgttcagatctgccagaagcatgcatacatactcaacactcctggtgccaatggataacgcgaagaaccacgataaccaatgcgctggatcgacacaccctgatacttatcagtacatggaagtagaacatcagactgagtaaatactcggttcaacgtcacaacatcaacaccgattccagcatgcaaatgcagctgcatcgcaatattgaagaccacatcctccatcccaccaattgacggatgaaattgacgaacaacatgcaccactttcatgatcgttcaccaaccacaccaaccgttgtatacgtcatttcaaccactcgaacgttgcatttgggttgctcggatggcggctcatcagtaagcagagcagcaatctgacgctgcgtatagctaccaataatttttctgttaatcaccatcccacgtgaaggccaagcagctgcaacacaggggaacagagcacagataaaaataaagtaactcaggataagaagcaccggttttcgttgagaaccatacatcat

SEQ ID NO: 7 - DNA, 758 bp, artificial
  Sequence of the first arm of the XpsE/HolC/LesA dsRNA used to concomitantly target XpsE, HolC, LesA  genes and regions from xylella fastidiosa
gtggaaaataggaataacgacaatacgttgttgtgcgttgttggcggaagtatgaatgtaagtatggaagcatccatcgttgagtcgttgttgcagcgtcgttgcttgaaagaaagtgatctgtatcgtgcgcgtcagttgcaggccgaatccggtgttgggttgttacctttgttgggccgtcttggtttggtttctgagcgcgatcacgctgaagtatgtgcggaggtgatggggctgttgttgatacgatcatggcacgcgccgacttctatttgattgccaaaccgcgctttcttgagaaaccgctgcaattagtctgcgaattagtccgcaaagcctacgacacccatcagcccacactgattctggcccgcgaccaggcccaggccgaagcactggatgacctactgtgggcgttcgacccggacgcctacatcccccaccaaatcgctggcagcgacgaagacgacgacatcacccccgtattgattgccaccccggatgcatgatgtatggttctcaacgaaaaccggtgcttcttatcctgagttactttatttttatctgtgctctgttcccctgtgttgcagctgcttggccttcacgtgggatggtgattaacagaaaaattattggtagctatacgcagcgtcagattgctgctctgcttactgatgagccgccatccgagcaacccaaatgcaacgttcgagtggttgaaatgacgtatacaacggttggtgtggttggtgaac

SEQ ID NO: 8 - DNA, 758 bp, artificial
  Sequence of the second arm of the XpsE/HolC/LesA dsRNA used to concomitantly target XpsE, HolC, LesA genes and regions from xylella fastidiosa
gttcaccaaccacaccaaccgttgtatacgtcatttcaaccactcgaacgttgcatttgggttgctcggatggcggctcatcagtaagcagagcagcaatctgacgctgcgtatagctaccaataatttttctgttaatcaccatcccacgtgaaggccaagcagctgcaacacaggggaacagagcacagataaaaataaagtaactcaggataagaagcaccggttttcgttgagaaccatacatcatgcatccggggtggcaatcaatacgggggtgatgtcgtcgtcttcgtcgctgccagcgatttggtgggggatgtaggcgtccgggtcgaacgcccacagtaggtcatccagtgcttcggcctgggcctggtcgcgggccagaatcagtgtgggctgatgggtgtcgtaggctttgcggactaattcgcagactaattgcagcggtttctcaagaaagcgcggtttggcaatcaaatagaagtcggcgcgtgccatgatcgtatcaacaacagccccatcacctccgcacatacttcagcgtgatcgcgctcagaaaccaaaccaagacggcccaacaaaggtaacaacccaacaccggattcggcctgcaactgacgcgcacgatacagatcactttctttcaagcaacgacgctgcaacaacgactcaacgatggatgcttccatacttacattcatacttccgccaacaacgcacaacaacgtattgtcgttattcctattttccac

SEQ ID NO: 9 - DNA, 759 bp, artificial
  Sequence of the first arm of the CheA/pglA/LesA dsRNA used to concomitantly target CheA, pglA, lesA  genes and regions from xylella fastidiosa
tgaacgcacttcgtgatgcgatgagccatgctgtgcttggctgggtaaagcctgatctagatgaagctttgggccaagcacgccaggagattgagttattcgttgagaaccctgtagacactagtcggatactttcttgcgctgattacctgcatcaggtgcagggtaccttgcgaatgcttgaattatatgctcccgcaatggtggccgaggaaatggaacggctagctaaggtgatgcaagtcgatgggatcatgaaccttgaccgtttcctccccctagtgtttggactgcactgttttgccgcaatgggcggcactgctgatgtctttcgagtgtcacagctgtcctcatcagatctttccgagccggtatcggtgaaaacgctatggggcgaggtgcaacgtcccagcctacctacccacgtgtgtacggtattgcctgcgcgcctgactcctaaacatggctccattgacgcactcgatgccaatccaaaggtttccaaatgcatgatgtatggttctcaacgaaaaccggtgcttcttatcctgagttactttatttttatctgtgctctgttcccctgtgttgcagctgcttggccttcacgtgggatggtgattaacagaaaaattattggtagctatacgcagcgtcagattgctgctctgcttactgatgagccgccatccgagcaacccaaatgcaacgttcgagtggttgaaatgacgtatacaacggttggtgtggttggtgaac

SEQ ID NO: 10 - DNA, 759 bp, artificial
  Sequence of the second arm of the CheA/pglA/LesA dsRNA used to concomitantly target CheA, pglA, lesA genes and regions from xylella fastidiosa
gttcaccaaccacaccaaccgttgtatacgtcatttcaaccactcgaacgttgcatttgggttgctcggatggcggctcatcagtaagcagagcagcaatctgacgctgcgtatagctaccaataatttttctgttaatcaccatcccacgtgaaggccaagcagctgcaacacaggggaacagagcacagataaaaataaagtaactcaggataagaagcaccggttttcgttgagaaccatacatcatgcatttggaaacctttggattggcatcgagtgcgtcaatggagccatgtttaggagtcaggcgcgcaggcaataccgtacacacgtgggtaggtaggctgggacgttgcacctcgccccatagcgttttcaccgataccggctcggaaagatctgatgaggacagctgtgacactcgaaagacatcagcagtgccgcccattgcggcaaaacagtgcagtccaaacactagggggaggaaacggtcaaggttcatgatcccatcgacttgcatcaccttagctagccgttccatttcctcggccaccattgcgggagcatataattcaagcattcgcaaggtaccctgcacctgatgcaggtaatcagcgcaagaaagtatccgactagtgtctacagggttctcaacgaataactcaatctcctggcgtgcttggcccaaagcttcatctagatcaggctttacccagccaagcacagcatggctcatcgcatcacgaagtgcgttca

SEQ ID NO: 11 - DNA, 758 bp, artificial
  Sequence of the first arm of the cheA/engXCA1/engXCA2 dsRNA used to concomitantly target cheA, engXCA1, engXCA2 genes and regions from xylella fastidiosa
tgaacgcacttcgtgatgcgatgagccatgctgtgcttggctgggtaaagcctgatctagatgaagctttgggccaagcacgccaggagattgagttattcgttgagaaccctgtagacactagtcggatactttcttgcgctgattacctgcatcaggtgcagggtaccttgcgaatgcttgaattatatgctcccgcaatggtggccgaggaaatggaacggctagctaaggtgatgcaagtcgatgggatcgtggcctccgccatgtacggtggaacggtgggtgcacaaaatgcagtgtctgatactcactttgttgaaccactccgcggcgttaactggcgtggtttggaaacggcgcagcacctgccgcaaggtttaaatcaacgtccctggcgtgaagtactcgaccaaatgcagagcctaggcattaacgcaatacgcctgcccctgtgctcggacaccctacatggcgccatgcctaccaatctggatctagtgcatgcatgtcgttttccaaacacttcggcaaactcatgttggccacgtgcctatccttatctgcggcaccagcattcagttacagcatcagtcacgggaaggtcgtcgatgacaaaggcaaccaaatccaattaaggggcgtcaactggtttggttttgaaaccggagatcatgtggtaaatggactatgggcacgtaattggaaagaattcatcacacagctacagggcatgggattcaacgcgatacgtttac

SEQ ID NO: 12 - DNA, 758 bp, artificial
  Sequence of the second arm of the cheA/engXCA1/engXCA2 dsRNA used to concomitantly target cheA, engXCA1, engXCA2 genes and regions from xylella fastidiosa
gtaaacgtatcgcgttgaatcccatgccctgtagctgtgtgatgaattctttccaattacgtgcccatagtccatttaccacatgatctccggtttcaaaaccaaaccagttgacgccccttaattggatttggttgcctttgtcatcgacgaccttcccgtgactgatgctgtaactgaatgctggtgccgcagataaggataggcacgtggccaacatgagtttgccgaagtgtttggaaaacgacatgcatgcactagatccagattggtaggcatggcgccatgtagggtgtccgagcacaggggcaggcgtattgcgttaatgcctaggctctgcatttggtcgagtacttcacgccagggacgttgatttaaaccttgcggcaggtgctgcgccgtttccaaaccacgccagttaacgccgcggagtggttcaacaaagtgagtatcagacactgcattttgtgcacccaccgttccaccgtacatggcggaggccacgatcccatcgacttgcatcaccttagctagccgttccatttcctcggccaccattgcgggagcatataattcaagcattcgcaaggtaccctgcacctgatgcaggtaatcagcgcaagaaagtatccgactagtgtctacagggttctcaacgaataactcaatctcctggcgtgcttggcccaaagcttcatctagatcaggctttacccagccaagcacagcatggctcatcgcatcacgaagtgcgttca

SEQ ID NO: 13 - DNA, 759 bp, artificial
  Sequence of the first arm of the gacA/pglA/engXCA2 dsRNA used to concomitantly target gacA, pglA, engXCA2 genes and regions from xylella fastidiosa
atgactattaagatttttctgattgatgatcatactctcgtgcgtgttggcatgaagatgatcttatccaatgaattagatctcgaagtgataggggaagcggagacaggggaggcggctttaccacagatccgtgagctacgcccgaatgtcgtattgtgtgacatgcatctccctggggtgagtgggctggagattacagaaaaattggtgaaagggaattatggtagccgtgtaattattgtttcgggatcatgaaccttgaccgtttcctccccctagtgtttggactgcactgttttgccgcaatgggcggcactgctgatgtctttcgagtgtcacagctgtcctcatcagatctttccgagccggtatcggtgaaaacgctatggggcgaggtgcaacgtcccagcctacctacccacgtgtgtacggtattgcctgcgcgcctgactcctaaacatggctccattgacgcactcgatgccaatccaaaggtttccaaatgcatgtcgttttccaaacacttcggcaaactcatgttggccacgtgcctatccttatctgcggcaccagcattcagttacagcatcagtcacgggaaggtcgtcgatgacaaaggcaaccaaatccaattaaggggcgtcaactggtttggttttgaaaccggagatcatgtggtaaatggactatgggcacgtaattggaaagaattcatcacacagctacagggcatgggattcaacgcgatacgtttac

SEQ ID NO: 14 - DNA, 759 bp, artificial
  Sequence of the second arm of the gacA/pglA/engXCA2 dsRNA used to concomitantly target gacA, pglA, engXCA2 genes and regions from xylella fastidiosa
gtaaacgtatcgcgttgaatcccatgccctgtagctgtgtgatgaattctttccaattacgtgcccatagtccatttaccacatgatctccggtttcaaaaccaaaccagttgacgccccttaattggatttggttgcctttgtcatcgacgaccttcccgtgactgatgctgtaactgaatgctggtgccgcagataaggataggcacgtggccaacatgagtttgccgaagtgtttggaaaacgacatgcatttggaaacctttggattggcatcgagtgcgtcaatggagccatgtttaggagtcaggcgcgcaggcaataccgtacacacgtgggtaggtaggctgggacgttgcacctcgccccatagcgttttcaccgataccggctcggaaagatctgatgaggacagctgtgacactcgaaagacatcagcagtgccgcccattgcggcaaaacagtgcagtccaaacactagggggaggaaacggtcaaggttcatgatcccgaaacaataattacacggctaccataattccctttcaccaatttttctgtaatctccagcccactcaccccagggagatgcatgtcacacaatacgacattcgggcgtagctcacggatctgtggtaaagccgcctcccctgtctccgcttcccctatcacttcgagatctaattcattggataagatcatcttcatgccaacacgcacgagagtatgatcatcaatcagaaaaatcttaatagtcat

SEQ ID NO: 15 - DNA, 759 bp, artificial
  Sequence of the first arm of the cheA/tolC/engXCA1 dsRNA used to concomitantly target cheA, tolC, engXCA1 genes and regions from xylella fastidiosa
tgaacgcacttcgtgatgcgatgagccatgctgtgcttggctgggtaaagcctgatctagatgaagctttgggccaagcacgccaggagattgagttattcgttgagaaccctgtagacactagtcggatactttcttgcgctgattacctgcatcaggtgcagggtaccttgcgaatgcttgaattatatgctcccgcaatggtggccgaggaaatggaacggctagctaaggtgatgcaagtcgatgggatcatgatccgccgctgcctcgccttggccgtgaccaccgccctatttcccgtcgtttcacaagcgacaaacctattacaagtctacgaaatggcacgcgctgctgaccctcaattaagcgcagccgaatccactcggctatacagcgttgaagggcaagttcaggcacgtgcagcgctgctgccgcagatgagcggcacagccaccctggcacgtagccgcactgaatacgacggtatcagcggatcagccatatgcgtggcctccgccatgtacggtggaacggtgggtgcacaaaatgcagtgtctgatactcactttgttgaaccactccgcggcgttaactggcgtggtttggaaacggcgcagcacctgccgcaaggtttaaatcaacgtccctggcgtgaagtactcgaccaaatgcagagcctaggcattaacgcaatacgcctgcccctgtgctcggacaccctacatggcgccatgcctaccaatctggatctagtgc

SEQ ID NO: 16 - DNA, 759 bp, artificial
  Sequence of the second arm of the cheA/tolC/engXCA1 dsRNA used to concomitantly target cheA, tolC, engXCA1 genes and regions from xylella fastidiosa
gcactagatccagattggtaggcatggcgccatgtagggtgtccgagcacaggggcaggcgtattgcgttaatgcctaggctctgcatttggtcgagtacttcacgccagggacgttgatttaaaccttgcggcaggtgctgcgccgtttccaaaccacgccagttaacgccgcggagtggttcaacaaagtgagtatcagacactgcattttgtgcacccaccgttccaccgtacatggcggaggccacgcatatggctgatccgctgataccgtcgtattcagtgcggctacgtgccagggtggctgtgccgctcatctgcggcagcagcgctgcacgtgcctgaacttgcccttcaacgctgtatagccgagtggattcggctgcgcttaattgagggtcagcagcgcgtgccatttcgtagacttgtaataggtttgtcgcttgtgaaacgacgggaaatagggcggtggtcacggccaaggcgaggcagcggcggatcatgatcccatcgacttgcatcaccttagctagccgttccatttcctcggccaccattgcgggagcatataattcaagcattcgcaaggtaccctgcacctgatgcaggtaatcagcgcaagaaagtatccgactagtgtctacagggttctcaacgaataactcaatctcctggcgtgcttggcccaaagcttcatctagatcaggctttacccagccaagcacagcatggctcatcgcatcacgaagtgcgttca

SEQ ID NO: 17 - DNA, 799 bp, artificial
  Sequence of the first arm of the gyrB dsRNA used to concomitantly target gyrase subunit B gene from xanthomonas hortorum vitians
atgaccgaagatcaaacaccccgaccacacccaacagcacctacgattccagcaagatcaccgtgctgcgtggcctggaagccgtccgcaagcgccccggcatgtacatcggcgatgtccatgacggcaccggcctgcatcacatggtgttcgaggtggtcgacaactcggtcgacgaggccctggccggccatgccgacgacatcgtggtcaagatcctggtcgatggctcggtcgccgtgtcggataacggccgcggcgtgccggtcgacatccacaaggaagaaggcgtgtcggcggcggaggtgatcctcaccgtgctgcatgccggcggcaagttcgacgataacagttacaaggtttccggcggcctgcacggcgtgggcgtgtcggtggtcaacgcgctgtccgagcacctgtggctggacatctggcgcgacggcttccactaccagcaggaatacgcgctgggtgagccgcagtacccgctcaagcaactcgaagcctcgaccaagcgcggcaccacgctgcgcttcaagccggccgtggccatcttcagcgacgtcgagttccattacgacatcctggcgcgccgtctgcgtgagctgtcgttcctcaattccggcgtcaagatcgcgctgatcgacgaacgtggcgaaggccgccgcgacgatttccattacgaaggcggcatccgcagcttcgtggagcatctggcccagctgaagtcgccgctgcaccccaacgtgatctcggtgaccggcgagcacaacggcatcgtggtggacgtggccctgca

SEQ ID NO: 18 - DNA, 798 bp, artificial
  Sequence of the second arm of the gyrB dsRNA used to concomitantly target gyrase subunit B gene from xanthomonas hortorum vitians
tgcagggccacgtccaccacgatgccgttgtgctcgccggtcaccgagatcacgttggggtgcagcggcgacttcagctgggccagatgctccacgaagctgcggatgccgccttcgtaatggaaatcgtcgcggcggccttcgccacgttcgtcgatcagcgcgatcttgacgccggaattgaggaacgacagctcacgcagacggcgcgccaggatgtcgtaatggaactcgacgtcgctgaagatggccacggccggcttgaagcgcagcgtggtgccgcgcttggtcgaggcttcgagttgcttgagcgggtactgcggctcacccagcgcgtattcctgctggtagtggaagccgtcgcgccagatgtccagccacaggtgctcggacagcgcgttgaccaccgacacgcccacgccgtgcaggccgccggaaaccttgtaactgttatcgtcgaacttgccgccggcatgcagcacggtgaggatcacctccgccgccgacacgccttcttccttgtggatgtcgaccggcacgccgcggccgttatccgacacggcgaccgagccatcgaccaggatcttgaccacgatgtcgtcggcatggccggccagggcctcgtcgaccgagttgtcgaccacctcgaacaccatgtgatgcaggccggtgccgtcatggacatcgccgatgtacatgccggggcgcttgcggacggcttccaggccacgcagcacggtgatcttgctggaatcgtaggtgctgttgggtgtggtcgggggtttgatcttcggtcat

SEQ ID NO: 19 - DNA, 801 bp, artificial
  Sequence of the first arm of the fusA dsRNA used to concomitantly target fusa elongator factor G gene from xanthomonas hortorum vitians
gtggccgcaaccaacccctatcgagcgttaccgcaacttcggcatcatggctcacatcgatgccggcaagaccaccacctccgagcgcatcctgttttacaccggcgtcagtcacaagatcggcgaagtccatgacggcgccgcggtgatggactggatggagcaggagcaggagcgtggtatcaccatcacctccgctgccaccaccgcgttctggtcgggtatggacaagtccatgccgcagcatcgcctcaacatcatcgacacccccgggcacgtcgacttcaccatcgaggtggagcgctccttgcgcgtgctcgacggtgcggtgttcgtgctgtgcgccgtgggtggcgtgcagccgcagtccgagaccgtgtggcgccaggccaacaagtattccgtgccgcgtatggccttcgtcaacaagatggaccgtaccggtgccaacttcgacaaggtcgtcgagcagctgaaggcccgtctgggagcctacgccgtgccgatgcaggtgccgatcggtgctgaagacggtttcgagggcgtggtcgacctgctgaagatgaaggcgatccattgggatgttgcctcgcagggcaccaccttcgaatacggtgacatcccggccgatctggtcgagattgcgaccgaggcgcgttcgttcatggttgaagcggctgcagaagccagcgaagacctgatggacaagtacctcaacgaaggcgacctgaccgaagaagagatcctggtgggtctgcgcgagcgcaccctgaaggtcgagatcgtgccggtgttctgtgg

SEQ ID NO: 20 - DNA, 801 bp, artificial; identical to SEQ ID NO: 19
  Sequence of the second arm of the fusA dsRNA used to concomitantly target fusa elongator factor G gene from xanthomonas hortorum vitians

SEQ ID NO: 21 - DNA, 733 bp, artificial
  Sequence of the first arm of the ZipA dsRNA used to concomitantly target ZipA gene from xanthomonas hortorum vitians
atgtccgaacatggccatgatccggatcgggatcctgatcgctggactgctgctggtcgcggccatcttcctgtttggtcgcccgaaaaaatcgccgcaagggcgccgggtcgacaaggacgacgggcagccgcgcgaacgccgcgagccggtgatttccggcgagcctggggtggagggcgacccgttcaagcgcactgaaggcgccgagcagagcgaactcaatctggacgatgccgatgcggccggcggcaacgatgtcggcaagcgtccgaatcaggacttcgacaagatcgtctcgctgttcgtcgcggccaaggccggacaggtgctgcgcggcgaagacgtggttgttgctgccgagaagacggggttggtgttcggccacatgaatgtcttccaccgcttggtggaagggcacccggagcgcgggccgatcttcagcatggccagcatcctcaagcccggcagtttcgacatggccaacatccgcgagatgcagaccccggcgatcgcatttttcctcacgctgccggcgccgatgactgcgctggatgcctgggaaaaaatgctgccgaccgtgcagcgcatggccgaactactcgatggcgtggtgctggacgacagccgcaatgcgctgggccgtcaacgtgtggcgcatatccgcgatgagctgcgcgcctacgatcgccagcatcaggcgccgccgctaaccaagtcgccgcgctggtga

SEQ ID NO: 22 - DNA, 733 bp, artificial
  Sequence of the second arm of the ZipA dsRNA used to concomitantly target ZipA gene from xanthomonas hortorum vitians
tcaccagcgcggcgacttggttagcggcggcgcctgatgctggcgatcgtaggcgcgcagctcatcgcggatatgcgccacacgttgacggcccagcgcattgcggctgtcgtccagcaccacgccatcgagtagttcggccatgcgctgcacggtcggcagcattttttcccaggcatccagcgcagtcatcggcgccggcagcgtgaggaaaaatgcgatcgccggggtctgcatctcgcggatgttggccatgtcgaaactgccgggcttgaggatgctggccatgctgaagatcggcccgcgctccgggtgcccttccaccaagcggtggaagacattcatgtggccgaacaccaaccccgtcttctcggcagcaacaaccacgtcttcgccgcgcagcacctgtccggccttggccgcgacgaacagcgagacgatcttgtcgaagtcctgattcggacgcttgccgacatcgttgccgccggccgcatcggcatcgtccagattgagttcgctctgctcggcgccttcagtgcgcttgaacgggtcgccctccaccccaggctcgccggaaatcaccggctcgcggcgttcgcgcggctgcccgtcgtccttgtcgacccggcgcccttgcggcgattttttcgggcgaccaaacaggaagatggccgcgaccagcagcagtccagcgatcaggatcccgatccggatcatggccatgttcggacat

SEQ ID NO: 23 - DNA, 919 bp, artificial
  Sequence of the first arm of the rimM/rsgA/rbfA/mreB/gyrB/ftsZ dsRNA used to concomitantly target rimM,rsgA,rbfA,mreB,gyrB,ftsZ genes from acidovorax valerianella
atgcctaacctccccgttcttgagcctgccgaattgccgcccgacgcggtggagatcggccgtattgccgatgcctggggtgtgaagggctggttcaaggtgatggcatacagtgccgaccccgaagcgctgttttcgtccaagcgctgggatcatggctgagcgcagcgccctgtttgacggactggtggtggccagccatggccggcactgccttgtcgagtcgcccgacggccagcgccgcatctgccacccccgcggaaagaaaagccaggcggtggtgggagaccatgtttttggcagatgcatggctgagcgcagcgccctgtttgacggactggtggtggccagccatggccggcactgccttgtcgagtcgcccgacggccagcgccgcatctgccacccccgcggaaagaaaagccaggcggtggtgggagaccatgttctttggcaggtgcatgttcggagctttccgtcggtacttctccaccgaccttgccattgaccttggcacagccaacaccctgatttttgcccgcgacaagggcatcgtgctcgatgaaccctcggtcgttgccatccgccacgagggcggtccccacggaaagaagcatgaccgctgagaacaccctgcccgagccttccaccgacggcgcgggcgaagcacagcccttcaagatcgacacccagcaagtgggcgccagcgaaggctacggcgaaggcgcgatccagatcctggaaggcctggaggccgtgcgcaagtccaatgaccatcgaaatgatcgaagccgaagaattcaatcagggcacccagatcaaggtgatcggtgtcggcggcggcggcggcaatgccgtggcccacatgatcgcgcgcaatgttcagggcgtggagttcgtctgcgccaataccgacgcg

SEQ ID NO: 24 - DNA, 919 bp, artificial
  Sequence of the second arm of the rimM/rsgA/rbfA/mreB/gyrB/ftsZ dsRNA used to concomitantly target rimM,rsgA,rbfA,mreB,gyrB,ftsZ genes from acidovorax valerianella
cgcgtcggtattggcgcagacgaactccacgccctgaacattgcgcgcgatcatgtgggccacggcattgccgccgccgccgccgacaccgatcaccttgatctgggtgccctgattgaattcttcggcttcgatcatttcgatggtcattggacttgcgcacggcctccaggccttccaggatctggatcgcgccttcgccgtagccttcgctggcgcccacttgctgggtgtcgatcttgaagggctgtgcttcgcccgcgccgtcggtggaaggctcgggcagggtgttctcagcggtcatgcttctttccgtggggaccgccctcgtggcggatggcaacgaccgagggttcatcgagcacgatgcccttgtcgcgggcaaaaatcagggtgttggctgtgccaaggtcaatggcaaggtcggtggagaagtaccgacggaaagctccgaacatgcacctgccaaagaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccatgcatctgccaaaaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccatgatcccagcgcttggacgaaaacagcgcttcggggtcggcactgtatgccatcaccttgaaccagcccttcacaccccaggcatcggcaatacggccgatctccaccgcgtcgggcggcaattcggcaggctcaagaacggggaggttaggcat

SEQ ID NO: 25 - DNA, 758 bp, artificial
  Sequence of the first arm of the rimM/rbfA/ftsZ dsRNA used to concomitantly target rimM, rbfA,ftsZ genes from acidovorax valerianella
atgcctaacctccccgttcttgagcctgccgaattgccgcccgacgcggtggagatcggccgtattgccgatgcctggggtgtgaagggctggttcaaggtgatggcatacagtgccgaccccgaagcgctgttttcgtccaagcgctggtatctgcagcccagcgagaagggcgccaagagctttttcgaaggcaccgtggtgctggcggtgcgacaggcgcgcgagcactccgacacggtcgtggcctgatcatggctgagcgcagcgccctgtttgacggactggtggtggccagccatggccggcactgccttgtcgagtcgcccgacggccagcgccgcatctgccacccccgcggaaagaaaagccaggcggtggtgggagaccatgttctttggcaggcggccccgcccggccagggtgatgagggcaccatcgagaaggtcaaggaacgccggaacctgttttaccggcaagacgaaattcgcaccaaatcctttgatgcatgaccatcgaaatgatcgaagccgaagaattcaatcagggcacccagatcaaggtgatcggtgtcggcggcggcggcggcaatgccgtggcccacatgatcgcgcgcaatgttcagggcgtggagttcgtctgcgccaataccgacgcgcaagccctcacccgcagccccgcgcaccgcaccatccagctgggcggcagcggcctgggcgccggcagcaagcccgacaagggccgcgacgccgctgaag

SEQ ID NO: 26 - DNA, 758 bp, artificial
  Sequence of the second arm of the rimM/rbfA/ftsZ dsRNA used to concomitantly target rimM, rbfA,ftsZ genes from acidovorax valerianella
cttcagcggcgtcgcggcccttgtcgggcttgctgccggcgcccaggccgctgccgcccagctggatggtgcggtgcgcggggctgcgggtgagggcttgcgcgtcggtattggcgcagacgaactccacgccctgaacattgcgcgcgatcatgtgggccacggcattgccgccgccgccgccgacaccgatcaccttgatctgggtgccctgattgaattcttcggcttcgatcatttcgatggtcatgcatcaaaggatttggtgcgaatttcgtcttgccggtaaaacaggttccggcgttccttgaccttctcgatggtgccctcatcaccctggccgggcggggccgcctgccaaagaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccatgatcaggccacgaccgtgtcggagtgctcgcgcgcctgtcgcaccgccagcaccacggtgccttcgaaaaagctcttggcgcccttctcgctgggctgcagataccagcgcttggacgaaaacagcgcttcggggtcggcactgtatgccatcaccttgaaccagcccttcacaccccaggcatcggcaatacggccgatctccaccgcgtcgggcggcaattcggcaggctcaagaacggggaggttaggcat

SEQ ID NO: 27 - DNA, 758 bp, artificial
  Sequence of the first arm of the RsgA/gyrB/MreB dsRNA used to concomitantly target RsgA, gyrB, MreB genes from acidovorax valerianella
atggctgagcgcagcgccctgtttgacggactggtggtggccagccatggccggcactgccttgtcgagtcgcccgacggccagcgccgcatctgccacccccgcggaaagaaaagccaggcggtggtgggagaccatgttctttggcaggcggccccgcccggccagggtgatgagggcaccatcgagaaggtcaaggaacgccggaacctgttttaccggcaagacgaaattcgcaccaaatcctttggatcatgaccgctgagaacaccctgcccgagccttccaccgacggcgcgggcgaagcacagcccttcaagatcgacacccagcaagtgggcgccagcgaaggctacggcgaaggcgcgatccagatcctggaaggcctggaggccgtgcgcaagcgccccggcatgtacatcggcgacacctccgacggcacgggcctgcaccacctcgtcttcgaagtcgtggacaactccatcgacgaggcgctggccgggcatgcatgttcggagctttccgtcggtacttctccaccgaccttgccattgaccttggcacagccaacaccctgatttttgcccgcgacaagggcatcgtgctcgatgaaccctcggtcgttgccatccgccacgagggcggtccccacggaaagaaagtcatccaggccgtcggccgtgaagccaaggccatgctgggcaaggtgcccggcaacatcgaagccatccgcccgatgaaagacggggtgatcgcgg

SEQ ID NO: 28 - DNA, 758 bp, artificial
  Sequence of the second arm of the RsgA/gyrB/MreB dsRNA used to concomitantly target RsgA, gyrB, MreB genes from acidovorax valerianella
ccgcgatcaccccgtctttcatcgggcggatggcttcgatgttgccgggcaccttgcccagcatggccttggcttcacggccgacggcctggatgactttctttccgtggggaccgccctcgtggcggatggcaacgaccgagggttcatcgagcacgatgcccttgtcgcgggcaaaaatcagggtgttggctgtgccaaggtcaatggcaaggtcggtggagaagtaccgacggaaagctccgaacatgcatgcccggccagcgcctcgtcgatggagttgtccacgacttcgaagacgaggtggtgcaggcccgtgccgtcggaggtgtcgccgatgtacatgccggggcgcttgcgcacggcctccaggccttccaggatctggatcgcgccttcgccgtagccttcgctggcgcccacttgctgggtgtcgatcttgaagggctgtgcttcgcccgcgccgtcggtggaaggctcgggcagggtgttctcagcggtcatgatccaaaggatttggtgcgaatttcgtcttgccggtaaaacaggttccggcgttccttgaccttctcgatggtgccctcatcaccctggccgggcggggccgcctgccaaagaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccat

SEQ ID NO: 29 - DNA, 758 bp, artificial
  Sequence of the first arm of the RsgA/rbfA/FtsZ dsRNA used to concomitantly target RsgA, rbfA, FtsZ genes from acidovorax valerianella
atggctgagcgcagcgccctgtttgacggactggtggtggccagccatggccggcactgccttgtcgagtcgcccgacggccagcgccgcatctgccacccccgcggaaagaaaagccaggcggtggtgggagaccatgttctttggcaggcggccccgcccggccagggtgatgagggcaccatcgagaaggtcaaggaacgccggaacctgttttaccggcaagacgaaattcgcaccaaatcctttggatcatggctgagcgcagcgccctgtttgacggactggtggtggccagccatggccggcactgccttgtcgagtcgcccgacggccagcgccgcatctgccacccccgcggaaagaaaagccaggcggtggtgggagaccatgttctttggcaggcggccccgcccggccagggtgatgagggcaccatcgagaaggtcaaggaacgccggaacctgttttaccggcaagacgaaattcgcaccaaatcctttgatgcatgaccatcgaaatgatcgaagccgaagaattcaatcagggcacccagatcaaggtgatcggtgtcggcggcggcggcggcaatgccgtggcccacatgatcgcgcgcaatgttcagggcgtggagttcgtctgcgccaataccgacgcgcaagccctcacccgcagccccgcgcaccgcaccatccagctgggcggcagcggcctgggcgccggcagcaagcccgacaagggccgcgacgccgctgaag

SEQ ID NO: 30 - DNA, 758 bp, artificial
  Sequence of the second arm of the RsgA/rbfA/FtsZ dsRNA used to concomitantly target RsgA, rbfA, FtsZ genes from acidovorax valerianella
cttcagcggcgtcgcggcccttgtcgggcttgctgccggcgcccaggccgctgccgcccagctggatggtgcggtgcgcggggctgcgggtgagggcttgcgcgtcggtattggcgcagacgaactccacgccctgaacattgcgcgcgatcatgtgggccacggcattgccgccgccgccgccgacaccgatcaccttgatctgggtgccctgattgaattcttcggcttcgatcatttcgatggtcatgcatcaaaggatttggtgcgaatttcgtcttgccggtaaaacaggttccggcgttccttgaccttctcgatggtgccctcatcaccctggccgggcggggccgcctgccaaagaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccatgatccaaaggatttggtgcgaatttcgtcttgccggtaaaacaggttccggcgttccttgaccttctcgatggtgccctcatcaccctggccgggcggggccgcctgccaaagaacatggtctcccaccaccgcctggcttttctttccgcgggggtggcagatgcggcgctggccgtcgggcgactcgacaaggcagtgccggccatggctggccaccaccagtccgtcaaacagggcgctgcgctcagccat

SEQ ID NO: 31 - DNA, 920 bp, artificial
  Sequence of the first arm of the mreB/ybeY/rbfA/gyrB/ftsZ/rsgA dsRNA used to concomitantly target mreB,ybeY,rbfA,gyrB,ftsZ,rsgA genes from acidovorax citrulli
atgttcggagctttccgtcggtacttctccaccgaccttgccattgatcttggcacagccaacacgctgatcttcgcccgcgacaagggcatcgtgctcgacgaaccctccgtggtcgccatccgccacgaaggcggcccccacggcaaggatcatggccctgaaccagctttccctgtccctgcagttcgcgcggtttcccggcgtggaggcgcaccgcgccgccctgccccggcatgccgtcgtccgctggatccgccacgcgctctcgctggacgcggagatcaccgtgcgcatcgtcgatatgcatggctgctaaaaaatcctccacgcccaaccgcggcttcaaggtcgcggaccagatccagcgcgatctgaccgagctgatccgcgagttgaaggacccgcgcatcggcatggtcacgctgcaggccgtggaagtgacgcccgactacgcggtgcatgaccgctgagaacaccctgcccgagcccacccttcccacgggcgacagccaagcgcagtccgtcgcagcccccgactccgtgcccgcccaggccgccgcgcccagcgacggctacggcgaaggcgcgatccagatcctggaaggcctgaagcatgaccatcgaaatgatcgaagccgaagaattcaaccagggcacgcagatcaaggtgatcggtgtcggcggcggcggcggcaatgccgtcgagcacatgatctcgcgccaggtacagggcgtggaattcgtatgcgccaataccgatgcgtccaatgtctgagcgctccggccccctcgaaggcctggtggtggccagccacggccggcactgcctcgtcgagagccccgacggcgcgcggcgcatctgccatccgcgcggcaagaagagccaggcggtggtcggcgaccgcgtgctctggcag

SEQ ID NO: 32 - DNA, 920 bp, artificial
  Sequence of the second arm of the mreB/ybeY/rbfA/gyrB/ftsZ/rsgA dsRNA used to concomitantly target mreB,ybeY,rbfA,gyrB,ftsZ,rsgA genes from acidovorax citrulli
ctgccagagcacgcggtcgccgaccaccgcctggctcttcttgccgcgcggatggcagatgcgccgcgcgccgtcggggctctcgacgaggcagtgccggccgtggctggccaccaccaggccttcgagggggccggagcgctcagacattggacgcatcggtattggcgcatacgaattccacgccctgtacctggcgcgagatcatgtgctcgacggcattgccgccgccgccgccgacaccgatcaccttgatctgcgtgccctggttgaattcttcggcttcgatcatttcgatggtcatgcttcaggccttccaggatctggatcgcgccttcgccgtagccgtcgctgggcgcggcggcctgggcgggcacggagtcgggggctgcgacggactgcgcttggctgtcgcccgtgggaagggtgggctcgggcagggtgttctcagcggtcatgcaccgcgtagtcgggcgtcacttccacggcctgcagcgtgaccatgccgatgcgcgggtccttcaactcgcggatcagctcggtcagatcgcgctggatctggtccgcgaccttgaagccgcggttgggcgtggaggattttttagcagccatgcatatcgacgatgcgcacggtgatctccgcgtccagcgagagcgcgtggcggatccagcggacgacggcatgccggggcagggcggcgcggtgcgcctccacgccgggaaaccgcgcgaactgcagggacagggaaagctggttcagggccatgatccttgccgtgggggccgccttcgtggcggatggcgaccacggagggttcgtcgagcacgatgcccttgtcgcgggcgaagatcagcgtgttggctgtgccaagatcaatggcaaggtcggtggagaagtaccgacggaaagctccgaacat

SEQ ID NO: 33 - DNA, 758 bp, artificial
  Sequence of the first arm of the mreB/ybeY/rbfA dsRNA used to concomitantly target mreB,ybeY,rbfA genes from acidovorax citrulli
atgttcggagctttccgtcggtacttctccaccgaccttgccattgatcttggcacagccaacacgctgatcttcgcccgcgacaagggcatcgtgctcgacgaaccctccgtggtcgccatccgccacgaaggcggcccccacggcaagaaagtcatccaggccgtcggccacgaggccaaggccatgctcggcaaggtgcccggcaacatcgaggccatccgcccgatgaaggatggcgtgatcgccggatcatggccctgaaccagctttccctgtccctgcagttcgcgcggtttcccggcgtggaggcgcaccgcgccgccctgccccggcatgccgtcgtccgctggatccgccacgcgctctcgctggacgcggagatcaccgtgcgcatcgtcgatgccgaagagggccaggccctgaaccgcgagtaccggcacaaggactacgccaccaacgtgctcaccttcgactacgcgcaggagcccgtggtcatggccgatgcatggctgctaaaaaatcctccacgcccaaccgcggcttcaaggtcgcggaccagatccagcgcgatctgaccgagctgatccgcgagttgaaggacccgcgcatcggcatggtcacgctgcaggccgtggaagtgacgcccgactacgcgcatgcgaaggtgttcttcagcgtgctcgtgggcgacgccgacgcgacgcaggatgcgctgaaccaggccgcgggcttcctgcgcaacggcctgttcaagc

SEQ ID NO: 34 - DNA, 758 bp, artificial
  Sequence of the second arm of the mreB/ybeY/rbfA dsRNA used to concomitantly target mreB,ybeY,rbfA genes from acidovorax citrulli
gcttgaacaggccgttgcgcaggaagcccgcggcctggttcagcgcatcctgcgtcgcgtcggcgtcgcccacgagcacgctgaagaacaccttcgcatgcgcgtagtcgggcgtcacttccacggcctgcagcgtgaccatgccgatgcgcgggtccttcaactcgcggatcagctcggtcagatcgcgctggatctggtccgcgaccttgaagccgcggttgggcgtggaggattttttagcagccatgcatcggccatgaccacgggctcctgcgcgtagtcgaaggtgagcacgttggtggcgtagtccttgtgccggtactcgcggttcagggcctggccctcttcggcatcgacgatgcgcacggtgatctccgcgtccagcgagagcgcgtggcggatccagcggacgacggcatgccggggcagggcggcgcggtgcgcctccacgccgggaaaccgcgcgaactgcagggacagggaaagctggttcagggccatgatccggcgatcacgccatccttcatcgggcggatggcctcgatgttgccgggcaccttgccgagcatggccttggcctcgtggccgacggcctggatgactttcttgccgtgggggccgccttcgtggcggatggcgaccacggagggttcgtcgagcacgatgcccttgtcgcgggcgaagatcagcgtgttggctgtgccaagatcaatggcaaggtcggtggagaagtaccgacggaaagctccgaacat

SEQ ID NO: 35 - DNA, 758 bp, artificial
  Sequence of the first arm of the rsgA/GyrB/MreB dsRNA used to concomitantly target RsgA, GyrB, MreB genes from acidovorax citrulli
atgtctgagcgctccggccccctcgaaggcctggtggtggccagccacggccggcactgcctcgtcgagagccccgacggcgcgcggcgcatctgccatccgcgcggcaagaagagccaggcggtggtcggcgaccgcgtgctctggcaggccccgccgcccggccagggcgacgaaggcacgatcgagaaagtgctggagcgccgcaacctgttctaccggcaggacgagatccgcaccaaatcgttcggatcatgaccgctgagaacaccctgcccgagcccacccttcccacgggcgacagccaagcgcagtccgtcgcagcccccgactccgtgcccgcccaggccgccgcgcccagcgacggctacggcgaaggcgcgatccagatcctggaaggcctggaggccgtgcgcaagcgccccggcatgtacatcggcgacacgtcggacggcacgggcctgcaccacctcgtgttcgaagtggtggacaactccatcgacgatgcatgttcggagctttccgtcggtacttctccaccgaccttgccattgatcttggcacagccaacacgctgatcttcgcccgcgacaagggcatcgtgctcgacgaaccctccgtggtcgccatccgccacgaaggcggcccccacggcaagaaagtcatccaggccgtcggccacgaggccaaggccatgctcggcaaggtgcccggcaacatcgaggccatccgcccgatgaaggatggcgtgatcgccg

SEQ ID NO: 36 - DNA, 758 bp, artificial
  Sequence of the second arm of the rsgA/GyrB/MreB dsRNA used to concomitantly target RsgA, GyrB, MreB genes from acidovorax citrulli
cggcgatcacgccatccttcatcgggcggatggcctcgatgttgccgggcaccttgccgagcatggccttggcctcgtggccgacggcctggatgactttcttgccgtgggggccgccttcgtggcggatggcgaccacggagggttcgtcgagcacgatgcccttgtcgcgggcgaagatcagcgtgttggctgtgccaagatcaatggcaaggtcggtggagaagtaccgacggaaagctccgaacatgcatcgtcgatggagttgtccaccacttcgaacacgaggtggtgcaggcccgtgccgtccgacgtgtcgccgatgtacatgccggggcgcttgcgcacggcctccaggccttccaggatctggatcgcgccttcgccgtagccgtcgctgggcgcggcggcctgggcgggcacggagtcgggggctgcgacggactgcgcttggctgtcgcccgtgggaagggtgggctcgggcagggtgttctcagcggtcatgatccgaacgatttggtgcggatctcgtcctgccggtagaacaggttgcggcgctccagcactttctcgatcgtgccttcgtcgccctggccgggcggcggggcctgccagagcacgcggtcgccgaccaccgcctggctcttcttgccgcgcggatggcagatgcgccgcgcgccgtcggggctctcgacgaggcagtgccggccgtggctggccaccaccaggccttcgagggggccggagcgctcagacat

SEQ ID NO: 37 - DNA, 758 bp, artificial
  Sequence of the first arm of the YbeY/ RsgA/ MreB dsRNA used to concomitantly target YbeY, RsgA, MreB genes from acidovorax citrulli
atggccctgaaccagctttccctgtccctgcagttcgcgcggtttcccggcgtggaggcgcaccgcgccgccctgccccggcatgccgtcgtccgctggatccgccacgcgctctcgctggacgcggagatcaccgtgcgcatcgtcgatgccgaagagggccaggccctgaaccgcgagtaccggcacaaggactacgccaccaacgtgctcaccttcgactacgcgcaggagcccgtggtcatggccggatcatgtctgagcgctccggccccctcgaaggcctggtggtggccagccacggccggcactgcctcgtcgagagccccgacggcgcgcggcgcatctgccatccgcgcggcaagaagagccaggcggtggtcggcgaccgcgtgctctggcaggccccgccgcccggccagggcgacgaaggcacgatcgagaaagtgctggagcgccgcaacctgttctaccggcaggacgagatccgcaccaaatcgttcgatgcatgttcggagctttccgtcggtacttctccaccgaccttgccattgatcttggcacagccaacacgctgatcttcgcccgcgacaagggcatcgtgctcgacgaaccctccgtggtcgccatccgccacgaaggcggcccccacggcaagaaagtcatccaggccgtcggccacgaggccaaggccatgctcggcaaggtgcccggcaacatcgaggccatccgcccgatgaaggatggcgtgatcgccg

SEQ ID NO: 38 - DNA, 758 bp, artificial
  Sequence of the second arm of the YbeY/ RsgA/ MreB dsRNA used to concomitantly target YbeY, RsgA, MreB genes from acidovorax citrulli
cggcgatcacgccatccttcatcgggcggatggcctcgatgttgccgggcaccttgccgagcatggccttggcctcgtggccgacggcctggatgactttcttgccgtgggggccgccttcgtggcggatggcgaccacggagggttcgtcgagcacgatgcccttgtcgcgggcgaagatcagcgtgttggctgtgccaagatcaatggcaaggtcggtggagaagtaccgacggaaagctccgaacatgcatcgaacgatttggtgcggatctcgtcctgccggtagaacaggttgcggcgctccagcactttctcgatcgtgccttcgtcgccctggccgggcggcggggcctgccagagcacgcggtcgccgaccaccgcctggctcttcttgccgcgcggatggcagatgcgccgcgcgccgtcggggctctcgacgaggcagtgccggccgtggctggccaccaccaggccttcgagggggccggagcgctcagacatgatccggccatgaccacgggctcctgcgcgtagtcgaaggtgagcacgttggtggcgtagtccttgtgccggtactcgcggttcagggcctggccctcttcggcatcgacgatgcgcacggtgatctccgcgtccagcgagagcgcgtggcggatccagcggacgacggcatgccggggcagggcggcgcggtgcgcctccacgccgggaaaccgcgcgaactgcagggacagggaaagctggttcagggccat

SEQ ID NO: 39 - DNA, 758 bp, artificial
  Sequence of the first arm of the P1/ HC-Pro/ cp dsRNA used to concomitantly target P1, HC-Pro, coat protein genes from PIPVgp1 sequene from plume pox virus
tcagacccaggcaaacaattttcggatggtttcaccaattcatttatgcagtgcaagctacgcgaaactgatcatcagtgcacatctgacctggacgtgaaggagtgtggttatgtcgcagcacttgtgtgccaagcgataatcccttgcggaaaaatcacgtgtctgcaatgtgctcaaaagtattcttacatgtcacaacaggaaatacgtgatagattttcgacagtaattgagcagcatgagaaaagatccactttaaatgacttaggattctcagtactcagacaggcaacaccccacttggtagaaaaaagttatctccaggaattggagcaagcttggaaagagttaagctggtcggaaaaattctctgcaatcttggaatcgcagcgatggcgaaaacatataccaaaacctttcatcccaaaagacggcgcagatttaggaggcaggtacgacatctccgttcggtcattacttggcaaccagtacaaacgcctgatgctcagacccaggcaaacaattttcggatggtttcaccaattcatttatgcagtgcaagctacgcgaaactgatcatcagtgcacatctgacctggacgtgaaggagtgtggttatgtcgcagcacttgtgtgccaagcgataatcccttgcggaaaaatcacgtgtctgcaatgtgctcaaaagtattcttacatgtcacaacaggaaatacgtgatagattttcgacagtaattgagcagcatgagaaaa

SEQ ID NO: 40 - DNA, 758 bp, artificial
  Sequence of the second arm of the P1/ HC-Pro/ cp dsRNA used to concomitantly target P1, HC-Pro, coat protein genes from PIPVgp1 sequene from plume pox virus
ttttctcatgctgctcaattactgtcgaaaatctatcacgtatttcctgttgtgacatgtaagaatacttttgagcacattgcagacacgtgatttttccgcaagggattatcgcttggcacacaagtgctgcgacataaccacactccttcacgtccaggtcagatgtgcactgatgatcagtttcgcgtagcttgcactgcataaatgaattggtgaaaccatccgaaaattgtttgcctgggtctgagcatcaggcgtttgtactggttgccaagtaatgaccgaacggagatgtcgtacctgcctcctaaatctgcgccgtcttttgggatgaaaggttttggtatatgttttcgccatcgctgcgattccaagattgcagagaatttttccgaccagcttaactctttccaagcttgctccaattcctggagataacttttttctaccaagtggggtgttgcctgtctgagtactgagaatcctaagtcatttaaagtggatcttttctcatgctgctcaattactgtcgaaaatctatcacgtatttcctgttgtgacatgtaagaatacttttgagcacattgcagacacgtgatttttccgcaagggattatcgcttggcacacaagtgctgcgacataaccacactccttcacgtccaggtcagatgtgcactgatgatcagtttcgcgtagcttgcactgcataaatgaattggtgaaaccatccgaaaattgtttgcctgggtctga

SEQ ID NO: 41 - DNA, 750 bp, artificial
  Sequence of the first arm of the P1 dsRNA used to concomitantly target P1 gene from PIPVgp1 sequene from plume pox virus
tcagacccaggcaaacaattttcggatggtttcaccaattcatttatgcagtgcaagctacgcgaaactgatcatcagtgcacatctgacctggacgtgaaggagtgtggttatgtcgcagcacttgtgtgccaagcgataatcccttgcggaaaaatcacgtgtctgcaatgtgctcaaaagtattcttacatgtcacaacaggaaatacgtgatagattttcgacagtaattgagcagcatgagaaaacagtgatggataactatccacaattttcacatgttctcgcttttctaaagagatatcgtgaactaatgcgcgtggaaaatcagaattatgaagctttcaaggatatcacgcacatgataggcgagcgtaaagaagcacctttttcccatctcaacaaaatcaatgaattaatcattaagggtggtatgatgagcgcacaagactatatagaagcctcggatcatctacgcgaactagcgcgatatcagaagaatcgcacggagaacattaggagcggatctataaaggctttcaggaataaaatctcatcaaaagcacatgtcaatatgcagcttatgtgtgacaatcaacttgatactaatggcaatttcgtgtggggacagagagagtatcatgctaaacgcttctttaggaattacttcgatgtgatcgatgttagcgagggctacagacgtcatattgttcgtgaaaatcctagaggtatccgcaaattggccatt

SEQ ID NO: 42 - DNA, 750 bp, artificial
  Sequence of the second arm of the P1 dsRNA used to concomitantly target P1 gene from PIPVgp1 sequene from plume pox virus
aatggccaatttgcggatacctctaggattttcacgaacaatatgacgtctgtagccctcgctaacatcgatcacatcgaagtaattcctaaagaagcgtttagcatgatactctctctgtccccacacgaaattgccattagtatcaagttgattgtcacacataagctgcatattgacatgtgcttttgatgagattttattcctgaaagcctttatagatccgctcctaatgttctccgtgcgattcttctgatatcgcgctagttcgcgtagatgatccgaggcttctatatagtcttgtgcgctcatcataccacccttaatgattaattcattgattttgttgagatgggaaaaaggtgcttctttacgctcgcctatcatgtgcgtgatatccttgaaagcttcataattctgattttccacgcgcattagttcacgatatctctttagaaaagcgagaacatgtgaaaattgtggatagttatccatcactgttttctcatgctgctcaattactgtcgaaaatctatcacgtatttcctgttgtgacatgtaagaatacttttgagcacattgcagacacgtgatttttccgcaagggattatcgcttggcacacaagtgctgcgacataaccacactccttcacgtccaggtcagatgtgcactgatgatcagtttcgcgtagcttgcactgcataaatgaattggtgaaaccatccgaaaattgtttgcctgggtctga

SEQ ID NO: 43 - DNA, 750 bp, artificial
  Sequence of the first arm of the HC-Pro dsRNA used to concomitantly target HC-Pro gene from PIPVgp1 sequene from plume pox virus
ggtcttgaagtggataagtgtgacgaattcaaaaatgtaaagctcttgatcagaagcatttacaagccacaaatcatggaacaggtgcttaaggaagaaccatatttactgctcatgagcgttttgtcacctggcgtcttgatggcgctgttcaatagtggttcattggagaaagccacacaatattggatcacacgatctcatagcttggcagcgatcacatcaatgttatcagcacttgcagccaaagtttcactcgcaagtacactgaatgcacagatgagtgtcattgacgaacatgcagcagttctatatgatagtgtttttgttggaacgcagccatacgcatcctacatgatggcagtgaaaactttggaaaggatgaaagcacggacggaatccgatcacactttaaatgacttaggattctcagtactcagacaggcaacaccccacttggtagaaaaaagttatctccaggaattggagcaagcttggaaagagttaagctggtcggaaaaattctctgcaatcttggaatcgcagcgatggcgaaaacatataccaaaacctttcatcccaaaagacggcgcagatttaggaggcaggtacgacatctccgttcggtcattacttggcaaccagtacaaacgcctgagagacgtagtccgatggaaaagagacgatgtggtttgctatacataccagtcgatggggaagctattttgcaaagccatcggaatctcacca

SEQ ID NO: 44 - DNA, 750 bp, artificial
  Sequence of the second arm of the HC-Pro dsRNA used to concomitantly target HC-Pro gene from PIPVgp1 sequene from plume pox virus
tggtgagattccgatggctttgcaaaatagcttccccatcgactggtatgtatagcaaaccacatcgtctcttttccatcggactacgtctctcaggcgtttgtactggttgccaagtaatgaccgaacggagatgtcgtacctgcctcctaaatctgcgccgtcttttgggatgaaaggttttggtatatgttttcgccatcgctgcgattccaagattgcagagaatttttccgaccagcttaactctttccaagcttgctccaattcctggagataacttttttctaccaagtggggtgttgcctgtctgagtactgagaatcctaagtcatttaaagtgtgatcggattccgtccgtgctttcatcctttccaaagttttcactgccatcatgtaggatgcgtatggctgcgttccaacaaaaacactatcatatagaactgctgcatgttcgtcaatgacactcatctgtgcattcagtgtacttgcgagtgaaactttggctgcaagtgctgataacattgatgtgatcgctgccaagctatgagatcgtgtgatccaatattgtgtggctttctccaatgaaccactattgaacagcgccatcaagacgccaggtgacaaaacgctcatgagcagtaaatatggttcttccttaagcacctgttccatgatttgtggcttgtaaatgcttctgatcaagagctttacatttttgaattcgtcacacttatccacttcaagacc

SEQ ID NO: 45 - DNA, 250 bp, artificial
  Sequence of the first arm of the CP dsRNA used to concomitantly target coat protein gene from PIPVgp1 sequene from plume pox virus
cactttaaatgacttaggattctcagtactcagacaggcaacaccccacttggtagaaaaaagttatctccaggaattggagcaagcttggaaagagttaagctggtcggaaaaattctctgcaatcttggaatcgcagcgatggcgaaaacatataccaaaacctttcatcccaaaagacggcgcagatttaggaggcaggtacgacatctccgttcggtcattacttggcaaccagtacaaacgcctg

SEQ ID NO: 46 - DNA, 250 bp, artificial
  Sequence of the second arm of the CP dsRNA used to concomitantly target coat protein gene from PIPVgp1 sequene from plume pox virus
caggcgtttgtactggttgccaagtaatgaccgaacggagatgtcgtacctgcctcctaaatctgcgccgtcttttgggatgaaaggttttggtatatgttttcgccatcgctgcgattccaagattgcagagaatttttccgaccagcttaactctttccaagcttgctccaattcctggagataacttttttctaccaagtggggtgttgcctgtctgagtactgagaatcctaagtcatttaaagtg

SEQ ID NO: 47 - DNA, 919 bp, artificial
  Sequence of the first arm of the gyrB/ MreB/ rbfA/ RsgA/ FliA/ QseC dsRNA used to concomitantly target GyrB, MreB, RbfA, RsgA, FliA, QseC genes from erwinia carotovora
atgtcgaattcttatgactcctcaagtatcaaggtattgaaagggctggatgcggtacgtaaacgcccaggtatgtatatcggcgatacggatgacggtaccggcctgcatcacatggtattcgaggttgtggacaacgcttcgacgaagatcatgtttaagaaatttcgtggcatgttttccaacgacttgtccatcgacctgggtaccgccaataccctgatttatgttaaagggcagggcatcatactgaatgaaccctctgtggttgcgattcgtcaggatcgtgctggttcccaaaaaatgcatggcaaaagaattcagccgcacccagcgcgttgcgcaggaaatgcaaaaagaaatcgccattatcattcagcgtgaagtaaaagatccgcgtatcggcatggcgaccgtatccggtgtggaagtttctcgcgatctggcgtatgccaaagtgcgtgagcaaaaagaaactgtcgaaaggtcagcagcgtcgggttagcgcaaaccaccagcgccgcctgaaaaaaacggaaagcaaagtcgaatgggaagatagccaactgggcgacgcacaggaaggcattatcatcagccgattcggtatgaagcgtgagcgatttgtataccgccgaaggcacaatggataaaaattccctttggaagcgctatgttccactagtgcgccatgaagccttgcgtttacaggttcgtctccccgcgagtgttgagcttgacgatctgctacaggctggtggtatctccaatgaaccgactcagcctgcgcctacgcctgattaccggttttacgctgctgaccttgatctgctggggcgtagccagcctgctttcgtggtatcaaacacgccataatatcaatgagctatttgatacccagcaaatgctgttcgccaaa

SEQ ID NO: 48 - DNA, 919 bp, artificial
  Sequence of the second arm of the gyrB/ MreB/ rbfA/ RsgA/ FliA/ QseC dsRNA used to concomitantly target GyrB, MreB, RbfA, RsgA, FliA, QseC genes from erwinia carotovora
tttggcgaacagcatttgctgggtatcaaatagctcattgatattatggcgtgtttgataccacgaaagcaggctggctacgccccagcagatcaaggtcagcagcgtaaaaccggtaatcaggcgtaggcgcaggctgagtcggttcattggagataccaccagcctgtagcagatcgtcaagctcaacactcgcggggagacgaacctgtaaacgcaaggcttcatggcgcactagtggaacatagcgcttccaaagggaatttttatccattgtgccttcggcggtatacaaatcgctcacgcttcataccgaatcggctgatgataatgccttcctgtgcgtcgcccagttggctatcttcccattcgactttgctttccgtttttttcaggcggcgctggtggtttgcgctaacccgacgctgctgacctttcgacagtttctttttgctcacgcactttggcatacgccagatcgcgagaaacttccacaccggatacggtcgccatgccgatacgcggatcttttacttcacgctgaatgataatggcgatttctttttgcatttcctgcgcaacgcgctgggtgcggctgaattcttttgccatgcattttttgggaaccagcacgatcctgacgaatcgcaaccacagagggttcattcagtatgatgccctgccctttaacataaatcagggtattggcggtacccaggtcgatggacaagtcgttggaaaacatgccacgaaatttcttaaacatgatcttcgtcgaagcgttgtccacaacctcgaataccatgtgatgcaggccggtaccgtcatccgtatcgccgatatacatacctgggcgtttacgtaccgcatccagccctttcaataccttgatacttgaggagtcataagaattcgacat

SEQ ID NO: 49 - DNA, 758 bp, artificial
  Sequence of the first arm of the gyrB/ rbfA/ QseC dsRNA used to concomitantly target GyrB, RbfA, QseC genes from erwinia carotovora
atgtcgaattcttatgactcctcaagtatcaaggtattgaaagggctggatgcggtacgtaaacgcccaggtatgtatatcggcgatacggatgacggtaccggcctgcatcacatggtattcgaggttgtggacaacgctatcgacgaagcgctcgctggctattgtaaagacattatcatcaccatccatgccgataactcggtgtcggtgcaggatgatggccgtggtattccgaccggcattcatggatcatggcaaaagaattcagccgcacccagcgcgttgcgcaggaaatgcaaaaagaaatcgccattatcattcagcgtgaagtaaaagatccgcgtatcggcatggcgaccgtatccggtgtggaagtttctcgcgatctggcgtatgccaaagtttttgtgaccttcctgaatgataacgagccagaacaagttaaaacggcgctgaaagcactgcaggatgcttcaggttttattcgtacactggttggtaatgcatgaaccgactcagcctgcgcctacgcctgattaccggttttacgctgctgaccttgatctgctggggcgtagccagcctgctttcgtggtatcaaacacgccataatatcaatgagctatttgatacccagcaaatgctgttcgccaaacgtctggccaccatgaatcctgatgaattgcgaatccagccggtgtccctgcccaaaaccaagagtctggtacataagaaccgcggcaagcaggacgatg

SEQ ID NO: 50 - DNA, 758 bp, artificial
  Sequence of the second arm of the gyrB/ rbfA/ QseC dsRNA used to concomitantly target GyrB, RbfA, QseC genes from erwinia carotovora
catcgtcctgcttgccgcggttcttatgtaccagactcttggttttgggcagggacaccggctggattcgcaattcatcaggattcatggtggccagacgtttggcgaacagcatttgctgggtatcaaatagctcattgatattatggcgtgtttgataccacgaaagcaggctggctacgccccagcagatcaaggtcagcagcgtaaaaccggtaatcaggcgtaggcgcaggctgagtcggttcatgcattaccaaccagtgtacgaataaaacctgaagcatcctgcagtgctttcagcgccgttttaacttgttctggctcgttatcattcaggaaggtcacaaaaactttggcatacgccagatcgcgagaaacttccacaccggatacggtcgccatgccgatacgcggatcttttacttcacgctgaatgataatggcgatttctttttgcatttcctgcgcaacgcgctgggtgcggctgaattcttttgccatgatccatgaatgccggtcggaataccacggccatcatcctgcaccgacaccgagttatcggcatggatggtgatgataatgtctttacaatagccagcgagcgcttcgtcgatagcgttgtccacaacctcgaataccatgtgatgcaggccggtaccgtcatccgtatcgccgatatacatacctgggcgtttacgtaccgcatccagccctttcaataccttgatacttgaggagtcataagaattcgacat

SEQ ID NO: 51 - DNA, 758 bp, artificial
  Sequence of the first arm of the fliA/ MreB/ QseC dsRNA used to concomitantly target FliA, reB, QseC genes from erwinia carotovora
gtgagcgatttgtataccgccgaaggcacaatggataaaaattccctttggaagcgctatgttccactagtgcgccatgaagccttgcgtttacaggttcgtctccccgcgagtgttgagcttgacgatctgctacaggctggtggtatcggtttgctcagtgcagtcgagcgttatgattcgttgcaaggtacggcgtttactacctatgccgttcaacgaatccgcggttcgatgctggatgaattacgatcatgtttaagaaatttcgtggcatgttttccaacgacttgtccatcgacctgggtaccgccaataccctgatttatgttaaagggcagggcatcatactgaatgaaccctctgtggttgcgattcgtcaggatcgtgctggttcccaaaaaagcgtggcagcagtaggccacgacgctaagcagatgctgggcagaacgcctgggaatatcgtggcgattcgcccgatgaaagacggcgttattgccgactatgcatgaaccgactcagcctgcgcctacgcctgattaccggttttacgctgctgaccttgatctgctggggcgtagccagcctgctttcgtggtatcaaacacgccataatatcaatgagctatttgatacccagcaaatgctgttcgccaaacgtctggccaccatgaatcctgatgaattgcgaatccagccggtgtccctgcccaaaaccaagagtctggtacataagaaccgcggcaagcaggacgatg

SEQ ID NO: 52 - DNA, 758 bp, artificial
  Sequence of the second arm of the fliA/ MreB/ QseC dsRNA used to concomitantly target FliA, MreB, QseC genes from erwinia carotovora
catcgtcctgcttgccgcggttcttatgtaccagactcttggttttgggcagggacaccggctggattcgcaattcatcaggattcatggtggccagacgtttggcgaacagcatttgctgggtatcaaatagctcattgatattatggcgtgtttgataccacgaaagcaggctggctacgccccagcagatcaaggtcagcagcgtaaaaccggtaatcaggcgtaggcgcaggctgagtcggttcatgcatagtcggcaataacgccgtctttcatcgggcgaatcgccacgatattcccaggcgttctgcccagcatctgcttagcgtcgtggcctactgctgccacgcttttttgggaaccagcacgatcctgacgaatcgcaaccacagagggttcattcagtatgatgccctgccctttaacataaatcagggtattggcggtacccaggtcgatggacaagtcgttggaaaacatgccacgaaatttcttaaacatgatcgtaattcatccagcatcgaaccgcggattcgttgaacggcataggtagtaaacgccgtaccttgcaacgaatcataacgctcgactgcactgagcaaaccgataccaccagcctgtagcagatcgtcaagctcaacactcgcggggagacgaacctgtaaacgcaaggcttcatggcgcactagtggaacatagcgcttccaaagggaatttttatccattgtgccttcggcggtatacaaatcgctcac

SEQ ID NO: 53 - DNA, 758 bp, artificial
  Sequence of the first arm of the GyrB/ MreB/ RsgA dsRNA used to concomitantly target GyrB, MreB, RsgA genes from erwinia carotovora
atgtcgaattcttatgactcctcaagtatcaaggtattgaaagggctggatgcggtacgtaaacgcccaggtatgtatatcggcgatacggatgacggtaccggcctgcatcacatggtattcgaggttgtggacaacgctatcgacgaagcgctcgctggctattgtaaagacattatcatcaccatccatgccgataactcggtgtcggtgcaggatgatggccgtggtattccgaccggcattcatggatcatgtttaagaaatttcgtggcatgttttccaacgacttgtccatcgacctgggtaccgccaataccctgatttatgttaaagggcagggcatcatactgaatgaaccctctgtggttgcgattcgtcaggatcgtgctggttcccaaaaaagcgtggcagcagtaggccacgacgctaagcagatgctgggcagaacgcctgggaatatcgtggcgattcgcccgatgaaagacggcgttattgccgactatgcgtgagcaaaaagaaactgtcgaaaggtcagcagcgtcgggttagcgcaaaccaccagcgccgcctgaaaaaaacggaaagcaaagtcgaatgggaagatagccaactgggcgacgcacaggaaggcattatcatcagccgattcggtatgcacgccgatgtggaagccaccgatggcgtggtgcaccgctgcaacattcgtcggaccctttcatcgctggttactggcgatcgcgtcgtctggcgcccag

SEQ ID NO: 54 - DNA, 758 bp, artificial
  Sequence of the second arm of the GyrB/ MreB/ RsgA dsRNA used to concomitantly target GyrB, MreB, RsgA genes from erwinia carotovora
ctgggcgccagacgacgcgatcgccagtaaccagcgatgaaagggtccgacgaatgttgcagcggtgcaccacgccatcggtggcttccacatcggcgtgcataccgaatcggctgatgataatgccttcctgtgcgtcgcccagttggctatcttcccattcgactttgctttccgtttttttcaggcggcgctggtggtttgcgctaacccgacgctgctgacctttcgacagtttctttttgctcacgcatagtcggcaataacgccgtctttcatcgggcgaatcgccacgatattcccaggcgttctgcccagcatctgcttagcgtcgtggcctactgctgccacgcttttttgggaaccagcacgatcctgacgaatcgcaaccacagagggttcattcagtatgatgccctgccctttaacataaatcagggtattggcggtacccaggtcgatggacaagtcgttggaaaacatgccacgaaatttcttaaacatgatccatgaatgccggtcggaataccacggccatcatcctgcaccgacaccgagttatcggcatggatggtgatgataatgtctttacaatagccagcgagcgcttcgtcgatagcgttgtccacaacctcgaataccatgtgatgcaggccggtaccgtcatccgtatcgccgatatacatacctgggcgtttacgtaccgcatccagccctttcaataccttgatacttgaggagtcataagaattcgacat

SEQ ID NO: 55 - DNA, 920 bp, artificial
  sequence of the first arm of Wp012778355/Wp015452784/Wp012778510/Wp015452939/act56857/wp012778 668 dsRNA used to concommitantly target the sequences encoding these proteins from candidatus liberibacter asiaticus
gctatcgaccacagtaattgtaaactgactaaaccaattagttataacattacgtgcaagacttttggctataggcatgacattctcgtcagaattaatttcataatatttagatggatcacttgcacattgtcttaaagtatatcgcgtgatcgtgataaaagaattaggtttgtctatgttcataatgacgactatctctggatgtggtttggctagcagagaaaagaaaaaggtatttttgcataaatcgaatgatacagatatcgtcaataaaagatttgggagctctttagataaagcaatgcatgtcaaaaacaaaacacttgaaaaaaacaatatttttatcgaaaaaaggtaatataacaatattaacagcgataatcatcccattgattattaccttaataactatatcgacaacatgtgcaaatattttataccatagggctagcatagtgcatgaccgcaattataatttcggtatgctttttgtttatcacctatgctatcgatttagcgcacattatgtacatacggaatcaaatgcaatctgctttagatgctgctgttctttctggatgtgcatctattgtatcagaccgaacaataaagcatggtaaaaatacgatctcaatacttttgtcaatcctgtggtgaaatccattcgcgttggattggaaaatgtgattcatgtaatgcatggaataccatcatcgaggatagttccagaaaagaaagcgatcaaagatttagcaaaacaatttccaatgaatatttcagaaattaacaaatatttccctcccaataataatatagagaggaaagagatcgctgataaattaatcaaaaatatctcaattgttgacaaaacgatggacgtattgcctctgtatcatcaagttcgcgaattgacacaa

SEQ ID NO: 56 - DNA, 920 bp, artificial
  sequence of the second arm of Wp012778355/Wp015452784/Wp012778510/Wp015452939/act56857/wp012778 668 dsRNA used to concommitantly target the sequences encoding these proteins from candidatus liberibacter asiaticus
ttgtgtcaattcgcgaacttgatgatacagaggcaatacgtccatcgttttgtcaacaattgagatatttttgattaatttatcagcgatctctttcctctctatattattattgggagggaaatatttgttaatttctgaaatattcattggaaattgttttgctaaatctttgatcgctttcttttctggaactatcctcgatgatggtattccatgcattacatgaatcacattttccaatccaacgcgaatggatttcaccacaggattgacaaaagtattgagatcgtatttttaccatgctttattgttcggtctgatacaatagatgcacatccagaaagaacagcagcatctaaagcagattgcatttgattccgtatgtacataatgtgcgctaaatcgatagcataggtgataaacaaaaagcataccgaaattataattgcggtcatgcactatgctagccctatggtataaaatatttgcacatgttgtcgatatagttattaaggtaataatcaatgggatgattatcgctgttaatattgttatattaccttttttcgataaaaatattgtttttttcaagtgttttgtttttgacatgcattgctttatctaaagagctcccaaatcttttattgacgatatctgtatcattcgatttatgcaaaaatacctttttcttttctctgctagccaaaccacatccagagatagtcgtcattatgaacatagacaaacctaattcttttatcacgatcacgcgatatactttaagacaatgtgcaagtgatccatctaaatattatgaaattaattctgacgagaatgtcatgcctatagccaaaagtcttgcacgtaatgttataactaattggtttagtcagtttacaattactgtggtcgatagc

SEQ ID NO: 57 - DNA, 758 bp, artificial
  Sequence of the first arm of the Wp012778355/ wp015452784/WP012778510 dsRNA used to concomitantly target the sequences encoding the proteins Wp012778355/wp015452784/WP012778510 from candidatus liberibacter
gctatcgaccacagtaattgtaaactgactaaaccaattagttataacattacgtgcaagacttttggctataggcatgacattctcgtcagaattaatttcataatatttagatggatcacttgcacattgtcttaaagtatatcgcgtatcttgatccggagaaaatcctacagaaaatatggtaattgcacctcttccagttctatgatgcttatgcatgtaatagcagtcttgcaaaattctatctgatctgataaaagaattaggtttgtctatgttcataatgacgactatctctggatgtggtttggctagcagagaaaagaaaaaggtatttttgcataaatcgaatgatacagatatcgtcaataaaagatttgggagctctttagataaagcagaagatgaatttcagatgcaattacaggatacgggtatagttgttagtcgtataggtgatatgattacatgttatataccagtacatgtttcttttgtatcatgcatgtcaaaaacaaaacacttgaaaaaaacaatatttttatcgaaaaaaggtaatataacaatattaacagcgataatcatcccattgattattaccttaataactatatcgacaacatgtgcaaatattttataccatagggctagcatagaagcatccgctgacgaagccttaaatcatggaatagtcctgctatgtaaggattctgatcttacaccgcaagacatcacccctcccgtattaaaagatc

SEQ ID NO: 58 - DNA, 758 bp, artificial
  Sequence of the second arm of the Wp012778355/ wp015452784/WP012778510 dsRNA used to concomitantly target the sequences encoding the proteins Wp012778355/wp015452784/WP012778510 from candidatus liberibacter
gatcttttaatacgggaggggtgatgtcttgcggtgtaagatcagaatccttacatagcaggactattccatgatttaaggcttcgtcagcggatgcttctatgctagccctatggtataaaatatttgcacatgttgtcgatatagttattaaggtaataatcaatgggatgattatcgctgttaatattgttatattaccttttttcgataaaaatattgtttttttcaagtgttttgtttttgacatgcatgatacaaaagaaacatgtactggtatataacatgtaatcatatcacctatacgactaacaactatacccgtatcctgtaattgcatctgaaattcatcttctgctttatctaaagagctcccaaatcttttattgacgatatctgtatcattcgatttatgcaaaaatacctttttcttttctctgctagccaaaccacatccagagatagtcgtcattatgaacatagacaaacctaattcttttatcagatcagatagaattttgcaagactgctattacatgcataagcatcatagaactggaagaggtgcaattaccatattttctgtaggattttctccggatcaagatacgcgatatactttaagacaatgtgcaagtgatccatctaaatattatgaaattaattctgacgagaatgtcatgcctatagccaaaagtcttgcacgtaatgttataactaattggtttagtcagtttacaattactgtggtcgatagc

SEQ ID NO: 59 - DNA, 758 bp, artificial
  Sequence of the first arm of the wp015452939/act56857/wp012778668 dsRNA used to concomitantly target the sequences encoding the proteins wp015452939/act56857/wp012778668 from candidatus liberibacter asiaticus
atgaccgcaattataatttcggtatgctttttgtttatcacctatgctatcgatttagcgcacattatgtacatacggaatcaaatgcaatctgctttagatgctgctgttctttctggatgtgcatctattgtatcagaccgaacaataaaagatccgacgacaaaaaaggatcaaacaagtactatcttcaaaaaacagatcaaaaagcatctaaaacaaggatcttatataagagaaaacgcaggaggatcatggtaaaaatacgatctcaatacttttgtcaatcctgtggtgaaatccattcgcgttggattggaaaatgtgattcatgtaatgcatggaataccatcatcgaggatagttccagaaaagaaagcgatcaaagatttagcaaaacaattaaacaaggtcactctatctctctttttatgttatcagaagaatcaatagaagaagaatcacgcattcaaacccatattgatgaactcgaccgtgtcacagatgcatgaatatttcagaaattaacaaatatttccctcccaataataatatagagaggaaagagatcgctgataaattaatcaaaaatatctcaattgttgacaaaacgatggacgtattgcctctgtatcatcaagttcgcgaattgacacaaaacaaagcttccactgaacaagtcatcgatatctcaacaaaagtaaaagacgtcgcagtagacgtcgcagtgagcatgattcctatttatggcacttacc

SEQ ID NO: 60 - DNA, 758 bp, artificial
  Sequence of the second arm of the wp015452939/act56857/wp012778668 dsRNA used to concomitantly target the sequences encoding the proteins wp015452939/act56857/wp012778668 from candidatus liberibacter
ggtaagtgccataaataggaatcatgctcactgcgacgtctactgcgacgtcttttacttttgttgagatatcgatgacttgttcagtggaagctttgttttgtgtcaattcgcgaacttgatgatacagaggcaatacgtccatcgttttgtcaacaattgagatatttttgattaatttatcagcgatctctttcctctctatattattattgggagggaaatatttgttaatttctgaaatattcatgcatctgtgacacggtcgagttcatcaatatgggtttgaatgcgtgattcttcttctattgattcttctgataacataaaaagagagatagagtgaccttgtttaattgttttgctaaatctttgatcgctttcttttctggaactatcctcgatgatggtattccatgcattacatgaatcacattttccaatccaacgcgaatggatttcaccacaggattgacaaaagtattgagatcgtatttttaccatgatcctcctgcgttttctcttatataagatccttgttttagatgctttttgatctgttttttgaagatagtacttgtttgatccttttttgtcgtcggatcttttattgttcggtctgatacaatagatgcacatccagaaagaacagcagcatctaaagcagattgcatttgattccgtatgtacataatgtgcgctaaatcgatagcataggtgataaacaaaaagcataccgaaattataattgcggtcat

SEQ ID NO: 61 - DNA, 921 bp, artificial
  Sequence of the first arm of the GyrB/ Hfq/ HrpR/HRPS/MreB/ RpoD dsRNA used to concomitantly target GyrB, Hfq, HrpR, HrpS, MreB, RpoD genes from pseudomonas syringae pv actinidiae
atgagcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgatcatgtcaaaagggcattcgctacaagacccttacttgaacaccttgcgaaaggagaaggtcggcgtctcgatctacctggtcaacggtatcaagctgcagggaacgatcgagtctttcgaccagttcgtcattttgctgaaaaacactgtcatgcatgaatagcgcgttcctggatatggacttgctgttgtgcggggaaaccggcaccggcaaggacacactggccaaccgcattcacgagttgtccagcaggtcgggaccctttgtgggcatgaactgcgcagccattcccgagtcgctggcagtgcatgagtcttgatgaaaggtttgaggatgatctggacgaggagcgggttccgaatctggggatagttgccgaaagtatttcgcaactgggtatcgacgtgctgctgtcgggtgagaccggcacgggcaaagacacgattgcccgacggattaagcatgttcaagaaactgcgtggcatgttttccagtgatctatccatcgacctgggcactgccaacacccttatttacgtgcgtgagcgcggtatcgtcctgaatgaaccctctgtcgttgccattcgtactcacggcaatcagaagagtgtcgtccaatgtccggaaaagcgcaacagcagtctcgtatcaaagagttgatcaccctgggtcgtgagcagaaatatctgacttacgcagaggtcaacgaccacctgcccgaggatatttcagatcccgagcaggtggaagacatcatccgcatgatc

SEQ ID NO: 62 - DNA, 921 bp, artificial
  Sequence of the second arm of the GyrB/ Hfq/ HrpR/HRPS/MreB/ RpoD dsRNA used to concomitantly target GyrB, Hfq, HrpR, HrpS, MreB, RpoD genes from pseudomonas syringae pv actinidiae
gatcatgcggatgatgtcttccacctgctcgggatctgaaatatcctcgggcaggtggtcgttgacctctgcgtaagtcagatatttctgctcacgacccagggtgatcaactctttgatacgagactgctgttgcgcttttccggacattggacgacactcttctgattgccgtgagtacgaatggcaacgacagagggttcattcaggacgataccgcgctcacgcacgtaaataagggtgttggcagtgcccaggtcgatggatagatcactggaaaacatgccacgcagtttcttgaacatgcttaatccgtcgggcaatcgtgtctttgcccgtgccggtctcacccgacagcagcacgtcgatacccagttgcgaaatactttcggcaactatccccagattcggaacccgctcctcgtccagatcatcctcaaacctttcatcaagactcatgcactgccagcgactcgggaatggctgcgcagttcatgcccacaaagggtcccgacctgctggacaactcgtgaatgcggttggccagtgtgtccttgccggtgccggtttccccgcacaacagcaagtccatatccaggaacgcgctattcatgcatgacagtgtttttcagcaaaatgacgaactggtcgaaagactcgatcgttccctgcagcttgataccgttgaccaggtagatcgagacgccgaccttctcctttcgcaaggtgttcaagtaagggtcttgtagcgaatgcccttttgacatgatcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgctcat

SEQ ID NO: 63 - DNA, 758 bp, artificial
  Sequence of the first arm of the GyrB/ Hfq/ MreB dsRNA used to concomitantly target GyrB, Hfq, MreB genes from pseudomonas syringae pv actinidiae
atgagcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcgacaacggccgcggtattccggtagacggatcatgtcaaaagggcattcgctacaagacccttacttgaacaccttgcgaaaggagaaggtcggcgtctcgatctacctggtcaacggtatcaagctgcagggaacgatcgagtctttcgaccagttcgtcattttgctgaaaaacactgtcagccagatggtctacaaacacgccatttccaccgtcgtaccggttcgcccgattcgcctgcctagcgctaccgacgctgacggcgctgacgccgagccagatgcatgttcaagaaactgcgtggcatgttttccagtgatctatccatcgacctgggcactgccaacacccttatttacgtgcgtgagcgcggtatcgtcctgaatgaaccctctgtcgttgccattcgtactcacggcaatcagaagagtgtcgtcgctgtcggtaccgaagccaagcgtatgctgggccgcactccaggcaacattgccgccattcgcccaatgaaagacggcgtcatcgccgatttcagcg

SEQ ID NO: 64 - DNA, 758 bp, artificial
  Sequence of the second arm of the GyrB/ Hfq/ MreB dsRNA used to concomitantly target GyrB, Hfq, MreB genes from pseudomonas syringae pv actinidiae
cgctgaaatcggcgatgacgccgtctttcattgggcgaatggcggcaatgttgcctggagtgcggcccagcatacgcttggcttcggtaccgacagcgacgacactcttctgattgccgtgagtacgaatggcaacgacagagggttcattcaggacgataccgcgctcacgcacgtaaataagggtgttggcagtgcccaggtcgatggatagatcactggaaaacatgccacgcagtttcttgaacatgcatctggctcggcgtcagcgccgtcagcgtcggtagcgctaggcaggcgaatcgggcgaaccggtacgacggtggaaatggcgtgtttgtagaccatctggctgacagtgtttttcagcaaaatgacgaactggtcgaaagactcgatcgttccctgcagcttgataccgttgaccaggtagatcgagacgccgaccttctcctttcgcaaggtgttcaagtaagggtcttgtagcgaatgcccttttgacatgatccgtctaccggaataccgcggccgttgtcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgctcat

SEQ ID NO: 65 - DNA, 758 bp, artificial
  Sequence of the first arm of the HrpR/ HrpS/Hfq dsRNA used to concomitantly target HrpR, HrpS, Hfq genes from pseudomonas syringae pv actinidiae
atgaatagcgcgttcctggatatggacttgctgttgtgcggggaaaccggcaccggcaaggacacactggccaaccgcattcacgagttgtccagcaggtcgggaccctttgtgggcatgaactgcgcagccattcccgagtcgctggcagagagccagttattcggtgtggtcaacggcgcattcaccggcgtatgccgggctcgcgagggctacatagaggcctccagtggtggcacgttgtacctgggatcatgagtcttgatgaaaggtttgaggatgatctggacgaggagcgggttccgaatctggggatagttgccgaaagtatttcgcaactgggtatcgacgtgctgctgtcgggtgagaccggcacgggcaaagacacgattgcccgacggattcatgagatgtcaggccgcaaagggcgcctggtggcgatgaattgcgcggccattccggagtccctcgccgagagcgagttattcggtgtggtcagcggtgatgcatgtcaaaagggcattcgctacaagacccttacttgaacaccttgcgaaaggagaaggtcggcgtctcgatctacctggtcaacggtatcaagctgcagggaacgatcgagtctttcgaccagttcgtcattttgctgaaaaacactgtcagccagatggtctacaaacacgccatttccaccgtcgtaccggttcgcccgattcgcctgcctagcgctaccgacgctgacggcgctgacgccgagccag

SEQ ID NO: 66 - DNA, 758 bp, artificial
  Sequence of the second arm of the HrpR/ HrpS/Hfq dsRNA used to concomitantly target HrpR, HrpS, Hfq genes from pseudomonas syringae pv actinidiae
ctggctcggcgtcagcgccgtcagcgtcggtagcgctaggcaggcgaatcgggcgaaccggtacgacggtggaaatggcgtgtttgtagaccatctggctgacagtgtttttcagcaaaatgacgaactggtcgaaagactcgatcgttccctgcagcttgataccgttgaccaggtagatcgagacgccgaccttctcctttcgcaaggtgttcaagtaagggtcttgtagcgaatgcccttttgacatgcatcaccgctgaccacaccgaataactcgctctcggcgagggactccggaatggccgcgcaattcatcgccaccaggcgccctttgcggcctgacatctcatgaatccgtcgggcaatcgtgtctttgcccgtgccggtctcacccgacagcagcacgtcgatacccagttgcgaaatactttcggcaactatccccagattcggaacccgctcctcgtccagatcatcctcaaacctttcatcaagactcatgatcccaggtacaacgtgccaccactggaggcctctatgtagccctcgcgagcccggcatacgccggtgaatgcgccgttgaccacaccgaataactggctctctgccagcgactcgggaatggctgcgcagttcatgcccacaaagggtcccgacctgctggacaactcgtgaatgcggttggccagtgtgtccttgccggtgccggtttccccgcacaacagcaagtccatatccaggaacgcgctattcat

SEQ ID NO: 67 - DNA, 758 bp, artificial
  Sequence of the first arm of the HrpR/ GyrB/ RpoD dsRNA used to concomitantly target HrpR, GyrB, RpoD genes from pseudomonas syringae pv actinidiae
atgaatagcgcgttcctggatatggacttgctgttgtgcggggaaaccggcaccggcaaggacacactggccaaccgcattcacgagttgtccagcaggtcgggaccctttgtgggcatgaactgcgcagccattcccgagtcgctggcagagagccagttattcggtgtggtcaacggcgcattcaccggcgtatgccgggctcgcgagggctacatagaggcctccagtggtggcacgttgtacctgggatcatgagcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcgacaacggccgcggtattccggtagacgatgcatgtccggaaaagcgcaacagcagtctcgtatcaaagagttgatcaccctgggtcgtgagcagaaatatctgacttacgcagaggtcaacgaccacctgcccgaggatatttcagatcccgagcaggtggaagacatcatccgcatgatcaacgacatggggatcccggtacacgagagtgctccggatgcggacgccttgatgctggccgacgccgataccgacgaggcagccgcagaagaagcggctg

SEQ ID NO: 68 - DNA, 932 bp, artificial
  Sequence of the first arm of the NPP1/INF1/GK4/piacwp1-1/piacwp1-2/piacwp1-3 dsRNA used to concomitantly target NPP1, INF1, GK4, piacwp1-1, piacwp 1-2 and piacwp1-3 genes from phytophtora infestans
cgcctccaagtcctcgagactctccaccgacatgaacatccttcaactcttcgcatcggccgcagcagctcttactgtcgcgcatgccgatgtgatttcacacgatgcagtgatcccgttcgcccaacccacaaccaccactacaacagagcaggatcgtttgctgacccttccagtcctagctccacaacttccattgtgcaatttgctctgttatccccacccactcctcctcactccgtccacgatgaactttcgtgctctgttcgctgccaccgttgctgccctcgtcggctccacttccgccaatgcatggcgttgtgcatccctgggtcgtcgcaaaacgcgtcggcagccgcgagtgtagccagcacgagcgacagcaccaatccgttcggccgacggctactaaccagtgtcgccagctccgcgagcgcaggtgacgatggctacgtgcagctcgaggtgcaggagagcgaggaattgacgaatgaacttggcgctttctttttgcaaatcccggagtatagcgagctgacacacccagcctgcctgtcggaattgtcccctggcccaccttttcacggaggagacagcggcgctgtctccgaccgcagactaagcctggactctgcaagcactcagatggagattggcagtggcaggagagcgaggaattgaagaatgaacttggcgctttctctttgcaaatcccggagtatagcgagctgacacacccagcctgcctgtcggaattgtcccctggcccaccttttctccagcactcagatggagattggcagtggcaggagagcgaggaattgacgaatgaacttggcgctttctttttgcaaatcccggagtatagcgagctgacacacccagcctgcctgtcggaattgtcccctggcccaccttttcacggaggagac

SEQ ID NO: 69 - DNA, 932 bp, artificial
  Sequence of the second arm of the NPP1/INF1/GK4/piacwp1-1/piacwp1-2/piacwp1-3 dsRNA used to concomitantly target NPP1, INF1, GK4, piacwp1-1, piacwp 1-2 and piacwp1-3 genes from Phytophthora infestans
gtctcctccgtgaaaaggtgggccaggggacaattccgacaggcaggctgggtgtgtcagctcgctatactccgggatttgcaaaaagaaagcgccaagttcattcgtcaattcctcgctctcctgccactgccaatctccatctgagtgctggagaaaaggtgggccaggggacaattccgacaggcaggctgggtgtgtcagctcgctatactccgggatttgcaaagagaaagcgccaagttcattcttcaattcctcgctctcctgccactgccaatctccatctgagtgcttgcagagtccaggcttagtctgcggtcggagacagcgccgctgtctcctccgtgaaaaggtgggccaggggacaattccgacaggcaggctgggtgtgtcagctcgctatactccgggatttgcaaaaagaaagcgccaagttcattcgtcaattcctcgctctcctgcacctcgagctgcacgtagccatcgtcacctgcgctcgcggagctggcgacactggttagtagccgtcggccgaacggattggtgctgtcgctcgtgctggctacactcgcggctgccgacgcgttttgcgacgacccagggatgcacaacgccatgcattggcggaagtggagccgacgagggcagcaacggtggcagcgaacagagcacgaaagttcatcgtggacggagtgaggaggagtgggtggggataacagagcaaattgcacaatggaagttgtggagctaggactggaagggtcagcaaacgatcctgctctgttgtagtggtggttgtgggttgggcgaacgggatcactgcatcgtgtgaaatcacatcggcatgcgcgacagtaagagctgctgcggccgatgcgaagagttgaaggatgttcatgtcggtggagagtctcgaggacttggaggcg

SEQ ID NO: 70 - DNA, 790 bp, artificial
  Sequence of the first arm of the piacwp1-1/piacwp1-2/piacwp1-3 dsRNA used to concomitantly target piacwp1-1, piacwp 1-2 and piacwp1-3 genes from Phytophthora infestans
aggagagcgaggaattgacgaatgaacttggcgctttctttttgcaaatcccggagtatagcgagctgacacacccagcctgcctgtcggaattgtcccctggcccaccttttcacggaggagacagcggcgctgtctccgcaccgcagactctggcccagagagcttactggccattttacaattcgcacgctgagactcaccctgacatttctcgttctttgcaccatggtcttctcctctactacctgtattctgatcctggactctgcaagcactcagatggagattggcagtggcaggagagcgaggaattgaagaatgaacttggcgctttctctttgcaaatcccggagtatagcgagctgacacacccagcctgcctgtcggaattgtcccctggcccaccttttcacggaggagacagcggcgctgtctccgcaccgcagactctggcccagagagcttactggccattttacaattcgcacgctgagactcaccctgacatttctcgttctttgcaccatgccactcagatggagattggcagtggcaggagagcgaggaattgacgaatgaacttggcgctttctttttgcaaatcccggagtatagcgagctgacacacccagcctgcctgtcggaattgtcccctggcccaccttttcacggaggagacagcggcgctgtctccgcaccgcagactctggcccagagagcttactggccattttacaattcgcacgctgagactcaccctgacatttctcgttctttgcaccatggt

SEQ ID NO: 71 - DNA, 790 bp, artificial
  Sequence of the second arm of the piacwp1-1/piacwp1-2/piacwp1-3 dsRNA used to concomitantly target piacwp1-1, piacwp 1-2 and piacwp1-3 genes from Phytophthora infestans
accatggtgcaaagaacgagaaatgtcagggtgagtctcagcgtgcgaattgtaaaatggccagtaagctctctgggccagagtctgcggtgcggagacagcgccgctgtctcctccgtgaaaaggtgggccaggggacaattccgacaggcaggctgggtgtgtcagctcgctatactccgggatttgcaaaaagaaagcgccaagttcattcgtcaattcctcgctctcctgccactgccaatctccatctgagtggcatggtgcaaagaacgagaaatgtcagggtgagtctcagcgtgcgaattgtaaaatggccagtaagctctctgggccagagtctgcggtgcggagacagcgccgctgtctcctccgtgaaaaggtgggccaggggacaattccgacaggcaggctgggtgtgtcagctcgctatactccgggatttgcaaagagaaagcgccaagttcattcttcaattcctcgctctcctgccactgccaatctccatctgagtgcttgcagagtccaggatcagaatacaggtagtagaggagaagaccatggtgcaaagaacgagaaatgtcagggtgagtctcagcgtgcgaattgtaaaatggccagtaagctctctgggccagagtctgcggtgcggagacagcgccgctgtctcctccgtgaaaaggtgggccaggggacaattccgacaggcaggctgggtgtgtcagctcgctatactccgggatttgcaaaaagaaagcgccaagttcattcgtcaattcctcgctctcct

SEQ ID NO: 72 - DNA, 781 bp, artificial
  Sequence of the first arm of the GK4/ INF1/NPP1 dsRNA used to concomitantly target GK4, INF1, NPP1 genes from Phytophtora infestans
atggcgttgtgcatccctgggtcgtcgcaaaacgcgtcggcagccgcgagtgtagccagcacgagcgacagcaccaatccgttcggccgacggctactaaccagtgtcgccagctccgcgagcgcaggtgacgatggctacgtgcagctcgagaccgagttccctggcgccatagctatctccatggtcgtgaacgcactgtccctgcttggcttcgctttcattctcaccacgtacctgttgttcccacccgtgcgaaagcgatcgtttgctgacccttccagtcctagctccacaacttccattgtgcaatttgctctgttatccccacccactcctcctcactccgtccacgatgaactttcgtgctctgttcgctgccaccgttgctgccctcgtcggctccacttccgccaccacgtgcaccacctcgcagcagaccgtagcgtacgtggcgctcgtaagcatcctctcggacacgtcgtttaatcagtgctcgacggactccggctactcgaatgccgcctccaagtcctcgagactctccaccgacatgaacatccttcaactcttcgcatcggccgcagcagctcttactgtcgcgcatgccgatgtgatttcacacgatgcagtgatcccgttcgcccaacccacaaccaccactacaacagagcagaaggctggtatcaagttcaagccccagatccacatctcgaacggctgccacccgtaccctgcagtggatgcagacggcaacaccagcggcggtctaaaccctacg

SEQ ID NO: 73 - DNA, 781 bp, artificial
  Sequence of the second arm of the GK4/ INF1/NPP1 dsRNA used to concomitantly target GK4, INF1, NPP1 genes from Phytophthora infestans
cgtagggtttagaccgccgctggtgttgccgtctgcatccactgcagggtacgggtggcagccgttcgagatgtggatctggggcttgaacttgataccagccttctgctctgttgtagtggtggttgtgggttgggcgaacgggatcactgcatcgtgtgaaatcacatcggcatgcgcgacagtaagagctgctgcggccgatgcgaagagttgaaggatgttcatgtcggtggagagtctcgaggacttggaggcggcattcgagtagccggagtccgtcgagcactgattaaacgacgtgtccgagaggatgcttacgagcgccacgtacgctacggtctgctgcgaggtggtgcacgtggtggcggaagtggagccgacgagggcagcaacggtggcagcgaacagagcacgaaagttcatcgtggacggagtgaggaggagtgggtggggataacagagcaaattgcacaatggaagttgtggagctaggactggaagggtcagcaaacgatcgctttcgcacgggtgggaacaacaggtacgtggtgagaatgaaagcgaagccaagcagggacagtgcgttcacgaccatggagatagctatggcgccagggaactcggtctcgagctgcacgtagccatcgtcacctgcgctcgcggagctggcgacactggttagtagccgtcggccgaacggattggtgctgtcgctcgtgctggctacactcgcggctgccgacgcgttttgcgacgacccagggatgcacaacgccat

SEQ ID NO: 74 - DNA, 802 bp, artificial
  Sequence of the first arm of the GK4/ INF1/piacwp1.1 dsRNA used to concomitantly target GK4, INF1, piacwp1.1 genes from Phytophthora infestans
atggcgttgtgcatccctgggtcgtcgcaaaacgcgtcggcagccgcgagtgtagccagcacgagcgacagcaccaatccgttcggccgacggctactaaccagtgtcgccagctccgcgagcgcaggtgacgatggctacgtgcagctcgagaccgagttccctggcgccatagctatctccatggtcgtgaacgcactgtccctgcttggcttcgctttcattctcaccacgtacctgttgttcccacccgtgcgaaagcgatcgtttgctgacccttccagtcctagctccacaacttccattgtgcaatttgctctgttatccccacccactcctcctcactccgtccacgatgaactttcgtgctctgttcgctgccaccgttgctgccctcgtcggctccacttccgccaccacgtgcaccacctcgcagcagaccgtagcgtacgtggcgctcgtaagcatcctctcggacacgtcgtttaatcagtgctcgacggactccggctactcgaatgcaggagagcgaggaattgacgaatgaacttggcgctttctttttgcaaatcccggagtatagcgagctgacacacccagcctgcctgtcggaattgtcccctggcccaccttttcacggaggagacagcggcgctgtctccgcaccgcagactctggcccagagagcttactggccattttacaattcgcacgctgagactcaccctgacatttctcgttctttgcaccatggtcttctcctctactacctgtattctgggtgtggcggcgctcgcgtcgg

SEQ ID NO: 75 - DNA, 802 bp, artificial
  Sequence of the second arm of the GK4/ INF1/piacwp1.1 dsRNA used to concomitantly target GK4, INF1, piacwp1.1 genes from Phytophthora infestans
ccgacgcgagcgccgccacacccagaatacaggtagtagaggagaagaccatggtgcaaagaacgagaaatgtcagggtgagtctcagcgtgcgaattgtaaaatggccagtaagctctctgggccagagtctgcggtgcggagacagcgccgctgtctcctccgtgaaaaggtgggccaggggacaattccgacaggcaggctgggtgtgtcagctcgctatactccgggatttgcaaaaagaaagcgccaagttcattcgtcaattcctcgctctcctgcattcgagtagccggagtccgtcgagcactgattaaacgacgtgtccgagaggatgcttacgagcgccacgtacgctacggtctgctgcgaggtggtgcacgtggtggcggaagtggagccgacgagggcagcaacggtggcagcgaacagagcacgaaagttcatcgtggacggagtgaggaggagtgggtggggataacagagcaaattgcacaatggaagttgtggagctaggactggaagggtcagcaaacgatcgctttcgcacgggtgggaacaacaggtacgtggtgagaatgaaagcgaagccaagcagggacagtgcgttcacgaccatggagatagctatggcgccagggaactcggtctcgagctgcacgtagccatcgtcacctgcgctcgcggagctggcgacactggttagtagccgtcggccgaacggattggtgctgtcgctcgtgctggctacactcgcggctgccgacgcgttttgcgacgacccagggatgcacaacgccat

SEQ ID NO: 76 - DNA, 1079 bp, artificial
  Sequence of the first arm of the TOR/ CGF1/ DCL1/DCL2/ LTF1/HBF1 dsRNA used to concomitantly target TOR/ CGF1/ DCL1/DCL2/ LTF1/HBF1  genes from Botrytis cinerea
atgtcacaacaatctaatcaagcatttgatgctttgaatcgcattgtggcagatttaagatcacggagtgatgaacagcgaaagagagcggccctggagttaagagagttgcttgaggttgcttctagagactttccccaggaacgatacacggaattttccaatattgtaagtggaaagattagatcatgcatactccatccatcaatatcttgctcgctgtgggccttctcttctctaccccattcacatcagctcttactattccttcgattgacaagaagtctatcgctggcacaggcatagaatccattgagactcgtgatattcctgctctcatcaatcgtcaccacaccgatgcgagtttacatgttttatatatcttcaagctttctacattgggaaagatcttgggcatatttcaaccgcccgcaggagcacaagcgaatgacgtctcgcagatgacgagagacgcagcagcagcaaaaagtctctaccattggcgttgggtttctcctatcggatgaaacgctgggggtgtgcgactgcctgtaccgacattctcagcatttcttatcgaaattgagaatgtcgtacaaaggtaaaaaaaaaaaaatgacaaatgaaagtccataaattcccaaaatggacaactattcgcggaaacaaaagaccccggtcgcaaaaacaaacgcctcatcttaaccacgactctctatctctcaaacaagcatggagggtgcggaaagtggcagtaggtcaagtggcatcctctccatcatgaaccgtgaacatatcgagcgcataaataccgacacgagatatatgcaatcaaatccaatggctgcaccaccgactgccagtcctgcgtccgctcactactcaaacggtgcaccatactccgcaggatggccctccagagcgcacaacgtaacaatggaaacttttcgaatttttctgaaattctcgtcgatttcgacaaagaacgtcctaagctccttcttccggtatcattcaaagaagattttatgaaaaaaaaaacaaagtttgtaatgttctacgctcacgatcaacctttg

SEQ ID NO: 77 - DNA, 1079 bp, artificial
  Sequence of the second arm of the TOR/ CGF1/ DCL1/DCL2/ LTF1/HBF1 dsRNA used to concomitantly target TOR, CGF1, DCL1, DCL2, LTF1, HBF1 genes from Botrytis cinerea
caaaggttgatcgtgagcgtagaacattacaaactttgttttttttttcataaaatcttctttgaatgataccggaagaaggagcttaggacgttctttgtcgaaatcgacgagaatttcagaaaaattcgaaaagtttccattgttacgttgtgcgctctggagggccatcctgcggagtatggtgcaccgtttgagtagtgagcggacgcaggactggcagtcggtggtgcagccattggatttgattgcatatatctcgtgtcggtatttatgcgctcgatatgttcacggttcatgatggagaggatgccacttgacctactgccactttccgcaccctccatgcttgtttgagagatagagagtcgtggttaagatgaggcgtttgtttttgcgaccggggtcttttgtttccgcgaatagttgtccattttgggaatttatggactttcatttgtcatttttttttttttacctttgtacgacattctcaatttcgataagaaatgctgagaatgtcggtacaggcagtcgcacacccccagcgtttcatccgataggagaaacccaacgccaatggtagagactttttgctgctgctgcgtctctcgtcatctgcgagacgtcattcgcttgtgctcctgcgggcggttgaaatatgcccaagatctttcccaatgtagaaagcttgaagatatataaaacatgtaaactcgcatcggtgtggtgacgattgatgagagcaggaatatcacgagtctcaatggattctatgcctgtgccagcgatagacttcttgtcaatcgaaggaatagtaagagctgatgtgaatggggtagagaagagaaggcccacagcgagcaagatattgatggatggagtatgcatgatctaatctttccacttacaatattggaaaattccgtgtatcgttcctggggaaagtctctagaagcaacctcaagcaactctcttaactccagggccgctctctttcgctgttcatcactccgtgatcttaaatctgccacaatgcgattcaaagcatcaaatgcttgattagattgttgtgacat

SEQ ID NO: 78 - DNA, 797 bp, artificial
  Sequence of the first arm of the TOR/ DCL1/DCL2 dsRNA used to concomitantly target TOR, DCL1, DCL2 genes from Botrytis cinerea
atgtcacaacaatctaatcaagcatttgatgctttgaatcgcattgtggcagatttaagatcacggagtgatgaacagcgaaagagagcggccctggagttaagagagttgcttgaggttgcttctagagactttccccaggaacgatacacggaattttccaatattgtaagtggaaagattaccagcctcatcggtcaccatggagatgttgagcgaactggtggtatatatgctatcgatgcgctggtcgattttgacggtgatcgagtttacatgttttatatatcttcaagctttctacattgggaaagatcttgggcatatttcaaccgcccgcaggagcacaagcgaatgacgtctcgcagatgacgagagacgcagcagcagcaaaaagtctctaccattggcgttgggtttctcctatcggatgaaacgctgggggtgaggggacgttcacttcttctgaataacacaaccagataacaataacaagaattcaatttctcccgcctctcacctcccaatgcgactgcctgtaccgacattctcagcatttcttatcgaaattgagaatgtcgtacaaaggtaaaaaaaaaaaaatgacaaatgaaagtccataaattcccaaaatggacaactattcgcggaaacaaaagaccccggtcgcaaaaacaaacgcctcatcttaaccacgactctctatctctcaaacccacatcgctcttccacaattcctcattcccagttagggtttttagtctaacgaatcattttcgctctccggcttctttcga

SEQ ID NO: 79 - DNA, 797 bp, artificial
  Sequence of the second arm of the TOR/ DCL1/DCL2 dsRNA used to concomitantly target TOR, DCL1, DCL2 genes from Botrytis cinerea
tcgaaagaagccggagagcgaaaatgattcgttagactaaaaaccctaactgggaatgaggaattgtggaagagcgatgtgggtttgagagatagagagtcgtggttaagatgaggcgtttgtttttgcgaccggggtcttttgtttccgcgaatagttgtccattttgggaatttatggactttcatttgtcatttttttttttttacctttgtacgacattctcaatttcgataagaaatgctgagaatgtcggtacaggcagtcgcattgggaggtgagaggcgggagaaattgaattcttgttattgttatctggttgtgttattcagaagaagtgaacgtcccctcacccccagcgtttcatccgataggagaaacccaacgccaatggtagagactttttgctgctgctgcgtctctcgtcatctgcgagacgtcattcgcttgtgctcctgcgggcggttgaaatatgcccaagatctttcccaatgtagaaagcttgaagatatataaaacatgtaaactcgatcaccgtcaaaatcgaccagcgcatcgatagcatatataccaccagttcgctcaacatctccatggtgaccgatgaggctggtaatctttccacttacaatattggaaaattccgtgtatcgttcctggggaaagtctctagaagcaacctcaagcaactctcttaactccagggccgctctctttcgctgttcatcactccgtgatcttaaatctgccacaatgcgattcaaagcatcaaatgcttgattagattgttgtgacat

SEQ ID NO: 80 - DNA, 811 bp, artificial
  Sequence of the first arm of the CGF1/HBF1/LTF1 dsRNA used to concomitantly target CGF1, HBF1, LTF1 genes from Botrytis cinerea
atgcatactccatccatcaatatcttgctcgctgtgggccttctcttctctaccccattcacatcagctcttactattccttcgattgacaagaagtctatcgctggcacaggcatagaatccattgagactcgtgatattcctgctctcatcaatcgtcaccacaccgaggcccagatcgccgctaaggcgggaaaggcaaaaggaaaaggacagcgcgatgttttagagagagagccgcatcacactgaagcacagattgcagcaaagaaagcaaaaggatcgagcgcacaacgtaacaatggaaacttttcgaatttttctgaaattctcgtcgatttcgacaaagaacgtcctaagctccttcttccggtatcattcaaagaagattttatgaaaaaaaaaacaaagtttgtaatgttctacgctcacgatcaacctttgacttcaacggattgccatcacatacacaaaattctcctggcacattatcaacgattcattcaagtctaccgtcaactcgtagacagctaaaagctgtaaaatgcatggagggtgcggaaagtggcagtaggtcaagtggcatcctctccatcatgaaccgtgaacatatcgagcgcataaataccgacacgagatatatgcaatcaaatccaatggctgcaccaccgactgccagtcctgcgtccgctcactactcaaacggtgcaccatactccgcaggatggcccagtacttctcatggcggtttaatatctcctccagagtcgcggaggacgtccaacgacaaagcgcacccgcatccattgca

SEQ ID NO: 81 - DNA, 811 bp, artificial
  Sequence of the second arm of the CGF1/HBF1/LTF1 dsRNA used to concomitantly target CGF1, HBF1, LTF1 genes from Botrytis cinerea
tgcaatggatgcgggtgcgctttgtcgttggacgtcctccgcgactctggaggagatattaaaccgccatgagaagtactgggccatcctgcggagtatggtgcaccgtttgagtagtgagcggacgcaggactggcagtcggtggtgcagccattggatttgattgcatatatctcgtgtcggtatttatgcgctcgatatgttcacggttcatgatggagaggatgccacttgacctactgccactttccgcaccctccatgcattttacagcttttagctgtctacgagttgacggtagacttgaatgaatcgttgataatgtgccaggagaattttgtgtatgtgatggcaatccgttgaagtcaaaggttgatcgtgagcgtagaacattacaaactttgttttttttttcataaaatcttctttgaatgataccggaagaaggagcttaggacgttctttgtcgaaatcgacgagaatttcagaaaaattcgaaaagtttccattgttacgttgtgcgctcgatccttttgctttctttgctgcaatctgtgcttcagtgtgatgcggctctctctctaaaacatcgcgctgtccttttccttttgcctttcccgccttagcggcgatctgggcctcggtgtggtgacgattgatgagagcaggaatatcacgagtctcaatggattctatgcctgtgccagcgatagacttcttgtcaatcgaaggaatagtaagagctgatgtgaatggggtagagaagagaaggcccacagcgagcaagatattgatggatggagtatgcat

SEQ ID NO: 82 - DNA, 771 bp, artificial
  Sequence of the first arm of the DCL1/ HBF1/TOR dsRNA used to concomitantly target DCL1, HBF1, TOR genes from Botrytis cinerea
gagtttacatgttttatatatcttcaagctttctacattgggaaagatcttgggcatatttcaaccgcccgcaggagcacaagcgaatgacgtctcgcagatgacgagagacgcagcagcagcaaaaagtctctaccattggcgttgggtttctcctatcggatgaaacgctgggggtgaggggacgttcacttcttctgaataacacaaccagataacaataacaagaattcaatttctcccgcctctcacctcccaacagatcgagcgcacaacgtaacaatggaaacttttcgaatttttctgaaattctcgtcgatttcgacaaagaacgtcctaagctccttcttccggtatcattcaaagaagattttatgaaaaaaaaaacaaagtttgtaatgttctacgctcacgatcaacctttgacttcaacggattgccatcacatacacaaaattctcctggcacattatcaacgattcattcaagtctaccgtcaactcgtatgcatgtcacaacaatctaatcaagcatttgatgctttgaatcgcattgtggcagatttaagatcacggagtgatgaacagcgaaagagagcggccctggagttaagagagttgcttgaggttgcttctagagactttccccaggaacgatacacggaattttccaatattgtaagtggaaagattaccagcctcatcggtcaccatggagatgttgagcgaactggtggtatatatgctatcgatgcgctggtcgattttgacg

SEQ ID NO: 83 - DNA, 771 bp, artificial
  Sequence of the second arm of the DCL1/ HBF1/TOR dsRNA used to concomitantly target DCL1, HBF1, TOR genes from Botrytis cinerea
cgtcaaaatcgaccagcgcatcgatagcatatataccaccagttcgctcaacatctccatggtgaccgatgaggctggtaatctttccacttacaatattggaaaattccgtgtatcgttcctggggaaagtctctagaagcaacctcaagcaactctcttaactccagggccgctctctttcgctgttcatcactccgtgatcttaaatctgccacaatgcgattcaaagcatcaaatgcttgattagattgttgtgacatgcatacgagttgacggtagacttgaatgaatcgttgataatgtgccaggagaattttgtgtatgtgatggcaatccgttgaagtcaaaggttgatcgtgagcgtagaacattacaaactttgttttttttttcataaaatcttctttgaatgataccggaagaaggagcttaggacgttctttgtcgaaatcgacgagaatttcagaaaaattcgaaaagtttccattgttacgttgtgcgctcgatctgttgggaggtgagaggcgggagaaattgaattcttgttattgttatctggttgtgttattcagaagaagtgaacgtcccctcacccccagcgtttcatccgataggagaaacccaacgccaatggtagagactttttgctgctgctgcgtctctcgtcatctgcgagacgtcattcgcttgtgctcctgcgggcggttgaaatatgcccaagatctttcccaatgtagaaagcttgaagatatataaaacatgtaaactc

SEQ ID NO: 84 - DNA, 947 bp, artificial
  Sequence of the first arm of the dsRNA used to concomitantly target CclA/ ACS1/ ACS2/ ELP1/ ELP2/ MOB2 genes from Colletotrichum higginsianum
atggcgtccgactcgggtactccccctcctccttgggagccgactctgagcggttcccttcctgcctcaaccgcgatcccgttgaagcgcgccgccctcgaggaggattttactccctctgtaccatctcctctcaatccagacgtccgatcaacaccgatctgaccaaaagccgtgattggtggtggcacgaggaggtcgagaagtggcctgcctacttcccccccgagcgtgtcaactccgaggaccccttgttcctcctctacacctccggctctaccggtaagcccaaaggtgtcatgcacaccaccggagatgcatgggcgacttacaccctcaagacgaggtcctgcgccgttccctcgaggaccccgaggggttctgggcccaccaggccgagcatctgcactggcacaagaaaccctcgtccgcattgaaagtctccaaaaagacgctcaagggcggcatcagtgcatgcagatctccgaactcttctccatcctggctgtcgtcggcgttgccgccgccctccctcaggctaccccgaccacgcccactcccaccagcgcgtcggccgcggccacaatttcggccacctcctcgccgacctgctcccccggcccggtcataagcatgcagttctccatcttcaccgtccttgccgctgctgccagcttcgctgttgctctccctgtctgtgacgccactaccacggccactactaccagcgccgctgcgaacccctcccctactaccagcggtgctgcgaacccctcccctacttgctccaatggacgaccagggtagcttgaccgacggagacagcatcctcaactcctaccatctccctcggcccatgcctatctggctcaactccaactatggcaagcatattgtcaagggcaacttcatgaccctcagcgcgagacccaagacggtcgagcagg

SEQ ID NO: 85 - DNA, 947 bp, artificial
  Sequence of the second arm of the dsRNA used to concomitantly target CclA/ ACS1/ ACS2/ ELP1/ ELP2/ MOB2 genes from Colletotrichum higginsianum
cctgctcgaccgtcttgggtctcgcgctgagggtcatgaagttgcccttgacaatatgcttgccatagttggagttgagccagataggcatgggccgagggagatggtaggagttgaggatgctgtctccgtcggtcaagctaccctggtcgtccattggagcaagtaggggaggggttcgcagcaccgctggtagtaggggaggggttcgcagcggcgctggtagtagtggccgtggtagtggcgtcacagacagggagagcaacagcgaagctggcagcagcggcaaggacggtgaagatggagaactgcatgcttatgaccgggccgggggagcaggtcggcgaggaggtggccgaaattgtggccgcggccgacgcgctggtgggagtgggcgtggtcggggtagcctgagggagggcggcggcaacgccgacgacagccaggatggagaagagttcggagatctgcatgcactgatgccgcccttgagcgtctttttggagactttcaatgcggacgagggtttcttgtgccagtgcagatgctcggcctggtgggcccagaacccctcggggtcctcgagggaacggcgcaggacctcgtcttgagggtgtaagtcgcccatgcatctccggtggtgtgcatgacacctttgggcttaccggtagagccggaggtgtagaggaggaacaaggggtcctcggagttgacacgctcgggggggaagtaggcaggccacttctcgacctcctcgtgccaccaccaatcacggcttttggtcagatcggtgttgatcggacgtctggattgagaggagatggtacagagggagtaaaatcctcctcgagggcggcgcgcttcaacgggatcgcggttgaggcaggaagggaaccgctcagagtcggctcccaaggaggagggggagtacccgagtcggacgccat

SEQ ID NO: 86 - DNA, 792 bp, artificial
  Sequence of the first arm of the CclA/ ACS1/ ACS2 dsRNA used to concomitantly target CclA/ ACS1/ ACS2 genes from Colletotrichum higginsianum
atggcgtccgactcgggtactccccctcctccttgggagccgactctgagcggttcccttcctgcctcaaccgcgatcccgttgaagcgcgccgccctcgaggaggattttactccctctgtaccatctcctctcaatccagacgtccgatcaacaccgaaacctcaaggcgtcgacgacatgcctgctaagcgcaacaaaaaggagtcgctgaagaagcgcgagtccaagggcgctttcggcggcgacagcaaccgatcatgaccaaaagccgtgattggtggtggcacgaggaggtcgagaagtggcctgcctacttcccccccgagcgtgtcaactccgaggaccccttgttcctcctctacacctccggctctaccggtaagcccaaaggtgtcatgcacaccaccggaggctacctggtcggcgccgccgccaccggcaagtacgttttcgacatccacgacggcgaccgctacttttgtggtggtgacgttggctggattaccggtcacacctacgttatgcatgggcgacttacaccctcaagacgaggtcctgcgccgttccctcgaggaccccgaggggttctgggcccaccaggccgagcatctgcactggcacaagaaaccctcgtccgcattgaaagtctccaaaaagacgctcaagggcggcatcacacacgacacatgggaatggttcccggacggcgagatctcgacctgcttcaactgcgtcgaccgccacgtccacgacggcaaaggcgatgccccggcgatcttctacgacagc

SEQ ID NO: 87 - DNA, 792 bp, artificial
  Sequence of the second arm of the CclA/ ACS1/ ACS2 dsRNA used to concomitantly target CclA/ ACS1/ ACS2 genes from Colletotrichum higginsianum
gctgtcgtagaagatcgccggggcatcgcctttgccgtcgtggacgtggcggtcgacgcagttgaagcaggtcgagatctcgccgtccgggaaccattcccatgtgtcgtgtgtgatgccgcccttgagcgtctttttggagactttcaatgcggacgagggtttcttgtgccagtgcagatgctcggcctggtgggcccagaacccctcggggtcctcgagggaacggcgcaggacctcgtcttgagggtgtaagtcgcccatgcataacgtaggtgtgaccggtaatccagccaacgtcaccaccacaaaagtagcggtcgccgtcgtggatgtcgaaaacgtacttgccggtggcggcggcgccgaccaggtagcctccggtggtgtgcatgacacctttgggcttaccggtagagccggaggtgtagaggaggaacaaggggtcctcggagttgacacgctcgggggggaagtaggcaggccacttctcgacctcctcgtgccaccaccaatcacggcttttggtcatgatcggttgctgtcgccgccgaaagcgcccttggactcgcgcttcttcagcgactcctttttgttgcgcttagcaggcatgtcgtcgacgccttgaggtttcggtgttgatcggacgtctggattgagaggagatggtacagagggagtaaaatcctcctcgagggcggcgcgcttcaacgggatcgcggttgaggcaggaagggaaccgctcagagtcggctcccaaggaggagggggagtacccgagtcggacgccat

SEQ ID NO: 88 - DNA, 792 bp, artificial
  Sequence of the first arm of the ELP1/ ELP2/ MOB2 dsRNA used to concomitantly target ELP1/ ELP2/ MOB2 genes from Colletotrichum higginsianum
atgcagatctccgaactcttctccatcctggctgtcgtcggcgttgccgccgccctccctcaggctaccccgaccacgcccactcccaccagcgcgtcggccgcggccacaatttcggccacctcctcgccgacctgctcccccggcccggtcattgactacaccgtcgtctcgggcgacaccctgaccatcatctcgcagagattcagctccggcatctgcaacatcgccgacgcgagcggcatcaccaaccccaactgatcatgcagttctccatcttcaccgtccttgccgctgctgccagcttcgctgttgctctccctgtctgtgacgccactaccacggccactactaccagcgccgctgcgaacccctcccctactaccagcggtgctgcgaacccctcccctacttgcggcaagcttggagatttccacaagaccaccgttaaggctggccagaccttgacgaccatcgccgagcgcttccactccggtatctgtgacatcgcctggcagaatgcatggacgaccagggtagcttgaccgacggagacagcatcctcaactcctaccatctccctcggcccatgcctatctggctcaactccaactatggcaagcatattgtcaagggcaacttcatgaccctcagcgcgagacccaagacggtcgagcagggcgagtggatcgctcaccaggtcgtcgagcactacagaaacctctggaattttgttcgtgttctccacgagaaggaggaggatggctcgactatctgcaatgccaccacctg

SEQ ID NO: 89 - DNA, 792 bp, artificial
  Sequence of the second arm of the ELP1/ ELP2/ MOB2 dsRNA used to concomitantly target ELP1, ELP2, and MOB2 genes from Colletotrichum higginsianum
caggtggtggcattgcagatagtcgagccatcctcctccttctcgtggagaacacgaacaaaattccagaggtttctgtagtgctcgacgacctggtgagcgatccactcgccctgctcgaccgtcttgggtctcgcgctgagggtcatgaagttgcccttgacaatatgcttgccatagttggagttgagccagataggcatgggccgagggagatggtaggagttgaggatgctgtctccgtcggtcaagctaccctggtcgtccatgcattctgccaggcgatgtcacagataccggagtggaagcgctcggcgatggtcgtcaaggtctggccagccttaacggtggtcttgtggaaatctccaagcttgccgcaagtaggggaggggttcgcagcaccgctggtagtaggggaggggttcgcagcggcgctggtagtagtggccgtggtagtggcgtcacagacagggagagcaacagcgaagctggcagcagcggcaaggacggtgaagatggagaactgcatgatcagttggggttggtgatgccgctcgcgtcggcgatgttgcagatgccggagctgaatctctgcgagatgatggtcagggtgtcgcccgagacgacggtgtagtcaatgaccgggccgggggagcaggtcggcgaggaggtggccgaaattgtggccgcggccgacgcgctggtgggagtgggcgtggtcggggtagcctgagggagggcggcggcaacgccgacgacagccaggatggagaagagttcggagatctgcat

SEQ ID NO: 90 - DNA, 500 bp, artificial
  Sequence of the first arm of the CFA6/HRPL dsRNA used to concomitantly target HrpL and Cfa6 genes of Pto DC3000
atgaacaagcgctacggcaacaacttgccgggatcctgttgcgcgctgagcaaccggatcgactggacgtcaccttcgcgcagcacgccatctggcaaaaaggccgccgttaatcgtttccaatatttctgcaggagtctgcccgtgaccaccttcaccgtgcccctgattctgtcggatgcaagccccacggcactcgccgctaccgcgcaacgtctgcgcgccgagcacgagacgcgctccttcgccgctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 91 - DNA, 500 bp, artificial
  Sequence of the second arm of the CFA6/HRPL dsRNA used to concomitantly target HrpL and Cfa6 genes of Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcagcggcgaaggagcgcgtctcgtgctcggcgcgcagacgttgcgcggtagcggcgagtgccgtggggcttgcatccgacagaatcaggggcacggtgaaggtggtcacgggcagactcctgcagaaatattggaaacgattaacggcggcctttttgccagatggcgtgctgcgcgaaggtgacgtccagtcgatccggttgctcagcgcgcaacaggatcccggcaagttgttgccgtagcgcttgttcat

SEQ ID NO: 92 - DNA, 348 bp, artificial
  Sequence of the first arm of the HRPL dsRNA used to target the HrpL gene of Pto DC3000
ctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctgagcatctgaatctgatcggccgtcatttggcgaataccggcggacgaagatggctgacggggctgggttgagtcgaggatcacaatcttctgaaacat

SEQ ID NO: 93 - DNA, 348 bp, artificial
  Sequence of the second arm of the HRPL dsRNA used to target the HrpL gene of Pto DC3000
atgtttcagaagattgtgatcctcgactcaacccagccccgtcagccatcttcgtccgccggtattcgccaaatgacggccgatcagattcagatgctcagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcag

SEQ ID NO: 94 - DNA, 400 bp, artificial
  Sequence of the first arm of the AvrPto/AvrPtoB dsRNA used to target the AvrPto and AvrPtoB genes of Pto DC3000
atgggaaatatatgtgtcggcggatccaggatggcccatcaggtgaactccccagaccgagttagtaacaactcgggtgacgaagataacgtaacgtccagtcaactgctgagcgtcagacatcaacttgcggagtctgctggtgtaccaagagatcagcatgaatttgttagtaaccaagcacctcaaagcctgagaaaatggcgggtatcaatagagcgggaccatcgggcgcttattttgttggccacacagaccccgagccagtatcggggcaagcacacggatccggcagcggcgccagctcctcgaacagtccgcaggttcagccgcgaccctcgaatactcccccgtcgaacgcgcccgcaccgccgccaaccggacgtgagaggctttcacg

SEQ ID NO: 95 - DNA, 400 bp, artificial
  Sequence of the second arm of the AvrPto/AvrPtoB dsRNA used to target the AvrPto and AvrPtoB genes of Pto DC3000
cgtgaaagcctctcacgtccggttggcggcggtgcgggcgcgttcgacgggggagtattcgagggtcgcggctgaacctgcggactgttcgaggagctggcgccgctgccggatccgtgtgcttgccccgatactggctcggggtctgtgtggccaacaaaataagcgcccgatggtcccgctctattgatacccgccattttctcaggctttgaggtgcttggttactaacaaattcatgctgatctcttggtacaccagcagactccgcaagttgatgtctgacgctcagcagttgactggacgttacgttatcttcgtcacccgagttgttactaactcggtctggggagttcacctgatgggccatcctggatccgccgacacatatatttcccat

SEQ ID NO: 96 - DNA, 752 bp, artificial
  Sequence of the first arm of the CYP51 dsRNA used to target the FgCYP51A, FgCYP51B and FgCYP51C genes of Fusarium graminearum
cagcaagtttgacgagtccctggccgctctctaccacgacctcgatatgggcttcacccccatcaacttcatgcttcactgggcccctctcccctggaaccgtaagcgcgaccacgcccagcgcactgttgccaagatctacatggacactatcaaggagcgccgcgccaagggcaacaacgaatccgagcatgacatgatgaagcaccttatgaactctcggtccattgacaatccccgtctttggtagcgatgtcgtatacgattgtcccaactcgaagctcatggaacaaaagaagtttgtcaagtttggccttacgcaaaaagcactcgagtcacacgtccagttaatcgagcgagaggttcttgactacgtcgaaactgatccatccttttctggcagaactagcaccatcgatgtccccaaggcaatggctgagataacaatctttactgcctcacgttctttgcagggtgaggaagttcggagaaaactcactgccgagtttgctgcattggaagcaccgtacaatatggcatcgacccgtacgcttttttcttcgactgcagagataaatacggcgactgctttacctttattctccttggcaaatcaacgactgtctttcttggtcccaagggcaatgactttatcctcaacggcaaacacgccgatctcaacgccgaggacgtttatgggaaacttaccacgcccgtgtttggtgaggaggttgtttatgactgctccaatg

SEQ ID NO: 97 - DNA, 752 bp, artificial
  Sequence of the second arm of the CYP51 dsRNA used to target the FgCYP51A, FgCYP51B and FgCYP51C genes of Fusarium graminearum
cattggagcagtcataaacaacctcctcaccaaacacgggcgtggtaagtttcccataaacgtcctcggcgttgagatcggcgtgtttgccgttgaggataaagtcattgcccttgggaccaagaaagacagtcgttgatttgccaaggagaataaaggtaaagcagtcgccgtatttatctctgcagtcgaagaaaaaagcgtacgggtcgatgccatattgtacggtgcttccaatgcagcaaactcggcagtgagttttctccgaacttcctcaccctgcaaagaacgtgaggcagtaaagattgttatctcagccattgccttggggacatcgatggtgctagttctgccagaaaaggatggatcagtttcgacgtagtcaagaacctctcgctcgattaactggacgtgtgactcgagtgctttttgcgtaaggccaaacttgacaaacttcttttgttccatgagcttcgagttgggacaatcgtatacgacatcgctaccaaagacggggattgtcaatggaccgagagttcataaggtgcttcatcatgtcatgctcggattcgttgttgcccttggcgcggcgctccttgatagtgtccatgtagatcttggcaacagtgcgctgggcgtggtcgcgcttacggttccaggggagaggggcccagtgaagcatgaagttgatgggggtgaagcccatatcgaggtcgtggtagagagcggccagggactcgtcaaacttgctg

SEQ ID NO: 98 - DNA, 667 bp, artificial
  Sequence of the first arm of the HRPG/HRPB/HRCC dsRNA used to target concomitantly the HrpG, HrpB and HrcC genes of Ralstonia solanacearum
cgcatgcctggccgtgcagaacgtggaatgtgtccggttcgacgatgccctgacgctgttgcgtgcacggcgtaccgaggcattcagtctgctgctgatcgatgcccagcagttccgcagcgcgggacagctggtgctgtcctggcgcgagtgcaacgccgacatgtgctggccgacgctggtgttcggccagttcgcagtgaagaagcggaagaaggcttccgcctggcgcagcgcctgatccgccattcggatgacgcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgcctcgaccgcgcaggtcaagcgggcgctgac

SEQ ID NO: 99 - DNA, 667 bp, artificial
  Sequence of the second arm of the HRPG/HRPB/HRCC dsRNA used to target concomitantly the HrpG, HrpB and HrcC genes of Ralstonia solanacearum
gtcagcgcccgcttgacctgcgcggtcgaggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgcgtcatccgaatggcggatcaggcgctgcgccaggcggaagccttcttccgcttcttcactgcgaactggccgaacaccagcgtcggccagcacatgtcggcgttgcactcgcgccaggacagcaccagctgtcccgcgctgcggaactgctgggcatcgatcagcagcagactgaatgcctcggtacgccgtgcacgcaacagcgtcagggcatcgtcgaaccggacacattccacgttctgcacggccaggcatgcg

SEQ ID NO: 100 - DNA, 705 bp, artificial
  Sequence of the first arm of the HRPB/HRCC/TssB/XpsR dsRNA used to target concomitantly the HrpB, HrcC, XpsR and TssB genes of Ralstonia solanacearum
gcgcgatcgctacgccgcggcggcgagctgcttctcgcgcatggccgaagacgatggcgcgacctggacccagcaggtcgagggcctgatcggcccgcggctggctggccaccatcgatctgatcatctacgttttcgccgtgcaggccggcatccgctgctccaaccgcctgctcgagcatgcgttctggcaatcggccgaaatgggcatctcgacgcaggtcgagggcagcgtgacgggctcgttcaacgagacgccgcagaagtttctcgaccgcatggccggcacgttcggctttgcgtggtactacgacggcgcggtgctgcgcgtgaccagcgccaacgaagcgcagagcgccaccatcgcgctgacccgcgccggccgcgcgtgcggagacccatcggcggccgcagagcgaatgcgactggaagcattactttgcggacctgctctatgcgccgaacggcgccgagttcaagctcagcctgtttccgctgcccgcgcagcagatcggcgacacgccctggtcgcgggcattccgcgggcagccggcgatgcttgacaaccaatccgccgcgctcgcgcagagcgcaccggcagccgagtccgtcaacctgctcgacgagatcgtcgagcagagccgcgtcgccaagtcggacgccgagcacgcgcgcgccaaggacatcatcggcgagctggtcaac

SEQ ID NO: 101 - DNA, 705 bp, artificial
  Sequence of the second arm of the HRPB/HRCC/TssB/XpsR dsRNA used to target concomitantly the HrpB, HrcC, XpsR and TssB genes of Ralstonia solanacearum
gttgaccagctcgccgatgatgtccttggcgcgcgcgtgctcggcgtccgacttggcgacgcggctctgctcgacgatctcgtcgagcaggttgacggactcggctgccggtgcgctctgcgcgagcgcggcggattggttgtcaagcatcgccggctgcccgcggaatgcccgcgaccagggcgtgtcgccgatctgctgcgcgggcagcggaaacaggctgagcttgaactcggcgccgttcggcgcatagagcaggtccgcaaagtaatgcttccagtcgcattcgctctgcggccgccgatgggtctccgcacgcgcggccggcgcgggtcagcgcgatggtggcgctctgcgcttcgttggcgctggtcacgcgcagcaccgcgccgtcgtagtaccacgcaaagccgaacgtgccggccatgcggtcgagaaacttctgcggcgtctcgttgaacgagcccgtcacgctgccctcgacctgcgtcgagatgcccatttcggccgattgccagaacgcatgctcgagcaggcggttggagcagcggatgccggcctgcacggcgaaaacgtagatgatcagatcgatggtggccagccagccgcgggccgatcaggccctcgacctgctgggtccaggtcgcgccatcgtcttcggccatgcgcgagaagcagctcgccgccgcggcgtagcgatcgcgc

SEQ ID NO: 102 - DNA, 633 bp, artificial
  Sequence of the first arm of the HRPG/HRPX/RsmA dsRNA used to target concomitantly the HrpG, HrpX, and RsmA genes of Xanthomonas campestris pv. campestris
gtgatggacgccgctgcgaatgaccgccaaggatcggcattcgtactgacgcaggacgcgcggctggcatcacagatcaacgccagcctggcgactctggtcccagaggtcaccatcttttcagacgaactcgaattgctgcgctgcctgcgccactcgccgtgcgagcttctggttttcgatgcccattgcgtggcatcggacgacagtatgatcctttcgacctacttcgcagcgatctctgcgttgtcttacgcagaccgtcttccgatctatacgagcaggatgcttgttggtgcttggccacagggtctgcagcacctccagcaacgtcgcgaggggcaggcagctccggatggcagcgacgacgaagtcgcattgctgggcgccagcgccgatgccttgttgattctggagtatatgttgatcctcactcgccgagtaggcgaaaccctgatgatcggcgacttggtcaccgtgaccgtgctcggcgtcaagggaaatcaggtgcgcattggcattgacgcgcctaaggatgttgccgtgcatcgcgaagaaatctatcagcgcatccagcgcggtgacgagccggttgcgtccggtgcgcatcacaacgacgattgttcggactga

SEQ ID NO: 103 - DNA, 633 bp, artificial
  Sequence of the second arm of the HRPG/HRPX/RsmA dsRNA used to target concomitantly the HrpG, HrpX, and RsmA genes of Xanthomonas campestris pv. campestris
tcagtccgaacaatcgtcgttgtgatgcgcaccggacgcaaccggctcgtcaccgcgctggatgcgctgatagatttcttcgcgatgcacggcaacatccttaggcgcgtcaatgccaatgcgcacctgatttcccttgacgccgagcacggtcacggtgaccaagtcgccgatcatcagggtttcgcctactcggcgagtgaggatcaacatatactccagaatcaacaaggcatcggcgctggcgcccagcaatgcgacttcgtcgtcgctgccatccggagctgcctgcccctcgcgacgttgctggaggtgctgcagaccctgtggccaagcaccaacaagcatcctgctcgtatagatcggaagacggtctgcgtaagacaacgcagagatcgctgcgaagtaggtcgaaaggatcatactgtcgtccgatgccacgcaatgggcatcgaaaaccagaagctcgcacggcgagtggcgcaggcagcgcagcaattcgagttcgtctgaaaagatggtgacctctgggaccagagtcgccaggctggcgttgatctgtgatgccagccgcgcgtcctgcgtcagtacgaatgccgatccttggcggtcattcgcagcggcgtccatcac

SEQ ID NO: 104 - DNA, 718 bp, artificial
  Sequence of the first arm of the NadHb/NadHd/NadHe dsRNA used to target concomitantly the NadHb, NadHd and NadHe genes of Xanthomonas campestris pv. campestris (and likely other Xanthomonas species)
aacagaagaagggagcgctggaatgggagtgattcagaccctggatcgtctgatgaccaacccgatgccggaaggccgggtcgaagacatcctgcgcccggaaggcgaaaacccgttgctcgaaaagggctacgtgaccaccagcgtcgatgcgctgttgaactgggcgcgtacaggttcgatgtggccgatgacctttggtctggcctgctgtgcagtcgagatgatgcagatcgtgagtgagtaccgccaggcaaccgatgccttcgcgagcaatcctgtggaaagcaagcaggaaatccgcaattacacgatgaacttcggcccgcagcatcctgcggcgcacggcgtattgcgcctgatcctggaaatggacggcgaaaccgtggtgcgtgccgacccgcatatcggcctgctgcaccgtggcaccgagaagctggccgagtccaagccgttcaaccagtcggttccgtacagcatcgacgggtaatttcgaagcggcgcgcgacgtcgatccgcaggtagtgctgagcgacaagacgcgcgcgcacatcgatcattggctgagcaagttcccgcccgaccgcaagcgttcggccgtgttgcagggtctgcatgccgcgcaggaacagaaccagggttggttgaccgacgagctgatcgtgggcgtggccaagtatctggagctgccgccggtgtgggcctacgaagtggggct

SEQ ID NO: 105 - DNA, 718 bp, artificial
  Sequence of the second arm of the NadHb/NadHd/NadHe dsRNA used to target concomitantly the NadHb, NadHd and NadHe genes of Xanthomonas campestris pv. campestris (and likely other Xanthomonas species).
tcagccacttcgtaggcccacaccggcggcagctccagatacttggccacgcccacgatcagctcgtcggtcaaccaaccctggttctgttcctgcgcggcatgcagaccctgcaacacggccgaacgcttgcggtcgggcgggaacttgctcagccaatgatcgatgtgcgcgcgcgtcttgtcgctcagcactacctgcggatcgacgtcgcgcgccgcttcgaaattacccgtcgatgctgtacggaaccgactggttgaacggcttggactcggccagcttctcggtgccacggtgcagcaggccgatatgcgggtcggcacgcaccacggtttcgccgtccatttccaggatcaggcgcaatacgccgtgcgccgcaggatgctgcgggccgaagttcatcgtgtaattgcggatttcctgcttgctttccacaggattgctcgcgaaggcatcggttgcctggcggtactcactcacgatctgcatcatctcgactgcacagcaggccagaccaaaggtcatcggccacatcgaacctgtacgcgcccagttcaacagcgcatcgacgctggtggtcacgtagcccttttcgagcaacgggttttcgccttccgggcgcaggatgtcttcgacccggccttccggcatcgggttggtcatcagacgatccagggtctgaatcactcccattccagcgctcccttcttcctgc

SEQ ID NO: 106 - DNA, 677 bp, artificial
  Sequence of the first arm of the DnaA/DnaE1/DnaE2 dsRNA used to target concomitantly the DnaA, DnaE1 and DnaE2 genes of Xanthomonas campestris pv. campestris (and likely other species)
aacaatgcttggccccgctgtctggaacgtctcgaagctgaattcccgcccgaggatgtccacacctggttgaaacccctgcaggccgaagatcgcggcgacagcatcgtgctgtacgcgccgaacgccttcattgttgagcaggtccgcgagcgatacctgccgcgcatccgcgagttgctggcatatttcgccggcaatggcgaggtggcgctggcggtcggctcccgtcgatcgcgcgaagccttcctcaagccgttggcccaggtggtcaatgtcgtcgaacggcgtcagacattgccggtactggcgaacttgctggtgcaggtgaacaacggccagctgtcgctgacggggaccgacctggaagtcgaaatgatctcgcgcaccatggtcgaggacgcccaggacggcgaaaccacgatcccggcgcgcaagctgttcgacatcctggcatatgtccacttcccgcttcgtccatctgcacgtccacaccgagttctcgttggcggattccaccatccgcgtgcccgagaaaccggatcaggctgacccgaaaaaagccaagcaggccaacctgctgagccgcgcggtcgaactcgacttgcccgcgctggcggtcaccgacctgaacaacctgttcgccctggtcaagttctacaaggccgccgaaggct

SEQ ID NO: 107 - DNA, 677 bp, artificial
  Sequence of the second arm of the DnaA/DnaE1/DnaE2 dsRNA used to target concomitantly the DnaA, DnaE1 and DnaE2 genes of Xanthomonas campestris pv. campestris (and likely other Xanthomonas species)
tcagttcggcggccttgtagaacttgaccagggcgaacaggttgttcaggtcggtgaccgccagcgcgggcaagtcgagttcgaccgcgcggctcagcaggttggcctgcttggcttttttcgggtcagcctgatccggtttctcgggcacgcggatggtggaatccgccaacgagaactcggtgtggacgtgcagatggacgaagcgggaagtggacatatgccaggatgtcgaacagcttgcgcgccgggatcgtggtttcgccgtcctgggcgtcctcgaccatggtgcgcgagatcatttcgacttccaggtcggtccccgtcagcgacagctggccgttgttcacctgcaccagcaagttcgccagtaccggcaatgtctgacgccgttcgacgacattgaccacctgggccaacggcttgaggaaggcttcgcgcgatcgacgggagccgaccgccagcgccacctcgccattgccggcgaaatatgccagcaactcgcggatgcgcggcaggtatcgctcgcggacctgctcaacaatgaaggcgttcggcgcgtacagcacgatgctgtcgccgcgatcttcggcctgcaggggtttcaaccaggtgtggacatcctcgggcgggaattcagcttcgagacgttccagacagcggggccaagcatctgc

SEQ ID NO: 108 - DNA, 884 bp, artificial
  Sequence of the first arm of the Br1A2/stuA/F1bc/pks1/PPT1 dsRNA used to concomitantly target Br1A2/stuA/F1bc/pks1/PPT1 genes from zymoseptoria tritici
atgccaccaccgcagccacgatccgtgcagcccgaaacattcctcctggaccaggatgcacagcagagcttgcctcaagattcagtggtcgcgctgcagcaggtggacaatctgaaatactttctcatatctgctcctgtggactggagacaagactcgtactgcaggagagatcagttccttcgaccagaccggacaagtcgcgccgcatggtatgaagcctagggtcactgcgaccttgtgggaagatgagggtagcttgtgcttccaggtagaagcgaacggcgtgtgcgtcgcgcggcgcgaagacaaccacatgatcaatggcacgaaactgttgaacgatgcatggcaaccatggccgtcgacaaccgccaacaattcagcagcatgggtggctacgacgcaaagttctacccgcacccgccgcagttcacgaatccttggacgtctggggcttccaacccaagtcaaatgtacgcgacatccatgccacatacatcaggtggcagcgatgcgcgggtgcatgtcgaacatacttctcttcggcgatcaaaccgccgaacaatacccgctcctccatcgaatcgttctccgaaaggacaatgtcttgctgctcagctttgtcgagcgttgcgctcttgctctccgcgaagagactcgcaccctgtcccgaagccaacgcgaagtgatgccggactaagcatggcccaacaactgcagtcgctgtcagacagcctgccgagtgtctcgactctggtcaatggaataaccaacgccttccacaatgccacatctctggtcagtggcgctccgcacgctggcgaaattacctgctggctgatcgatactcgaaagctgtggcctggaaccaaaatctggga

SEQ ID NO: 109 - DNA, 884 bp, artificial
  Sequence of the second arm of the Br1A2/stuA/F1bc/pks1PPT1 dsRNA used to concomitantly target Br1A2/stuA/F1bc/pks1/PPT1 genes from zymoseptoria tritici
tcccagattttggttccaggccacagctttcgagtatcgatcagccagcaggtaatttcgccagcgtgcggagcgccactgaccagagatgtggcattgtggaaggcgttggttattccattgaccagagtcgagacactcggcaggctgtctgacagcgactgcagttgttgggccatgcttagtccggcatcacttcgcgttggcttcgggacagggtgcgagtctcttcgcggagagcaagagcgcaacgctcgacaaagctgagcagcaagacattgtcctttcggagaacgattcgatggaggagcgggtattgttcggcggtttgatcgccgaagagaagtatgttcgacatgcacccgcgcatcgctgccacctgatgtatgtggcatggatgtcgcgtacatttgacttgggttggaagccccagacgtccaaggattcgtgaactgcggcgggtgcgggtagaactttgcgtcgtagccacccatgctgctgaattgttggcggttgtcgacggccatggttgccatgcatcgttcaacagtttcgtgccattgatcatgtggttgtcttcgcgccgcgcgacgcacacgccgttcgcttctacctggaagcacaagctaccctcatcttcccacaaggtcgcagtgaccctaggcttcataccatgcggcgcgacttgtccggtctggtcgaaggaactgatctctcctgcagtacgagtcttgtctccagtccacaggagcagatatgagaaagtatttcagattgtccacctgctgcagcgcgaccactgaatcttgaggcaagctctgctgtgcatcctggtccaggaggaatgtttcgggctgcacggatcgtggctgcggtggtggcat

SEQ ID NO: 110 - DNA, 796 bp, artificial
  Sequence of the first arm of the Br1A2/stuA/F1bc dsRNA used to concomitantly target Br1A2/stuA/F1bc/ genes from zymoseptoria tritici
atgccaccaccgcagccacgatccgtgcagcccgaaacattcctcctggaccaggatgcacagcagagcttgcctcaagattcagtggtcgcgctgcagcaggtggacaatctgaaatactttctcatatctgctcctgtggactggagacaagactcgtactgcaggagatttctcttgccaaccggcgaatacgtgtcttgcgtgttatggaacaatctcttccacatctccgggaccgacattgtccgatgcttgtcgttccgtttcgatcgttccttcgaccagaccggacaagtcgcgccgcatggtatgaagcctagggtcactgcgaccttgtgggaagatgagggtagcttgtgcttccaggtagaagcgaacggcgtgtgcgtcgcgcggcgcgaagacaaccacatgatcaatggcacgaaactgttgaacgttgccgggatgacacgcggtcggcgcgatggcatcttgaagagcgagaagactcgccatgtggtcaagattggtccgatgcacctcaaaggtgatgctggcaaccatggccgtcgacaaccgccaacaattcagcagcatgggtggctacgacgcaaagttctacccgcacccgccgcagttcacgaatccttggacgtctggggcttccaacccaagtcaaatgtacgcgacatccatgccacatacatcaggtggcagcgatgcgcggtacgtccaggcgcccaatgtttctgctgcatacggtggtagcatcccagctcccgctatggcatcaggtatagatctactggac

SEQ ID NO: 111 - DNA, 796 bp, artificial
  Sequence of the second arm of the Br1A2/stuA/F1bc dsRNA used to concomitantly target Br1A2/stuA/F1bc/ genes from zymoseptoria tritici
gtccagtagatctatacctgatgccatagcgggagctgggatgctaccaccgtatgcagcagaaacattgggcgcctggacgtaccgcgcatcgctgccacctgatgtatgtggcatggatgtcgcgtacatttgacttgggttggaagccccagacgtccaaggattcgtgaactgcggcgggtgcgggtagaactttgcgtcgtagccacccatgctgctgaattgttggcggttgtcgacggccatggttgccagcatcacctttgaggtgcatcggaccaatcttgaccacatggcgagtcttctcgctcttcaagatgccatcgcgccgaccgcgtgtcatcccggcaacgttcaacagtttcgtgccattgatcatgtggttgtcttcgcgccgcgcgacgcacacgccgttcgcttctacctggaagcacaagctaccctcatcttcccacaaggtcgcagtgaccctaggcttcataccatgcggcgcgacttgtccggtctggtcgaaggaacgatcgaaacggaacgacaagcatcggacaatgtcggtcccggagatgtggaagagattgttccataacacgcaagacacgtattcgccggttggcaagagaaatctcctgcagtacgagtcttgtctccagtccacaggagcagatatgagaaagtatttcagattgtccacctgctgcagcgcgaccactgaatcttgaggcaagctctgctgtgcatcctggtccaggaggaatgtttcgggctgcacggatcgtggctgcggtggtggcat

SEQ ID NO: 112 - DNA, 782 bp, artificial
  Sequence of the first arm of the stuA/pks1/PPT1 dsRNA used to concomitantly target stuA/ /pks1/PPT1 genes from zymoseptoria tritici
agttccttcgaccagaccggacaagtcgcgccgcatggtatgaagcctagggtcactgcgaccttgtgggaagatgagggtagcttgtgcttccaggtagaagcgaacggcgtgtgcgtcgcgcggcgcgaagacaaccacatgatcaatggcacgaaactgttgaacgttgccgggatgacacgcggtcggcgcgatggcatcttgaagagcgagaagactcgccatgtggtcaagattggtccgatgcacctcaaagatcatgtcgaacatacttctcttcggcgatcaaaccgccgaacaatacccgctcctccatcgaatcgttctccgaaaggacaatgtcttgctgctcagctttgtcgagcgttgcgctcttgctctccgcgaagagactcgcaccctgtcccgaagccaacgcgaagtgatgccggacttcctcaccctcagcaatctgatcgaggcatactaccagaagggtgaaaaagtcctcatgttggaatcagcatttcttacgattgcatgctggcccaacaactgcagtcgctgtcagacagcctgccgagtgtctcgactctggtcaatggaataaccaacgccttccacaatgccacatctctggtcagtggcgctccgcacgctggcgaaattacctgctggctgatcgatactcgaaagctgtggcctggaaccaaaatctgggatgctcctggcgcagcagaagccatgggtttgatttctgtggccgagcagaatgccatctcgacgaagatgttcattca

SEQ ID NO: 113 - DNA, 782 bp, artificial
  Sequence of the second arm of the stuA/pks1/PPT1 dsRNA used to concomitantly target stuA/ /pks1/PPT1 genes from zymoseptoria tritici
tgaatgaacatcttcgtcgagatggcattctgctcggccacagaaatcaaacccatggcttctgctgcgccaggagcatcccagattttggttccaggccacagctttcgagtatcgatcagccagcaggtaatttcgccagcgtgcggagcgccactgaccagagatgtggcattgtggaaggcgttggttattccattgaccagagtcgagacactcggcaggctgtctgacagcgactgcagttgttgggccagcatgcaatcgtaagaaatgctgattccaacatgaggactttttcacccttctggtagtatgcctcgatcagattgctgagggtgaggaagtccggcatcacttcgcgttggcttcgggacagggtgcgagtctcttcgcggagagcaagagcgcaacgctcgacaaagctgagcagcaagacattgtcctttcggagaacgattcgatggaggagcgggtattgttcggcggtttgatcgccgaagagaagtatgttcgacatgatctttgaggtgcatcggaccaatcttgaccacatggcgagtcttctcgctcttcaagatgccatcgcgccgaccgcgtgtcatcccggcaacgttcaacagtttcgtgccattgatcatgtggttgtcttcgcgccgcgcgacgcacacgccgttcgcttctacctggaagcacaagctaccctcatcttcccacaaggtcgcagtgaccctaggcttcataccatgcggcgcgacttgtccggtctggtcgaaggaact

SEQ ID NO: 114 - DNA, 795 bp, artificial
  Sequence of the first arm of the chitinase/ elicitin/glucanase inhibitor, transglutaminase elicitor/ secretory protein dsRNA used to concomitantly target genes producing the protein defined by PITG_17947, PITG_10772, PITG_13671, PITG_16956, PITG_00891
atgaaattcgtcggggttatcgcgtcaagtcttctggttgtgccttctgcggtctctggtgacgctgatagctcgagtttcgcgctcttcttcgatcaggatcgtttccaggaggttttcccggacgctgtggagctctacaacttcaacggtcttgtgggatcatggccaagttcactctcgctgtttcgacgcttcttgtcgcgtcggctgccgtctcggcccacgaggggcacgaccactcgtcgtcgacccacgacgaacatgagcactcaggctctgccgcttcaggcaccgttggttcggagtgctcgatgcccatgaagatcatctctgctgtcgccgcctcctcaattgcgcttggagccgtctctgccactactgaccatgtcagccgaatgctggttcttggtggagcagttgttcccagcggcacgaagacgtacacgaccggaatccgccctaccattgatggcggtgccgtcgagaagattacaaagccttcctccgttccaggttgacgcgctatcccttccggatctctagatcgactctctttgcaacatggtattctcgcccagcacctatctcgtctccgctgccgtcgctgtcgtggcgctgcagatgcagcaagcgtggagcagcgacttgtttattcaacaatgcaaccttatttgctagtagcgacattactgggtgctgccacgacggcaaatgcaattccagtgcacttcaacaatcactgcaatgctgcaattgagctgtacaacaacacatacatcgaggtgatcaatc

SEQ ID NO: 115 - DNA, 795 bp, artificial
  Sequence of the second arm of the chitinase/ elicitin/glucanase inhibitor, transglutaminase elicitor/ secretory protein dsRNA used to concomitantly target genes producing the protein defined by PITG_17947, PITG_10772, PITG_13671, PITG_16956, PITG_00891
gattgatcacctcgatgtatgtgttgttgtacagctcaattgcagcattgcagtgattgttgaagtgcactggaattgcatttgccgtcgtggcagcacccagtaatgtcgctactagcaaataaggttgcattgttgaataaacaagtcgctgctccacgcttgctgcatctgcagcgccacgacagcgacggcagcggagacgagataggtgctgggcgagaataccatgttgcaaagagagtcgatctagagatccggaagggatagcgcgtcaacctggaacggaggaaggctttgtaatcttctcgacggcaccgccatcaatggtagggcggattccggtcgtgtacgtcttcgtgccgctgggaacaactgctccaccaagaaccagcattcggctgacatggtcagtagtggcagagacggctccaagcgcaattgaggaggcggcgacagcagagatgatcttcatgggcatcgagcactccgaaccaacggtgcctgaagcggcagagcctgagtgctcatgttcgtcgtgggtcgacgacgagtggtcgtgcccctcgtgggccgagacggcagccgacgcgacaagaagcgtcgaaacagcgagagtgaacttggccatgatcccacaagaccgttgaagttgtagagctccacagcgtccgggaaaacctcctggaaacgatcctgatcgaagaagagcgcgaaactcgagctatcagcgtcaccagagaccgcagaaggcacaaccagaagacttgacgcgataaccccgacgaatttcat

SEQ ID NO: 116 - DNA, 781 bp, artificial
  Sequence of the first arm of the chitinase/ elicitin/glucanase inhibitor, dsRNA used to concomitantly target genes producing the protein defined by PITG_17947, PITG_10772, PITG_13671 from plasmopora viticola
atgaaattcgtcggggttatcgcgtcaagtcttctggttgtgccttctgcggtctctggtgacgctgatagctcgagtttcgcgctcttcttcgatcaggatcgtttccaggaggttttcccggacgctgtggagctctacaacttcaacggtcttgtggatgcggccagcaagtacagcgaattcgctaatacgggcaacgacgacaatgacaagcgtgagctggcagcgttcctggctcaaacagctcacgagtgcggatcatggccaagttcactctcgctgtttcgacgcttcttgtcgcgtcggctgccgtctcggcccacgaggggcacgaccactcgtcgtcgacccacgacgaacatgagcactcaggctctgccgcttcaggcaccgttggttcggagtgctcggccgacgtgagcgaggcgttcatcgcgactgtcgacaacagcacctacttcgaaacgtgcgcagaaggcatgaccttcaacatcacgtcggtcttcgacgtactcaaatgcgccatgaagatcatctctgctgtcgccgcctcctcaattgcgcttggagccgtctctgccactactgaccatgtcagccgaatgctggttcttggtggagcagttgttcccagcggcacgaagacgtacacgaccggaatccgccctaccattgatggcgacaatttttgcggaggatcgctgatctccccaacgcatgttcttacgacgacggcgtgtcttcgtggtatcaagccgccgaattgggtgtctgtggg

SEQ ID NO: 117 - DNA, 781 bp, artificial
  Sequence of the second arm of the chitinase/ elicitin/glucanase inhibitor, dsRNA used to concomitantly target genes producing the protein defined by PITG_17947, PITG_10772, PITG_13671 from plasmopora viticola
cccacagacacccaattcggcggcttgataccacgaagacacgccgtcgtcgtaagaacatgcgttggggagatcagcgatcctccgcaaaaattgtcgccatcaatggtagggcggattccggtcgtgtacgtcttcgtgccgctgggaacaactgctccaccaagaaccagcattcggctgacatggtcagtagtggcagagacggctccaagcgcaattgaggaggcggcgacagcagagatgatcttcatggcgcatttgagtacgtcgaagaccgacgtgatgttgaaggtcatgccttctgcgcacgtttcgaagtaggtgctgttgtcgacagtcgcgatgaacgcctcgctcacgtcggccgagcactccgaaccaacggtgcctgaagcggcagagcctgagtgctcatgttcgtcgtgggtcgacgacgagtggtcgtgcccctcgtgggccgagacggcagccgacgcgacaagaagcgtcgaaacagcgagagtgaacttggccatgatccgcactcgtgagctgtttgagccaggaacgctgccagctcacgcttgtcattgtcgtcgttgcccgtattagcgaattcgctgtacttgctggccgcatccacaagaccgttgaagttgtagagctccacagcgtccgggaaaacctcctggaaacgatcctgatcgaagaagagcgcgaaactcgagctatcagcgtcaccagagaccgcagaaggcacaaccagaagacttgacgcgataaccccgacgaatttcat

SEQ ID NO: 118 - DNA, 767 bp, artificial
  Sequence of the first arm of the glucanase inhibitor, transglutaminase elicitor/ secretory protein dsRNA used to concomitantly target genes producing the protein defined by PITG_13671, PITG_16956, PITG_00891 from plasmopora viticola
gccatgaagatcatctctgctgtcgccgcctcctcaattgcgcttggagccgtctctgccactactgaccatgtcagccgaatgctggttcttggtggagcagttgttcccagcggcacgaagacgtacacgaccggaatccgccctaccattgatggcgacaatttttgcggaggatcgctgatctccccaacgcatgttcttacgacgacggcgtgtcttcgtggtatcaagccgccgaattgggtgtctgtgggcacgatctcgtcgagaagattacaaagccttcctccgttccaggttgacgcgctatcccttccggatctctagatcgactctctttgcaacatggtattctcgcccagcacctatctcgtctccgctgccgtcgctgtcgtggcgctgcagatgcagcacgcttctgccgcgtcgctgtactacggagtgaccaccgtgtcaaagacggccgatacgatctccaaatcgtcaccatttgctggtggcgaagttcctgaccaggattatgcgtggagcagcgacttgtttattcaacaatgcaaccttatttgctagtagcgacattactgggtgctgccacgacggcaaatgcaattccagtgcacttcaacaatcactgcaatgctgcaattgagctgtacaacaacacatacatcgaggtgatcaatccaggcagcaccactactcgcaatcttgaggagggattcagtgggatgtttcggaacggatggaatccccaagcaacacgt

SEQ ID NO: 119 - DNA, 767 bp, artificial
  Sequence of the second arm of the glucanase inhibitor, transglutaminase elicitor/ secretory protein dsRNA used to concomitantly target genes producing the protein defined by PITG_13671, PITG_16956, PITG_00891 from plasmopora viticola
acgtgttgcttggggattccatccgttccgaaacatcccactgaatccctcctcaagattgcgagtagtggtgctgcctggattgatcacctcgatgtatgtgttgttgtacagctcaattgcagcattgcagtgattgttgaagtgcactggaattgcatttgccgtcgtggcagcacccagtaatgtcgctactagcaaataaggttgcattgttgaataaacaagtcgctgctccacgcataatcctggtcaggaacttcgccaccagcaaatggtgacgatttggagatcgtatcggccgtctttgacacggtggtcactccgtagtacagcgacgcggcagaagcgtgctgcatctgcagcgccacgacagcgacggcagcggagacgagataggtgctgggcgagaataccatgttgcaaagagagtcgatctagagatccggaagggatagcgcgtcaacctggaacggaggaaggctttgtaatcttctcgacgagatcgtgcccacagacacccaattcggcggcttgataccacgaagacacgccgtcgtcgtaagaacatgcgttggggagatcagcgatcctccgcaaaaattgtcgccatcaatggtagggcggattccggtcgtgtacgtcttcgtgccgctgggaacaactgctccaccaagaaccagcattcggctgacatggtcagtagtggcagagacggctccaagcgcaattgaggaggcggcgacagcagagatgatcttcatggc

SEQ ID NO: 120 - DNA, 934 bp, artificial
  Sequence of the first arm of the GyrB/ FusA/ MreB/HrpG/PhoP/FhaB dsRNA used to concomitantly target GyrB/ FusA/ MreB/HrpG/PhoP/FhaB genes from xanthomonas citri pv fuscan
tgaccgacgaacaaaacaccccgccaacacccaacggcacctacgactccagcaagatcaccgtgctgcgtggcctggaagccgttcgcaagcgccccggcatgtatatcggcgacgtccatgacggcaccggcctgcatcacatggtgtgatcgtggcccgcaccacccctatcgagcgttaccgcaacttcggcatcatggcccacattgatgccggcaagaccaccacgtccgaacgcatcctgttctacaccggtgtgagtcacaagatcggcgaagtccatgacggcgctgcagtgatgatgcatgttcaagaaactccgcggcatgttctccaacgacctgtccatcgatttgggcacggccaatacgctgatctacgtgcgcggacagggcatcgtgctgaacgagccgtcggtggttgcggtgcgtcaggatcgcgcgatcggcggcacgcggtgcatgaacgaccactctccccccaacgccggatcggtgttcctgttgacgcaggatgcgcgcctggtctcgcaggtcaacgccagcctcgcaccgcttgcgcgcaatgtctcgaccttttccgatgaactggagcttttgcggacgctgcggcattcaagcatgcgtatccttttggtcgaagacgaagctccgctgcgcgaaacccttgcggcgcgcctgaagcgcgaaggctttgcggtcgacgctgcacaggacggcgaagaaggcctctacatgggccgtgaagtgccatttgatgtcggcatcatcgtccaatggacaacgaagcgatgcgcgtgcgtggttacctggccgagcagggcaagcgtgcgcttgcactgttgctgtgctgcacgaccacctggacatcgtttccagcatgggcgcaggttgcgccaaccgccaaccctgcagggcagacgcccggtcag

SEQ ID NO: 121 - DNA, 934 bp, artificial
  Sequence of the second arm of the GyrB/ FusA/ MreB/HrpG/PhoP/FhaB dsRNA used to concomitantly target GyrB/ FusA/ MreB/HrpG/PhoP/FhaB genes from xanthomonas citri pv fuscan
ctgaccgggcgtctgccctgcagggttggcggttggcgcaacctgcgcccatgctggaaacgatgtccaggtggtcgtgcagcacagcaacagtgcaagcgcacgcttgccctgctcggccaggtaaccacgcacgcgcatcgcttcgttgtccattggacgatgatgccgacatcaaatggcacttcacggcccatgtagaggccttcttcgccgtcctgtgcagcgtcgaccgcaaagccttcgcgcttcaggcgcgccgcaagggtttcgcgcagcggagcttcgtcttcgaccaaaaggatacgcatgcttgaatgccgcagcgtccgcaaaagctccagttcatcggaaaaggtcgagacattgcgcgcaagcggtgcgaggctggcgttgacctgcgagaccaggcgcgcatcctgcgtcaacaggaacaccgatccggcgttggggggagagtggtcgttcatgcaccgcgtgccgccgatcgcgcgatcctgacgcaccgcaaccaccgacggctcgttcagcacgatgccctgtccgcgcacgtagatcagcgtattggccgtgcccaaatcgatggacaggtcgttggagaacatgccgcggagtttcttgaacatgcatcatcactgcagcgccgtcatggacttcgccgatcttgtgactcacaccggtgtagaacaggatgcgttcggacgtggtggtcttgccggcatcaatgtgggccatgatgccgaagttgcggtaacgctcgataggggtggtgcgggccacgatcacaccatgtgatgcaggccggtgccgtcatggacgtcgccgatatacatgccggggcgcttgcgaacggcttccaggccacgcagcacggtgatcttgctggagtcgtaggtgccgttgggtgttggcggggtgttttgttcgtcggtca

SEQ ID NO: 122 - DNA, 793 bp, artificial
  Sequence of the first arm of the GyrB/ RbfA/MreB dsRNA used to concomitantly target GyrB/ RbfA/ MreB genes from xanthomonas citri pv fuscan
atgaccgacgaacaaaacaccccgccaacacccaacggcacctacgactccagcaagatcaccgtgctgcgtggcctggaagccgttcgcaagcgccccggcatgtatatcggcgacgtccatgacggcaccggcctgcatcacatggtgttcgaggtggtcgacaactcggtcgacgaagcccttgccgggcatgccgacgacatcgtggtaaagatcatggccgatggctcggtggcagtttccgacaacgggcgcggcgtgccggatcatgccaaccaaatcattccatcgcaccgaccgcgtatctgcgcaggtgcgtcgcgacctcggcacgatcgtgcacgcggccgtgcgcgaccatggcctgccgtcggtcagcgtgtcagacgtcgaaatcagtcgcgacctggcgcacgccaaggtcttcgtcaccgcgctgcagcaggagcgttcggccgaagcggtcaaggggctcaaggaaatcgccggccagctgcgtacgcagttggcgcgcgcgaatgcatgttcaagaaactccgcggcatgttctccaacgacctgtccatcgatttgggcacggccaatacgctgatctacgtgcgcggacagggcatcgtgctgaacgagccgtcggtggttgcggtgcgtcaggatcgcgcgatcggcggcacgcgctcggtcgctgccgtcggtgcggaggccaagcagatgctcggccgtacccccggccacatcaccaccattcgcccgatgaaggacggcgtcatcgccgacttcacctacaccgaag

SEQ ID NO: 123 - DNA, 793 bp, artificial
  Sequence of the second arm of the GyrB/ RbfA/MreB dsRNA used to concomitantly target GyrB/ RbfA/ MreB genes from xanthomonas citri pv fuscan
cttcggtgtaggtgaagtcggcgatgacgccgtccttcatcgggcgaatggtggtgatgtggccgggggtacggccgagcatctgcttggcctccgcaccgacggcagcgaccgagcgcgtgccgccgatcgcgcgatcctgacgcaccgcaaccaccgacggctcgttcagcacgatgccctgtccgcgcacgtagatcagcgtattggccgtgcccaaatcgatggacaggtcgttggagaacatgccgcggagtttcttgaacatgcattcgcgcgcgccaactgcgtacgcagctggccggcgatttccttgagccccttgaccgcttcggccgaacgctcctgctgcagcgcggtgacgaagaccttggcgtgcgccaggtcgcgactgatttcgacgtctgacacgctgaccgacggcaggccatggtcgcgcacggccgcgtgcacgatcgtgccgaggtcgcgacgcacctgcgcagatacgcggtcggtgcgatggaatgatttggttggcatgatccggcacgccgcgcccgttgtcggaaactgccaccgagccatcggccatgatctttaccacgatgtcgtcggcatgcccggcaagggcttcgtcgaccgagttgtcgaccacctcgaacaccatgtgatgcaggccggtgccgtcatggacgtcgccgatatacatgccggggcgcttgcgaacggcttccaggccacgcagcacggtgatcttgctggagtcgtaggtgccgttgggtgttggcggggtgttttgttcgtcggtcat

SEQ ID NO: 124 - DNA, 779 bp, artificial
  Sequence of the first arm of the HrpG/PhoP/ FtsZ dsRNA used to concomitantly target HrpG/ PhoP/ FtsZ genes from xanthomonas citri pv fuscan
atgaacgaccactctccccccaacgccggatcggtgttcctgttgacgcaggatgcgcgcctggtctcgcaggtcaacgccagcctcgcaccgcttgcgcgcaatgtctcgaccttttccgatgaactggagcttttgcggacgctgcggcattcgccgtgcgagctgctgatcttcgatgccagctgtgtcgccgccgatgacagttcactgctggcctggcagcgctgccatagcggccagcccagatcatgcgtatccttttggtcgaagacgaagctccgctgcgcgaaacccttgcggcgcgcctgaagcgcgaaggctttgcggtcgacgctgcacaggacggcgaagaaggcctctacatgggccgtgaagtgccatttgatgtcggcatcatcgaccttggcttgcccaagatgtcggggatggagctgatcaaggccttgcgcgatgagggcaagaagtttccggtgctgatcctgaccgcgcgctcgagctggcaggacaatgcatggcacatttcgaactgattgaaaaaatggctcccaacgcggtcatcaaggtggttggcgtcggcggcggcggtggcaatgccgtcgcgcacatggtcaacaccaacgtcgatggcgtggaattcatcaccgccaacaccgactcgcaggcgatcaagaactgcggcgccaagctgcagctgcagctcggcaccaacgtcaccaagggcctgggcgcaggcgccaatccggaagtcggccgtcaggctgcgctggaagatcgcg

SEQ ID NO: 125 - DNA, 779 bp, artificial
  Sequence of the second arm of the HrpG/PhoP/ FtsZ dsRNA used to concomitantly target HrpG/ PhoP/ FtsZ genes from xanthomonas citri pv fuscan
cgcgatcttccagcgcagcctgacggccgacttccggattggcgcctgcgcccaggcccttggtgacgttggtgccgagctgcagctgcagcttggcgccgcagttcttgatcgcctgcgagtcggtgttggcggtgatgaattccacgccatcgacgttggtgttgaccatgtgcgcgacggcattgccaccgccgccgccgacgccaaccaccttgatgaccgcgttgggagccattttttcaatcagttcgaaatgtgccatgcattgtcctgccagctcgagcgcgcggtcaggatcagcaccggaaacttcttgccctcatcgcgcaaggccttgatcagctccatccccgacatcttgggcaagccaaggtcgatgatgccgacatcaaatggcacttcacggcccatgtagaggccttcttcgccgtcctgtgcagcgtcgaccgcaaagccttcgcgcttcaggcgcgccgcaagggtttcgcgcagcggagcttcgtcttcgaccaaaaggatacgcatgatctgggctggccgctatggcagcgctgccaggccagcagtgaactgtcatcggcggcgacacagctggcatcgaagatcagcagctcgcacggcgaatgccgcagcgtccgcaaaagctccagttcatcggaaaaggtcgagacattgcgcgcaagcggtgcgaggctggcgttgacctgcgagaccaggcgcgcatcctgcgtcaacaggaacaccgatccggcgttggggggagagtggtcgttcat

SEQ ID NO: 126 - DNA, 798 bp, artificial
  Sequence of the first arm of the FhaB/FusA/MreB dsRNA used to concomitantly target HrpG/ PhoP/ FtsZ genes from xanthomonas citri pv fuscan
atggacaacgaagcgatgcgcgtgcgtggttacctggccgagcagggcaagcgtgcgcttgcactgttgctgtgctgcacgaccacctggacatcgtttccagcatgggcgcaggttgcgccaaccgccaaccctgcagggcagacgcccggtcagcaggtcgctgccaatggtgtgccggtggtggacatcgtcgcgcccaatgcacgcggcatttcgcacaaccgctacagcaacttcaatgtcggcccgaatgggctgatcctcgatcgtggcccgcaccacccctatcgagcgttaccgcaacttcggcatcatggcccacattgatgccggcaagaccaccacgtccgaacgcatcctgttctacaccggtgtgagtcacaagatcggcgaagtccatgacggcgctgcagtgatggactggatggagcaggagcaggagcgtggtatcaccatcacctccgctgcgaccaccgcgttctggtcgggtatggacaagtccatgccgcagcaccgcttcaacatcatcgacacatgcatgttcaagaaactccgcggcatgttctccaacgacctgtccatcgatttgggcacggccaatacgctgatctacgtgcgcggacagggcatcgtgctgaacgagccgtcggtggttgcggtgcgtcaggatcgcgcgatcggcggcacgcgctcggtcgctgccgtcggtgcggaggccaagcagatgctcggccgtacccccggccacatcaccaccattcgcccgatgaaggacggcgtcatcgccgacttcac

SEQ ID NO: 127 - DNA, 798 bp, artificial
  Sequence of the second arm of the FhaB/FusA/MreB dsRNA used to concomitantly target FhaB/FusA/ MreB genes from xanthomonas citri pv fuscan
gtgaagtcggcgatgacgccgtccttcatcgggcgaatggtggtgatgtggccgggggtacggccgagcatctgcttggcctccgcaccgacggcagcgaccgagcgcgtgccgccgatcgcgcgatcctgacgcaccgcaaccaccgacggctcgttcagcacgatgccctgtccgcgcacgtagatcagcgtattggccgtgcccaaatcgatggacaggtcgttggagaacatgccgcggagtttcttgaacatgcatgtgtcgatgatgttgaagcggtgctgcggcatggacttgtccatacccgaccagaacgcggtggtcgcagcggaggtgatggtgataccacgctcctgctcctgctccatccagtccatcactgcagcgccgtcatggacttcgccgatcttgtgactcacaccggtgtagaacaggatgcgttcggacgtggtggtcttgccggcatcaatgtgggccatgatgccgaagttgcggtaacgctcgataggggtggtgcgggccacgatcgaggatcagcccattcgggccgacattgaagttgctgtagcggttgtgcgaaatgccgcgtgcattgggcgcgacgatgtccaccaccggcacaccattggcagcgacctgctgaccgggcgtctgccctgcagggttggcggttggcgcaacctgcgcccatgctggaaacgatgtccaggtggtcgtgcagcacagcaacagtgcaagcgcacgcttgccctgctcggccaggtaaccacgcacgcgcatcgcttcgttgtccat

SEQ ID NO: 128 - DNA, 784 bp, artificial
  Sequence of the first arm of the GyrB/ FusA/ZipA dsRNA used to concomitantly target GyrB, FusA, ZipA genes from xanthomonas hortorum pv vitians
atgaccgaagatcaaacaccccgaccacacccaacagcacctacgattccagcaagatcaccgtgctgcgtggcctggaagccgtccgcaagcgccccggcatgtacatcggcgatgtccatgacggcaccggcctgcatcacatggtgttcgaggtggtcgacaactcggtcgacgaggccctggccggccatgccgacgacatcgtggtcaagatcctggtcgatggctcggtcgccgtgtcggataacggccgcggatcgtggccgcaaccaacccctatcgagcgttaccgcaacttcggcatcatggctcacatcgatgccggcaagaccaccacctccgagcgcatcctgttttacaccggcgtcagtcacaagatcggcgaagtccatgacggcgccgcggtgatggactggatggagcaggagcaggagcgtggtatcaccatcacctccgctgccaccaccgcgttctggtcgggtatggacaagtccatgccgcagcatcgcctcaacatcatgcatgtccgaacatggccatgatccggatcgggatcctgatcgctggactgctgctggtcgcggccatcttcctgtttggtcgcccgaaaaaatcgccgcaagggcgccgggtcgacaaggacgacgggcagccgcgcgaacgccgcgagccggtgatttccggcgagcctggggtggagggcgacccgttcaagcgcactgaaggcgccgagcagagcgaactcaatctggacgatgccgatgcggccggcggcaacgat

SEQ ID NO: 129 - DNA, 784 bp, artificial
  Sequence of the second arm of the GyrB/ FusA/ZipA dsRNA used to concomitantly target GyrB, FusA, ZipA genes from xanthomonas hortorum pv vitians
atcgttgccgccggccgcatcggcatcgtccagattgagttcgctctgctcggcgccttcagtgcgcttgaacgggtcgccctccaccccaggctcgccggaaatcaccggctcgcggcgttcgcgcggctgcccgtcgtccttgtcgacccggcgcccttgcggcgattttttcgggcgaccaaacaggaagatggccgcgaccagcagcagtccagcgatcaggatcccgatccggatcatggccatgttcggacatgcatgatgttgaggcgatgctgcggcatggacttgtccatacccgaccagaacgcggtggtggcagcggaggtgatggtgataccacgctcctgctcctgctccatccagtccatcaccgcggcgccgtcatggacttcgccgatcttgtgactgacgccggtgtaaaacaggatgcgctcggaggtggtggtcttgccggcatcgatgtgagccatgatgccgaagttgcggtaacgctcgataggggttggttgcggccacgatccgcggccgttatccgacacggcgaccgagccatcgaccaggatcttgaccacgatgtcgtcggcatggccggccagggcctcgtcgaccgagttgtcgaccacctcgaacaccatgtgatgcaggccggtgccgtcatggacatcgccgatgtacatgccggggcgcttgcggacggcttccaggccacgcagcacggtgatcttgctggaatcgtaggtgctgttgggtgtggtcggggtgtttgatcttcggtcat

SEQ ID NO: 130 - DNA, 663 bp, artificial
  Sequence of the first arm of the avrPTO/avrPTOB/HopT1-1 dsRNA used to concomitantly target avrPTO/avrPTOB/HopT1-1 genes from pseudomonas syringae pto DC3000
atgggaaatatatgtgtcggcggatccaggatggcccatcaggtgaactccccagaccgagttagtaacaactcgggtgacgaagataacgtaacgtccagtcaactgctgagcgtcagacatcaacttgcggagtctgctggtgtaccaagagatcagcatgaatttgttagtaaccaagcacctcaaagcctgagaaaatggcgggtatcaatagagcgggaccatcgggcgcttattttgttggccacacagaccccgagccagtatcggggcaagcacacggatccggcagcggcgccagctcctcgaacagtccgcaggttcagccgcgaccctcgaatactcccccgtcgaacgcgcccgcaccgccgccaaccggacgtgagaggctttcacgatgcatgaaaacagtcagcaatcactcgatacccagtacaaatctcgtcgtggatgcgggaacggaaacttcggcgcagaaatcccagccggtttgcagcgaaatccagcgtaacagcaagatcgaaaaagcagtcatcgaacacattgccgaccacccggcagcgaaaatgacaataagcgcgctggttgacacgttgacagacgtttttgtcagggctcatggggaggttaaggggtgggccgaaatcgtccaggcagtct

SEQ ID NO: 131 - DNA, 663 bp, artificial
  Sequence of the second arm of the avrPTO/avrPTOB/HopT1-1 dsRNA used to concomitantly target avrPTO/avrPTOB/HopT1-1 genes from pseudomonas syringae pto DC3000
agactgcctggacgatttcggcccaccccttaacctccccatgagccctgacaaaaacgtctgtcaacgtgtcaaccagcgcgcttattgtcattttcgctgccgggtggtcggcaatgtgttcgatgactgctttttcgatcttgctgttacgctggatttcgctgcaaaccggctgggatttctgcgccgaagtttccgttcccgcatccacgacgagatttgtactgggtatcgagtgattgctgactgttttcatgcatcgtgaaagcctctcacgtccggttggcggcggtgcgggcgcgttcgacgggggagtattcgagggtcgcggctgaacctgcggactgttcgaggagctggcgccgctgccggatccgtgtgcttgccccgatactggctcggggtctgtgtggccaacaaaataagcgcccgatggtcccgctctattgatacccgccattttctcaggctttgaggtgcttggttactaacaaattcatgctgatctcttggtacaccagcagactccgcaagttgatgtctgacgctcagcagttgactggacgttacgttatcttcgtcacccgagttgttactaactcggtctggggagttcacctgatgggccatcctggatccgccgacacatatatttcccat

SEQ ID NO: 132 - DNA, 668 bp, artificial
  Sequence of the first arm of the rpoB/rpoC/fusA dsRNA used to concomitantly target the rpoB, RpoC and fusA genes from Pto DC3000
cgtatccgcaaggactttagcaagttgccggacgtaatggatgtgccgtatctcttggccatccagctggattcgtatcgcgaattcctgcaggcgggagcgaccaaagatcagttccgcgacgtcggtctgcatgcagccttcaaatccgttttcccgatcatcagctactccggcaatgctgcgctggagtatgtaggttatcgcttgggcgagatcttgaaagacctactgaatttgctgaaaaaccagggtcaagtcgaagagttcgacgcaatccgtatcggacttgcatcgcctgagatgatccgctcttggtcgttcggtgaagttaaaaagccggaaaccatcaactaccgtacgttcaagcctgagcgtgacggtctgttctgcgccaagatctttggtccggtaaaagattacgagtgcctgtgcggtaagtacaagatgcatggctcgtactactccgattagccgttaccgtaacatcggtatcgtcgctcacgtggatgctggtaaaaccaccaccaccgagcgcgtccttttttacaccggcaaaagccacaaaatgggcgaggtgcatgatggcgccgcgaccacggactggatggttcaggagcaggagcgtggtattaccattacttctgctgctatcactgccttttggc

SEQ ID NO: 133 - DNA, 668 bp, artificial
  Sequence of the second arm of the rpoB/rpoC/fusA dsRNA used to concomitantly target the rpoB, RpoC and fusA genes from Pto DC3000
gccaaaaggcagtgatagcagcagaagtaatggtaataccacgctcctgctcctgaaccatccagtccgtggtcgcggcgccatcatgcacctcgcccattttgtggcttttgccggtgtaaaaaaggacgcgctcggtggtggtggttttaccagcatccacgtgagcgacgataccgatgttacggtaacggctaatcggagtagtacgagccatgcatcttgtacttaccgcacaggcactcgtaatcttttaccggaccaaagatcttggcgcagaacagaccgtcacgctcaggcttgaacgtacggtagttgatggtttccggctttttaacttcaccgaacgaccaagagcggatcatctcaggcgatgcaagtccgatacggattgcgtcgaactcttcgacttgaccctggtttttcagcaaattcagtaggtctttcaagatctcgcccaagcgataacctacatactccagcgcagcattgccggagtagctgatgatcgggaaaacggatttgaaggctgcatgcagaccgacgtcgcggaactgatctttggtcgctcccgcctgcaggaattcgcgatacgaatccagctggatggccaagagatacggcacatccattacgtccggcaacttgctaaagtccttgcggatacg

SEQ ID NO: 134 - DNA, 673 bp, artificial
  Sequence of the first arm of the secE/rpoA/rplQ dsRNA used to concomitantly target the secE, RpoA and rplQ genes from Pto DC3000
tgaatcccaaggctgaagcatcagactctcgctttgatttgctgaaatggctcctggtagtcattttggtagtcgtgggtgttgtcggtaatcagtattactctgctgagccgatcctgtaccgtgttctcgctctccttgtgattgccgcagcagctgcatttgtagcgctgcagactggcaagggcaaagctttcttcgttttggcgaaagaagcgcgtgcagagatgatctgcagatttcggtaaatgagttcctgacaccccgccacattgatgtgcaggttgtcagtccaacccgcgccaaaatcactctcgagcctctcgagcgtggtttcggccataccctgggcaacgcgcttcgccgcattttgttgtcctccatgcccggctgtgcagtagtcgaggccgagattgacggtgtactccatgagtacagcgccatcgaaggtgtaatgcatgcgtcatcgtaaaagtggacgtcacctgagccgtaccagctctcaccgtaaagccatgtttcaaaacatggcggtgtcgctgttcgagcacgagctgatcaaaactaccctgccgaaagccaaggaactgcgccgcgttgccgagccgctgatcaccctggccaaggaagacagtgttgctaaccgtcgtctggctttcgaccgtactcgttc

SEQ ID NO: 135 - DNA, 673 bp, artificial
  Sequence of the second arm of the secE/rpoA/rplQ dsRNA used to concomitantly target the secE, RpoA and rplQ genes from Pto DC3000
gaacgagtacggtcgaaagccagacgacggttagcaacactgtcttccttggccagggtgatcagcggctcggcaacgcggcgcagttccttggctttcggcagggtagttttgatcagctcgtgctcgaacagcgacaccgccatgttttgaaacatggctttacggtgagagctggtacggctcaggtgacgtccacttttacgatgacgcatgcattacaccttcgatggcgctgtactcatggagtacaccgtcaatctcggcctcgactactgcacagccgggcatggaggacaacaaaatgcggcgaagcgcgttgcccagggtatggccgaaaccacgctcgagaggctcgagagtgattttggcgcgggttggactgacaacctgcacatcaatgtggcggggtgtcaggaactcatttaccgaaatctgcagatcatctctgcacgcgcttctttcgccaaaacgaagaaagctttgcccttgccagtctgcagcgctacaaatgcagctgctgcggcaatcacaaggagagcgagaacacggtacaggatcggctcagcagagtaatactgattaccgacaacacccacgactaccaaaatgactaccaggagccatttcagcaaatcaaagcgagagtctgatgcttcagccttgggattca

SEQ ID NO: 136 - DNA, 217 bp, artificial
  Sequence of the first arm of the fusA dsRNA used to target the fusA gene from Pto DC3000
atggctcgtactactccgattagccgttaccgtaacatcggtatcgtcgctcacgtggatgctggtaaaaccaccaccaccgagcgcgtccttttttacaccggcaaaagccacaaaatgggcgaggtgcatgatggcgccgcgaccacggactggatggttcaggagcaggagcgtggtattaccattacttctgctgctatcactgccttttggc

SEQ ID NO: 137 - DNA, 217 bp, artificial
  Sequence of the second arm of the fusA dsRNA used to target the fusA gene from Pto DC3000
gccaaaaggcagtgatagcagcagaagtaatggtaataccacgctcctgctcctgaaccatccagtccgtggtcgcggcgccatcatgcacctcgcccattttgtggcttttgccggtgtaaaaaaggacgcgctcggtggtggtggttttaccagcatccacgtgagcgacgataccgatgttacggtaacggctaatcggagtagtacgagccat

SEQ ID NO: 138 - DNA, 221 bp, artificial
  Sequence of the first arm of the gyrB dsRNA used to target the gyrB gene from Pto DC3000
agcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcga

SEQ ID NO: 139 - DNA, 221 bp, artificial
  Sequence of second arm of the gyrB dsRNA used to target the gyrB gene from Pto DC3000
tcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgct

SEQ ID NO: 140 - DNA, 442 bp, artificial
  Sequence of the first arm of the fusA/gyrB dsRNA used to concomitantly target the fusA and gyrB genes from Pto DC3000
atggctcgtactactccgattagccgttaccgtaacatcggtatcgtcgctcacgtggatgctggtaaaaccaccaccaccgagcgcgtccttttttacaccggcaaaagccacaaaatgggcgaggtgcatgatggcgccgcgaccacggactggatggttcaggagcaggagcgtggtattaccattacttctgctgctatcactgccttttggcgatcagcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcga

SEQ ID NO: 141 - DNA, 442 bp, artificial
  Sequence of the second arm of the fusA/gyrB dsRNA used to concomitantly target the fusA and gyrB genes from Pto DC3000
tcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgctgatcgccaaaaggcagtgatagcagcagaagtaatggtaataccacgctcctgctcctgaaccatccagtccgtggtcgcggcgccatcatgcacctcgcccattttgtggcttttgccggtgtaaaaaaggacgcgctcggtggtggtggttttaccagcatccacgtgagcgacgataccgatgttacggtaacggctaatcggagtagtacgagccat

SEQ ID NO: 142 - DNA, 229 bp, artificial
  Sequence of the first arm of the secE dsRNA used to target the secE gene from Pto DC3000
tgaatcccaaggctgaagcatcagactctcgctttgatttgctgaaatggctcctggtagtcattttggtagtcgtgggtgttgtcggtaatcagtattactctgctgagccgatcctgtaccgtgttctcgctctccttgtgattgccgcagcagctgcatttgtagcgctgcagactggcaagggcaaagctttcttcgttttggcgaaagaagcgcgtgcagagat

SEQ ID NO: 143 - DNA, 229 bp, artificial
  Sequence of the second arm of the secE dsRNA used to target the secE gene from Pto DC3000
atctctgcacgcgcttctttcgccaaaacgaagaaagctttgcccttgccagtctgcagcgctacaaatgcagctgctgcggcaatcacaaggagagcgagaacacggtacaggatcggctcagcagagtaatactgattaccgacaacacccacgactaccaaaatgactaccaggagccatttcagcaaatcaaagcgagagtctgatgcttcagccttgggattca

SEQ ID NO: 144 - DNA, 475 bp, artificial
  Sequence of the first arm of the gyrB/hrpL dsRNA used to concomitantly target the gyrB and hrpL genes from Pto DC3000
agcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcgagatcctgatgactgacatcaccgtgcccttccagctcggaatgcacttcgtcttcccagctttcctgatacggctgacgatacattttgcggaagtgattgcggatcaggttcagcgcgatgccacacagccaggtctgcggtttgctggcatgttgaaacttgtgctcgttacgcagggcttcaagaaacacgcactggagaatgtcatccacatcatcagggttcatcacccgcttttggataaacgccctg

SEQ ID NO: 145 - DNA, 475 bp, artificial
  Sequence of the second arm of the gyrB/hrpL dsRNA used to concomitantly target the gyrB and hrpL genes from Pto DC3000
cagggcgtttatccaaaagcgggtgatgaaccctgatgatgtggatgacattctccagtgcgtgtttcttgaagccctgcgtaacgagcacaagtttcaacatgccagcaaaccgcagacctggctgtgtggcatcgcgctgaacctgatccgcaatcacttccgcaaaatgtatcgtcagccgtatcaggaaagctgggaagacgaagtgcattccgagctggaagggcacggtgatgtcagtcatcaggatctcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgct

SEQ ID NO: 146 - DNA, 673 bp, artificial
  Sequence of the first arm of the dnaA/dnaN/gyrB dsRNA used to concomitantly target the dnaA, dnaN and gyrB genes from Pto DC3000
gtgtcagtggaactttggcagcagtgcgtggagcttttgcgcgacgagctgccggcccagcaattcaacacttggatccgtccgctacaggtcgaagccgaaggcgacgagttgcgtgtgtacgcacccaaccgctttgttctcgattgggtcaatgaaaaatacctcggccgtttgcttgagcttctcggcgaacgtggtcaaggcatggcgcccgctgatcatgcatttcaccattcaacgcgaagccctgttgaaacccctgcaattggtcgccggcgtggtagaacgccgccagaccttgccggttctttccaatgtgcttctggtcgtgcaaggccagcagctgtcgctgaccggtactgacctggaagtcgagctggttggccgtgttcagctggaagagcctgccgaacctggggaaatcaccgttcccgcgcgcaagcttatgcagcgaaaaccaaacgtacgactcgtccagcattaaagtgctgaaaggacttgatgccgtacgcaagcgtcccggaatgtacatcggtgatacagacgatggcagcggtctgcaccatatggtgttcgaggtggttgataactcgatcgacgaagcattggcaggccattgcgacgacatcagcatcatcatccatccagacgaatctatcaccgtgcgcga

SEQ ID NO: 147 - DNA, 673 bp, artificial
  Sequence of the second arm of the dnaA/dnaN/gyrB dsRNA used to concomitantly target the dnaA, dnaN and gyrB genes from Pto DC3000
tcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgctgcataagcttgcgcgcgggaacggtgatttccccaggttcggcaggctcttccagctgaacacggccaaccagctcgacttccaggtcagtaccggtcagcgacagctgctggccttgcacgaccagaagcacattggaaagaaccggcaaggtctggcggcgttctaccacgccggcgaccaattgcaggggtttcaacagggcttcgcgttgaatggtgaaatgcatgatcagcgggcgccatgccttgaccacgttcgccgagaagctcaagcaaacggccgaggtatttttcattgacccaatcgagaacaaagcggttgggtgcgtacacacgcaactcgtcgccttcggcttcgacctgtagcggacggatccaagtgttgaattgctgggccggcagctcgtcgcgcaaaagctccacgcactgctgccaaagttccactgacac

SEQ ID NO: 148 - DNA, 758 bp, artificial
  Sequence of the second arm of the HrpR/ GyrB/ RpoD dsRNA used to concomitantly target HrpR, GyrB, RpoD genes from pseudomonas syringae pv actinidiae
cagccgcttcttctgcggctgcctcgtcggtatcggcgtcggccagcatcaaggcgtccgcatccggagcactctcgtgtaccgggatccccatgtcgttgatcatgcggatgatgtcttccacctgctcgggatctgaaatatcctcgggcaggtggtcgttgacctctgcgtaagtcagatatttctgctcacgacccagggtgatcaactctttgatacgagactgctgttgcgcttttccggacatgcatcgtctaccggaataccgcggccgttgtcgcgcacggtgatagattcgtctggatggatgatgatgctgatgtcgtcgcaatggcctgccaatgcttcgtcgatcgagttatcaaccacctcgaacaccatatggtgcagaccgctgccatcgtctgtatcaccgatgtacattccgggacgcttgcgtacggcatcaagtcctttcagcactttaatgctggacgagtcgtacgtttggttttcgctcatgatcccaggtacaacgtgccaccactggaggcctctatgtagccctcgcgagcccggcatacgccggtgaatgcgccgttgaccacaccgaataactggctctctgccagcgactcgggaatggctgcgcagttcatgcccacaaagggtcccgacctgctggacaactcgtgaatgcggttggccagtgtgtccttgccggtgccggtttccccgcacaacagcaagtccatatccaggaacgcgctattcat

SEQ ID NO: 149 - DNA, 1410 bp, artificial
  Sequence of a specific intron sequence from the Petunia Chalcone synthase gene CHSA
ggcgcgcccaatcgatgatttaaatgtgtaagaatttcttatgttacattattacattcaacgttttatcttaattggctcttcatttgattgaaatttgacaattatttcttgtttttttttttgtcacactctttttgggttggggtggccgacgaattgtgggaaggtagaaagaggggaggacttttgttatactccattagtaattactgtttccgtttcaatttatgtgacaatatttcctttttagtcggttccaaaagaaaatgtcagcattataaacaatttaattttgaaattacaattttgccattaataaaatgatttacaaccacaaaagtatctatgagcctgtttgggtgggcttataagcagcttattttaagtggcttataagtcaaaaagtgacattttttgagaagttagaaaatcctaacttctcaaaaagtagcttttaagccacttatgacttataagtccaaaaatttttaagttaccaaacatatattaatgggtttataagcttataagccacttttaagctcacccaaacgggttctatgtctcactttagactacaaattttaaaagtcttcatttatttcttaatctccgtggcgagtaaaactataacacataaagtgaaacggagggaataagatggagtcataaactaatccaaatctatactctctccgttaatttgttttttagtttgatttggtacattaataaaacagatttttcgaaggttataaacacagacagatgtttcccagcgagctagcaaaattccaagatttctgtcgaaaattcgtgtgtttctagctagtacttgatgttatctttaaccttttagtaattttttgtccttttctttctatttttcatcttacaatgaattatgagcaagttccttaagtagcatcacacgtgagatgttttttatgatattgactaaatccaatctttaccattccttaactagtaaaatacaacacatgttaattgatacattgcttaacactgaggttagaaaattttagaaattagttgtccaaatgctttgaaattagaaatctttaatcccttatttttttttaaaatgttttttctcactccaaagaaagagaaactgacatgaaagctcaaaagatcatgaatcttactaactttgtggaactaaatgtacatcagaatgtttctgacatgtgaaaatgaaagctcttaattttcttcttttatttattgagggtttttgcatgctatgcattcaatttgagtactttaaagcacctataaacacttacttacacttgccttggagtttatgttttagtgttttcttcacatcttttttggtcaatttgcaggtatttggatcctaggtgagtctagaggcgcgcc

(19) United States
(12) Patent Application Publication
(10) Pub. No.: US 2024/0294864 A1
(43) Pub. Date: Sep. 5, 2024

(54) CHLORELLA-BASED PRODUCTION OF EXTRACELLULAR VESICLE-EMBEDDED SMALL RNAS FOR BIOCONTROL APPLICATIONS

(71) Applicants: IMMUNRISE, PARIS (FR); CENTRE NATIONAL DE LA RECHERCHE SCIENTIFIQUE (CNRS), Paris (FR)

(72) Inventors: Lionel NAVARRO, Antony (FR); Jerome ZERVUDACKI, BOURG-LA-REINE (FR); Magali CHARVIN, Paris (FR); Antonio Emidio FORTUNATO, Bagnolet (FR)

(21) Appl. No.: 18/044,891

(22) PCT Filed: Sep. 13, 2021
(86) PCT No.: PCT/EP2021/075121

§ 371 Date: Mar. 10, 2023

(30) Foreign Application Priority Data
Sep. 11, 2020 (EP) 20306016.5

(51) Int. Cl. C12N 1/12; A01N 63/60; A01N 65/03; A01P 1/00; C12N 15/11

(52) U.S. Cl. CPC C12N 1/12; A01N 63/60; A01N 65/03; A01P 1/00; C12N 15/111; C12N 2310/14; C12N 2330/50

(57) ABSTRACT

The invention relates to a novel method to produce small RNAs targeting virulence factors, essential genes and/or antimicrobial resistance genes of phytopathogens. More specifically, the invention involves the expression of exogenous RNA interference (RNAi) precursor(s) in Chlorella cells, which in turn express and release Extracellular Vesicle (EV)-embedded antimicrobial small RNAs. These EVs can be collected from the cell-free medium of Chlorella cultures, and further concentrated and purified for biocontrol applications. Importantly, Chlorella EVs protect small RNAs from ribonuclease-mediated digestion, indicating that these lipid-based particles not only act as natural vectors of small RNAs towards pathogenic cells, but also presumably limit their degradation in the environment. The invention can thus likely be used to reduce the pathogenicity and growth of a wide range of pathogens or, potentially, to enhance beneficial effects and growth of plant-associated symbiotic and commensal microbes. Furthermore, because the integrity of Chlorella EV-embedded antimicrobial siRNAs remains unaltered when produced in photobioreactors, and when stored frozen, this method has the potential to be further exploited for the industrialization and manufacturing of a novel generation of microalgae-based biologicals.

### SEQUENCE LISTING

The instant application contains a Sequence Listing, which has been submitted electronically in ASCII format and is hereby incorporated by reference in its entirety. Said ASCII copy, created on Sep. 21, 2023, is named 18044891_sequence.txt and is 187,036 bytes in size.

### SUMMARY OF THE INVENTION

The invention relates to a novel method to produce small RNAs targeting virulence factors, essential genes and/or antimicrobial resistance genes of phytopathogens. More specifically, the invention involves the expression of exogenous RNA interference (RNAi) precursor(s) in Chlorella cells, which in turn express and release Extracellular Vesicle (EV)-embedded and/or-associated antimicrobial small RNAs. These EVs can be collected from the cell-free medium of Chlorella cultures, and further concentrated and purified for biocontrol applications. Importantly, Chlorella EVs protect small RNAs from ribonuclease-mediated digestion, indicating that these lipid-based particles not only act as natural vectors of small RNAs towards pathogenic cells, but also presumably limit their degradation in the environment. The invention can thus likely be used to reduce the pathogenicity and growth of a wide range of pathogens or, potentially, to enhance beneficial effects and growth of plant-associated symbiotic and commensal microbes. Furthermore, because the integrity of Chlorella EV-embedded and/or—associated antimicrobial siRNAs remains unaltered when produced in photobioreactors, and when stored frozen, this method has the potential to be further exploited for the industrialization and manufacturing of a novel generation of microalgae-based biologicals.

### PRIOR ART DESCRIPTION

### RNA Silencing Controls Plant-Pathogen Interactions

In plants, mobile small RNAs can trigger non-cell autonomous silencing in adjacent cells as well as in distal tissues (Melynk et al., 2011). They are notably important to prime antiviral defense ahead of the infection front (Melynk et al., 2011). Non-cell autonomous silencing is also critical for the translocation of silencing signals between plant cells and their interacting non-viral pathogenic, parasitic or symbiotic organisms (Baulcombe, 2015). This natural cross-kingdom regulatory mechanism has been notably recently characterized in plant-fungal interactions (Weiberg et al., 2015, Koch et al., 2014, Zhang et al., 2016, Wang et al., 2016, Weiberg et al., 2013, Cai et al., 2018). For instance, specific plant miRNAs were found to be exported into the hyphae of the fungal pathogen Verticillium dahliae to trigger silencing of virulence factors (Zhang et al., 2016, Cai et al., 2018). On the other hand, endogenous B. cinerea small RNAs can be exported into plant cells to silence plant defense genes (Weiberg et al., 2013), highlighting bi-directional cross-kingdom RNAi between plant and fungal pathogens.

Although very little is known about the mechanisms of small RNA/dsRNA trafficking between host cells and fungal cells, the presence of numerous vesicles in the extrahaustorial matrix suggests that they may transfer silencing signals between the two organisms (Micali et al., 2011). Consistent with this hypothesis, two recent studies provide evidence that plant extracellular vesicles (EVs) are essential to deliver antifungal small RNAs into B. cinerea cells as well as anti-oomycete small RNAs into Phytophthora capsici cells (Cai et al., 2018, Koch et al., 2013).

### Cross-Kingdom RNAi can be Exploited to Confer Protection Against Pathogens Possessing a Canonical RNA Silencing Machinery

The biological relevance of cross-kingdom RNAi has been initially demonstrated by expressing dsRNAs bearing homologies to vital or pathogenicity factors from a given parasite or pest provided that they possess a canonical RNAi machinery (e.g., functional DCL and AGO proteins). So far, this Host-Induced Gene Silencing (HIGS) technology has been successfully used to protect plants from invasion and predation of insects, nematodes, oomycetes, fungi, bacteria and parasitic plants (Singla-Rastogi, Navarro, PCT/EP2019/072169, PCT/EP2019/072170; WO 2012/155112, WO 2012/155109, CA 2 799 453, EP 2 405 013, US 2013/177539, Wang et al., 2016, Koch et al., 2016, Wang et al., 2017). For example, HIGS confers full protection against Fusarium graminearum and B. cinerea and this phenomenon is fully recapitulated by spraying relevant exogenous dsRNAs or siRNAs onto wild type plants prior fungal infections (Wang et al., 2016, Koch et al., 2016, Wang et al., 2017). The latter phenomenon is referred to as Spray-Induced Gene Silencing (SIGS) and is reminiscent of ‘environmental RNAi’, a process involving the uptake of RNAs from the environment initially described in Caenorhabditis elegans and in some insects (Wang et al., 2016, Wang et al., 2017, Lacombe et al., 2010). HIGS/SIGS is thus considered as a powerful complement, or even sometimes an alternative, to conventional breeding or genetic engineering designed to introduce disease resistance genes in agriculturally relevant crops (Jones et al., 2014, Mansfield et al., 2012, Escobar et al., 2001). Furthermore, this technology provides a more durable and environmentally friendly plant protection solution that will likely contribute to a reduced use of agrochemicals, which can have, in some instances, significant impact on human health and on the environment.

However, for the industrialization and manufacturing of such RNA-based biologicals, there is a need to establish methods to produce, at a high-throughput level and in a cost-effective manner, vectorized small RNAs conferring efficient protection towards phytopathogens.

The present invention aims to fulfill this need. As a matter of fact, it is herein proposed a method to produce high amounts of EV-embedded small RNAs that can be effective on a wide range of phytopathogen(s), and that can be applied on various plant tissues, prior to and/or after an infection. It can also be delivered into woody plants by trunk injection or petiole absorption and systemically transported towards the targeted vascular phytopathogen(s). This approach will therefore have major agricultural applications in disease management through the use of a novel generation of microalgae-based biologicals.

Furthermore, as EV-embedded and/or-associated small RNAs were found resistant to ribonuclease-mediated digestion, it is anticipated that this method will be suitable to protect the integrity of antimicrobial small RNAs in the environment, before being internalized by the targeted pathogen(s), in which they will trigger the desired anti-virulence and/or anti-survival effects.

In addition, because Chlorella can be engineered to produce siRNA populations, bearing sequence homologies to large portions of virulence factors, essential genes and/or antimicrobial resistance genes of the targeted pathogen(s), the proposed approach should confer durable resistance. This is a major distinction from classical pesticides, which often become ineffective within a few years due to pathogen escape mutations.

Finally, it can be anticipated that the herein described technology will also be useful to control the expression of genes from beneficial microbes in order to enhance their multiplication and/or their beneficial effects towards host plants.

### DETAILED DESCRIPTION OF THE INVENTION

### Overview of the Results and the Invention

In the results below, the Inventors herein provide the first evidence that Chlorella cells can produce extracellular vesicles (EVs). They also demonstrate for the first time that Chlorella can be engineered to produce biologically active antibacterial small RNAs that are embedded into, and/or associated with, EVs. More specifically, by transforming C. vulgaris with inverted repeat transgenes bearing sequence homology with key virulence factors from a phytopathogenic bacterium, they show that Chlorella EVs are competent in delivering effective small RNAs in bacterial cells, resulting in the dampening of their pathogenicity. Furthermore, they show that Chlorella EVs efficiently protect these antibacterial small RNAs from digestion mediated by the non-specific micrococcal nuclease. These data therefore highlight the tremendous potential of Chlorella EVs as vehicles of small RNAs towards bacterial pathogens. Furthermore, because plant EVs are known to deliver effective antimicrobial small RNAs in phytopathogenic fungi and oomycetes (Cai et al., 2018; Hou et al., 2019), it is anticipated that Chlorella EVs will be employed to transfer active small RNAs in a wide range of phytopathogens including bacterial, fungal and oomycetal organisms.

A pre-requisite for the industrialization of Chlorella EV-embedded small RNA products is to demonstrate that they can maintain a full integrity when produced in photobioreactors (PBRs). To address this issue, the inventors have first grown a Chlorella reference line producing antibacterial siRNAs in a PBR of one liter, collected the corresponding extracellular medium, which was further stored frozen. The extracellular medium was subsequently thawed and subjected to filtration and ultracentrifugation, to recover purified EVs. Importantly, these Chlorella EVs were found to exhibit a normal size distribution. Furthermore, comparable results were obtained when the same Chlorella transgenic line was grown in a PBR of 150 liters, except that in those conditions, the yield of recovered EV particles was ˜20 times enhanced compared than the ones collected from flasks or 1 L PBRs. Furthermore, we found that the antibacterial activity of Chlorella EV-embedded and/or-associated small RNAs, recovered from 1 L and 150 L PBRs, was found unaltered. These findings indicate that the integrity and functionality of Chlorella EV-embedded and/or-associated small RNAs remain unaltered when produced in PBRs, despite being stored frozen. The Microalgae-Induced Gene Silencing (MIGS) technology is thus suitable for the production of EV-embedded antimicrobial small RNAs in PBRs.

Another pre-requisite for the industrialization of Chlorella EV-embedded antimicrobial small RNAs, is to verify—in a rapid, reliable and cost-effective manner—the efficacy of each batch produced from PBRs. To address this issue, the inventors have designed and engineered bacterially expressed small RNA reporter systems, which rely on the differential fluorescence or bioluminescence signal detection in the presence of effective Chlorella EV-embedded and/or-associated antimicrobial small RNAs. These quantitative reporters can be easily generated and manipulated to ensure that each batch produced is highly active prior product manufacturing.

They can additionally be used to rapidly select independent Chlorella transgenic lines expressing active EV-embedded and/or-associated small RNA species. Based on all these discoveries, the present Inventors propose to use this MIGS technology to produce Chlorella EV-embedded small RNAs directed against any phytopathogen(s) of interest. More precisely, they propose a method to produce high yields of Chlorella EV-embedded small RNAs targeting one or multiple target pathogen(s), i) by expressing iRNA molecules (precursors of siRNAs and miRNAs) in Chlorella cells, ii) collecting the EVs released by said Chlorella cells, iii) verifying the efficacy of Chlorella EV-embedded siRNAs prior product manufacturing, and iv) delivering the concentrated or purified EV products on plants (i.e. on leaf surface, seeds or in the vasculature, notably through trunk injection or petiole absorption in the case of woody plants). It is noteworthy that during such production pipeline, both the extracellular medium carrying the effective EVs, or purified EVs, can be stored frozen without major negative impact on the integrity and functionality of these EV-based anti-infective agents.

It is therefore anticipated that the MIGS technology will be extensively used for biocontrol applications against a wide spectrum of phytopathogens.

### The Following Features and Advantages of the Methods of the Invention are Worth to be Mentioned

### 1) Chlorella is an Ideal Biological System for the Production of Endogenous and Heterologous Molecules:

Chlorella belongs to a group of green microalgae (Chlorophyta, Trebuxiophyceae) able to adapt and grow in a variety of conditions. Chlorella is easy to maintain in laboratory conditions, possess a simple life cycle, a haploid genome and metabolic pathways similar to higher plants (Blanc et al., 2010). It also possesses the capacity to grow in auto-, hetero- or mixo-trophic conditions with high growth rates (Zuniga et al., 2016). The metabolic flexibility, the ease of maintenance and growth are features that enable Chlorella to be exploited as industrial production scaffold in PBRs for a variety of molecules of interest. In particular, Chlorella cells can be easily transformed with a disarmed Agrobacterium tumefaciens (Cha et al., 2012), and stable transformed transgenic lines can be selected within a 2 months period. This exceptional rapid selection process positions Chlorella as an ideal biological system to produce within a short timeframe any construct of interest. This feature is notably valuable in the context of outbreak situations, as Chlorella can be exploited to rapidly produce vectorized small RNAs against virulence factors, essential genes and/or antimicrobial resistance genes from any plant pathogen(s) of interest (which can nowadays be sequenced within a few days).

To summarize, the possibility to rapidly transform Chlorella with a transgene of interest, and to obtain large volumes of Chlorella extracellular media from PBRs, shows that this green microalga is suitable for the industrial production of EV-embedded and/or-associated antimicrobial small RNAs.

### 2) The MIGS Technology is Highly Versatile and Sequence-Specific.

The MIGS technology relies on the stable expression of inverted repeat, artificial miRNAs or sense-antisense, transgenes in Chlorella, which will be processed into siRNAs or miRNAs by the endogenous Dicer-like enzyme, and/or other endogenous RNases, and further internalized into EVs. The MIGS technology can also rely on the production of RNAi precursors from recombinant viruses that can infect Chlorella cells and likewise generate high yields of siRNA populations through Virus-Induced Gene Silencing (VIGS), as previously described in plants. These transgene- and viral-based RNAi precursors can notably be designed in such a way that they will target one or multiple genes of interest and trigger their selective silencing. This feature is particularly valuable for controlling the replication of one or multiple plant pathogens, while having no side effects on the cultivated plants, their associated-commensal microbes, or the animals that feed on those plants, including humans.

### 3) The MIGS Technology can be Used to Produce Antimicrobial Small RNA Populations, Likely Conferring Durable Disease Resistance.

Chlorella can be employed to produce small RNA populations targeting up to 1500 bp long regions from a single gene or up to a dozen genes. Chlorella is thus well-suited to produce small RNAs covering large portion of microbial gene(s), thereby maximizing the chance of detecting a potent silencing effect towards the targeted microbial gene(s). Furthermore, by targeting long sequence regions, the microbe will unlikely be able to mutate all along the targeted region, thereby resulting in long-lasting protection effects against the targeted plant pathogen(s). The MIGS technology is thus expected to overcome the recurrent problem of pathogen-directed escaping mutations and is therefore expected to confer durable disease resistance.

4) The MIGS Technology is Effective Against Prokaryotic Cells, which is not the Case of Other Platforms Producing Long dsRNAs.

The present inventors have previously reported that the exogenous application of siRNAs can target, in a sequence-specific manner, virulence factors in bacterial pathogens (Singla-Rastogi, Navarro, PCT/EP2019/072169, PCT/EP2019/072170). By contrast, long dsRNAs were not active in this process, suggesting that they are either not taken-up by, or not active in, bacterial cells. This is a major distinction from environmental RNAi previously reported in nematodes and plant herbivores, which exclusively relies on long dsRNAs (Bolognesi et al., 2012; Ivashuta et al., 2015; Whangbo et al., 2008), or in fungi and oomycetes, which is dependent on both small RNA and long dsRNA species (Koch et al., 2016; Wang et al., 2016). By producing small RNA species, the MIGS technology has therefore the potential of being exploited as a new production scaffold for antibacterial agents, which is not the case of other industrial biological systems currently exploited to produce long dsRNAs as fungicides, insecticides or nematicides for agricultural applications. The MIGS technology can also be employed to selectively target genes from symbiotic or commensal bacteria to enhance their beneficial effects for host plants. MIGS is therefore a unique RNAi production scaffold technology that can be exploited towards prokaryotic cells, and likely many other unrelated microbes and pests.

### Definitions

As used herein, the term “functional interfering RNA” (functional iRNA) refers to a RNA molecule capable of inducing the process of sequence-specific silencing of at least one gene(s). In particular, said functional interfering RNA molecule can be either i) a small interfering RNA, well-known in the art as small or short interfering RNA (siRNA) molecule (simplex or duplex), or a precursor thereof, or ii) a microRNA (miRNA) molecule (simplex or duplex) or a precursor thereof.

iRNA is a conserved gene regulatory mechanism that promotes antiviral resistance in plants, flies, worms and mammals (Guo et al., 2019). The core mechanism of antiviral silencing involves the recognition and processing of viral double-stranded RNAs (dsRNAs) by the RNAse III enzyme DICER leading to the production of 20-25 nt long short interfering RNA (siRNA) duplexes. These siRNA duplexes subsequently bind to a central component of the RNA Induced Silencing Complex (RISC), namely the Argonaute (AGO) protein, and one strand, the guide, remains bound to AGO to silence post-transcriptionally complementary viral transcripts. Recent studies have shown that plant and/or animal endogenous small RNAs can additionally directly target virulence or essential genes from fungi, bacterial and oomycete pathogens, supporting a broad role for RNAi in trans-kingdom gene regulation during host-pathogen interactions (Guo et al., 2019; Cai et al., 2018).

The term “precursor of siRNA” or “siRNA precursor” herein refers to a RNA molecule which can be, directly or indirectly, processed into siRNA duplex(es) in Chlorella cells (or Chlorella extracts). Examples of siRNA precursors that can be directly processed include long double-stranded RNA (long dsRNA), while examples of siRNA precursors that can be indirectly processed include long single-stranded RNA (long ssRNA) that can be used as template for the production of processable long dsRNAs.

The term “precursor of miRNA” or “miRNA precursor” herein refers to an RNA molecule which can be processed into miRNA duplex(es) in Chlorella cells (or Chlorella extracts). Examples of miRNA precursors include primary miRNA precursors (pri-miRNAs) and pre-miRNAs, comprising a hairpin loop.

Of note, plasmids or vectors and other DNA constructs or viral vectors encoding said precursor molecules are also encompassed in the definition of “functional interfering iRNA”.

For targeting multiple genes, the method of the invention can use i) a mixture of several different iRNAs which altogether target multiple genes of interest or ii) a chimeric iRNA targeting several different genes of interest or iii) a mixture of any of these chimeric iRNAs.

In one particular embodiment, the method/use of the invention comprises the introduction of one or several long functional iRNAs into Chlorella cells as precursors, and these cells will produce the small RNAs (such as siRNAs or miRNAs) that can be further formulated and used to prevent pathogenic infections.

These long functional iRNAs can be long single-stranded RNA molecules (named hereafter as “long ssRNAs”). These long ssRNA may be introduced in a Chlorella cell, converted into long dsRNA molecules, and further processed into siRNAs by the Chlorella DCL enzyme. Alternatively, long ssRNA may be produced by an RNA virus that can infect Chlorella cells and further converted into long dsRNA molecules during viral replication (as replicative intermediates). The resulting viral dsRNA is subsequently processed into siRNAs by the Chlorella DCL enzyme.

As used herein, the term “long ssRNA” designates single-stranded structures containing a single-strand of at least 50 bases, more preferably of 80 to 3000 bases. Long ssRNAs may contain 80 to 3000 bases when produced by a Chlorella transgene, but preferably contain 80 to 1500 bases when produced by a recombinant RNA virus.

These long functional iRNAs can also be double-stranded RNA molecules (named hereafter as “long dsRNAs”). These long dsRNAs act as miRNA or siRNA precursors and can be processed into miRNAs or siRNAs in Chlorella cells, thanks to the DCL proteins encoded by Chlorella genomes (see EXAMPLE 3).

As used herein, the term “long dsRNA” designates double-stranded structures containing a first (sense strand) and a second (antisense) strand of at least 50 base pairs, more preferably of 80 to 3000 base pairs.

The results of the present inventors show that, in Chlorella cells, long dsRNAs can be efficiently processed into effective small RNAs (EXAMPLE 3). Such long dsRNAs are advantageously chimeric dsRNA, i.e., they bear sequence homologies to multiple genes.

The long functional iRNA used in the method of the invention is preferably a long dsRNA that is cleavable by the DCL enzyme in Chlorella cells so as to generate miRNAs or siRNAs in Chlorella cells.

These long dsRNAs can be generated from a hairpin structure, through sense-antisense transcription constructs, through an artificial sense transcript construct further used as a substrate for the production of long dsRNAs, or through VIGS. More precisely, they may comprise bulges, loops or wobble base pairs to modulate the activity of the dsRNA molecule so as to mediate efficient RNA interference in the target cells. The complementary sense and antisense regions of these long dsRNA molecules may be connected by means of nucleic acid based or non-nucleic acid-based linker(s). These long dsRNA may also comprise one duplex structure and one loop structure to form a symmetric or asymmetric hairpin secondary structure.

In a particular embodiment, the precursor of the invention can simultaneously target several Xylella fastidiosa gene regions of ˜150-250 bp and have for example the following sequences: IR-cheA/gaCA/tolC/pglA/engXCA1/engXCA2=SEQ ID NO: 1-2; IR-cheA/GumH/GumD/XpsE/LesA/HolC=SEQ ID NO: 3-4; IR-LesA/gumH/gumD=SEQ ID NO: 5-6; IR-XpsE/HolC/LesA=SEQ ID NO: 7-8; IR-cheA/pglA/LesA=SEQ ID NO: 9-10; IR-cheA/engXCA1/engXCA2=SEQ ID NO: 11-12; IR-gacA/pglA/engXCA2=SEQ ID NO:13-14 and IR-cheA/tolC/engXCA1=SEQ ID NO:15-16.

In another particular embodiment (see EXAMPLE 9), the precursor of the invention can target essential genes from Candidatus Liberibacter asiaticus and have the following sequences: IR-wp015452784/WP012778510/wp015452939/act56857/wp012778668=SEQ ID NO: 55-56; IR-wp0127783551wp015452784/WP012778510=SEQ ID NO: 57-58 and IR-wp015452939 act56857 wp012778668=SEQ ID NO: 59-60.

In another particular embodiment, the precursor of the invention can target key virulence genes from Erwinia carotovora with the following sequences: IR-GyrB/MreB/rbfA/RsgA/FliA/QseC=SEQ ID NO: 47-48; IR-GyrB/rbfA/QseC=SEQ ID NO: 49-50; IR-fliA/MreB/QseC=SEQ ID NO: 51-52 and IR-GyrB/MreB/RsgA=SEQ ID NO: 53-54.

In another particular embodiment, the precursor of the invention can target key genes from Pseudomonas syringae pv. actinidiae and have the following sequences: IR-GyrB/Hfq/HrpR/HRPS/MreB/RpoD=SEQ ID NO:61-62; IR-GyrB/Hfq/MreB=SEQ ID NO:63-64; IR-HrpR/HrpS/Hfq=SEQ ID NO:65-66 and IR-HrpR/GyrB/RpoD=SEQ ID NO:67/148.

In another particular embodiment, the precursor of the invention can target essential genes of Pseudomonas syringae pv. tomato strain DC3000 have the following sequences IR-CFA6/HRPL SEQ ID NO:90-91; IR-HRPL SEQ ID NO:92-93; IR-FusA=SEQ ID NO:136-137; IR-GyrB=SEQ ID NO:138-139; IR-FusA/GyrB=SEQ ID NO:140-141; IR-AvrPto/AvrPtoB=SEQ ID NO:94-95; IR-AvrPto/AvrPtoB/HopT1-1=SEQ ID NO: 130-131; IR-rpoB/rpoC/FusA=SEQ ID NO:132-133; IR-secE/rpoA/rplQ=SEQ ID NO:134-135; IR-secE=SEQ ID NO: 142-143; IR-GyrB/hrpL=SEQ ID NO:144-145 and IR-dnaA/dna/NgyrB=SEQ ID NO: 146-147.

In another particular embodiment, the precursor of the invention can target virulence genes of Ralstonia solanacearum with the construct IR-HRPH/HRPB/HRCC=SEQ ID NO:99-98; IR-HRPB/HRCC TssB/XpsR—SEQ ID NO: 100-101.

In another particular embodiment, the precursor of the invention can target virulence genes of Xanthomonas campestris pv. campestris with the construct IR-HRPG/HRPX/RsmA=SEQ ID NO:102-103; IR-NadHb/NadHd/NadHe=SEQ ID NO: 104-105 and IR-DnaA/DnaE1/DnaE2=SEQ ID NO:106-107.

In another particular embodiment, the precursor of the invention can target essential genes from Xanthomonas hortorum pv. vitians and have the following sequences: IR-GyrB=SEQ ID NO:17-18; IR-FusA=SEQ ID NO:19-20; IR-ZDpA=SEQ ID NO:21-22 and IR-GyrB/FusA/ZipA=SEQ ID NO:128-129.

In another particular embodiment, the precursor of the invention can target essential genes from Xanthomonas citri pv. fucan and have the following sequences: IR-GyrB/FusA/MreB/HrpG/PhoP/FhaB=SEQ ID NO:120-121; IR-GyrB/RbfA/MreB=SEQ ID NO:122-123; IR-HrpG/PhoP/FtsZ=SEQ ID NO:124-125; IR-FhaB/FusA/MreB=SEQ ID NO:126-127 and IR-GyrB/FusA/ZipA=SEQ ID NO:128-129.

In another particular embodiment, the precursor of the invention can target essential genes from Acidovorax valerianella and have the following sequences: IR-rimM/rsgA/rbfA/MreB/gyrB/FtsZ=SEQ ID NO:23-24; IR-rimM/rbfA/FtsZ=SEQ ID NO:25-26; IR-RsgA/gyrB/MreB=SEQ ID NO:27-28 and IR-RsgA/rbfA/FtsZ=SEQ ID NO:29-30; In another particular embodiment, the precursor of the invention can essential genes from Acidovorax citrulli and have the following sequences: IR-MreB/ybeY/rbfA/gyrB/FtsZ/rsgA=SEQ ID NO:31-32; IR-MreB/ybeY/rbfA=SEQ ID NO:33-34; IR-rsgA/gyrB/MreB=SEQ ID NO:35-36 and IR-YbeY/RsgA/MreB=SEQ ID NO:37-38.

In another particular embodiment, the precursor of the invention can target essential genes from 5 Botrytis cinerea and have the following sequences: IR-TOR/CGF1/DCL1/DCL2/LTF1/HBF1=SEQ ID NO:76-77; IR-TOR/DCL1/DCL2=SEQ ID NO:78-79; IR-CGF1/HBF1/LTF1=SEQ ID NO:80-81 and IR-DCL1/HBF1/TOR=SEQ ID NO:82-83;

In another particular embodiment, the precursor of the invention can target essential genes from Zymoseptoria tritici and have the following sequences: IR-Br1A2/StuA/F1bc/PKS1/PPT1=SEQ ID NO:108-109; IR-Br1A2/StuA/F1bc=SEQ ID NO:110-111 and IR-StuA/PKS/PPT1=SEQ ID NO:112-113.

In another particular embodiment, the precursor of the invention can target essential genes from 15 Phytophthora infestans and have the following sequences: IR-NPP1/INF1/GK4/piacwp1-1/piacwp1-2/piacwp1-3=SEQ ID NO:68-69; IR-piacwp1-1/piacwp1-2/piacwp1-3=SEQ ID NO:70-71; IR-GK4/INF1/NPP1=SEQ ID NO:72-73 and IR-GK4/INF1/piacwp1-1=SEQ ID NO:74-75.

In another particular embodiment, the precursor of the invention can target essential genes from Plasmopara viticola and have the following sequences: IR-PITG_17947/PITG_10772/PITG_13671/PITG_16956/PITG_00891=SEQ ID NO:114-115; IR-PITG_17947/PITG_10772/PITG_13671=SEQ ID NO:116-117 and IR-PITG_13671/PITG_16956/PITG_00891=SEQ ID NO:118-119.

In another particular embodiment, the precursor of the invention can target viral genes from Plum Pox Virus (PPV) and have the following sequences: IR-P1/HC-Pro/CP=SEQ ID NO:39-40; IR-P1=SEQ ID NO:41-42; IR-HC-Pro=SEQ ID NO:43-44 and IR-CP=SEQ ID NO:45-46.

In another particular embodiment, the precursor of the invention can target essential genes from Colletotrichum species and have the following sequences: IR-CclA/ACS1/ACS2/ELP1/ELP2/MOB2=SEQ ID NO:84-85; IR-CclA/ACS1/ACS2=SEQ ID NO:86-87 and IR-ELP1/ELP2/MOB2=SEQ ID NO:88-89; In another particular embodiment, the precursor of the invention can target essential genes from Fusarium graminearum and have the following sequence: IR-CYP51A/CYP51B/CYP51C=SEQ ID NO:96-97.

The present invention targets the use of any of these siRNA precursors of SEQ ID NO:1-148 to produce a population of functional small iRNAs in Chlorella cells.

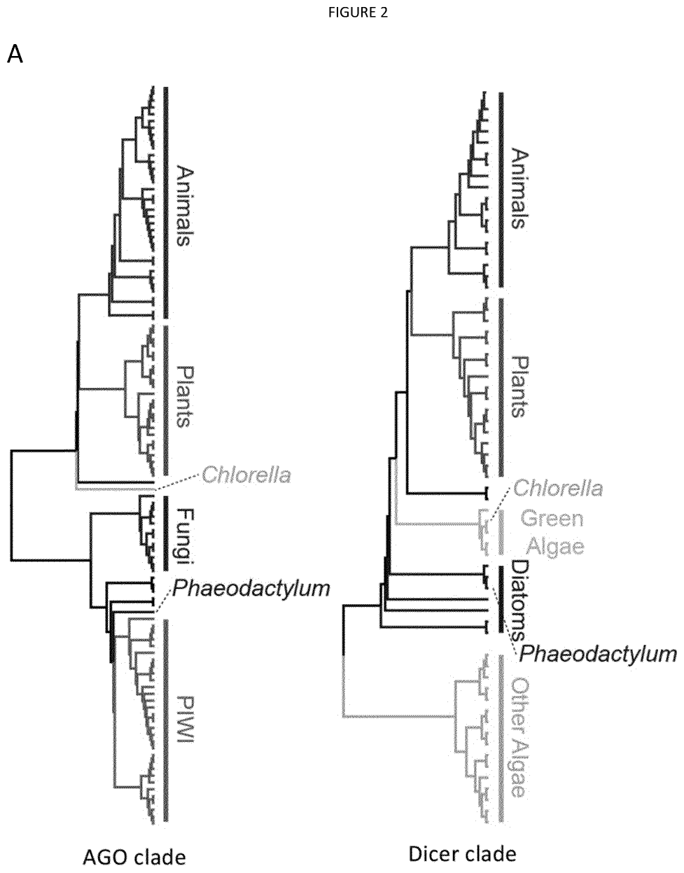

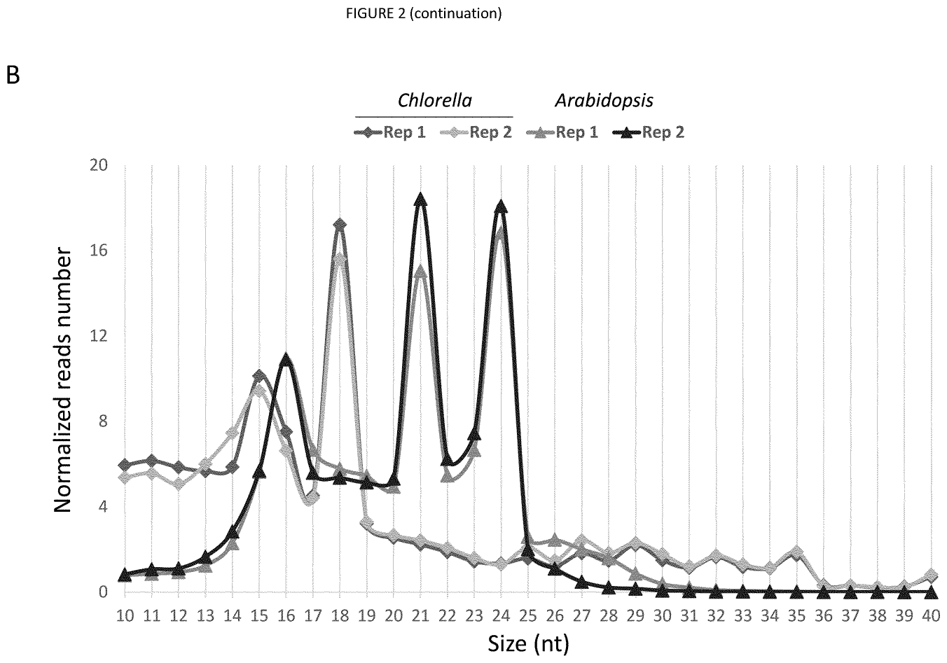

As demonstrated in the examples of the present application, the introduction of dsRNA into Chlorella cells triggers the production of small RNA molecules that are embedded into EVs and therefore protected from ribonuclease-mediated digestion (EXAMPLE 5). More precisely, the Chlorella cells of the invention are able to produce functional small iRNAs such as siRNAs or miRNAs. These small RNAs have a short size, which is less than 50 base pairs, preferably comprised between 10 and 30 base pairs, more preferably between 15 and 30 base pairs. More particularly, the small RNAs produced by Chlorella cells contain mainly 15 or 18 base pairs (cf. EXAMPLE 4 and FIG. 2).

In one particularly preferred embodiment, the functional interfering small RNA of the invention is a “siRNA”, which designates either a “siRNA duplex” or a “siRNA simplex”. This duplex or simplex siRNAs contain preferably 15 or 18 base pairs. They are therefore shorter than plant-produced siRNAs that typically contain 21 or 24 base pairs, or than mammalian-produced siRNAs that are ˜22 base pairs.

The functional small RNAs of the invention that are generated by Chlorella cells thus exhibit distrinct features from those produced by plants and other eukaryotic cells.

More specifically, the term “siRNA duplex” designates double-stranded structures or duplex molecules containing a first (sense strand) and a second (antisense) strand of at least 10 or 15 base pairs, and preferably of less than 20 base pairs; preferably, said antisense strand comprises a region of at least 15 contiguous nucleotides that are complementary to a transcript of the targeted gene. In a preferred embodiment, these molecules contain precisely 15 or 18 base pairs, as shown in the experimental part below. These siRNA duplexes can be produced from long dsRNA precursors that are processed by the Chlorella DCL enzyme and/or other endogenous RNases.

As used herein, the term “siRNA simplex” or “mature siRNA” designates simplex molecules (also known as “single-stranded” molecules) that originate from the siRNA duplex but have been matured in the RISC machinery of a microalgae cell and are loaded in the Chlorella AGO protein and/or associated with other RNA-binding proteins. They have a short size, which is less than 50 bases, preferably between 10 and 30 bases, more preferably between 15 and 30 bases, even more preferably between 10 and 18 bases (preferably not 16 bases), and contain even more preferably either 15 or 18 bases.

In another embodiment, the functional iRNA of the invention is a “miRNA”, which designates either a “miRNA duplex” or a “miRNA simplex”. In a preferred embodiment, the iRNAs of the invention are double-stranded miRNAs.

More specifically, the term “miRNA duplex” designates double-stranded structures or duplex molecules containing a first (sense strand) and a second (antisense) strand of at least 15 base pairs, preferably of at least 19 base pairs; preferably, said antisense strand comprises a region of at least 15 contiguous nucleotides that are complementary to a transcript of the targeted gene. These miRNA duplexes may also contain bulges. These miRNA duplexes can be produced from miRNA precursors that are processed by the Chlorella DCL enzyme. As the duplex siRNAs, they have short size, which is less than 50 base pairs, preferably comprised between 15 and 30 base pairs. More particularly, the small miRNAs produced by Chlorella cells contain mainly 18 base pairs. They can also contain 15 base pairs (cf. EXAMPLE 4 and FIG. 2).

As used herein, the term “miRNA simplex” or “mature miRNA” designates simplex molecules (also known as “single-stranded” molecules) that originate from the miRNA duplex but have been matured in the RISC machinery of a microalgae cell and are loaded in the Chlorella AGO protein and/or associated with other RNA-binding protein. These simplex miRNAs typically contain between 10 and 18 bases (preferably not 16 bases), preferably between 15 and 18 bases, even more preferably either 15 or 18 bases.

Methods to design iRNAs such as long dsRNAs that can be converted into siRNA/miRNA are available in the art and can be used to obtain the sequence of the precursors of the invention.

The inventors herein show (EXAMPLES 2-13) that it is possible to use long double-stranded inverted repeat constructs in order to (i) transform Chlorella cells efficiently and (ii) have them produce functional small iRNAs that can dampen pathogenicity, said small iRNAs being embedded into EVs, which protect these RNA entities from ribonuclease-mediated digestion (see FIGS. 2-8).

As used herein, the term “sequence homology” refers to sequences that have sequence similarity, i.e., a sufficient degree of identity or correspondence between nucleic acid sequences. In the context of the invention, two nucleotide sequences share “sequence homology” when at least about 80%, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, alternatively at least about 99% of the nucleotides are similar.

Conversely, nucleotide sequences that have “no sequence homology” are nucleotide sequences that have a degree of identity of less than about 10%, alternatively of less than about 5%, alternatively of less than 2%.

Preferably, the similar or homologous nucleotide sequences are identified by using the algorithm of Needleman and Wunsch. Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide residue matches and an identical percent sequence identity when compared to the corresponding alignment generated by GAP Version 10.

### Production Methods of the Invention

In a first aspect, the present invention is drawn to a method for producing functional interfering small RNAs, said method comprising at least the steps of:
a) transforming Chlorella cells with a siRNA or miRNA precursor comprising a fragment of at least one target gene, and
b) cultivating said Chlorella cells in appropriate conditions so that they express said precursor and release EV-embedded functional small iRNAs targeting said gene fragment.

The terms “interfering small RNA” and “siRNA or miRNA precursor” have been defined above, in the definition section.

In a preferred embodiment, said siRNA or miRNA precursor is a long single- or double-stranded RNA molecule. In a more preferred embodiment, said siRNA or miRNA precursor is a long double-stranded RNA molecule, said molecule comprising a fragment of at least one target gene, or a complementary sequence thereof.

As explained above, Chlorella cells can be transformed by large nucleotide constructs. More precisely, the targeted fragment contained in the said precursor can have a large size, e.g., up to 3000 bp.

The “fragment” contained in the precursor of the invention can in fact contain one or several portion(s) of one single gene, or several portion of several genes (see the EXAMPLES 11 and 12 below). After transformation, the Chlorella cells will then produce siRNA populations targeting one or various portions from a single gene or from several genes. This is a clear advantage over other iRNA producer cells, as covering large portions of microbial/parasitic gene(s) maximizes the chance of triggering an effective silencing effect towards the targeted gene(s) and reduces the chance that the microbe/parasite acquire resistance against the small iRNA population (to do so, it will have to mutate all along the small RNA targeted portions), thereby resulting in long-lasting protection effects against the targeted pathogen(s). It is also possible to design and use a precursor that contains one or more portions of genes from several pathogens (they will be called “chimeric precursors”, see below).

In a particular embodiment, the fragment of the target gene(s) contained in the precursor of the invention comprises between 50 and 3000 bp, preferably between 100 bp and 2000 bp, more preferably between 500 bp and 1500 bp.

Particular “target genes” are described below, in the appropriate sections.

Chlorella is a genus of single-celled green algae belonging to the division Chlorophyta. It is spherical in shape, about 2 to 10 m in diameter, and is without flagella. It contains the green photosynthetic pigments chlorophyll-a and -b in its chloroplast. In ideal conditions it multiplies rapidly, requiring only carbon dioxide, water, light, and a small amount of minerals to grow. Due to the elevated protein, vitamin, mineral and pigment content, various Chlorella cells are currently used as food complement for humans and livestock.

The Chlorella cells used in the method of the invention can be of any Chlorella species. In particular, they can be any cells that are currently used as food complement for humans and livestock (Safi et al, 2014). In a particular embodiment, they can belong to the species: Chlorella ellipsoidea, Chlorella pyrenoidosa, Chlorella sorokiniana, Chlorella vulgaris or Chlorella variabilis.

In a preferred embodiment, the Chlorella cells used in the method of the invention are from the vulgaris genus. As the other Chlorella cells, C. vulgaris cells are able to adapt and grow in a variety of conditions. They are easy to maintain in laboratory conditions, possess a simple life cycle, a haploid genome and metabolic pathways similar to higher plants. They also possess the capacity to grow in auto-, hetero- or mixo-trophic conditions with high growth rates (de Andrade et al, 2017). The metabolic flexibility, the ease of maintenance and growth are features that enable C. vulgaris to be exploited as industrial production scaffold in photobioreactors (PBRs) for a variety of molecules of interest (Lin et al., 2013; Blanc et al., 2010).

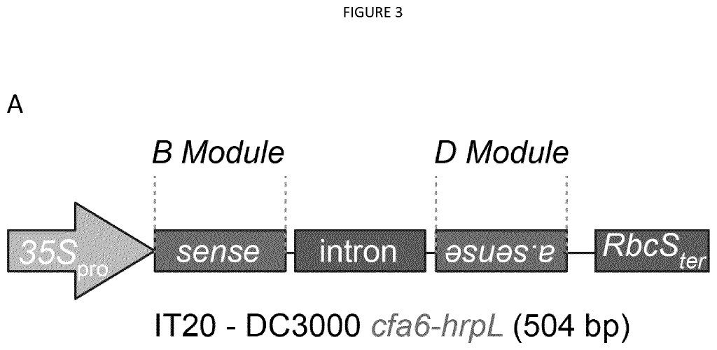

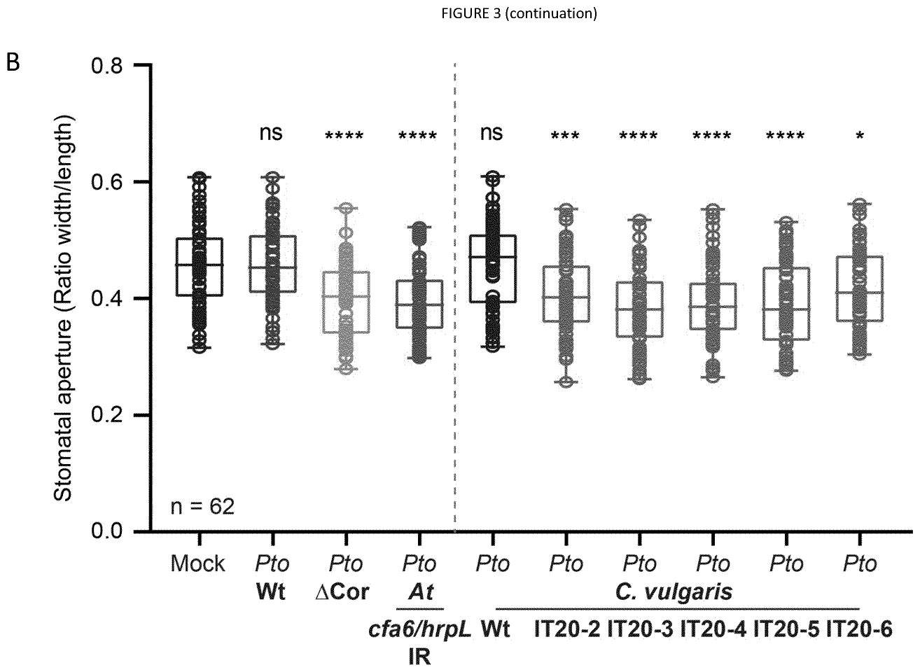

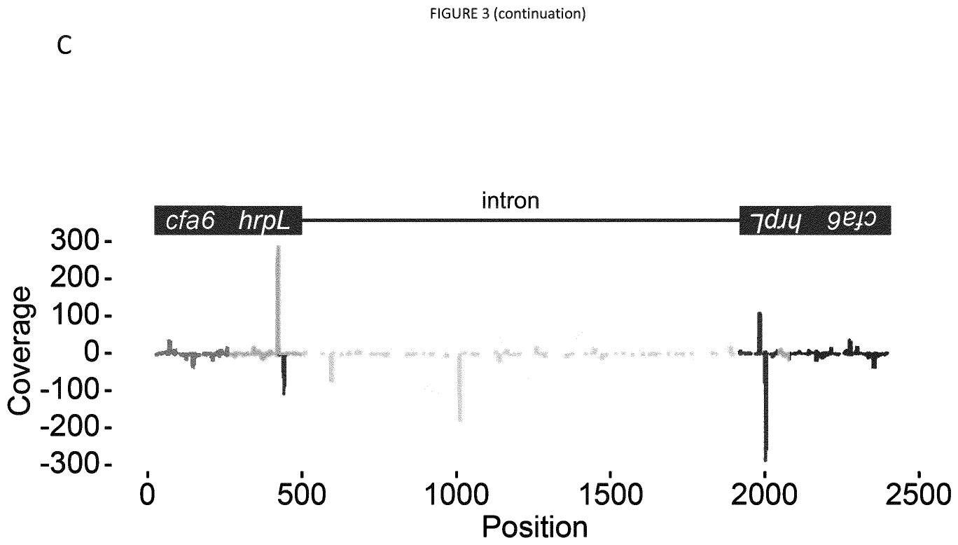

In a first step of the method of the invention, the siRNA or miRNA precursor of the invention is introduced in the selected Chlorella cells. Said siRNA or miRNA precursor will be processed into siRNA or miRNA duplexes by using the Chlorella DCL enzyme and other small RNA processing factors. Said small RNAs duplexes and/or mature small RNA guides (i.e. loaded into AGOs) are thereafter released in the extracellular medium, or at the surface of the Chlorella cells, embedded into Extracellular Vesicles. As demonstrated in the examples below (EXAMPLES 5 and 6 and 7 and FIGS. 3 to 5), the virulence of bacterial cells is decreased when placed in contact with Chlorella EVs containing siRNAs.

The term “introduced” in the context of inserting a nucleic acid into a cell, means “transfection” or “transformation” or “transduction” and includes reference to the incorporation of a nucleic acid into a eukaryotic cell where the nucleic acid may be stably incorporated into the genome of the cell (e.g., chromosome, plasmid), or transiently expressed (e.g., transient delivery of a gene construct via Agrobacterium tumefaciens, an infection with a recombinant virus).

The expression of the iRNAs of the invention in the host Chlorella cell may be transient or stable. Stable expression refers in particular to the preparation of transgenic Chlorella cell lineages using conventional techniques.

By way of non-limitative examples, step a) of the method of the invention can be performed by using electroporation, projectile bombardment, PEG-mediated protoplast transformation, virus-mediated transformation, conjugation, Agrobacterium-mediated transformation, and the like. These transformation methods are described e.g., in Kim et al., 2002; Cha et al., 2012; Lin et al., 2013; Yang et al., 2015; Bai et al., 2013; Niu et al., 2011; Chien et al., 2012; Run et al., 2016.

In a preferred embodiment, step a) of the method of the invention involves the delivery of the gene construct into Chlorella cells by means of Agrobacterium tumefaciens. This technic is well-known and do not need to be explained (Cha et al., 2012; Lin et al., 2013).

In a particular embodiment, the method of the invention comprises introducing into Chlorella cells one or several dsRNAs targeting one or multiple genes of different parasites, such as viruses, fungi, oomycetes, bacteria, insects or nematodes. In this embodiment, the EVs are directed to an essential gene, to a virulence gene or an antimicrobial/antiparasitic resistance gene of several pathogens or parasites.

Such methods are useful for concomitant prevention or treatment of diseases caused on plants by several pathogens and/or parasites. They can be carried out using chimeric EVs carrying sequence homologies with different pathogenic/parasitic genes, or a cocktail of EVs that have been produced separately, some containing iRNAs bearing homologies to genes of one pathogen/parasites and others containing iRNAs bearing homologies to genes from other pathogens/parasites.

In a second step, the transformed Chlorella cells containing the precursors of the invention are cultivated so as to express said precursor and release EV-embedded functional small iRNAs targeting said gene fragment.

In this step, one can use classical conditions well-known in the art for cultivating Chlorella cells (Kim et al., 2002; Cha et al., 2012; Lin et al., 2013; Yang et al., 2015; Bai et al., 2013; Niu et al., 2011; Chien et al., 2012; Run et al., 2016). A particular cultivating method is described in EXAMPLE 1 below.

The present inventors herein show that, like for plant cells, it is possible to enhance the yield of EV production by cultivating the Chlorella cells in conditions of abiotic or biotic stresses, for example by submitting the cells to specific temperature conditions, to bacterial or viral infections or by contacting them with phytohormones or other chemical compounds. As disclosed in EXAMPLE 13 below, it is possible to enhance the yield of the Chlorella EV production by treating them with supernatants of heat-killed bacteria, such as E. coli K12 TOP10 or Pto DC3000 Wt cells. The rationale for using supernatants from heat-killed bacterial cells is that these supernatants should contain cocktails of molecules, including MAMPs/PAMPs, which could be sensed by yet-unknown Chlorella Pattern Recognition Receptors (PRRs), thereby resulting in enhanced EVs production and/or secretion as found in plants.

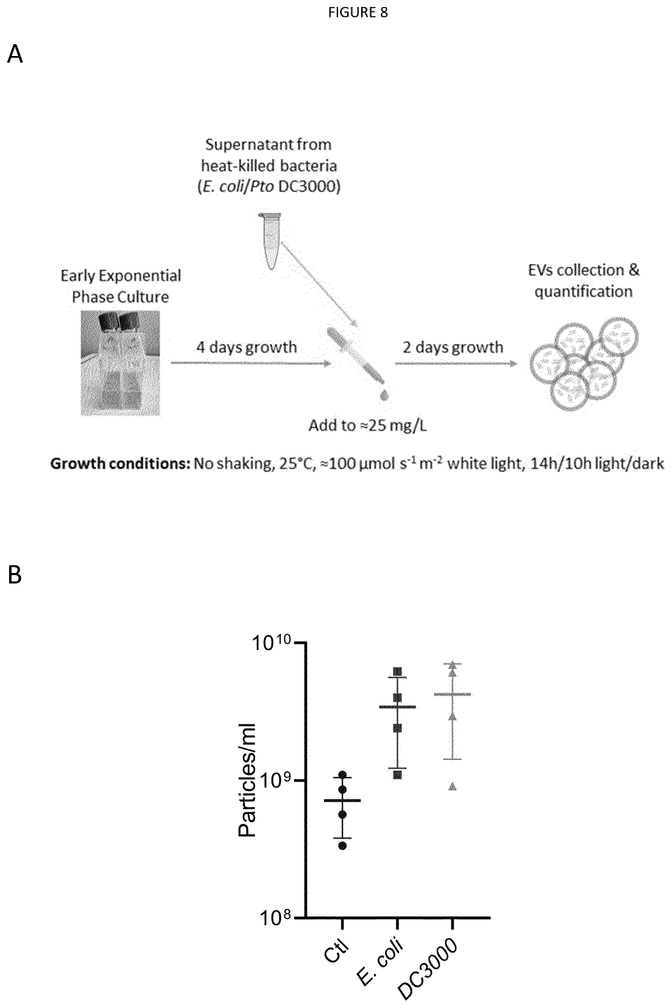

The inventors have found that the production of EVs by Chlorella can be increased several times by such a treatment, as found in plants. Consequently, it is proposed that some biotic stresses can thus be employed to increase Chlorella EVs production and/or secretion. A preferred treatment is to use the supernatants from heat-killed bacteria, that can be easily produced and in a cost-effective manner, and have been found suitable for enhancing the production of Chlorella EVs (EXAMPLE 13 and FIG. 8).

In one preferred embodiment, the small RNAs of the invention are isolated as free RNA molecules. These RNA molecules can be used directly for phytotherapeutic purposes (see EXAMPLES 5-8).

In this embodiment, the methods of the invention further comprise the step of recovering the expressed small iRNAs from the cultivated Chlorella cells.

Isolating intact RNA contained within Chlorella cells requires four steps: 1) Disruption of the Chlorella cells; 2) Inactivation of endogenous ribonuclease (RNase) activity; 3) Denaturation of nucleoprotein complexes; and 4) Removal of contaminating DNA and proteins. The most important step is the immediate inactivation of endogenous RNases that are released from membrane-bound organelles when cells are disrupted. RNA purification methods typically use silica membrane-based, resin-based and magnetic options for nucleic acid binding and incorporate DNase treatment to remove contaminating genomic DNA. Purified RNA is then eluted from the solid support.

RNA is notoriously susceptible to degradation and RNases are ubiquitous. Many commercially available RNA purification methods include specific chemicals to inactivate RNases present in cell or tissue lysates and may also include RNase inhibitors to safeguard against RNA degradation throughout the procedure. Any of these methods can be used to recover the small RNAs of the invention.

In another preferred embodiment, the small RNAs are not used as free RNA molecules, but they are embedded into extracellular vesicles (EVs). The present inventors have indeed shown that Chlorella cells can produce EVs which are in a size range that is similar to the one of plant exosomes (50-200 nm), and that these EVs can be taken-up by plant cells, where they can deliver their small iRNA content and have effective silencing effect (see EXAMPLE 7). These Chlorella derived iRNA-containing EVs can be used for biocontrol purposes, as mammalian and plant-derived EVs are.

In this particularly preferred embodiment, the method of the invention further comprises the step of recovering the Extracellular Vesicles (EV) released by Chlorella cells in the extracellular medium.

In the context of the invention, recovering EVs can be done by any conventional means described in the art. Isolation and purification means are for example discussed in Colao et al., 2018. Downstream processing for efficient purification can be used to enrich EVs from cell culture media, e.g., by size-exclusion (based on typica diameters), sedimentation force or flotation density, precipitation-based methods and affinity-based capture. While differential ultracentrifugation can be used, other purification methods will be preferred, such as filtration or chromatic separation. Tangential-flow filtration is more promising, due to tight and reproducible size distributions and the ease with which processes can be scaled. Immunoaffinity methods can also be adjusted to the particular EVs of the invention.

### EVs Obtained by the Method of the Invention

Extracellular Vesicles (EVs) are nanosized, membrane-bound vesicles that are released into the extracellular space and transport cargoes towards recipient cells. Mammalian EVs are in part composed of exosomes, which are formed by the fusion between multivesicular bodies (MVBs) and the plasma membrane, in which MVBs release vesicles whose diameters range from 40 to 150 nanometers (O'Brien et al., 2020). During the last decade, mammalian exosomes have been extensively characterized as vehicles of miRNAs. Interestingly, emerging evidence indicates that plant-derived EVs can also operate as carriers of miRNAs or siRNAs (Wang et al., 2013; Zhang et al., 2016; Cai et al., 2018; Hou et al., 2019).

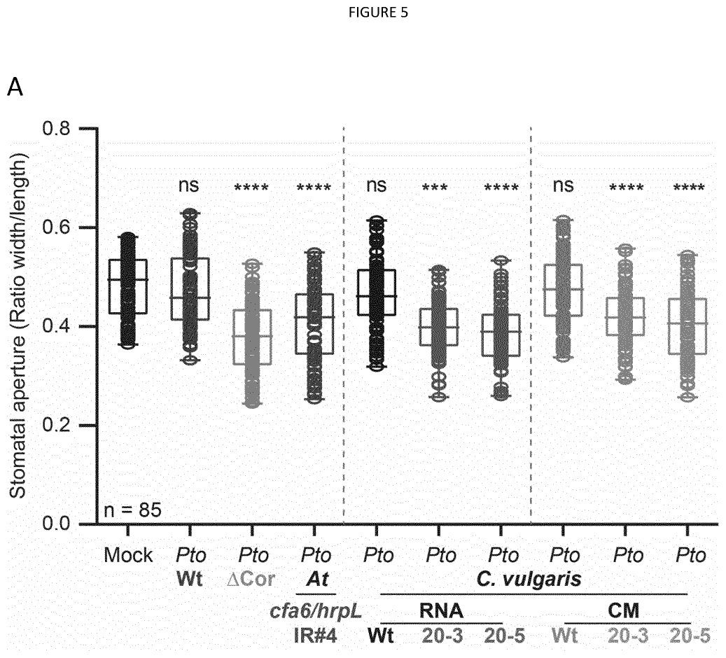

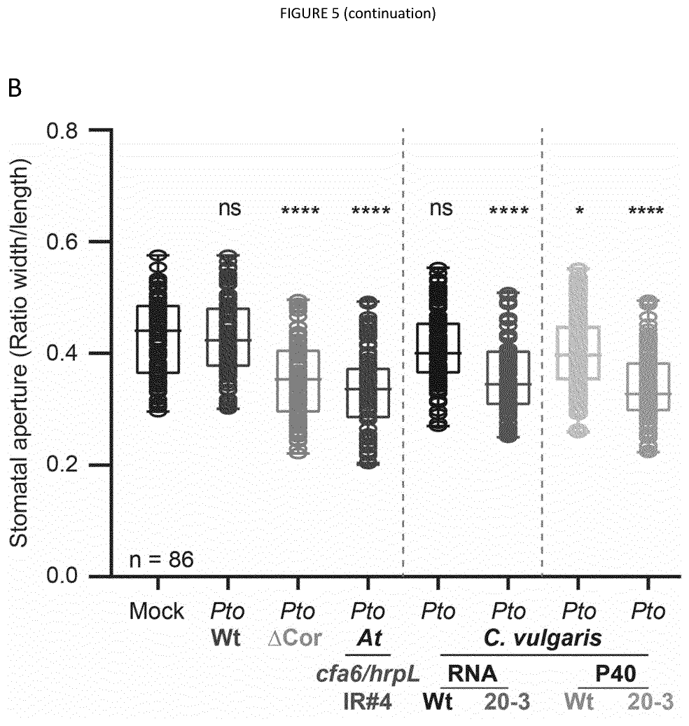

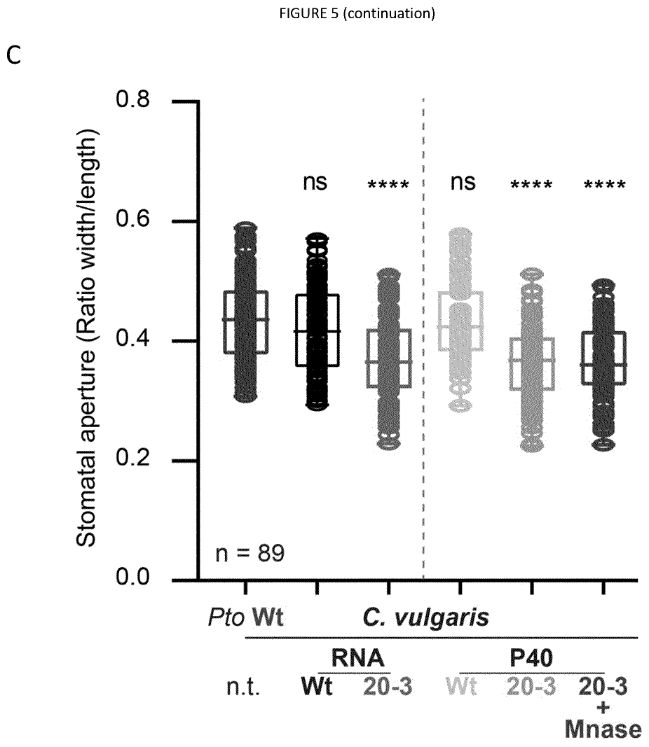

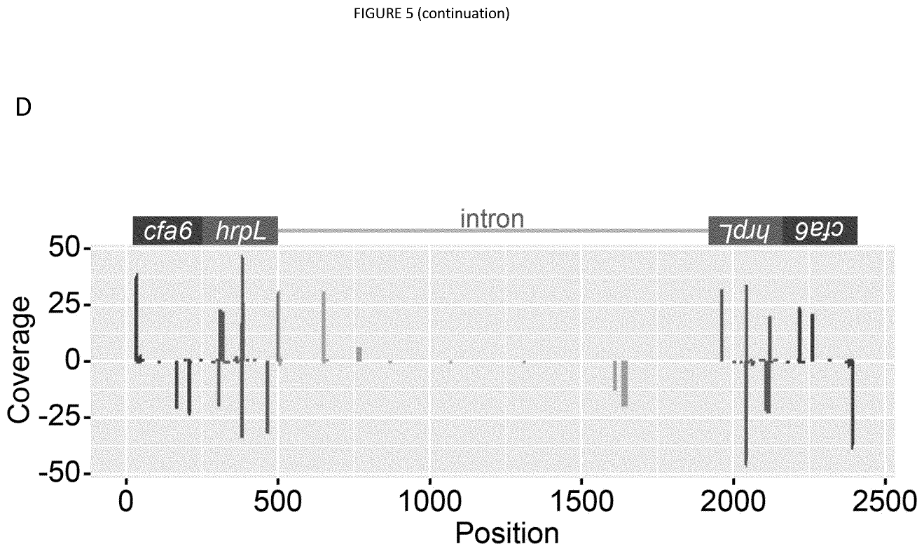

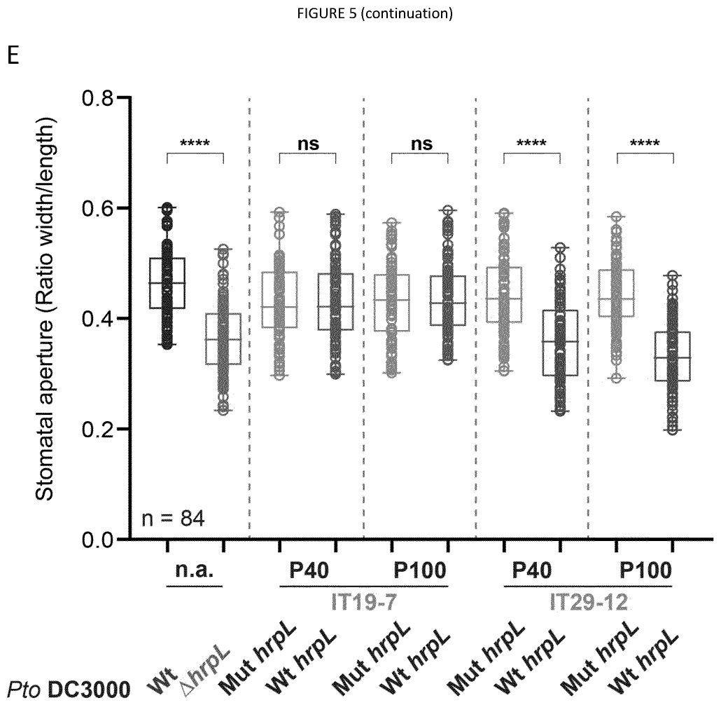

In another aspect, the present invention is drawn to the Extracellular Vesicles (EVs) obtained by the method of the invention, as disclosed above. These EVs contain a population of functional small iRNAs targeting one or several region(s) in the target gene(s) of interest. Interestingly, antibacterial small iRNAs can be detected from Mnase-treated Chlorella EVs (see EXAMPLE 7 and FIG. 5).

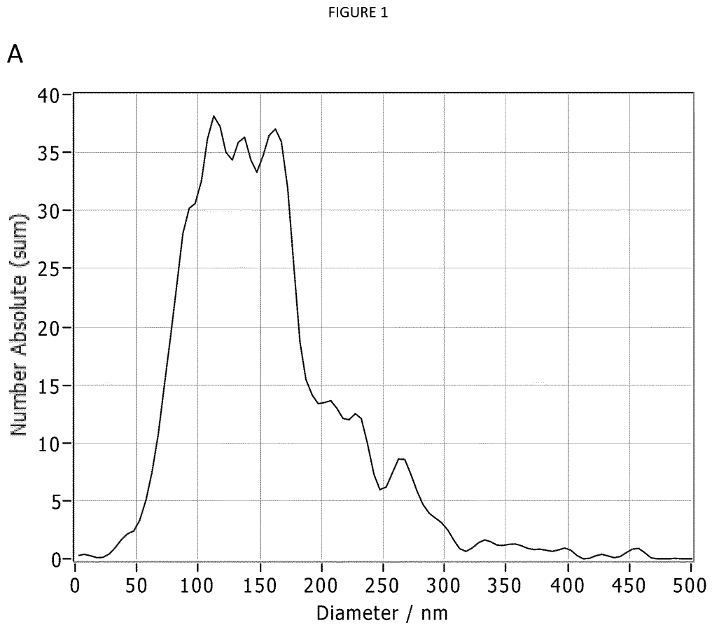

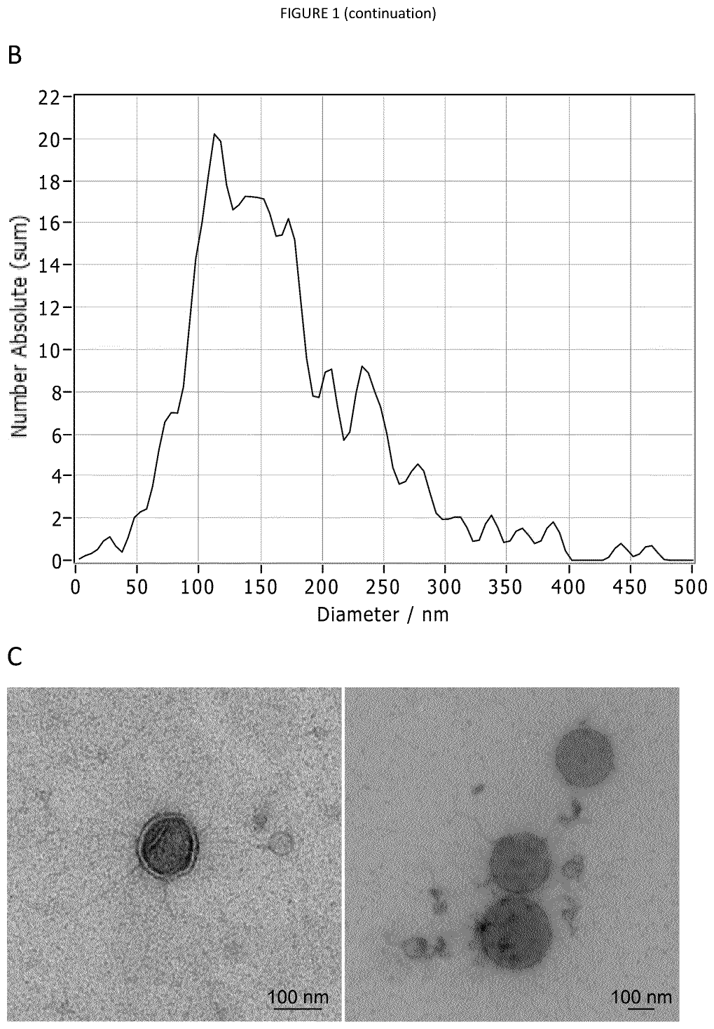

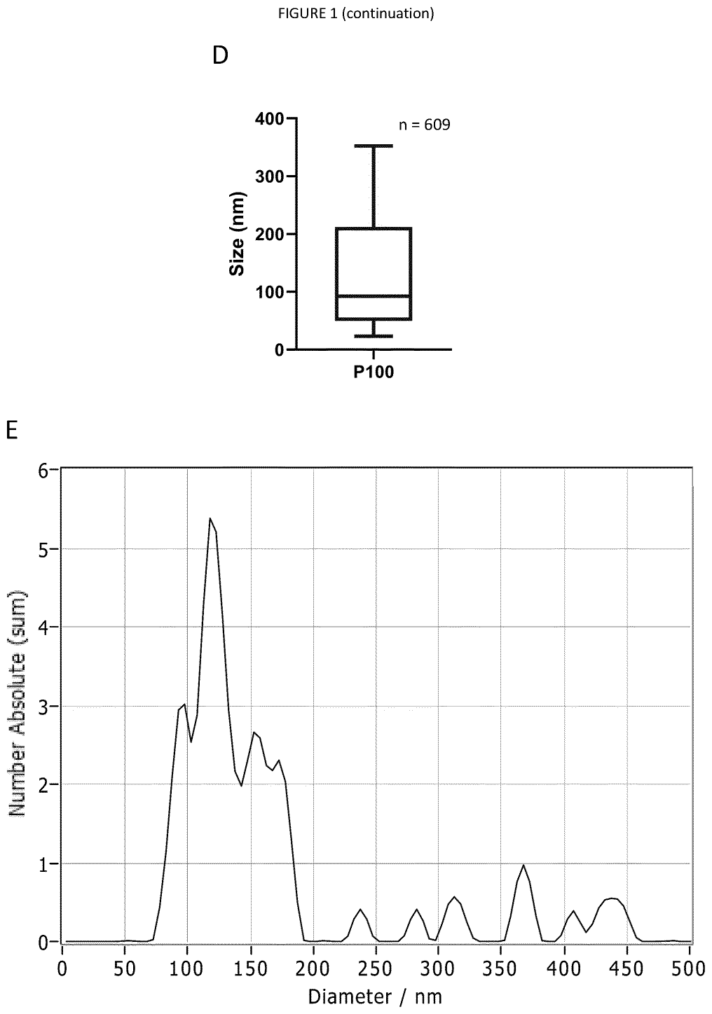

As shown in EXAMPLE 3 and FIG. 1, the present inventors were able to characterized the Chlorella EVs by Nanoparticle Tracking Analysis (NTA) and through labeling of lipid-based extracellular particles. This first analysis revealed that Chlorella EVs are in a size range between 50 and 200 nm. Further transmission electron microscopy (TEM) unveiled the presence of round shaped particles with an apparent lipidic bilayer and a ˜130 nm mean diameter.

The results of the inventors (EXAMPLE 2 and table 1) also show that the EVs produced by the Chlorella cells are not likely to contain CD63 tetraspanin in their membrane, since the Chlorella genome and transcriptome do not contain such factors. Yet, tetraspanin 8 is known to be present on plant EVs (Cai et al., 2018). Therefore, the EVs produced by Chlorella cells are different from those produced by plants.

In a preferred embodiment, the EVs of the invention preferably contain a population of functional small iRNAs, preferably of 10 to 18 base pairs, more preferably of 15 to 18 base pairs, that targets several regions in one or several viral gene(s). Accordingly, these EVs can be used as anti-viral agents.

For example, these anti-viral EVs can contain a population of functional small iRNAs, preferably of 10 to 18 base pairs, more preferably of 15 to 18 base pairs, that targets one or several regions of one or several viral gene(s) that are critical for the replication or the pathogenicity of the Plum pox virus, responsible of the Sharka disease.

In another preferred embodiment, these EVs of the invention preferably contain a population of functional small iRNAs, preferably of 10 to 18 base pairs, more preferably of 15 to 18 base pairs, that targets several regions in one or several bacterial gene(s). Accordingly, these EVs can be used as anti-bacterial agents.

For example, these anti-bacterial EVs can contain population of functional small iRNAs, preferably of 10 to 18 base pairs, more preferably of 15 to 18 base pairs, that targets one or several regions of one or several bacterial gene(s) that are critical for the replication or the pathogenicity of Xylella fastidiosa, Candidatus liberibacter, Pseudomonas syringae pv. actinidiae, Pseudomonas syringae pv. tomato strain DC3000, Xanthomonas campestris pv. campestris, Ralstonia solanacearum, Erwinia carotovora, Xanthomonas hortorum, Acidovorax vallerianellae, Acidovorax citrulli.

In another preferred embodiment, these EVs of the invention preferably contain a population of functional small iRNAs, preferably of 10 to 18 base pairs, more preferably of 15 to 18 base pairs, that targets several regions in one or several fungal gene(s). Accordingly, these EVs can be used as anti-fungal agents.

For example, these anti-fungal EVs can contain population of functional small iRNAs, preferably of 10 to 18 base pairs, more preferably of 15 to 18 base pairs, that targets one or several regions of one or several bacterial gene(s) that are critical for the replication or the pathogenicity of Fusarium graminearum, Botrytis cinerea, Colletrotrichum species, Zynoseptoria tritici.

In another preferred embodiment, these EVs of the invention preferably contain a population of functional small iRNAs, preferably of 10 to 18 base pairs, more preferably of 15 to 18 base pairs, that targets several regions in one or several oomycetal gene(s). Accordingly, these EVs can be used as anti-oomycetal agents.

For example, these anti-oomycetal EVs can contain population of functional small iRNAs, preferably of 10 to 18 base pairs, more preferably of 15 to 18 base pairs, that targets one or several regions of one or several bacterial gene(s) that are critical for the replication or the pathogenicity of Phytophthora infestans, Plasmopara viticola.

As explained above, purification of EVs can be performed by various methods. While differential ultracentrifugation can be used, other purification methods will be preferred for industrial purposes, such as filtration, chromatic separation, or affinity-purification methods.

### Phytotherapeutic Compositions of the Invention

It is noteworthy that the small RNAs of the invention contained within the natural Extracellular Vesicles (EVs) of the invention are protected from the action of RNases (EXAMPLE 7). iRNA-containing EVs can therefore be used efficiently and long lastingly in phytotherapeutic compositions as a tool to kill or dampen the infection of target pathogens.

In a further aspect, the present invention is thus drawn to phytotherapeutic compositions containing, as active principle, the small RNAs-embedded EVs of the invention. More precisely, they contain an effective amount of the Chlorella-derived EVs as defined above.

By “effective amount”, it is herein meant an amount that has been shown to have an antipathogenic effect (e.g., an antibacterial or antiviral effect) in a test such as described in the examples below. This amount is preferably comprised between 0.05 pM and 100 pM, preferably between 0.05 pM and 10 pM (for in vitro applications) or between 0.05 pM and 100 nM, preferably between 0.05 pM and 10 nM (for in vivo applications) of EVs containing effective small RNAs.

The phytotherapeutic compositions of the invention can be formulated in a physiological or agronomical acceptable carrier, excipient or diluent. Such carriers can be any material that the plant to be treated can tolerate. Furthermore, the carrier must be such that the composition remains effective at controlling the infection, and not toxic for animals or insects that feed on the treated plants. Examples of such carriers include water, saline, Ringer's solution, dextrose or other sugar solutions, Hank's solution, and other aqueous physiologically balanced salt solutions, phosphate buffer, bicarbonate buffer and Tris buffer.

The compositions of the invention can be supplied in a concentrated form, such as a concentrated aqueous solution. It may even be supplied in frozen form or in freeze-dried or lyophilized powder form. This latter may be more stable for long term storage and may be de-frosted and/or reconstituted with a suitable diluent immediately prior to use.

These compositions may furthermore contain a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaricides, plant growth regulators, harvest aids, and fertilizers, can be combined with carriers, surfactants or adjuvants customarily employed in the art of formulation or other components to facilitate product handling and application for particular target bacteria. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers.

The compositions of the invention can be solid slow-release formulations, surfactant diatomaceous earth formulations for pesticidal use in the form of dry spreadable granules, water-insoluble lipospheres formed of a solid hydrophobic core having a layer of a phospholipid embedded on the surface of the core, microcapsules, etc.

The nature of the excipients and the physical form of the composition may vary depending on the nature of the plant part that is desired to treat.

### Phytotherapeutic Methods and Uses of the Invention

In another aspect of the invention, the present invention is drawn to phytotherapeutic methods involving the use of the EVs of the invention. These EVs can be used for treating any parasitic infection and/or infectious disease in a plant.

Said parasitic infection and/or infectious disease can be caused e.g., by a virus, a bacterium, a fungus, an oomycete, or any other pathogens or parasites.

The EVs of the invention can target a gene of said pathogen and/or a gene of the diseased host cultivated plants, if this/these gene(s) is/are known to facilitate the infection or to act as negative regulator(s) of defense.

In one aspect, the present invention relates to a method for treating a target plant against a pathogenic or parasitic infection, said method comprising the step of applying the EVs of the invention on a part of said plant.

This method is useful to avoid the contamination of plants and ensure their adequate growth and high yield of production.

In another aspect, the present invention therefore relates to an EV-based biocontrol method for treating plants against a pathogen or parasite infection, said method comprising the step of delivering the EVs of the invention, or a composition comprising these EVs on plant tissues, seeds, fruits, vegetables, prior to and/or after infections. Such delivery can also be performed by trunk injection or petiole absorption in the case of woody plants, as previously showed for synthetic siRNAs (Dalakouras et al., 2018). This infection can be due to any pathogen, such as bacteria, virus, fungus, oomycetes, or other parasites associated with plant organisms.

All these pathogens are described below.

Another aspect of the invention relates to the use of EVs of the invention, as defined above, as a phytotherapeutic agent. Preferably, said EVs are used for treating a disease caused by a pathogenic bacterium in plants or for preventing a bacterial infection in plants.

In one embodiment, these phytotherapeutic EVs or compositions containing thereof contain siRNA duplex or miRNA duplex molecules, as defined above. In yet another embodiment, these EVs targets bacterial genes and genes of other non-bacterial pathogens or parasites, as defined above, for concomitant prevention or treatment of diseases caused by bacterial pathogens and other pathogens/parasites in plants. All the embodiments proposed above for the EVs, iRNAs, the vectors, and the transformation methods are herewith encompassed and do not need to be repeated.

The EVs of the present invention can be applied to the crop area, plant, reproductive organs, fruits, seed and roots to be infected or that is already infected.

Methods of applying the EVs or a composition that contains the EVs of the invention include, but are not limited to, petiole absorption, trunk injection, foliar application, seed coating, and soil application. The number of applications and the rate of application depend on the intensity of the infection.

Specifically, the compositions of the invention can be applied to the plants by, for example, spraying, atomizing, dusting, scattering, coating or pouring, introducing into or on the soil, introducing into irrigation water, by seed treatment or general application or dusting at the time when the bacterial infection has begun or before the bacterial infection as a protective measure.

The invention also relates to the use of said phytotherapeutic composition for controlling, inhibiting or preventing the growth or pathogenicity of any pathogen on target plants.

As explained in details below, the composition of the invention can more precisely be used for:
Treating and/or preventing and/or controlling a pathogen pathogenicity,
Treating and/or preventing and/or controlling a pathogen growth,
Reducing the antimicrobial resistance of pathogens,
Enhancing the growth or beneficial effects of symbiotic or some commensal microbes,
   thereby increasing plant yields.

The EVs of the invention are useful for silencing genes in any microbes: pathogenic or non-pathogenic bacteria; Gram-positive or Gram-negative bacteria, virus, fungi, oomycetes, or other organisms associated with plants. Examples of these different target pathogens are now disclosed.

### Pathogenic Bacteria

In a preferred embodiment, said pathogen is a plant pathogenic bacterium.

Non-limitative examples of plant pathogenic bacteria, which can be targeted using the EVs of the invention include: Ralstonia solanacearum, Xanthomonas oryzae pathovars, Xanthomonas campestris pathovars, Xanthomonas axonopodis pathovars, Xanthomonas euvesicatoria pathovars, Xanthomonas hostorum pathovars, Pseudomonas syringae pathovars, Pseudomonas viridiflava pathovars, Pseudomonas savastonoi pathovars, Candidatus liberibacter asiaticus, Candidatus liberibacter solanacearum, Acidovorax citrulli, Acidovorax avenae pathovars, Pectobacterium atrosepticum pathovars, Pectobacterium carotovorum pathovars, Pectobacterium sp., Agrobacterium tumefaciens, Dickeya (dadantii and solani), Erwinia amylovora, Clavibacter michiganensis (michiganensis and sepedonicus), Xylella fastidiosa, Pectobacterium (carotovorum and atrosepticum), Streptomyces scabies, Phytoplasma sp., Spiroplasma sp. (Zhang et al., 2009)

If ornamental plants are treated, the following bacteria can be targeted: Pseudomonas cichorii, known to infect Chrysanthemum, Geranium, and Impatiens; Xanthomonas campestris pv. Pelargoni, known to infect Geranium; Rhodococcus fascians, known to infect Chrysanthemum morifolium, Pelargonium, Phlox, and possibly Rhododendron; Ralstonia solanacearum, known to infect Geranium, Anthurium spp, Rose tree, Curcumas, and Anthuriums; Xanthomonas axonopodis, Xanthomonas hortorum, known to infect Geranium, Begonia, Anthurium, and Hibiscus rosa-sinensis; Pectobacterium carotovorum, known to infect Amaryllis, Begonia, Calla, Cyclamen, Dracaena and Impatiens.

### Beneficial Bacteria

In a particular embodiment, the EVs of the invention contain functional iRNA(s) targeting one or multiple genes of beneficial bacteria often referred to as Plant-growth-promoting rhizobacteria (PGPR). The purpose of this particular embodiment is to promote the beneficial effects of said PGPR. In this particular embodiment, the targeted bacterial genes are factors that, when silenced, promote the replication of the targeted bacterial cells or a pathway that is beneficial for the host and that positively regulate the production of a beneficial compound (e.g., a phytohormone), secondary metabolites that (i) alter the survival/pathogenicity of surrounding phytopathogens, (ii) activate plant defense responses (e.g., Induced Systemic Resistance), (iii) facilitate the uptake of nutrients from the environment (e.g., by enhancing the production of bacterial factors that are essential for Rhizobium-legume symbiosis), (iv) enhance the tolerance of the host organism to abiotic stress conditions etc. Silencing of such bacterial targeted genes would thus lead to an increased growth rate of the host organism and/or several other possible beneficial effects for the host organism.

In such an embodiment, the iRNAs contained in the EVs of the invention should have sequence homologies with beneficial bacterial genes but no sequence homology to pathogenic bacterial genomes, with the host genome or with other genomes of host colonizers and/or mammals that feed on the host organism.

Non-limitative examples of beneficial bacteria which can be targeted with the method of the invention include: Bacillus (e.g., Bacillus subtilis), Pseudomonas (e.g., Pseudomonas putida, Pseudomonas stuzeri, Pseudomonas fluorescens, Pseudomonas protegens, Pseudomonas brassicacearum), Rhizobia (Rhizobium meliloti), Burkholderia (e.g., Burkholderia phytofirmans), Azospirillum (e.g., Azospirillum lipoferum), Gluconacetobacter (e.g., Gluconacetobacter diazotrophicus), Serratia (e.g., Serratia proteamaculans), Stenotrophomonas (e.g., Stenotrophomonas maltophilia), Enterobacter (e.g. Enterobacter cloacae).

### Pathogenic Fungi or Oomycetes

In a particular embodiment, the EVs of the invention contain functional iRNA(s) targeting one or multiple genes of pathogenic fungi or oomycetes.

Said fungi or oomyctes can for example be chosen in the group consisting of: Acrocalymma, Acrocalymma medicaginis, Fusarium, Fusarium affine, Fusarium arthrosporioides, Fusarium crookwellense, Fusarium culmorum, Fusarium graminearum, Fusarium incarnatum, Fusarium solani, Fusarium langsethiae, Fusarium mangiferae, Fusarium oxysporum f.sp. albedinis, Fusarium oxysporum f.sp. asparagi, Fusarium oxysporum f.sp. batatas, Fusarium oxysporum f.sp. betae, Fusarium oxysporum f.sp. cannabis, Fusarium oxysporum f.sp. carthami, Fusarium oxysporum f.sp. cattleyae, Fusarium oxysporum f.sp. ciceris, Fusarium oxysporum f.sp. coffea, Fusarium oxysporum f.sp. cubense, Fusarium oxysporum f.sp. cyclaminis, Fusarium oxysporum f.sp. dianthi, Fusarium oxysporum f.sp. lentis, Fusarium oxysporum f.sp. lini, Fusarium oxysporum f.sp. lycopersici, Fusarium oxysporum f.sp. medicaginis, Fusarium oxysporum f.sp. pisi, Fusarium oxysporum f.sp. radicis-lycopersici, Fusarium oxysporum f.sp. spinacia, Fusarium oxysporum, Fusarium pallidoroseum, Fusarium patch, Fusarium proliferatum, Fusarium redolens, Fusarium sacchari, Fusarium solani, Fusarium subglutinans, Fusarium sulphureum, Fusarium wilt, Botrytis, Botrytis allii, Botrytis anthophila, Botrytis cinerea, Botrytis fabae, Botrytis narcissicola, Alternaria, Alternaria alternata, Alternaria brassicae, Alternaria brassicicola, Alternaria carthami, Alternaria cinerariae, Alternaria dauci, Alternaria dianthi, Alternaria dianthicola, Alternaria euphorbiicola, Alternaria helianthi, Alternaria helianthicola, Alternaria japonica, Alternaria leucanthemi, Alternaria limicola, Alternaria linicola, Alternaria padwickii, Alternaria panax, Alternaria radicina, Alternaria raphani, Alternaria saponariae, Alternaria senecionis, Alternaria solani, Alternaria tenuissima, Alternaria triticina, Alternaria zinniae, Erisyphe, Erisyphe necator, Erysiphe betae, Erysiphe brunneopunctata, Erysiphe cichoracearum, Erysiphe cruciferarum, Erysiphe graminis f.sp. Avenae, Erysiphe graminis f.sp. tritici, Erysiphe heraclei, Erysiphe pisi, Claviceps, Claviceps fusiformis, Claviceps purpurea, Claviceps sorghi, Claviceps zizaniae, Gaeumannomyces, Gaeumannomyces graminis, Leptosphaeria, Leptosphaeria nodorum, Leptosphaeria acuta, Leptosphaeria cannabina, Leptosphaeria coniothyrium, Leptosphaeria libanotis, Leptosphaeria lindquistii, Leptosphaeria maculans, Leptosphaeria musarum, Leptosphaeria pratensis, Leptosphaeria sacchari, Leptosphaeria woroninii, Microdochium, Microdochium spp. Microdochium bolleyi, Microdochium dimerum, Microdochium panattonianum, Microdochium phragmitis, Mycosphaerella, Mycosphaerella arachidis, Mycosphaerella areola, Mycosphaerella berkeleyi, Mycosphaerella bolleana, Mycosphaerella brassicicola, Mycosphaerella caricae, Mycosphaerella caryigena, Mycosphaerella cerasella, Mycosphaerella coffeicola, Mycosphaerella confusa, Mycosphaerella cruenta, Mycosphaerella dendroides, Mycosphaerella eumusae, Mycosphaerella gossypina, Mycosphaerella graminicola, Mycosphaerella henningsii, Mycosphaerella horii, Mycosphaerella juglandis, Mycosphaerella lageniformis, Mycosphaerella linicola, Mycosphaerella louisianae, Mycosphaerella musae, Mycosphaerella musicola, Mycosphaerella palmicola, Mycosphaerella pinodes, Mycosphaerella pistaciarum, Mycosphaerella pistacina, Mycosphaerella platanifolia, Mycosphaerella polymorpha, Mycosphaerella pomi, Mycosphaerella punctiformis, Mycosphaerella pyri, Oculimacula, Oculimacula acuformis, Oculimacula yallundae,Blumeria, Blumeria graminis, Pyrenophora, Pyrenophora avenae, Pyrenophora chaetomioides, Pyrenophora graminea, Pyrenophora seminiperda, Pyrenophora teres, Pyrenophora teres f. maculata, Pyrenophora teres f. teres, Pyrenophora tritici-repentis, Ramularia, Ramularia collo-cygni, Ramularia beticola, Ramularia coryli, Ramularia cyclaminicola, Ramularia macrospora, Ramularia menthicola, Ramularia necator, Ramularia primulae, Ramularia spinaciae, Ramularia subtilis, Ramularia tenella, Ramularia vallisumbrosae, Rhynchosporium, Rhynchosporium secalis, Cochliobolus, Cochliobolus, Cochliobolus carbonum, Cochliobolus cymbopogonis, Cochliobolus hawaiiensis, Cochliobolus heterostrophus, Cochliobolus lunatus, Cochliobolus miyabeanus, Cochliobolus ravenelii, Cochliobolus sativus, Cochliobolus setariae, Cochliobolus spicifer, Cochliobolus stenospilus, Cochliobolus tuberculatus, Cochliobolus victoriae, Microdochium, Microdochium oryzae, Pyricularia, Pyricularia oryzae, Sarocladium, Sarocladium oryzae, Ustilaginoides, Ustilaginoides virens, Cercospora, Cercospora, Cercospora apii, Cercospora apii f.sp. clerodendri, Cercospora apiicola, Cercospora arachidicola, Cercospora asparagi, Cercospora atrofiliformis, Cercospora beticola, Cercospora brachypus, Cercospora brassicicola, Cercospora brunkii, Cercospora cannabis, Cercospora cantuariensis, Cercospora capsici, Cercospora carotae, Cercospora corylina, Cercospora fuchsiae, Cercospora fusca, Cercospora fusimaculans, Cercospora gerberae, Cercospora halstedii, Cercospora handelii, Cercospora hayi, Cercospora hydrangeae, Cercospora kikuchii, Cercospora lentis, Cercospora liquidambaris, Cercospora longipes, Cercospora longissima, Cercospora mamaonis, Cercospora mangiferae, Cercospora medicaginis, Cercospora melongenae, Cercospora minuta, Cercospora nicotianae, Cercospora odontoglossi, Cercospora papayae, Cercospora penniseti, Cercospora pisa-sativae, Cercospora platanicola, Cercospora puderii, Cercospora pulcherrima, Cercospora rhapidicola, Cercospora rosicola, Cercospora sojina, Cercospora solani, Cercospora solani-tuberosi, Cercospora sorghi, Cercospora theae, Cercospora tuberculans, Cercospora vexans, Cercospora vicosae, Cercospora zeae-maydis, Cercospora zebrina, Cercospora zonata, Corynespora, Corynespora cassiicola, Phakospora, Phakospora pachyrhizi, Phakopsora gossypii, Colletotrichum, Colletotrichum acutatum, Colletotrichum arachidis, Colletotrichum capsici, Colletotrichum cereale, Colletotrichum crassipes, Colletotrichum dematium, Colletotrichum dematium f. spinaciae, Colletotrichum derridis, Colletotrichum destructivum, Colletotrichum glycines, Colletotrichum gossypii, Colletotrichum higginsianum, Colletotrichum kahawae, Colletotrichum lindemuthianum, Colletotrichum lini, Colletotrichum mangenotii, Colletotrichum musae, Colletotrichum nigrum, Colletotrichum orbiculare, Colletotrichum pisi, Colletotrichum sublineolum, Colletotrichum trichellum, Colletotrichum trifolii, Colletotrichum truncatum, Pythium spp., Diplodia, Diplodia allocellula, Diplodia laelio-cattleyae, Diplodia manihoti, Diplodia paraphysaria, Diplodia seriata, Diplodia theae-sinensis, Monilia, Monilinia azaleae, Monilinia fructicola, Monilinia fructigena, Monilinia laxa, Monilinia oxycocci, Pezzicula, Pezzicula alba, Pezzicula malicorticis, Zymoseptoria, Zymoseptoria tritici, Phytophthora, Phytophthora infestans, Guignardia, Guignardia bidwelli, Guignardia camelliae, Guignardia fulvida, Guignardia mangiferae, Guignardia musae, Guignardia philoprina, Plasmopara, Plasmopara viticola, Puccinia, Puccinia angustata, Puccinia arachidis, Puccinia aristidae, Puccinia asparagi, Puccinia cacabata, Puccinia campanulae, Puccinia carthami, Puccinia coronata, Puccinia dioicae, Puccinia erianthi, Puccinia extensicola, Puccinia helianthi, Puccinia hordei, Puccinia jaceae, Puccinia kuehnii, Puccinia malvacearum, Puccinia mariae-wilsoniae, Puccinia melanocephala, Puccinia menthae, Puccinia oxalidis, Puccinia pelargonii-zonalis, Puccinia pittieriana, Puccinia poarum, Puccinia purpurea, Puccinia recondita, Puccinia schedonnardii, Puccinia sessilis, Puccinia striiformis, Puccinia striiformis, Puccinia subnitens, Puccinia substriata, Puccinia verruca, Puccinia xanthii, Rhizoctonia, Rhizoctonia solani, Rhizoctonia oryzae, Rhizoctonia cerealis, Rhizoctonia leguminicola, Rhizoctonia rubi, Sclerotinia, Sclerotinia borealis, Sclerotinia bulborum, Sclerotinia minor, Sclerotinia ricini, Sclerotinia sclerotiorum, Sclerotinia spermophila, Sclerotinia trifoliorum, Septoria, Septoria ampelina, Septoria azaleae, Septoria bataticola, Septoria campanulae, Septoria cannabis, Septoria cucurbitacearum, Septoria darrowii, Septoria dianthi, Septoria eumusae, Septoria glycines, Septoria helianthi, Septoria humuli, Septoria hydrangeae, Septoria lactucae, Septoria lycopersici, Septoria lycopersici, Septoria menthae, Septoria passerinii, Septoria pisi, Septoria rhododendri, Septoria secalis, Septoria selenophomoides, Venturia, Venturia inaequalis. Venturia carpophila, Acrodontium, Acrodontium simplex, Acrophialophora, Acrophialophora fusispora, Acrosporium, Acrosporium tingitaninum, Aecidium, Aecidium aechmantherae, Aecidium amaryllidis, Aecidium breyniae, Aecidium campanulastri, Aecidium cannabis, Aecidium cantensis, Aecidium caspicum, Aecidium foeniculi, Aecidium narcissi, Ahmadiago, Albonectria, Albonectria rigidiuscula, Allodus podophylli, Amphobotrys ricini, Anguillosporella vermiformis, Anthostomella pullulans, Antrodia albida, Antrodia serialiformis, Antrodia serialis, Apiospora montagnei, Appendiculella, Armillaria heimii, Armillaria sinapina, Armillaria socialis, Armillaria tabescens, Arthrocladiella, Arthuriomyces peckianus, Ascochyta asparagina, Ascochyta bohemica, Ascochyta caricae, Ascochyta doronici, Ascochyta fabae f.sp. lentis, Ascochyta graminea, Ascochyta hordei, Ascochyta humuli, Ascochyta pisi, Ascochyta prasadii, Ascochyta sorghi, Ascochyta spinaciae, Ascochyta tarda, Ascochyta tritici, Ascospora ruborum, Aspergillus aculeatus, Aspergillus fischerianus, Aspergillus niger, Asperisporium caricae, Asteridiella, Asteroma caryae, Athelia arachnoidea, Athelia rolfsii, Aurantiporus fissilis, Aureobasidium pullulans, Bambusiomyces, Banana freckle, Bayoud disease, Beniowskia sphaeroidea, Bionectria ochroleuca, Bipolaris, Bipolaris cactivora, Bipolaris cookei, Bipolaris incurvata, Bipolaris sacchari, Biscogniauxia capnodes, Biscogniauxia marginata, Bjerkandera adusta, Black sigatoka, Blakeslea trispora, Botryodiplodia oncidii, Botryodiplodia ulmicola, Botryosphaeria cocogena, Botryosphaeria dothidea, Botryosphaeria marconii, Botryosphaeria obtusa, Botryosphaeria rhodina, Botryosphaeria ribis, Botryosphaeria stevensii, Botryosporium pulchrum, Botryotinia, Botryotinia fuckeliana, Botryotinia polyblastis, Boxwood blight, Brachybasidiaceae, Brasiliomyces malachrae, Briosia ampelophaga, Brown ring patch, Buckeye rot of tomato, Bulbomicrosphaera, Cadophora malorum, Caespitotheca, Calonectria ilicicola, Calonectria indusiata, Calonectria kyotensis, Calonectria pyrochroa, Calonectria quinqueseptata, Camarotella acrocomiae, Camarotella costaricensis, Canna rust, Capitorostrum cocoes, Capnodium footii, Capnodium mangiferum, Capnodium ramosum, Capnodium theae, Cephalosporium gramineum, Ceratobasidium cereale, Ceratobasidium cornigerum, Ceratobasidium noxium, Ceratobasidium ramicola, Ceratobasidium setariae, Ceratobasidium stevensii, Ceratocystis, Ceratocystis adiposa, Ceratocystis coerulescens, Ceratocystis fimbriata, Ceratocystis moniliformis, Ceratocystis oblonga, Ceratocystis obpyriformis, Ceratocystis paradoxa, Ceratocystis pilifera, Ceratocystis pluriannulata, Ceratocystis polyconidia, Ceratocystis tanganyicensis, Ceratocystis zombamontana, Ceratorhiza hydrophila, Ceratospermopsis, Cercoseptoria ocellata, Cercosporella rubi, Ceriporia spissa, Ceriporia xylostromatoides, Cerrena unicolor, Ceuthospora lauri, Choanephora, Choanephora cucurbitarum, Choanephora infundibulifera, Chrysanthemum white rust, Chrysomyxa cassandrae, Chrysomyxa himalensis, Chrysomyxa ledi, Chrysomyxa ledi var. rhododendri, Chrysomyxa ledicola, Chrysomyxa nagodhii, Chrysomyxa neoglandulosi, Chrysomyxa piperiana, Chrysomyxa pirolata, Chrysomyxa pyrolae, Chrysomyxa reticulata, Chrysomyxa roanensis, Chrysomyxa succinea, Cladosporium arthropodii, Cladosporium cladosporioides, Cladosporium cladosporioides f.sp. pisicola, Cladosporium cucumerinum, Cladosporium herbarum, Cladosporium musae, Cladosporium oncobae, Climacodon pulcherrimus, Climacodon septentrionalis, Clitocybe parasitica, Clonostachys rosea f. rosea, Clypeoporthe iliau, Coleosporium helianthi, Coleosporium ipomoeae, Coleosporium madiae, Coleosporium pacificum, Coleosporium tussilaginis, Conidiosporomyces, Coniella castaneicola, Coniella diplodiella, Coniella fragariae, Coniothecium chomatosporum, Coniothyrium celtidis-australis, Coniothyrium henriquesii, Coniothyrium rosarum, Coniothyrium wernsdorffiae, Coprinopsis psychromorbida, Cordana johnstonii, Cordana musae, Coriolopsis floccosa, Corn grey leaf spot, Corticium invisum, Corticium penicillatum, Corticium theae, Coryneopsis rubi, Coryneum rhododendri, Covered smut, Crinipellis sarmentosa, Cronartium ribicola, Cryphonectriaceae, Cryptobasidiaceae, Cryptocline cyclaminis, Cryptomeliola, Cryptosporella umbrina, Cryptosporiopsis tarraconensis, Cryptosporium minimum, Curvularia lunata, Curvularia caricae-papayae, Curvularia penniseti, Curvularia senegalensis, Curvularia trifolii, Cyclaneusma needle cast, Cylindrocarpon ianthothele var. ianthothele, Cylindrocarpon magnusianum, Cylindrocarpon musae, Cylindrocladiella camelliae, Cylindrocladiella parva, Cylindrocladium clavatum, Cylindrocladium lanceolatum, Cylindrocladium peruvianum, Cylindrocladium pteridis, Cylindrosporium cannabinum, Cylindrosporium juglandis, Cylindrosporium rubi, Cymadothea trifolii, Cytospora palmarum, Cytospora personata, Cytospora sacchari, Cytospora sacculus, Cytospora terebinthi, Cytosporina ludibunda, Dactuliophora elongata, Davidiella dianthi, Davidiella tassiana, Deightoniella papuana, Deightoniella torulosa, Dendrophoma marconii, Dendrophora erumpens, Denticularia mangiferae, Dermea pseudotsugae, Diaporthaceae, Diaporthe, Diaporthe arctii, Diaporthe dulcamarae, Diaporthe eres, Diaporthe helianthi, Diaporthe lagunensis, Diaporthe lokoyae, Diaporthe melonis, Diaporthe orthoceras, Diaporthe perniciosa, Diaporthe phaseolorum, Diaporthe phaseolorum var. caulivora, Diaporthe phaseolorum var. phaseolorum, Diaporthe phaseolorum var. soja, Diaporthe rudis, Diaporthe tanakae, Diaporthe toxica, Dicarpella dryina, Didymella applanata, Didymella bryoniae, Didymella fabae, Didymella lycopersici, Didymosphaeria arachidicola, Didymosphaeria taiwanensis, Dilophospora alopecuri, Dimeriella sacchari, Diplocarpon mespili, Diplocarpon rosae, Discosia artocreas, Discostroma corticola, Distocercospora livistonae, Dothiorella brevicollis, Dothiorella dominicana, Dothiorella dulcispinae, Dothiorella gregaria, Drechslera avenacea, Drechslera campanulata, Drechslera dematioidea, Drechslera gigantea, Drechslera glycines, Drechslera musae-sapientium, Drechslera teres f. maculata, Drechslera wirreganensis, Eballistra lineata, Eballistra oryzae, Eballistraceae, Echinodontium ryvardenii, Echinodontium tinctorium, Ectendomeliola, Elsinoe ampelina, Elsinoe batatas, Elsinoe brasiliensis, Elsinoe leucospila, Elsinoe randii, Elsinoe rosarum, Elsinoe sacchari, Elsinoe theae, Elsinoe veneta, Endomeliola, Endothia radicalis, Endothiellagyrosa, Entorrhizomycetes, Entyloma ageratinae, Entyloma dahliae, Entyloma ellisii, Epicoccum nigrum, Eremothecium coryli, Eremothecium gossypii, Erysiphales, Exobasidiaceae, Exobasidium burtii, Exobasidium reticulatum, Exobasidium vaccinii var. japonicum, Exobasidium vaccinii-uliginosi, Exobasidium vexans,xxophiala alcalophila, Exophiala angulospora, Exophiala attenuata, Exophiala calicioides, Exophiala castellanii, Exophiala dermatitidis, Exophiala dopicola, Exophiala exophialae, Exophiala heteromorpha, Exophiala hongkongensis, Exophiala jeanselmei, Exophiala lecanii-corni, Exophiala mansonii, Exophiala mesophila, Exophiala moniliae, Exophiala negronii, Exophiala phaeomuriformis, Exophiala pisciphila, Exophiala psychrophila, Exophiala salmonis, Exophiala spinfera, Fomes lamaensis, Fomitopsis rosea, Fusicladium pisicola, Fusicoccum aesculi, Fusicoccum amygdali, Fusicoccum quercus, Galactomyces candidum, Ganoderma brownii, Ganoderma lobatum, Ganoderma megaloma, Ganoderma meredithiae, Ganoderma orbiforme, Ganoderma philippii, Ganoderma sessile, Ganoderma tornatum, Ganoderma zonatum, Geastrumia polystigmatis, Georgefischeriaceae, Georgefischeriales, Geosmithia pallida, Geotrichum candidum, Geotrichum klebahnii, Gibberella acuminata, Gibberella avenacea, Gibberella baccata, Gibberella cyanogena, Gibberella fujikuroi, Gibberella intricans, Gibberella pulicaris, Gibberella stilboides, Gibberella tricincta, Gibberella xylarioides, Gibberella zeae, Gibellina cerealis, Gilbertella persicaria, Gjaerumiaceae, Gliocladiopsis tenuis, Gliocladium vermoeseni, Gloeocercospora sorghi, Gloeocystidiellum porosum, Gloeophyllum mexicanum, Gloeophyllum trabeum, Gloeoporus dichrous, Gloeosporium cattleyae, Gloeosporium theae-sinensis, Glomerella cingulata, Glomerella graminicola, Glomerella tucumanensis, Gnomonia caryae, Gnomonia comari, Gnomonia dispora, Gnomonia iliau, Gnomonia rubi, Golovinomyces cichoracearum, Graphiola phoenicis, Graphiolaceae, Graphium rigidum, Graphium rubrum, Graphyllium pentamerum, Grovesinia pyramidalis, Gymnoconia nitens, Gymnopus dryophilus, Gymnosporangium kernianum, Gymnosporangium libocedri, Gymnosporangium nelsonii, Gymnosporangium yamadae, Haematonectria haematococca, Hansenula subpelliculosa, Hapalosphaeria deformans, Haplobasidion musae, Helicobasidium compactum, Helicobasidium longisporum, Helicobasidium purpureum, Helicoma muelleri, Helminthosporium cookei, Helminthosporium solani, Hendersonia creberrima, Hendersonia theicola, Hericium coralloides, Heterobasidion irregulare, Heterobasidion occidentale, Hexagonia hydnoides, Hymenula affinis, Hyphodermella corrugata, Hyphodontia aspera, Hyphodontia sambuci, Hypoxylon tinctor, Inonotus arizonicus, Inonotus cuticularis, Inonotus dryophilus, Inonotus hispidus, Inonotus ludovicianus, Irpex destruens, Irpex lacteus, Kabatiella caulivora, Karnal bunt, Koa wilt, Kretzschmaria zonata, Kuehneola uredinis, Kutilakesa pironii, Laetiporus ailaoshanensis, Laetiporus baudonii, Laetiporus caribensis, Laetiporus conifericola, Laetiporus cremeiporus, Laetiporus gilbertsonii, Laetiporus huroniensis, Laetiporus montanus, Laetiporus portentosus, Laetiporus zonatus, Laxitextum bicolor, Leandria momordicae, Lentinus tigrinus, Lenzites betulina, Lenzites elegans, Leohumicola atra, Leohumicola incrustata, Leohumicola levissima, Leptodontidium elatius, Leptographium microsporum, Leptosphaerulina crassiasca, Leptosphaerulina trifolii, Leptothyrium nervisedum, Leptotrochila medicaginis, Leucocytospora leucostoma, Leucostoma auerswaldii, Leucostoma canker, Leucostoma kunzei, Leucostoma persoonii, Leveillula compositarum, Leveillula leguminosarum, Leveillula taurica, Limacinula tenuis, Linochora graminis, Loose smut, Lopharia crassa, Lophodermium aucupariae, Lophodermium schweinitzii, Macrophoma mangiferae, Macrophoma theicola, Macrosporium cocos, Magnaporthe grisea, Magnaporthe salvinii, Magnaporthiopsis, Mamianiella coryli, Marasmiellus cocophilus, Marasmiellus stenophyllus, Marasmius crinis-equi, Marasmius sacchari, Marasmius semiustus, Marasmius stenophyllus, Marasmius tenuissimus, Massarina walkeri, Mauginiella scaettae, Melampsora lini, Melampsora occidentalis, Melanconis carthusiana, Melanconium juglandinum, Meliola mangiferae, Meliola zangii, Meruliopsis ambigua, Microascus brevicaulis, Microbotryum silenes-dioicae, Microbotryum violaceum, Microsphaera coryli, Microsphaera diffusa, Microsphaera ellisii, Microsphaera euphorbiae, Microsphaera hommae, Microsphaera penicillata, Microsphaera vaccinii, Microsphaera verruculosa, Microstroma juglandis, Moesziomyces bullatus, Moniliophthora roreri, Monilochaetes infuscans, Monochaetia coryli, Monochaetia mali, Monographella albescens, Monographella cucumerina, Monographella nivalis, Monosporascus cannonballus, Monosporascus eutypoides, Monostichella coryli, Mucor circinelloides, Mucor hiemalis, Mucor mucedo, Mucor paronychius, Mucor pirformis, Mucor racemosus, Mycena citricolor, Mycocentrospora acerina, Mycoleptodiscus terrestris, Didymella rabiei, Mycosphaerella recutita, Mycosphaerella rosicola, Mycosphaerella rubi, Mycosphaerella stigmina-platani, Mycosphaerella striatiformans, Mycovellosiella concors, Passalora fulva, Mycovellosiella koepkei, Mycovellosiella vaginae, Myriogenospora aciculispora, Myrothecium rovidum, Myrothecium verrucaria, Naevala perexigua, Naohidemyces vaccinii, Nectria cinnabarina, Nectria ditissima, Nectria foliicola, Nectria mammoidea, Nectria mauritiicola, Nectria peziza, Nectria pseudotrichia, Nectria radicicola, Nectria ramulariae, Nectriella pironii, Nemania diffusa, Nemania serpens, Neocosmospora vasinfecta, Neodeightonia phoenicum, Neoerysiphe galeopsidis, Neofabraea perennans, Neofusicoccum mangiferae, Oidiopsis gossypii, Oidium arachidis, Oidium caricae-papayae, Oidium indicum, Oidium mangiferae, Oidium manihotis, Olpidium brassicae, Omphalia tralucida, Ophiobolus anguillides, Ophiobolus cannabinus, Ophioirenina, Ovulinia azaleae, Oxyporus corticola, Ozonium texanum, Peltaster fructicola, Penicillium expansum, Penicillium funiculosum, Peniophora, Periconia circinata, Periconiella cocoes, Peridermium californicum, Pestalosphaeria concentrica, Pestalotia longiseta, Pestalotia rhododendri, Pestalotiopsis, Pestalotiopsis adusta, Pestalotiopsis arachidis, Pestalotiopsis disseminata, Pestalotiopsis guepini, Pestalotiopsis leprogena, Pestalotiopsis longiseta, Pestalotiopsis mangiferae, Pestalotiopsis palmarum, Pestalotiopsis sydowiana, Pestalotiopsis theae, Peyronellaea curtisii, Phacidiopycnis padwickii, Phaeochoropsis mucosa, Phaeocytostroma iliau, Phaeocytostroma sacchari, Phaeoisariopsis bataticola, Phaeoramularia heterospora, Phaeoramularia indica, Phaeoramularia manihotis, Phaeoseptoria musae, Phaeosphaerella mangiferae, Phaeosphaerella theae, Phaeosphaeria avenaria, Phaeosphaeria herpotrichoides, Phaeosphaeria microscopica, Phaeosphaeria nodorum, Phaeosphaeriopsis obtusispora, Phaeotrichoconis crotalariae, Phialophora asteris, Phialophora cinerescens, Phialophora gregata, Phialophora tracheiphila, Phoma clematidina, Phoma costaricensis, Phoma cucurbitacearum, Phoma destructiva, Phoma draconis, Phoma exigua, Phoma exigua, Phoma exigua var. foveata, Phoma exigua, Phoma glomerata, Phoma glycinicola, Phoma herbarum, Phoma insidiosa, Phoma medicaginis, Phoma microspora, Phoma narcissi, Phoma nebulosa, Phoma oncidii-sphacelati, Phoma pinodella, Phoma sclerotioides, Phoma strasseri, Phomopsis asparagi, Phomopsis asparagicola, Phomopsis cannabina, Phomopsis coffeae, Phomopsis ganjae, Phomopsis javanica, Phomopsis longicolla, Phomopsis mangiferae, Phomopsis prunorum, Phomopsis sclerotioides, Phomopsis theae, Phragmidium mucronatum, Phragmidium rosae-pimpinellifoliae, Phragmidium rubi-idaei, Phragmidium violaceum, Phyllachora banksiae, Phyllachora cannabis, Phyllachora graminis, Phyllachora gratissima, Phyllachora musicola, Phyllachora pomigena, Phyllachora sacchari, Phyllactinia, Phyllosticta alliariaefoliae Phyllosticta arachidis-hypogaeae, Phyllosticta batatas, Phyllosticta capitalensis, Phyllosticta carpogena, Phyllosticta coffeicola, Phyllosticta concentrica, Phyllosticta coryli, Phyllosticta cucurbitacearum, Phyllosticta cyclaminella, Phyllosticta erratica, Phyllosticta hawaiiensis, Phyllosticta lentisci, Phyllosticta manihotis, Phyllosticta micropuncta, Phyllosticta mortonii, Phyllosticta nicotianae, Phyllosticta palmetto, Phyllosticta penicillariae, Phyllosticta perseae, Phyllosticta pseudocapsici, Phyllosticta sojaecola, Phyllosticta theae, Phyllosticta theicola, Phymatotrichopsis omnivora, Physalospora disrupta, Physalospora perseae, Physoderma alfalfae, Physoderma leproides, Physoderma trifolii, Physopella ampelopsidis, Pileolaria terebinthi, Piricaudiopsis punicae, Piricaudiopsis rhaphidophorae, Piricaudiopsis rosae, Plenodomus destruens, Plenodomus meliloti, Pleosphaerulina sojicola, Pleospora alfalfae, Pleospora betae, Pleospora herbarum, Pleospora lycopersici, Pleospora tarda, Pleospora theae, Pleuroceras, Podosphaera, Podosphaera fuliginea, Podosphaerafusca, Podosphaera leucotricha, Podosphaera macularis, Podosphaera pannosa, Polyscytalum pustulans, Poria hypobrunnea, Postia tephroleuca, Powdery mildew, Pseudocercospora arecacearum, Pseudocercospora cannabina, Pseudocercospora fuligena, Pseudocercosporella herpotrichoides, Pseudocercospora gunnerae, Pseudocercospora pandoreae, Pseudocercospora puderi, Pseudocercospora rhapisicola, Pseudocercospora theae, Pseudocercospora vitis, Pseudocercosporella capsellae, Pseudocochliobolus eragrostidis, Pseudoepicoccum cocos, Pseudopeziza jonesii, Pseudopeziza medicaginis, Pseudopeziza trifolii, Pseudoseptoria donacis, Pucciniaceae, Pucciniastrum americanum, Pucciniastrum arcticum, Pucciniastrum epilobii, Pucciniastrum hydrangeae, Pycnostysanus azaleae, Pyrenochaeta lycopersici, Pyrenochaeta terrestris, Pyrenopeziza brassicae, Ramichloridium musae, Ramulispora sorghi, Ramulispora sorghicola, Rhinocladium corticola, Rhizophydium graminis, Rhizopus arrhizus, Rhizopus circinans, Rhizopus microsporus, Rhizopus oryzae, Rhytisma punctatum, Rhytisma vitis, Rigidoporus vinctus, Rosellinia arcuata, Rosellinia bunodes, Rosellinia necatrix, Rosellinia pepo, Saccharicola taiwanensis, Schiffnerula cannabis, Schizophyllum commune, Schizopora flavipora, Schizothyrium pomi, Sclerophthora macrospora, Sclerotium cinnamomi, Sclerotium delphinii, Scytinostroma galactinum, Seimatosporium mariae, Seimatosporium rhododendri, Selenophoma linicola, Septobasidium bogoriense, Septobasidium euryae-groffii, Septobasidium gaoligongense, Septobasidium pilosum, Septobasidium polygoni, Septobasidium pseudopedicellatum, Septobasidium theae, Septocyta ruborum, Serpula lacrymans, Setosphaeria rostrata, Setosphaeria turcica, Spencermartinsia pretoriensis, Sphaceloma arachidis, Sphaceloma menthae, Sphaceloma perseae, Sphaceloma poinsettiae, Sphaceloma sacchari, Sphaceloma theae, Sphacelotheca reiliana, Sphaerotheca castagnei, Sphaerulina oryzina, Sphaerulina rehmiana, Sphaerulina rubi, Sphenospora kevorkianii, Spilocaea oleaginea, Sporisorium cruentum, Sporisorium ehrenbergii, Sporisorium scitamineum, Sporisorium sorghi, Sporonema phacidioides, Stagonospora avenae, Stagonospora meliloti, Stagonospora recedens, Stagonospora sacchari, Stagonospora tainanensis, Stagonosporopsis, Stegocintractia junci, Stemphylium, Stemphylium alfalfae, Stemphylium bolickii, Stemphylium cannabinum, Stemphylium globuliferum, Stemphylium lycopersici, Stemphylium sarciniforme, Stemphylium solani, Stemphylium vesicarium, Stenella anthuriicola, Stigmatomycosis, Stigmina carpophila, Stigmina palmivora, Stigmina platani-racemosae, Stromatinia cepivora, Sydowiella depressula, Sydowiellaceae, Synchytrium endobioticum, Tapesia acuformis, Tapesia yallundae, Taphrina coryli, Taphrina potentillae, Thanatephorus cucumeris, Thecaphora solani, Thielaviopsis, Thielaviopsis basicola, Thielaviopsis ceramica, Thyrostroma compactum, Tiarosporella urbis-rosarum, Tilletia barclayana, Tilletia caries, Tilletia controversa, Tilletia laevis, Tilletia tritici, Tilletia walkeri, Tilletiariaceae, Togniniaceae, Tranzschelia pruni-spinosae, Trichoderma koningii, Trichoderma paucisporum, Trichoderma songyi, Trichoderma theobromicola, Trichoderma viride, Tubercularia lateritia, Tunstallia aculeata, Typhula blight, Typhula idahoensis, Typhula incarnata, Typhula ishikariensis, Typhula variabilis, Ulocladium consortiale, Uncinula, Uredo behnickiana, Uredo kriegeriana, Uredo musae, Uredo nigropuncta, Uredo rangelii, Urocystis agropyri, Urocystis brassicae, Urocystis occulta, Uromyces apiosporus, Uromyces appendiculatus, Uromyces beticola, Uromyces ciceris-arietini, Uromyces dianthi, Uromyces euphorbiae, Uromyces graminis, Uromyces inconspicuus, Uromyces lineolatus, Uromyces musae, Uromyces oblongus, Uromyces pisi-sativi, Uromyces proeminens, Uromyces medicaginis, Uromyces trifolii-repentis, Uromyces viciae-fabae, Urophlyctis leproides, Urophlyctis trifolii, Ustilaginales, Ustilago avenae, Ustilago esculenta, Ustilago hordei, Ustilago maydis, Ustilago nigra, Ustilago nuda, Ustilago scitaminea, Ustilago tritici, Vankya ornithogali, Velvet blight, Veronaea musae, Verticillium albo-atrum, Verticillium alfalfae, Verticillium dahliae, Verticillium isaacii, Verticillium klebahnii, Verticillium longisporum, Verticillium nonalfalfae, Verticillium theobromae, Verticillium wilt, Verticillium zaregamsianum, Waitea circinata, Westea, Wheat leaf rust, Wheat mildew, Wuestneiopsis georgiana, Xeromphalina fraxinophila, Zopfia rhizophila, Zygosaccharomyces bailii, Zygosaccharomyces florentinus, and Zythiostroma.

Particular target genes of some of these fungi (Botrytis cinerea, Colletotrichum higginsianum, Fusarium graminearum, Zymoseptoria tritici) are disclosed in EXAMPLE 9 below.

Particular target genes of some of these oomyctes (Phytphtora infestans, Plasmopara viticola) are disclosed in EXAMPLE 9 below.

### Beneficial Fungi

In a particular embodiment, the EVs of the invention contain functional iRNA(s) targeting one or multiple genes of beneficial fungi. Beneficial fungi include classical arbuscular mycorrhizal fungi but also other commensal fungi including the recently characterized Colletotrichum tofieldiae (Hiruma et al., 2016).

### Targeted Virus

In a particular embodiment, the EVs of the invention contain functional iRNA(s) targeting one or multiple genes of a virus. In a particular embodiment, said virus is chosen in the group consisting of: Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus and Potato virus X, Citrus tristeza virus, Barley yellow dwarf virus, Potato leafroll virus and Tomato bushy stunt virus.

Particular target genes of one of these viruses (Plum Pox Virus) are disclosed in EXAMPLE 9 below.

### Chimeric EVs of the Invention

For protecting plants against diseases caused by several bacterial pathogens, the method of the invention advantageously uses functional EVs carrying sequence homologies with more than one plant pathogen or pest (hereafter referred to as “chimeric EVs”).

In this embodiment, the small RNAs contained in the EVs of the invention can target several genes of several pathogens or parasites. These “chimeric EVs” are not specific of one pathogen or pest but can affect the growth of several pathogens (e.g., a bacterium and a virus, or of two different bacteria, or of three different viruses, etc.).

All the embodiments proposed above for the EVs, the iRNAs, the vectors, and the transformation methods are herewith encompassed and do not need to be repeated.

When the EVs of the invention target several pathogens or parasites, the phytotherapeutic composition of the invention can concomitantly treat or prevent diseases caused by these different pathogens/parasites.

In a preferred embodiment, the EVs of the invention contain chimeric iRNAs inhibiting at least one gene encoding a virulence factor or an essential gene of bacterial cells as defined above, together with at least one other gene encoding a virulence factor or an essential gene of other pathogens or parasites known to be sensitive to host-induced gene silencing. It can be also a gene required for the biosynthesis of toxic secondary metabolites from non-bacterial pathogens or parasites.

In another preferred embodiment, the phytotherapeutic applications of the invention uses: (i) EVs containing one or more iRNAs targeting a widespread sequence region of an essential or virulence gene that is conserved in a large set of pathogens or (ii) EVs containing one or more iRNAs targeting genes that are essential or virulence factors from unrelated pathogens. Such particular embodiment confers broad-spectrum protection towards multiple pathogens.

The EVs of the invention are useful for silencing any gene(s) in any microbes. Examples of useful target genes are now disclosed.

### Bacterial Target Genes

For anti-bacterial applications, the EVs of the invention should contain effective small RNAs having a sufficient sequence homology with at least one bacterial gene in order to induce sequence-specific silencing of said at least one gene. In addition, to prevent unwanted off-target effects, the sequence homology of the dsRNAs, miRNAs or small RNA species contained in said EVs with the eukaryotic host genome or other genomes of beneficial bacteria, host colonizers and/or mammals that feed on the host organism should be quasi-inexistent (if not absent).

According to the invention, the term “bacterial gene” refers to any gene in bacteria including (natural) protein-coding genes or non-coding genes, present naturally in bacteria and artificial genes introduced in bacteria by recombinant DNA technology. Said target bacterial genes are either specific to a given bacterial species or conserved across multiple bacterial species. Preferably, it shares no homology with any gene of the eukaryotic host genome, host colonizers and/or mammals that feed on the host organism. This avoids collateral effects on the plant host, beneficial bacteria associated with the host, host colonizers and/or animals that feed on the host organism.

In a preferred embodiment, said at least one bacterial gene is a bacterial virulence factor or an essential gene for bacteria or an antibiotic resistance gene.

As used herein, the term “essential gene for bacteria” refers to any bacterial gene that is essential for bacterial cell viability. These genes are absolutely required to maintain bacteria alive, provided that all nutrients are available. It is thought that the absolutely required number of essential genes for bacteria is about 250-500 in number. The identification of such essential genes from unrelated bacteria is now becoming relatively easily accessible through the use of transposon sequencing approaches. These essential genes encode proteins to maintain a central metabolism, replicate DNA, ensure proper cell division, translate genes into proteins, maintain a basic cellular structure, and mediate transport processes into and out of the cell (Zhang et al., 2009). This is the case of GyrB and FusA.

As used therein, the term “virulence gene” refers to any bacterial gene that has been shown to play a critical role for at least one of the following activity: pathogenicity, disease development, colonization of a specific host tissues (e.g., vascular tissues) or host cell environment (e.g., the apoplast), suppression of plant defense responses, modulation of plant hormone signaling and/or biosynthesis to facilitate multiplication and/or disease development, interference with conserved host regulatory processes to facilitate multiplication and/or disease development, etc. All these activities help the bacteria to grow and/or promote disease symptoms in the host, although they are not essential for their survival in vitro. Well-known virulence factors are: adhesins, phytotoxins (e.g., coronatine, syringoline A), degrading enzymes (e.g., cellulases, cellobiosidases, lipases, xylanases, endoglucanases, polygalacturonases), factors required for the assembly of type I/II/III/IV or VI secretion systems, effector proteins, transcription factors required to promote the expression of Hrp genes upon contact with plant cells, machineries required for the proper expression of virulence factors (e.g., quorum sensing, two-component systems), post-transcriptional factors controlling the stability/translation of mRNAs from virulence factor genes.

Well-known bacterial essential genes or virulence factors are provided in the following tables, for several different phytopathogenic bacteria:
For most bacteria: central factors required for survival and/or virulence such as FtsZ, FtsA, cheA, gacA, tolC, pglA, engXCA1, engXCA2, GumH, GumD, XpsE, LesA, HolC, Wp012778355, wp015452784, WP012778510, wp015452939, act56857, wp012778668, GyrB, MreB, RbfA, RsgA, FliA, QseC, Hfq, HrpR, HrpS, RpoD, HrpL, Cfa6, fusA, gyrB, rpoB, RpoC, secE, RpoA, dnaA, dnaN, HrpG, HrpB, HrcC, XpsR, TssB, HrpG, HrpX, RsmA, NadHb, NadHd and NadHe, DnaA, DnaE1, DnaE2, ZipA, PhoP, FhaB, PhoP, rim, ybeY, rplQ.
For the bacteria Pseudomonas syringae pv. phaseolicola (strains Pph 1448A; Pph 1302A), known to infect common bean plants Phaseolus vulgaris and cause the halo blight disease:

| Essential | | |
|---|---|---|
| gene | Description | Function |
| cheA2 | Chemotaxis sensor histidine kinase CheA | Chemotaxis |
| cheZ | Chemotaxis protein CheZ | Chemotaxis |
| flhB | Flagellar biosynthetic protein FlhB | Flagellum |
| fliO | Flagellar protein FliO | Flagellum |
| fliN | Flagellar motor switch protein FliN | Flagellum |
| fliM | Flagellar motor switch protein FliM | Flagellum |
| fliK | Flagellar hook-length control protein FliK | Flagellum |
| fleS | Flagellar sensor histidine kinase FleS | Flagellum |
| flgJ | Peptidoglycan hydrolase FlgJ | Flagellum |
| flgE | Flagellar book protein FlgE | Flagellum |
| flgD | Basal-body rod modification protein FlgD | Flagellum |
| FlgA | Flagellar basal-body P-ring | Flagellum |
| | formation protein, putative | |
| MotA | MotA family motility protein | Flagellum |
| plsC | hdtS protein (100%) | quorum-sensing |
| | | molecule |
| | | Outer membrane |
| | | protein and |
| impL | OmpA domain protein (98%) | pathogenicity; |
| | | might be involved |
| | | in T65S |
| mdoG1 | periplasmic glucans | |
| | biosynthesis protein MdoG | |
| CobQ | cobyric acid synthase | cobalamin |
| | | (vitamin B12) |
| | | biosynthesis |
| carA | carbamoyl-phosphate synthase, | pyrimidine |
| | small subunit | biosynthesis |
| pyrB | aspartate carbamoyltransferase | pyrimidine |
| | | biosynthesis |
| carB | carbamoyl-phosphate synthase, | pyrimidine |
| | large subunit | biosynthesis |
| purH | bifunctional purine biosynthesis | purine |
| | protein PurH | biosynthesis |
| pyrD | dihydroorotate dehydrogenase | pyrimidine |
| | cobalamin synthesis | biosynthesis |
| | protein/P47K family protein | |

For the bacteria Pseudomonas syringae pv. actinidiae, known to infect kiwi plants and fruits (Actinidia spp.) and cause the kiwifruit canker:

| Essential gene | Description |
|---|---|
| fliR | Flagellar biosynthesis protein |
| flhA | Flagellar biosynthesis protein |
| flgC | Flagellar basal-body rod protein |
| flgH | Flagellar biosynthesis protein |
| FlgL | flagellar hook-associated protein |
| | Flagellar basal-body rod modification protein |

For the bacteria Pectobacterium carotovorum, known to infect the Chinese cabbage and cause the soft rot disease:

| Essential gene | Description |
|---|---|
| pyrD | Dihydroorotate dehydrogenase |
| purH | Bifunctional phosphoribosylaminoimidazole |
| | carboxamide formyltransferase/IMP cyclohydrolase |
| purD | Phosphoribosylamine-glycine ligase |
| leuA | 2-Isopropylmalate synthase |
| serB | Phosphoserine phosphatase |
| expl | Synthesis of N-(3-oxohexanoyl)-I-homoserine lactone |
| expR | Quorum-sensing transcriptional regulator |
| flgA | Flagellar basal body P-ring biosynthesis protein |
| fliA | Flagellar biosynthesis sigma factor |
| flhB | Flagellar biosynthesis protein |
| qseC | Sensor protein QseC bacterial adrenergic receptor |
| tolC | Outer membrane channel protein |
| PCC21_023220 | Putative DNA-binding protein |

For the bacteria Ralstonia solanacearum, known to infect for instance tomato, potato, tobacco, banana and soybean and cause the bacterial wilt:

| | |
|---|---|
| Essential gene | Description |

In Tomato

| | |
|---|---|
| RSc2735 (phcB) | Regulatory protein |
| RSc2748 (phcA) | A LysR family transcriptional regulator, |
| | virulence genes transcriptional regulator |
| RSp0852 (hrpG) | Transcription regulator protein |
| | Probable type 4 prepilin peptidase; |
| | bifunctional: leader |
| RSc2827 (pilD) | peptidase and N-methyltransferase |
| | transmembrane protein |
| RSc3111 (gspL) | Probable general secretory pathway |
| | L transmembrane protein |
| RSc3114 (gspD) | Probable general secretory pathway |
| | D transmembrane protein |
| RSp1007 | Putative acetyl transferase protein |
| RSc0571 (pgk) | Probable phosphoglycerate kinase protein |
| RSc1591 (hpal1) | Putative 2,4-dihydroxyhept-2-ene-1,7-dioic |
| | acid aldolase oxidoreductase protein |
| RSp1010 | hypothetical protein |
| RSc3115 (gspE) | Probable general secretion pathway protein E |

In Arabidopsis

| | |
|---|---|
| RSc0903 (serC) | Probable phosphoserine aminotransferase |
| | (PSAT) protein |
| RSc1981 (trpA) | Probable tryptophane synthase alpha chain protein |
| RSc0454 | Putative Fe-S oxidoreductase; |
| | FAD/FMN-containing |
| | dehydrogenase oxidoreductase protein |
| RSc0411 | Probable transmembrane protein |
| RSc0353 | Probable 2,3-bisphosphoglycerate-dependent |
| (gpmA) | phosphoglycerate mutase |
| RSc1956 (murl) | Probable glutamate racemase protein |
| RSc0565 (rfaF) | Probable ADP-heptose-lipopolysaccharide |
| | heptosyltransferase II protein |
| RSc1326 | Probable aspartate aminotransferase protein |
| RSc2464 (clpA) | Probable ATP-dependent protease (ATP-binding |
| | specificity subunit) protein |
| RSc0734 (tolA) | Probable TolA-related transport |
| | transmembrance protein |
| RSc0736 (pal) | Probable peptidoglycan-associated |
| | lipoprotein precursor |
| RSc0732 (tolQ) | Probable TolQ-related transport |
| | transmembrane protein |
| RSc2684 (proC) | Probable oxidoreductase pyrroline-5-carboxylate |
| | reductase signal peptide protein |
| RSc1206 | Probable lipoprotein NlpD |
| RSc0500 (ruvB) | Probable holiday junction DNA |
| | helicase RuvB protein |

For the bacteria Xanthomonas oryzae pv. oryzicola, known to infect rice (Oryza sativa) and cause the bacterial leaf streak of rice:

| Essential gene | Description |
|---|---|
| XOCORF_4443 (HrcU) | Type III secretion protein HrcU |
| XOCORF_4444 (HcrV) | Type III secretion protein HrcV |
| XOCORF_4434 (HrcC) | Type III secretion protein HrcC |
| XOCORF_3864 (wxocB) | Putative glycosyltransferase |
| XOCORF_2264 (rpfG) | Two-component system response |
| | regulator RpfG |
| XOCORF_2899 (pilY1) | RpfG |
| XOCORF_3636 (pilQ) | Fimbrial assembly protein |
| XOCORF_3640 (pilM) | Type IV pilus assembly protein |
| XOCORF_1181 (pilZ) | Type IV pilus assembly protein |
| XOCORF_1487 (pilT) | Twitching mobility protein |
| XOCORF_3589 (pgk) | Phosphoglycerate kinase |
| XOCORF_3592 (gapA) | Glyceraldehyde-3-phosphate |
| | dehydrogenase, type l |
| XOCORF_1172 | 3-Oxoacyl-[acyl-carrier-protein] |
| | synthase III |
| XOCORF_2877 | P protein |
| XOCORF_3805 (XpsE) | General secretion pathway |
| | protein E (XpsE) |
| XOCORF_4457 (HrpF) | |
| XOCORF_3672 (ThiE) | Thiamine-phosphate |
| | pyrophosphorylase (ThiE) |
| XOCORF_3439 (HrpX) | |
| XOCORF_3794 (XpsD) | General secretion pathway |
| | protein D (XpsD) |
| XOCORF_4434 (HrcC-) | HrcC |
| XOCORF_1450 | Conserved hypothetical protein |
| XOCORF_1282 | Dipeptidyl carboxypeptidase l |

For the bacteria Xanthomonas campestris pv. campestris, known to infect all the Brassicaceae (cabbage, broccoli, cauliflower, kale, turnip, oilseed rape, mustard, radish, etc.) and cause the crucifer black rot disease:

| Essential gene | Description |
|---|---|
| B109F06 (pilC) | Fimbrial assembly protein |
| B20SG02 (pilB) | Plus biogenesis protein |
| B202G01 (iroN) | TonB-dependent receptor |
| B302H04 (wxcM) | Bifunctional acetyl transferase/isomerase |
| A404F11 (wrcC) | Glycosyltransferase |
| C302F11 (wxcA) | Glycosyltransferase |
| A105E01 (nagA) | N-acetylglucosamine-6-phosphate deacetylase |
| C110C10 (mlA) | Glucose-1-phosphate thymidylyltransferase |
| A202H06 (xpsM) | General secretion pathway protein M |
| C406A04 (xpsK) | General secretion pathway protein K |
| B502F09 (xpsF) | General secretion pathway protein F |
| B405E02 (xpsE) | General secretion pathway protein B |
| B401G05 (virB8) | VirB8 protein |
| C109G03 (engXCA) | Cellulase |
| B101F11 (rpfG) | Response regulator |
| B203D06 | Transducer protein car |
| A308G04 (trpE) | Anthranilate synthase component I |
| B605H08 (trpD) | Anthranilate synthase component II |
| B504A10 (metA) | Homoserine O-acetyltransferase |
| A506A08 (hisF) | Cyclase |
| C206E04 | Probable restriction modification |
| | system specificity subunit |
| CH11B03 (cypC) | Fatty acid alpha hydroxylase |
| A507C08 (fadB) | P-hydroxycinnamoyl CoA hydratase/lyase |
| B302H05 (uptB) | Maleylacetoacetate isomerase |
| C307G11 (catB) | Catalase (precursor) |
| B104E02 (icd) | Isocitrate dehydrogenase |
| B106G06 (ppsA) | Phosphoenolpyruvate synthase |
| B401E10 | Conserved hypothetical protein |
| C20SC01 | Conserved hypothetical proteins |
| C301C03 | Conserved hypothetical protein |
| B407F08 | Conserved hypothetical proteins |
| B410B02 | Conserved hypothetical proteins |
| B606A8 | Inserted between XC0132 and XC0133 |
| B310E10 | Inserted between XC0350 and XC0351 |
| B203A9 | Inserted between XC0408 and XC0409 (bfeA) |
| A505G9 | Inserted between XC0509 (purD) |
| | and XC0510 (purH) |
| A301D7 | Inserted between XC2898 and XC2899 (btuB) |
| B306H2 | Inserted between XC3602 and XC3603 (alkB) |
| A105E4 | Inserted between XC3604 (etf-qo) and XC3605 |
| C401H6 | Inserted between XC3604 (etf-qo) and XC3605 |
| B203C8 | Inserted between XC4037 (catB) |
| | and XC4038 (dinG) |

For the bacteria Xanthomonas axonopodis pathovars (e.g., pv. citri and pv. manihotis), known to infect the citrus tree and cassava respectively and cause the citrus canker and the cassava bacterial blight respectively:

| Essential gene | Description |
|---|---|
| XAC1211 (KatE) | monofunctional catalase |
| Xac1815 (XacFhaB) | putative hemagglutinin transporter protein |
| XAC3600 (wzt) | LPS biosynthesis |
| AvrBs2 | Type three secretion effector |
| XopX | Type three secretion effector |
| XopZ | Type three secretion effector |
| XopAO1 | Type three secretion effector |
| XopN | Type three secretion effector |
| XopQ | Type three secretion effector |

For the bacteria Erwinia amylovora, known to infect the apple and pear tree and cause the fire blight disease:

Essential gene
A7/74SmSp-A20
Ea7/74SmSp-A29
Ea7/74Sm-A33
Ea7174SmSp-A38
Ea7/74Sm-A41
Ea7/74SmSp-A56
Ea7/74SmSp-A64
Ea7/74SmSp-A72
Ea7/74SmSp-A75
Ea7/745mSp-A76
Ea7/74SmSp-A83

For the bacteria Dickeya dadantii, known to infect potato, tomato, eggplant, chicory and cause the soft rot disease:

| Essential gene | Description |
|---|---|
| Dda3937_00335 (glpD) | glycerol-3-phosphate dehydrogenase |
| Dda3937_03379 (purL) | phosphoribosylformyl-glycineamide synthetase |
| Dda3937_03564 (opgG) | Glucans biosynthesis protein G precursor |
| | phosphoribosylaminoimidazolecarboxamide |
| Dda3937_00244 (purH) | formyltransferase/IMP cyclohydrolase |
| Dda3937_00532 (hflK) | FtsH protease regulator |
| Dda3937_02515 (purM) | phosphoribosylaminoimidazole synthetase |
| Dda3937_02627 | 4-hydroxythreonine-4-phosphate dehydrogenase |
| Dda3937_00004 (guaB) | IMP dehydrogenase |
| Dda3937_03563 (opgH) | Glucans biosynthesis glucosyltransferase H |
| Dda3937_01284 (pyrB) | aspartate carbamoyltransferase |
| Dda3937_03924 (rffG) | dTDP-glucose 4,6-dehydratase |
| Dda3937_01389 (carB) | carbamoyl-phosphate synthase large subunit |
| | MexE family multidrug efflux RND transporter |
| Dda3937_03299 (acrA) | periplasmic adaptator subunit |
| Dda3937_03300 (acrB) | Multidrug efflux system protein |
| Dda3937_03258 (pyrE) | orotate phosphoribosyltransferase |
| Dda3937_02336 (nlpI) | lipoprotein |
| Dda3937_02506 (nlpB) | outer membrane protein assembly factor BamC |
| Dda3937_04018 (pta) | phosphate acetyltransferase |
| Dda3937_03554 (pyrC) | dihydro-orotase |
| Dda3937_04573 (lpxM) | acyl (myristate) transferase |
| Dda3937_01116 (glnG) | Nitrogen regulation protein NR(I), |
| | Two-component system |
| Dda3937_02099 (purF) | amidophosphoribosyltransferase |

For the bacteria Xylella fastidiosa, known to infect grapevine and olive trees and cause the pierce's disease on grapevine, the citrus variegated chlorosis, or the olive quick decline syndrome:

| Essential gene | Description |
|---|---|
| rpfC | two-component regulatory protein |
| (PD1709) MopB | major outer membrane protein |
| LesA | putative lipase/esterase |
| PD1703 (LipA) | lipase |
| PD0928 (Zot) | Zonula occludens toxin |
| PD0986 | hemagglutinin-like protein |
| gumD | Exopolysaccharides biosynthesis gene |
| gumH | Exopolysaccharides biosynthesis gene |
| xhpT | response regulators |
| PD0528 (XatA) | autotransporter protein |
| PD0279 (cgsA) | cyclic di-GMP synthase A |
| PD0062 (FimA) | Fimbrial adhesions protein |
| PD0058 (FimF) | Fimbrial adhesions protein |
| PD0731 (XadA) | encoding Xanthomonas adhesin-like protein A |
| PD1792 (HxfB) | hemagglutins |

For the bacteria Candidatus Liberibacter solanacearum, known to infect potato and to cause Zebra Chip disease, and Candidatus Liberibacter asiaticus (or americanus africanus), known to infect citrus and to cause Citrus greening disease:

Candidatus Liberibacter solanacearum

| Essential gene* | Description |
|---|---|
| WP_013462130 | VWA domain-containing protein |
| WP_076969829 | unknown protein |
| WP_013461676 | endonuclease/exonuclease/phosphatase |
| | family protein |
| WP_076969883 | unknown protein |
| WP_013462162 | OmpA family protein |
| WP_080550991 | TadE/TadG family protein |
| WP_076969829 | unknown protein |
| WP_076970537 | unknown protein |
| WP_034443047 | unknown protein |
| WP_034441878 | unknown protein |
| WP_076970544 | VWA domain-containing protein |
| WP_076969422 | unknown protein |
| WP_103846864 | VWA domain-containing protein |
| ONI59292 | serralysin |
| WP_013462515 | unknown protein |
| WP_045960760 | unknown protein |
| WP_013462289 | unknown protein |
| WP_076969829 | unknown protein |
| WP_013461834 | Na+/H+ antiporter NhaA |
| WP_013461833 | ferredoxin-NADP+ reductase protein |

*Genes denoted as WP_XXXXXXXXX correspond to “protein_id” of the gene in Candidatus Liberibacter solanacearum CLso-ZC1, complete genome with Genbank accession number NC_014774.1 of Apr. 17, 2017

Candidatus Liberibacter asiaticus

| | |
|---|---|
| WP_012778355 | unknown protein |
| WP_012778427 | unknown protein |
| WP_012778510 | unknown protein |
| WP_012778517 | unknown protein |
| ACT56857 | Serralysin |
| WP_012778607 | VWA domain-containing protein |
| WP_012778668 | unknown protein |
| WP_015452668 | VWA domain-containing protein |
| WP_015452678 | unknown protein |
| WP_015452727 | unknown protein |
| WP_015824938 | unknown protein |
| WP_015824940 | unknown protein |
| WP_015452765 | VWA domain-containing protein |
| WP_015452784 | OmpA family protein |
| WP_015452797 | unknown protein |
| WP_015452837 | unknown protein |
| WP_015824957 | unknown protein |
| WP_015452939 | VWA domain-containing protein |
| ACT56835 | Na+/H+ antiporter NhaA |
| ACT56837 | ferredoxin-NADP+ reductase protein |

*Genes denoted as WP_XXXXXXXXX correspond to “protein_id” of the gene in Candidatus Liberibacter asiaticus str. psy62, complete genome with Genbank accession number NC_012985.3 of Mar. 30, 2017

Any of these genes can be the target of the EVs of the invention.

In beneficial bacteria, the bacterial genes to be targeted are for examples: genes required for phage production that negatively regulate bacterial survival (e.g., phage baseplate assembly protein GpV), NolA and NodD2 genes from Bradyrhizobium japonicum that are known to reduce the expression of nod genes at high population densities and therefore to decrease Nod production, a bacterial signal that is essential for symbiotic invasion (knocking-down these genes from inoculant strains should thus result in competitive nodulation), the small non-coding RNA spot42 encoded by the spot forty-two (spf) gene that controls carbohydrate metabolism and uptake (knocking-down this gene from a given bacterium should result in an increased bacterial titer).

In vascular bacteria, the bacterial genes to be targeted are for example type III secretion genes.

When the targeted bacteria are phytopathogenic bacteria, said essential or virulence bacterial genes can be structural genes of secretion systems including the type III secretion system (e.g., HrcC),
genes that are required for the proper function of the type VI secretion system (e.g., TssB),
genes encoding master regulators of bacterial effector expression (e.g., HrpL, HrpG, HrpX),
genes encoding factors required for the biosynthesis of phytotoxins (e.g., Cfa6, which is important for coronatine biosynthesis in some Pseudomonas syringae pathovars),
genes required for the biosynthesis of virulence compounds (e.g., XpsR, which is important for the biosynthesis of exopolysaccharide EPS1, two-component system RavS/RavR).

The EVs of the invention contain small RNAs that share advantageously sequence homologies with any of these essential genes or virulence genes from the targeted bacterial pathogen species.

In a preferred embodiment, said virulence factor gene or bacterial viability gene is therefore chosen in the group consisting of: AvrPto, AvrPtoB, HopT1-1, FtsZ, FtsA, cheA, gacA, tolC, pglA, engXCA1, engXCA2, GumH, GumD, XpsE, LesA, HolC, Wp012778355, wp015452784, WP012778510, wp015452939, act56857, wp012778668, GyrB, MreB, RbfA, RsgA, FliA, QseC, Hfq, HrpR, HrpS, RpoD, HrpL, Cfa6, fusA, gyrB, rpoB, RpoC, secE, RpoA, dnaA, dnaN, HrpG, HrpB, HrcC, XpsR, TssB, HrpG, HrpX, RsmA, NadHb, NadHd and NadHe, DnaA, DnaE1, DnaE2, ZipA, PhoP, FhaB, PhoP, rim, ybeY, and rplQ.

In anti-bacterial applications, the EVs of the invention have advantageously sequence homologies with essential genes for the viability or virulence genes from bacterial pathogen species but no sequence homology with commensal bacteria genomes. Such advantageous embodiment of the method avoids collateral effects on the commensal bacteria present in the host.

Particular useful sequences targeting some of these bacterial genes are provided in the EXAMPLE 9 below and in the enclosed sequence listing, notably in SEQ ID NO:1-16 (IR constructs targeting Xylella fastidiosa genes), SEQ ID NO:55-60 (IR constructs that target Candidatus Liberibacter asiaticus genes), SEQ ID NO:47-54 (IR constructs that target Erwinia carotovora genes), SEQ ID NO: 61-67 and 148 (IR constructs targeting Pseudomonas syringae pv. actinidiae genes), SEQ ID NO:90-95 and SEQ ID NO:130-145 (IR constructs targeting genes from Pseudomonas syringae pv. tomato strain DC3000), SEQ ID NO:98-101 (IR constructs targeting genes from Ralstonia solanacearum), SEQ ID NO:102-107 (IR constructs targeting genes from Xanthomonas campestris pv. Campestris), SEQ ID NO:17-22 and SEQ ID NO:128-129 (IR constructs targeting genes from Xanthomonas hortorum pv. Vitians), SEQ ID NO:120-129 (IR constructs targeting genes from Xanthomonas citri pv. Fucan), SEQ ID NO:23-30 (IR constructs target genes from Acidovorax valerianella), and in SEQ ID NO:31-38 (IR constructs targeting genes from Acidovorax citrulli).

### Viral Target Genes

For anti-viral applications, the EVs of the invention should contain effective small RNAs having a sufficient sequence homology with at least one viral gene in order to induce sequence-specific silencing of said at least one gene. Said viral gene can for example be chosen in the group consisting of: P1, HC-Pro, CP, RdR, MP, N, 5′LTR, 3′LTR, 5′UTR, 3′UTR, P3, 6k1, CI, 6k2, NIa, Nib, VAP, pro-pol, viroplasmin, CPm, HEL, HSP70h, HSP70 L1, L2, MT, CP-RTD, VSR, VPg, P2, P3, P4, P5, Rap1, p33, p92, p41, p19, p22, AV2, AC4, TrAP, and Ben.

Particular useful sequences targeting viral regions are provided in EXAMPLE 9 below and in the enclosed sequence listing, notably in SEQ ID NO:39-46 (IR constructs targeting genes from Plum Pox Virus (PPV)).

### Fungal and Oomycete Target Genes

For anti-fungal applications, the EVs of the invention should contain effective small RNAs having a sufficient sequence homology with at least one fungal gene in order to induce sequence-specific silencing of said at least one gene. Said fungal gene can for example be chosen in the group consisting of: CYP51A, CYP51B, CYP51C, TOR, CGF1, DCL1, DCL2, LTF1, HBF1, CclA, ACS1, ToxA, ACS2, ELP1, ELP2, MOB2, Br1A2, stuA, F1bc, pks1, PPT1, NPP1, INF1, GK4, piacwp1-1, piacwp 1-2, piacwp1-3, PITG 17947, PITG 10772, PITG 13671, PITG 16956, PITG 00891.

Particular useful sequences targeting fungal or oomycetes regions are provided in EXAMPLE 9 below and in the enclosed sequence listing, notably in SEQ ID NO:96-97 (IR constructs targeting genes from Fusarium graminearum), SEQ ID NO:76-83 (IR constructs target genes from Botrytis cinereal), SEQ ID NO:84-89 (IR constructs targeting genes from Colletotrichum species), SEQ ID NO: 108-113 (IR constructs targeting genes from Zymoseptoria tritici), SEQ ID NO:68-75 (IR constructs targeting genes from Phytophthora infestans), and in SEQ ID NO:114-119 (IR constructs targeting genes from Plasmopara viticola).

Kit of part with compounds having biocide effects

The EVs of the invention may be applied simultaneously or in succession with other compounds.

In a preferred embodiment, the phytotherapeutic composition of the invention contains, in addition to the EVs of the invention, a biocide compound. This is particularly appropriate when the silencing element of the invention inhibits the expression of a gene that triggers the resistance to said bactericidal compound.

In this case, the composition of the invention may be supplied as a “kit of parts”, comprising the EVs of the invention and the corresponding biocide compound in a separate container.

Said kit-of-part preferably contains the phytotherapeutic composition of the invention containing Chlorella-derived EVs carrying small RNAs and the corresponding biocide compound.

The invention also relates to the use of said combination product, for inhibiting or preventing the growth or pathogenicity of pathogen(s) on target plants.

In other terms, the present invention relates to a method for treating target plants against pathogen infection, said method comprising the step of introducing into a cell of said target plant a long dsRNA molecule targeting at least one antibacterial resistance gene, and delivering to said plant the corresponding biocide compound.

The invention also relates to a method for treating target plants against pathogen infection, said method comprising the step of delivering the EVs of the invention, or a composition containing same, as well as the corresponding biocide compound, on target plant tissues prior to and/or after bacterial infection.

In this embodiment, the EVs of the invention are preferably applied prior to the biocide compound, for example few hours before, typically, two hours before.

### Transgenic Chlorella Cells of the Invention

The Chlorella cells transformed with the iRNAs of the invention and able to generate the EVs of the invention are hereafter designated as “transgenic Chlorella cells of the invention” or “recombinant Chlorella cells of the invention” or “host cells of the invention”.

These producer cells can be of any Chlorella species. In particular, they can be any cells that are currently used as food complement for humans and livestock. In a particular embodiment, they can belong to the species: Chlorella ellipsoidea, Chlorella pyrenoidosa, Chlorella sorokiniana, Chlorella vulgaris or Chlorella variabilis.

In another aspect, the present invention relates to an isolated Chlorella cell or to a transgenic Chlorella stably or transiently expressing at least one functional iRNA of the invention. It also relates to an isolated Chlorella cell containing a DNA or a viral vector containing the precursor of the invention. Said Chlorella cell may be a genetically modified cell obtained by transformation with said DNA construct or vector.

Examples of transformation processes are Agrobacterium-mediated transformation or shot-gun-mediated transformation, as described above.

All the embodiments proposed above for the Chlorella cells, the iRNAs, the precursor, the vectors, and the transformation methods are herewith encompassed and do not need to be repeated.

Methods to generate such transgenic Chlorella are disclosed in the example part below (EXAMPLE 1). They contain the steps of:
i) transforming a Chlorella cell with a DNA vector expressing at least one long functional interfering RNA of the invention, as explained above, or,
ii) infecting a Chlorella cell with a virus, preferably selected from RNA viruses able to infect Chlorella cells, engineered to express at least one functional interfering RNA of the invention from their genomes,
for a sufficient time (typically 3 to 8 days) for the Chlorella cell to stably or transiently express a significant amount of small RNAs.

By “significant amount”, it is herein meant an amount that has been shown to have an antimicrobial/antiparasitic effect in a test such as described above. This significant amount is preferably comprised between 0.05 to 100 pM, preferably between 0.05 pM and 10 pM (for in vitro applications) or between 0.05 to 100 nM, preferably between 0.05 pM and 10 nM (for in vivo applications) of EVs containing the effective small RNAs of the invention.

In particular, said transgenic Chlorella is capable of triggering host-induced gene silencing of a pathogen (e.g., a virus or a bacterium), and contains an expressible iRNA, capable of down-regulating or suppressing the expression of at least one gene of said pathogen.

In another aspect, the present invention relates to a target transgenic Chlorella stably or transiently expressing the small RNAs described above. In one embodiment, said target transgenic Chlorella contains the precursors of the invention, described above.

More precisely, the invention relates to recombinant Chlorella cells expressing a siRNA or miRNA precursor comprising a fragment of at least one target gene, said Chlorella cells releasing EV-embedded functional small iRNAs targeting said gene fragment.

In one preferred embodiment, said at least one target gene is an oomycete gene, a viral gene, a bacterial gene, or a fungus gene or a gene of any other pathogens or parasites.

In one preferred embodiment, said Chlorella cells are chosen from: Chlorella ellipsoidea, Chlorella pyrenoidosa, Chlorella sorokiniana, Chlorella vulgaris or Chlorella variabilis.

In a further aspect, the present invention is also drawn to phytotherapeutic compositions containing, as active principles, the transgenic Chlorella cells stably or transiently expressing the small RNAs described above. More precisely, these phytotherapeutic compositions advantageously contain an effective amount of the transgenic Chlorella cells that are able to produce the small RNAs of the invention in situ, once applicated on the target plant. In this aspect, the biomass of Chlorella cells can serve as a biocontrol tool for treating any parasitic infection and/or infectious disease in a plant. These Chlorella cells are preferably the recombinant cells disclosed above, that have been more preferably transformed into a powder. As a matter of fact, the phytotherapeutic composition of this part of the invention is preferably under a powder form, that can notably be easily dispersed in soil.

It is preferably used on all the plants disclosed above.

To obtain a high yield of these transgenic cells or to enhance the ability of these transgenic cells to produce EVs, it is possible to follow the recommendations disclosed above in the specific part entitled “production methods of the invention”. They apply here, mutatis mutandis, and need not be repeated.

As disclosed previously, the phytotherapeutic compositions of the invention can be formulated in a physiological or agronomical acceptable carrier, excipient or diluent. Such carriers can be any material that the plant to be treated can tolerate. Furthermore, the carrier must be such that the composition remains effective at controlling the infection, and not toxic for animals or insects that feed on the treated plants. Examples of such carriers include water, saline, Ringer's solution, dextrose or other sugar solutions, Hank's solution, and other aqueous physiologically balanced salt solutions, phosphate buffer, bicarbonate buffer and Tris buffer.

### Platform of the Invention

In a final aspect, the invention relates to a versatile platform for producing high throughput amount of functional EV-embedded interfering small RNAs, said platform using the recombinant Chlorella cells as defined above.

By “versatile”, it is meant that this platform is able to adapt or be adapted to many different functions or activities, generating modulators of a number of different pathogens, in a rapid manner.

This platform is called “MIGS platform”. It is useful for producing high amounts of siRNA populations targeting up to 1500 bp long regions in up to a dozen gene. These siRNAs are thus effective against any pathogens, in particular essential genes, which cannot easily accumulate pathogen escape mutations. They are embedded in extracellular vesicles (EVs) that protect small RNAs from ribonuclease-mediated digestion. All the advantages of this platform have been highlighted above.

The inventors are generating reporters for rapidly evaluating the biological activity of each P40 fraction batch produced from transformed Chlorella reference lines. More precisely, they engineered bacteria (here the Escherichia coli K12 strain) to express a reporter system that exhibits a differential siRNA targeted reporter gene expression when EV-embedded siRNAs are internalized and active in bacterial cells.

Five different reporter systems are herein proposed:

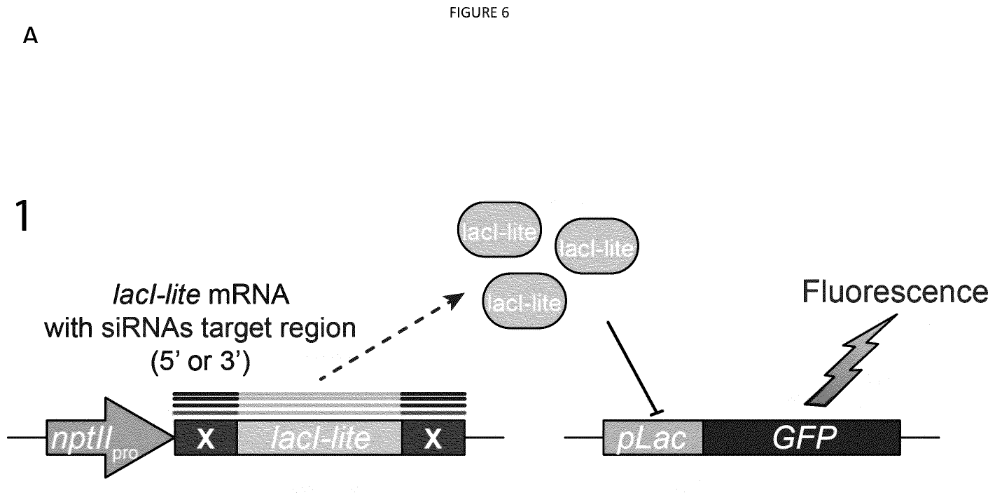

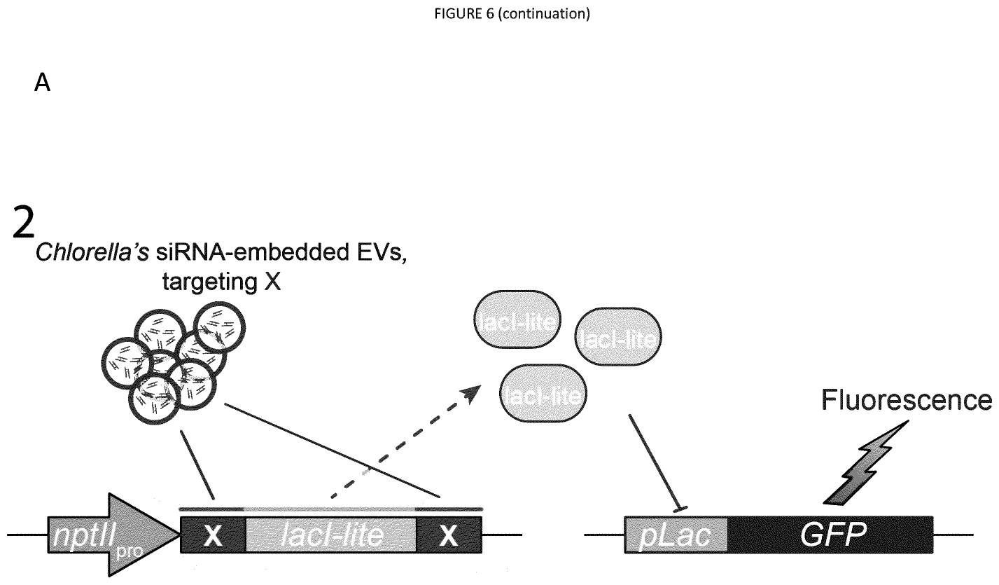

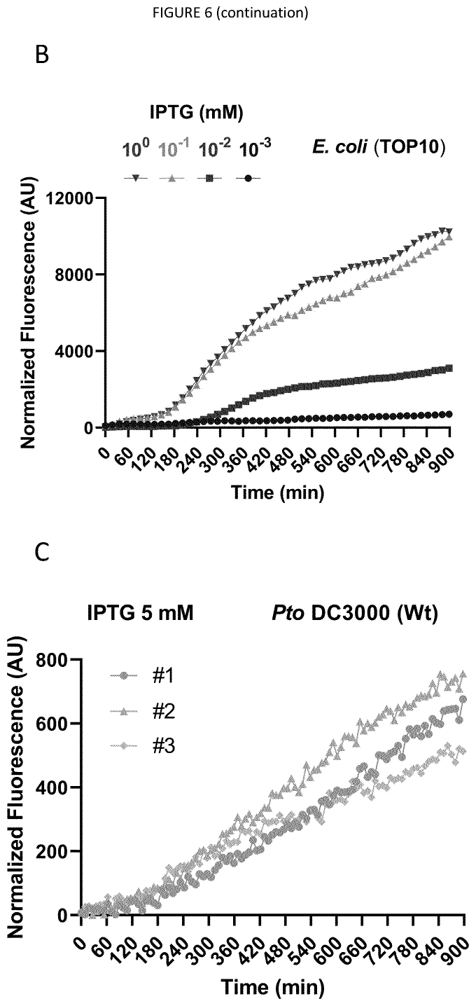

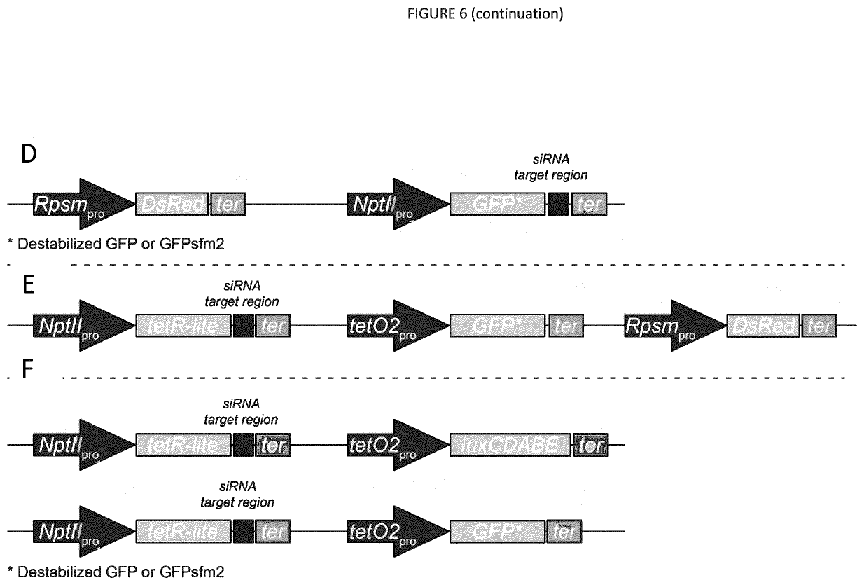

A first reporter system family is based on the plasmid expression of a bipartite cassette composed of a first construct expressing a short-lived variant of the transcriptional repressor, namely LacI-lite, carrying in its 5′ or 3′ ends the antimicrobial siRNA target region of interest, and a second construct composed of an intermediate stability variant of the GFP (Andersen et al., 1998; Elowitz & Leibler., 2000), whose transcriptional activity is directed by the pLac promoter and regulated by the lacO operator (FIG. 6A). In the absence of EV-embedded and/or associated small RNAs, LacI-lite proteins should be constitutively produced in bacteria and in turn shut-down the expression of the GFP, resulting in an absence of GFP fluorescence signal. By contrast, when a given small RNA population is internalized and active in bacterial cells, the silencing of LacI-lite results in the derepression of the GFP expression, leading to the detection of GFP fluorescence signal (FIG. 6A). Of note, other systems than LacI-lacO can also be used for the same purpose, such as the TetR-lite/tetO2 or cI-lite/λPR systems. The bipartite reporter system in destination plasmids can be assembled with backbones adapted for expression and replication in both E. coli and Pto DC3000. It is then possible to introduce a small RNA target sequence specific to a target gene (here for example the Pto DC3000 gene fusA) at the 3′ end of the LacI-lite repressor. The transformed bacterial cells can be incubated with IPTG the OD₆₀₀ and GFP fluorescence can be measured. The GFP fluorescence of the +IPTG conditions can be normalized using the −IPTG and a chloramphenicol control, in order to determine the correct induction kinetics once the background signal was removed.

A second reporter system can be generated a GFP-based reporter by assembling the strong constitutive promoter pCMV to a GFP transgene fused to a small RNA target sequence at the 3′ end of the coding sequence. This reporter can be further transfected into human cells treated with the candidate EVs population and the silencing of the GFP protein can be further monitored by different approaches including western blot analysis at 24- and 48-hours post treatments.

A third reporter system family relies on the plasmid-based expression of a cassette composed of a first construct constitutively expressing a non-targeted DsRed reporter that is used as an internal control for normalization, and a second construct carrying a destabilized GFP reporter, containing in its downstream region (or upstream region) the antimicrobial siRNA target region of interest (FIG. 6D). When expressed in bacteria (e.g., E. coli), this system will result in a specific decrease in GFP expression and fluorescence signal upon internalization of a given effective EV-embedded siRNA population.

A fourth reporter system family is based on the plasmid expression of a tripartite cassette composed of a first construct expressing a short-lived variant of the transcriptional repressor, namely TetR-lite, carrying in its downstream region the antimicrobial siRNA target region of interest, a second construct composed of an intermediate stability variant of the GFP (Andersen et al., 1998; Elowitz & Leibler, 2000), whose transcriptional activity is controlled by the tetO2 (or tetO1) operator, and a third construct expressing a non-targeted DsRed reporter, which serves as internal control for normalization (FIG. 6E). In the absence of EV-embedded small RNA, TetR-lite proteins should be constitutively produced in bacteria and in turn shut-down the expression of the GFP, resulting in an absence of GFP fluorescence signal (only the fluorescence of the DsRed reporter should be detected). By contrast, when a given siRNA population is internalized and active in bacterial cells, the silencing of TetR-lite results in the derepression of the GFP expression, leading to the detection of GFP fluorescence signal.

A fifth family of reporter system relies on the plasmid-based expression of a bipartite cassette composed of a first construct expressing a short-lived variant of the transcriptional repressor, namely TetR-lite, carrying in its downstream region (or upstream region) the antimicrobial siRNA target region of interest and a second construct composed of the GFP (Andersen et al., 1998; Elowitz & Leibler, 2000), or a bioluminescence reporter (e.g., the Photorhabdus luminescens operon luxCDABE (Meighen, 1991), whose transcriptional activity is controlled by the tetO2 (or tetO1) operator (FIG. 6F). When a given siRNA population is internalized and active in bacterial cells, the silencing of TetR-lite results in the derepression of the GFP or luxCDABE operon expression, leading to the detection of GFP fluorescence or bioluminescence signals. Of note, other systems than TetR-tetO2 could also be used for the same purpose, such as the lacI-lite/lacO or cl-lite/λPR systems.

These reporter systems are also part of the invention and will be instrumental to validate the biological activities or EV-embedded siRNA batches prior product manufacturing.

### Sequence Table

In addition to the above arrangements, the invention also comprises other arrangements, which will emerge from the description that follows, which refers to exemplary embodiments of the subject of the present invention, with reference to the attached drawings and Table of sequences in which:

| SEQ | |
|---|---|
| ID NO: | Name/details |
| 1 | Sequence of the first arm of the cheA/gaCA/tolC/ |
| | pglA/engXCA1/engXCA2 dsRNA used to |
| | concomitantly target cheA, gaCA, tolC, pglA, |
| | engXCA1, engXCA2 genes and regions from |
| | Xylella fastidiosa |
| 2 | Sequence of the second arm of the cheA/gaCA/tolC/ |
| | pglA/engXCA1/engXCA2 dsRNA used to |
| | concomitantly target cheA, gaCA, tolC, pglA, |
| | engXCA1, engXCA2 genes and regions from |
| | Xylella fastidiosa |
| 3 | Sequence of the first arm of the cheA/GumH/ |
| | GumD/XpsE/LesA/HolC dsRNA used to |
| | concomitantly target cheA, GumH, GumD, Xpse, LesA, |
| | HolC genes and regions from Xylella |
| | fastidiosa |
| 4 | Sequence of the second arm of the cheA/GumH/ |
| | GumD/XpsE/LesA/HolC dsRNA used to |
| | concomitantly target cheA, GumH, GumD, Xpse, |
| | LesA, HolC genes and regions from Xylella |
| | fastidiosa |
| 5 | Sequence of the first arm of the LesA/GumH/GumD |
| | dsRNA used to concomitantly target LesA, |
| | GumH, GumD genes and regions from Xylella fastidiosa |
| 6 | Sequence of the second arm of the LesA/GumH/ |
| | GumD dsRNA used to concomitantly target |
| | LesA, GumH, GumD genes and regions |
| | from Xylella fastidiosa |
| 7 | Sequence of the first arm of the XpsE/HolC/ |
| | LesA dsRNA used to concomitantly target XpsE, |
| | HolC, LesA genes and regions from Xylella fastidiosa |
| 8 | Sequence of the second arm of the XpsE/HolC/LesA |
| | dsRNA used to concomitantly target |
| | XpsE, HolC, LesA genes and regions from |
| | Xylella fastidiosa |
| 9 | Sequence of the first arm of the CheA/pglA/LesA |
| | dsRNA used to concomitantly target CheA, |
| | pglA, lesA genes and regions from Xylella fastidiosa |
| 10 | Sequence of the second arm of the CheA/pglA/LesA |
| | dsRNA used to concomitantly target CheA, |
| | pglA, lesA genes and regions from Xylella fastidiosa |
| 11 | Sequence of the first arm of the cheA/engXCAl/ |
| | engXCA2 dsRNA used to concomitantly target |
| | cheA, engXCA1, engXCA2 genes and regions |
| | from Xylella fastidiosa |
| 12 | Sequence of the second arm of the cheA/engXCA1/ |
| | engXCA2 dsRNA used to concomitantly |
| | target cheA, engXCA1, engXCA2 genes |
| | and regions from Xylella fastidiosa |
| 13 | Sequence of the first arm of the gacA/pglA/ |
| | engXCA2 dsRNA used to concomitantly target |
| | gacA, pglA, engXCA2 genes and regions |
| | from Xylella fastidiosa |
| 14 | Sequence of the second arm of the gacA/pglA/ |
| | engXCA2 dsRNA used to concomitantly target |
| | gacA, pglA, engXCA2 genes and regions |
| | from Xylella fastidiosa |
| 15 | Sequence of the first arm of the cheA/tolC/engXCA1 |
| | dsRNA used to concomitantly target |
| | cheA, tolC, engXCA1 genes and regions |
| | from Xylella fastidiosa |
| 16 | Sequence of the second arm of the cheA/tolC/ |
| | engXCA1 dsRNA used to concomitantly target |
| | cheA, tolC, engXCA1 genes and regions |
| | from Xylella fastidiosa |
| 17 | Sequence of the first arm of the gyrB dsRNA |
| | used to target the gyrase subunit B gene from |
| | Xanthomonas hortorum vitians |
| 18 | Sequence of the second arm of the gyrB dsRNA |
| | used to target the gyrase subunit B gene from |
| | Xanthomonas hortorum vitians |
| 19 | Sequence of the first arm of the fusA dsRNA used |
| | to target the fusA elongator factor G gene |
| | from Xanthomonas hortorum vitians |
| 20 | Sequence of the second arm of the fusA dsRNA |
| | used to target the fusA elongator factor G gene |
| | from Xanthomonas hortorum vitians |
| 21 | Sequence of the first arm of the ZipA dsRNA used |
| | to target the ZipA gene from Xanthomonas |
| | hortorum vitians |
| 22 | Sequence of the second arm of the ZipA |
| | dsRNA used to target the ZipA gene from |
| | Xanthomonas hortorum vitians |
| 23 | Sequence of the first arm of the rimM/rsgA/rbfA/ |
| | mreB/gyrB/ftsZ dsRNA used to concomitantly |
| | target rimM, rsgA, rbfA, mreB, gyrB, ftsZ |
| | genes from Acidovorax valerianella |
| 24 | Sequence of the second arm of the rimM/rsgA/ |
| | rbfA/mreB/gyrB/ftsZ dsRNA used to |
| | concomitantly target rimM, rsgA, rbfA, mreB, gyrB, |
| | ftsZ genes from Acidovorax valerianella |
| 25 | Sequence of the first arm of the rimM/rbfA/ftsZ |
| | dsRNA used to concomitantly target rimM, |
| | rbfA, ftsZ genes from Acidovorax valerianella |
| 26 | Sequence of the second arm of the rimM/rbfA/ftsZ |
| | dsRNA used to concomitantly target rimM, |
| | rbfA, ftsZ genes from Acidovorax valerianella |
| 27 | Sequence of the first arm of the RsgA/gyrB/MreB |
| | dsRNA used to concomitantly target RsgA, |
| | gyrB, MreB genes from Acidovorax valerianella |
| 28 | Sequence of the second arm of the RsgA/gyrB/ |
| | MreB dsRNA used to concomitantly target |
| | RsgA, gyrB, MreB genes from Acidovorax valerianella |
| 29 | Sequence of the first arm of the RsgA/rbfA/FtsZ |
| | dsRNA used to concomitantly target RsgA, |
| | rbfA, FtsZ genes from Acidovorax valerianella |
| 30 | Sequence of the second arm of the RsgA/rbfA/FtsZ |
| | dsRNA used to concomitantly target RsgA, |
| | rbfA, FtsZ genes from Acidovorax valeianella |
| 31 | Sequence of the first arm of the mreB/ybeY/rbfA/ |
| | gyrB/ftsZ/rsgA dsRNA used to concomitantly |
| | target the mreB, ybeY, rbfA, gyrB, ftsZ, rsgA |
| | genes from Acidovorax citrulli |
| 32 | Sequence of the second arm of the mreB/ybeY/ |
| | rbfA/gyrB/ftsZ/rsgA dsRNA used to |
| | concomitantly target the mreB, ybeY, rbfA, gyrB, |
| | ftsZ, rsgA genes from Acidovorax citrulli |
| 33 | Sequence of the first arm of the mreB/ybeY/rbfA |
| | dsRNA used to concomitantly target the mreB, |
| | ybeY, rbfA genes from Acidovorax citrulli |
| 34 | Sequence of the second arm of the mreB/ybeY/rbfA |
| | dsRNA used to concomitantly target the |
| | mreB, ybeY, rbfA genes from Acidovorax citrulli |
| 35 | Sequence of the first arm of the rsgA/GyrB/MreB |
| | dsRNA used to concomitantly target the |
| | RsgA, GyrB, MreB genes from Acidovorax citrulli |
| 36 | Sequence of the second arm of the rsgA/GyrB/MreB |
| | dsRNA used to concomitantly target the |
| | RsgA, GyrB, MreB genes from Acidovorax citrulli |
| 37 | Sequence of the first arm of the YbeY/RsgA/MreB |
| | dsRNA used to concomitantly target the |
| | YbeY, RsgA, MreB genes from Acidovorax citrulli |
| 38 | Sequence of the second arm of the YbeY/RsgA/MreB |
| | dsRNA used to concomitantly target the |
| | YbeY, RsgA, MreB genes from Acidovorax citrulli |
| 39 | Sequence of the first arm of the P1/HC-Pro/CP |
| | dsRNA used to concomitantly target the P1, |
| | HC-Pro, coat protein genes from the PIPVgp1 |
| | sequence of the Plum pox virus |
| 40 | Sequence of the second arm of the P1/HC-Pro/CP |
| | dsRNA used to concomitantly target the P1, |
| | HC-Pro, coat protein genes from the PIPVgp1 |
| | sequence of the Plum pox virus |
| 41 | Sequence of the first arm of the P1 dsRNA used |
| | to target the P1 gene from the PIPVgp1 |
| | sequence of the Plum pox virus |
| 42 | Sequence of the second arm of the P1 dsRNA |
| | used to target the P1 gene from the PIPVgp1 |
| | sequence of the Plum pox virus |
| 43 | Sequence of the first arm of the HC-Pro dsRNA |
| | used to target the HC-Pro gene from the |
| | PIPVgp1 sequence of the Plum pox virus |
| 44 | Sequence of the second arm of the HC-Pro dsRNA |
| | used to target the HC-Pro gene from the |
| | PIPVgp1 sequence of the Plum pox virus |
| 45 | Sequence of the first arm of the CP dsRNA used |
| | to target the coat protein gene from the |
| | PIPVgp1 sequence of the Plum pox virus |
| 46 | Sequence of the second arm of the CP dsRNA |
| | used to target the coat protein gene from the |
| | PIPVgp1 sequence of the Plum pox virus |
| 47 | Sequence of the first arm of the gyrB/MreB/ |
| | rbfA/RsgA/FliA/QseC dsRNA used to |
| | concomitantly target the GyrB, MreB, RbfA, RsgA, |
| | FliA, QseC genes from Erwinia carotovora |
| 48 | Sequence of the second arm of the gyrB/ |
| | MreB/rbfA/RsgA/FliA/QseC dsRNA used to |
| | concomitantly target the GyrB, MreB, RbfA, RsgA, |
| | FliA, QseC genes from Erwinia carotovora |
| 49 | Sequence of the first arm of the gyrB/rbfA/QseC |
| | dsRNA used to concomitantly target the |
| | GyrB, RbfA, QseC genes from Erwinia carotovora |
| 50 | Sequence of the second arm of the gyrB/rbfA/QseC |
| | dsRNA used to concomitantly target the |
| | GyrB, RbfA, QseC genes from Erwinia carotovora |
| 51 | Sequence of the first arm of the fliA/MreB/QseC |
| | dsRNA used to concomitantly target the FliA, |
| | MreB, QseC genes from Erwinia carotovora |
| 52 | Sequence of the second arm of the fliA/MreB/QseC |
| | dsRNA used to concomitantly target the |
| | FliA, MreB, QseC genes from Erwinia carotovora |
| 53 | Sequence of the first arm of the GyrB/MreB/RsgA |
| | dsRNA used to concomitantly target the |
| | GyrB, MreB, RsgA genes from Erwinia carotovora |
| 54 | Sequence of the second arm of the GyrB/MreB/RsgA |
| | dsRNA used to concomitantly target the |
| | GyrB, MreB, RsgA genes from Erwinia carotovora |
| 55 | Sequence of the first arm of the |
| | Wp012778355/wp015452784/WP012778510/ |
| | wp015452939/act56857/wp012778668 dsRNA |
| | used to concomitantly target the |
| | sequences encoding the proteins |
| | Wp012778355/wp015452784/WP012778510/ |
| | wp015452939/act56857/wp012778668 from |
| | Candidatus liberibacter asiaticus |
| 56 | Sequence of the second arm of the |
| | Wp012778355/wp015452784/WP012778510/ |
| | wp015452939/act56857/wp012778668 dsRNA |
| | used to concomitantly target the |
| | sequences encoding the proteins |
| | Wp012778355/wp015452784/WP012778510/ |
| | wp015452939/act56857/wp012778668 from |
| | Candidatus liberibacter asiaticus |
| 57 | Sequence of the first arm of the Wp012778355/ |
| | wp015452784/WP012778510 dsRNA used to |
| | concomitantly target the sequences encoding the proteins |
| | Wp012778355/wp015452784/WP012778510 |
| | from Candidatus liberibacter asiaticus |
| 58 | Sequence of the first arm of the Wp012778355/ |
| | wp015452784/WP012778510 dsRNA used to |
| | concomitantly target the sequences encoding the proteins |
| | Wp012778355/wp015452784/WP012778510 |
| | from Candidatus liberibacter asiaticus |
| 59 | Sequence of the first arm of the wp015452939/ |
| | act56857/wp012778668 dsRNA used to |
| | concomitantly target the sequences encoding the proteins |
| | wp015452939/act56857/wp012778668 |
| | from Candidatus liberibacter asiaticus |
| 60 | Sequence of the second arm of the wp015452939/ |
| | act56857/wp012778668 dsRNA used to |
| | concomitantly target the sequences encoding the proteins |
| | wp015452939/act56857/wp012778668 |
| | from Candidatus liberibacter asiaticus |
| 61 | Sequence of the first arm of the GyrB/Hfq/ |
| | HrpR/HRPS/MreB/RpoD dsRNA used to |
| | concomitantly target the GyrB, Hfq, HrpR, HrpS, |
| | MreB, RpoD genes from Pseudomonas |
| | syringae pv actinidiae |
| 62 | Sequence of the second arm of the GyrB/Hfq/ |
| | HrpR/HRPS/MreB/RpoD dsRNA used to |
| | concomitantly target the GyrB, Hfq, HrpR, HrpS, |
| | MreB, RpoD genes from Pseudomonas |
| | syringae pv actinidiae |
| 63 | Sequence of the first arm of the GyrB/Hfq/MreB |
| | dsRNA used to concomitantly target the |
| | GyrB, Hfq, MreB genes from |
| | Pseudomonas syringae pv actinidiae |
| 64 | Sequence of the second arm of the GyrB/Hfq/MreB |
| | dsRNA used to concomitantly target the |
| | GyrB, Hfq, MreB genes from |
| | Pseudomonas syringae pv actinidiae |
| 65 | Sequence of the first arm of the HrpR/HrpS/Hfq |
| | dsRNA used to concomitantly target the HrpR, |
| | HrpS, Hfq genes from Pseudomonas |
| | syringae pv actinidiae |
| 66 | Sequence of the second arm of the HrpR/HrpS/Hfq |
| | dsRNA used to concomitantly target the |
| | HrpR, HrpS, Hfq genes from |
| | Pseudomonas syringae pv actinidiae |
| 67 | Sequence of the first arm of the HrpR/GyrB/RpoD |
| | dsRNA used to concomitantly target the |
| | HrpR, GyrB, RpoD genes from Pseudomonas |
| | syringae pv actinidiae |
| 68 | Sequence of the first arm of the NPP1/INF1/GK4/ |
| | piacwp1-1/piacwp1-2/piacwp1-3 dsRNA |
| | used to concomitantly target the NPP1, INF1, GK4, |
| | piacwp1-1, piacwp 1-2 and piacwp1-3 |
| | genes from Phytophtora infestans |
| 69 | Sequence of the second arm of the NPP1/INF1/GK4/ |
| | piacwp1-1/piacwp1-2/piacwp1-3 dsRNA |
| | used to concomitantly target the NPP1, INF1, GK4, |
| | piacwp1-1, piacwp 1-2 and piacwp1-3 |
| | genes from Phytophtora infestans |
| 70 | Sequence of the first arm of the piacwp1-1/ |
| | piacwp1-2/piacwp1-3 dsRNA used to |
| | concomitantly target the piacwp1-1, piacwp 1-2 |
| | and piacwp1-3 genes from Phytophthora |
| | infestans |
| 71 | Sequence of the second arm of the piacwp1-1/ |
| | piacwp1-2/piacwp1-3 dsRNA used to |
| | concomitantly target the piacwp1-1, piacwp 1-2 |
| | and piacwp1-3 genes from Phytophthora |
| | infestans |
| 72 | Sequence of the first arm of the GK4/INF1/NPP1 |
| | dsRNA used to concomitantly target the |
| | GK4, INF1, NPP1 genes from Phytophtora infestans |
| 73 | Sequence of the second arm of the GK4/INF1/NPP1 |
| | dsRNA used to concomitantly target the |
| | GK4, INF1, NPP1 genes from Phytophtora infestans |
| 74 | Sequence of the first arm of the GK4/INF1/piacwp1.1 |
| | dsRNA used to concomitantly target the |
| | GK4, INF1, piacwp1.1 genes from |
| | Phytophthora infestans |
| 75 | Sequence of the second arm of the GK4/INF1/ |
| | piacwp1.1 dsRNA used to concomitantly target |
| | the GK4, INF1, piacwp1.1 genes |
| | from Phytophthora infestans |
| 76 | Sequence of the first arm of the TOR/CGF1/ |
| | DCLI/DCL2/LTF1/HBF1 dsRNA used to |
| | concomitantly target the TOR/CGF1/DCL1/ |
| | DCL2/LTF1/HBF1 genes from Botrytis cinerea |
| 77 | Sequence of the second arm of the TOR/CGF1/ |
| | DCLI/DCL2/LTF1/HBF1 dsRNA used to |
| | concomitantly target the TOR/CGF1/DCL1/DCL2/ |
| | LTF1/HBF1 genes from Botrytis cinerea |
| 78 | Sequence of the first arm of the TOR/DCL1/DCL2 |
| | dsRNA used to concomitantly target the |
| | TOR, DCL1, DCL2 genes from Botrytis cinerea |
| 79 | Sequence of the second arm of the TOR/DCL1/DCL2 |
| | dsRNA used to concomitantly target the |
| | TOR, DCL1, DCL2 genes from Botrytis cinerea |
| 80 | Sequence of the first arm of the CGF1/HBF1/LTF1 |
| | dsRNA used to concomitantly target the |
| | CGF1, HBF1, LTF1 genes from Botrytis cinerea |
| 81 | Sequence of the first arm of the CGF1/HBF1/LTF1 |
| | dsRNA used to concomitantly target the |
| | CGF1, HBF1, LTF1 genes from Botrytis cinerea |
| 82 | Sequence of the first arm of the DCL1/HBF1/TOR |
| | dsRNA used to concomitantly target the |
| | DCL1, HBF1, TOR genes from Botrytis cinerea |
| 83 | Sequence of the second arm of the DCL1/HBF1/TOR |
| | dsRNA used to concomitantly target the |
| | DCL1, HBF1, TOR genes from Botrytis cinerea |
| 84 | Sequence of the first arm of the CclA/ACS1/ACS2/ |
| | ELP1/ELP2/MOB2 dsRNA used to |
| | concomitantly target the CclA/ACS1/ACS2/ELP1/ |
| | ELP2/MOB2 genes from Colletotrichum |
| | higginsianum |
| 85 | Sequence of the second arm of the CclA/ACS1/ |
| | ACS2/ELP1/ELP2/MOB2 dsRNA used to |
| | concomitantly target the CclA/ACS1/ACS2/ELP1/ |
| | ELP2/MOB2 genes from Colletotrichum |
| | higginsianum |
| 86 | Sequence of the first arm of the CclA/ACS1/ACS2 |
| | dsRNA used to concomitantly target the |
| | CclA/ACS1/ACS2 genes from |
| | Colletotrichum higginsianum |
| 87 | Sequence of the second arm of the CclA/ACS1/ACS2 |
| | dsRNA used to concomitantly target the |
| | CclA/ACS1/ACS2 genes from |
| | Colletotrichum higginsianum |
| 88 | Sequence of the first arm of the ELP1/ELP2/MOB2 |
| | dsRNA used to concomitantly target the |
| | ELP1/ELP2/MOB2 genes from |
| | Colletotrichum higginsianum |
| 89 | Sequence of the second arm of the ELP1/ELP2/MOB2 |
| | dsRNA used to concomitantly target the |
| | ELP1/ELP2/MOB2 genes from |
| | Colletotrichum higginsianum |
| 90 | Sequence of the first arm of the CFA6/HRPL |
| | dsRNA used to concomitantly target the HrpL |
| | and Cfa6 genes of Pto DC3000 |
| 91 | Sequence of the second arm of the CFA6/HRPL |
| | dsRNA used to concomitantly target the HrpL |
| | and Cfa6 genes of Pto DC3000 |
| 92 | Sequence of the first arm of the HRPL dsRNA |
| | used to target the HrpL gene of Pto DC3000 |
| 93 | Sequence of the second arm of the HRPL dsRNA |
| | used to target the HrpL gene of Pto DC3000 |
| 94 | Sequence of the first arm of the AvrPto/AvrPtoB |
| | dsRNA used to target the AvrPto and AvrPtoB |
| | genes of Pto DC3000 |
| 95 | Sequence of the second arm of the AvrPtoAvrPtoB |
| | dsRNA used to target the AvrPto and |
| | AvrPtoB genes of Pto DC3000 |
| 96 | Sequence of the first arm of the CYP51 dsRNA |
| | used to target the FgCYP51A, FgCYP51B and |
| | FgCYP51C genes of Fusarium graminearum |
| 97 | Sequence of the second arm of the CYP51 dsRNA |
| | used to target the FgCYP51A, FgCYP51B |
| | and FgCYP51C genes of Fusarium graminearum |
| 98 | Sequence of the first arm of the HRPG/HRPB/HRCC |
| | dsRNA used to target concomitantly the |
| | HrpG, HrpB and HrcC genes of Ralstonia solanacearum |
| 99 | Sequence of the second arm of the HRPG/HRPB/HRCC |
| | dsRNA used to target concomitantly |
| | the HrpG, HrpB and HrcC genes |
| | of Ralstonia solanacearum |
| 100 | Sequence of the first arm of the HRPB/HRCC/ |
| | TssB/XpsR dsRNA used to target concomitantly |
| | the HrpB, HrcC, XpsR and TssB genes |
| | of Ralstonia solanacearum |
| 101 | Sequence of the second arm of the |
| | HRPB/HRCC/TssB/XpsR dsRNA used to target |
| | concomitantly the HrpB, HrcC, XpsR and TssB |
| | genes of Ralstonia solanacearum |
| 102 | Sequence of the first arm of the HRPG/HRPX/ |
| | RsmA dsRNA used to target concomitantly the |
| | HrpG, HrpX, and RsmA genes of |
| | Xanthomonas campestris pv. campestris |
| 103 | Sequence of the second arm of the HRPG/HRPX/ |
| | RsmA dsRNA used to target concomitantly |
| | the HrpG, HrpX, and RsmA genes |
| | of Xanthomonas campestris pv. campestris |
| 104 | Sequence of the first arm of the NadHb/NadHd/NadHe |
| | dsRNA used to target concomitantly |
| | the NadHb, NadHd and NadHe genes of |
| | Xanthomonas campestris pv. campestris (and likely |
| | other Xanthomonas species) |
| 105 | Sequence of the second arm of the NadHb/NadHd/ |
| | NadHe dsRNA used to target concomitantly |
| | the NadHb, NadHd and NadHe genes |
| | of Xanthomonas campestris pv. campestris (and likely |
| | other Xanthomonas species) |
| 106 | Sequence of the first arm of the DnaA/DnaE1/DnaE2 |
| | dsRNA used to target concomitantly the |
| | DnaA, DnaE1 and DnaE2 genes of |
| | Xanthomonas campestris pv. campestris (and likely other |
| | species) |
| 107 | Sequence of the second arm of the DnaA/DnaE1/ |
| | DnaE2 dsRNA used to target concomitantly |
| | the DnaA, DnaE1 and DnaE2 genes of |
| | Xanthomonas campestris pv. campestris (and likely |
| | other species) |
| 108 | Sequence of the first arm of the Br1A2/stuA/ |
| | F1bc/pks1/PPT1 dsRNA used to concomitantly |
| | target the Br1A2/stuA/F1bc/pks1/PPT1 genes |
| | from Zymoseptoria tritici |
| 109 | Sequence of the second arm of the Br1A2/ |
| | stuA/F1bc/pks1/PPT1 dsRNA used to concomitantly |
| | target the Br1A2/stuA/F1bc/pks1/PPT1 |
| | genes from Zymoseptoria tritici |
| 110 | Sequence of the first arm of the Br1A2/stuA/ |
| | F1bc dsRNA used to concomitantly target the |
| | Br1A2/stuA/F1bc genes from Zymoseptoria tritici |
| 111 | Sequence of the second arm of the Br1A2/stuA/ |
| | F1bc dsRNA used to concomitantly target the |
| | Br1A2/stuA/F1bc genes from Zymoseptoria tritici |
| 112 | Sequence of the first arm of the stuA/pks1/PPT1 |
| | dsRNA used to concomitantly target the |
| | stuA/pks1/PPT1 genes from Zymoseptoria tritici |
| 113 | Sequence of the second arm of the stuA/pks1/PPT1 |
| | dsRNA used to concomitantly target the |
| | stuA/pks1/PPT1 genes from Zymoseptoria tritici |
| 114 | Sequence of the first arm of the chitinase/elicitin/ |
| | glucanase inhibitor, transglutaminase elicitor/ |
| | secretory protein dsRNA used to concomitantly |
| | target genes producing the protein defined by |
| | PITG_17947, PITG_10772, PITG_13671, |
| | PITG_16956, PITG_00891 from Plasmopora |
| | viticola |
| 115 | Sequence of the second arm of the chitinase/elicitin/ |
| | glucanase inhibitor, transglutaminase |
| | elicitor/secretory protein dsRNA used to |
| | concomitantly target genes producing the protein |
| | defined by PITG_17947, PITG_10772, |
| | PITG_13671, PITG_16956, PITG_00891 from |
| | Plasmopora viticola |
| 116 | Sequence of the first arm of the chitinase/ |
| | elicitin/glucanase inhibitor dsRNA used to |
| | concomitantly target genes producing the protein |
| | defined by PITG_17947, PITG_10772, |
| | PITG_13671 from Plasmopora viticola |
| 117 | Sequence of the second arm of the chitinase/ |
| | elicitin/glucanase inhibitor dsRNA used to |
| | concomitantly target genes producing the protein |
| | defined by PITG_17947, PITG_10772, |
| | PITG_13671 from Plasmopora viticola |
| 118 | Sequence of the first arm of the glucanase inhibitor, |
| | transglutaminase elicitor/secretory protein |
| | dsRNA used to concomitantly target genes |
| | producing the protein defined by PITG_13671, |
| | PITG_16956, PITG_00891 from Plasmopora viticola |
| 119 | Sequence of the second arm of the glucanase |
| | inhibitor, transglutaminase elicitor/secretory |
| | protein dsRNA used to concomitantly target |
| | genes producing the protein defined by |
| | PITG_13671, PITG_16956, PITG_00891 |
| | from Plasmopora viticola |
| 120 | Sequence of the first arm of the GyrB/FusA/ |
| | MreB/HrpG/PhoP/FhaB dsRNA used to |
| | concomitantly target the GyrB, FusA, MreB, |
| | HrpG, PhoP, FhaB genes from Xanthomonas citri |
| | pv fuscan |
| 121 | Sequence of the second arm of the GyrB/ |
| | FusA/MreB/HrpG/PhoP/FhaB dsRNA used to |
| | concomitantly target the GyrB, FusA, MreB, |
| | HrpG, PhoP, FhaB genes from Xanthomonas citri |
| | pv fuscan |
| 122 | Sequence of the first arm of the GyrB/RbfA/ |
| | MreB dsRNA used to concomitantly target the |
| | GyrB, RbfA, MreB genes from |
| | Xanthomonas citri pv fuscan |
| 123 | Sequence of the second arm of the GyrB/RbfA/ |
| | MreB dsRNA used to concomitantly target the |
| | GyrB, RbfA, MreB genes from |
| | Xanthomonas citri pv fuscan |
| 124 | Sequence of the first arm of the HrpG/PhoP/ |
| | FtsZ dsRNA used to concomitantly target the |
| | HrpG, PhoP, FtsZ genes from Xanthomonas citri pv fuscan |
| 125 | Sequence of the second arm of the HrpG/ |
| | PhoP/FtsZ dsRNA used to concomitantly target the |
| | HrpG, PhoP, FtsZ genes from Xanthomonas citri pv fuscan |
| 126 | Sequence of the first arm of the FhaB/FusA/ |
| | MreB dsRNA used to concomitantly target the |
| | HrpG, PhoP, FtsZ genes from Xanthomonas citri pv fuscan |
| 127 | Sequence of the second arm of the FhaB/ |
| | FusA/MreB dsRNA used to concomitantly target the |
| | HrpG, PhoP, FtsZ genes from Xanthomonas citri pv fuscan |
| 128 | Sequence of the first arm of the GyrB/FusA/ |
| | ZipA dsRNA used to concomitantly target GyrB, |
| | FusA, ZipA genes from Xanthomonas hortorum pv vitians |
| 129 | Sequence of the second arm of the GyrB/FusA/ |
| | ZipA dsRNA used to concomitantly target |
| | GyrB, FusA, ZipA genes from Xanthomonas |
| | hortorum pv vitians |
| 130 | Sequence of the first arm of the AvrPto/ |
| | AvrPtoB/HopT1-1 dsRNA used to concomitantly target |
| | the AvrPto, AvrPtoB, HopT1-1 genes from |
| | Pseudomonas syringae pv. tomato strain DC3000 |
| 131 | Sequence of the second arm of the AvrPto/ |
| | AvrPtoB/HopT1-1 dsRNA used to concomitantly |
| | target the AvrPto, AvrPtoB, HopT1-1 genes |
| | from Pseudomonas syringae pv. tomato strain |
| | DC3000 |
| 132 | Sequence of the first arm of the rpoB/rpoC/fusA |
| | dsRNA used to concomitantly target the rpoB, |
| | RpoC and fusA genes from Pto DC3000 |
| 133 | Sequence of the second arm of the rpoB/rpoC/fusA |
| | dsRNA used to concomitantly target the |
| | rpoB, RpoC and fusA genes from Pto DC3000 |
| 134 | Sequence of the first arm of the secE/rpoA/rplQ |
| | dsRNA used to concomitantly target the secE, |
| | RpoA and rplQ genes from Pto DC3000 |
| 135 | Sequence of the second arm of the secE/rpoA/rplQ |
| | dsRNA used to concomitantly target the |
| | secE, RpoA and rplQ genes from Pto DC3000 |
| 136 | Sequence of the first arm of the fusA dsRNA used |
| | to target the fusA gene from Pto DC3000 |
| 137 | Sequence of the second arm of the fusA dsRNA |
| | used to target the fusA gene from Pto DC3000 |
| 138 | Sequence of the first arm of the gyrB dsRNA |
| | used to target the gyrB gene from Pto DC3000 |
| 139 | Sequence of the second arm of the gyrB dsRNA |
| | used to target the gyrB gene from Pto DC3000 |
| 140 | Sequence of the first arm of the fusA/gyrB dsRNA |
| | used to concomitantly target the fusA and |
| | gyrB genes from Pto DC3000 |
| 141 | Sequence of the second arm of the fusA/gyrB dsRNA |
| | used to concomitantly target the fusA and |
| | gyrB genes from Pto DC3000 |
| 142 | Sequence of the first arm of the secE dsRNA used |
| | to target the secE gene from Pto DC3000 |
| 143 | Sequence of the second arm of the secE dsRNA |
| | used to target the secE gene from Pto DC3000 |
| 144 | Sequence of the first arm of the gyrB/hrpL dsRNA |
| | used to concomitantly target the gyrB and |
| | hrpL genes from Pto DC3000 |
| 145 | Sequence of the second arm of the gyrB/hrpL |
| | dsRNA used to concomitantly target the gyrB |
| | and hrpL genes from Pto DC3000 |
| 146 | Sequence of the first arm of the dnaA/dnaN/gyrB |
| | dsRNA used to concomitantly target the |
| | dnaA, dnaN and gyrB genes from Pto DC3000 |
| 147 | Sequence of the second arm of the dnaA/dnaN/gyrB |
| | dsRNA used to concomitantly target the |
| | dnaA, dnaN and gyrB genes from Pto DC3000 |
| 148 | Sequence of the second arm of the HrpR/GyrB/RpoD |
| | dsRNA used to concomitantly target the |
| | HrpR, GyrB, RpoD genes from |
| | Pseudomonas syringae pv. actinidiae |
| 149 | Sequence of a specific intron sequence from |
| | the Petunia Chalcone synthase gene CHSA |

### FIGURE LEGENDS

FIG. 1. Scattering and fluorescence NTA analyses of Chlorella EVs
A) Size distribution of overall Chlorella particles from P40 fractions, determined through scattering analysis (using the Particle Metrix ZetaView system).
B) Size distribution of overall Chlorella particles from P100 fractions, determined through scattering analysis (using the Particle Metrix ZetaView system).
C) Transmission electron microscopy (TEM) images of P100 Chlorella EVs.
D) Size distribution of P100 Chlorella EVs (n=609) measured from TEM images.
E) Size distribution of PKH26-labeled Chlorella particles from P40 fractions (using the Particle Metrix ZetaView system). The measurements were performed using the 488 nm laser.

FIG. 2. Phylogenetic analysis of Chlorella variabilis AGO and DCL proteins and RNA-sequencing analysis of small RNAs from a Chlorella vulgaris reference transgenic line
A) NJ trees (1000 bootstraps) including 111 AGO and 77 DCL sequences, respectively, from plants, animals, fungi and algae. The position of the C. variabilis AGO and DCL proteins are shown. The trees are midpoint rooted.
B) Comparative size distribution profile between the Arabidopsis IR-CFA6/HRPL #4 and the Chlorella reference transgenic line IT20-3 small RNAs. The most aboundant small RNA population from Chlorella is of 18 nt long, whilst in Arabidopsis they are of 21 and 24 nt long. It is also noteworthy that Chlorella and Arabidopsis exhibit minor peaks of small RNA species at 15 and 16 nt, respectively. Two biological replicates are shown separately in this analysis.

FIG. 3. Chlorella vulgaris can be engineered to produce active small RNAs targeting the Pto DC3000 virulence factors cfa6 and hrpL
A) Schematic representation of the IT20 construct designed to express small RNAs targeting the Pto DC3000 cfa6 and hrpL mRNAs, under the control of the constitutive Cauliflower Mosaic Virus (CaMV) 35S promoter. The chimeric 504 bp region targeting the two virulence genes has been cloned in sense (B module) and antisense (D module) orientations using the Green Gate assembly strategy.
B) Stomatal reopening assay at 3 hours post-infection (hpi) on Arabidopsis (Col-0 accession) leaf sections incubated with water (Mock) or total RNAs (20 pg) from Arabidopsis thaliana (At), wild type Chlorella or transgenic Chlorella lines expressing the IR-CFA6/HRPL transgene. Leaves were incubated with wild type (Pto/Pto Wt) or mutant Pto DC3000 strains (of note, Pto ΔCor, is deleted of the cfa6 gene and is thus altered in the reopening of stomata). N, total number of analyzed stomata. Statistical analyses were performed using 2way ANOVA and compared with the mock condition (p-value: ns>0.05; ***<0.001; ****<0.0001).
C) Coverage of small RNAs reads showing the total count of mapped reads across the IR-CFA6/HRPL inverted repeat (on both the minus and plus strands). The reads in black map to the cfa6 sequence region, while the reads in dark grey map to the hrpL sequence region.

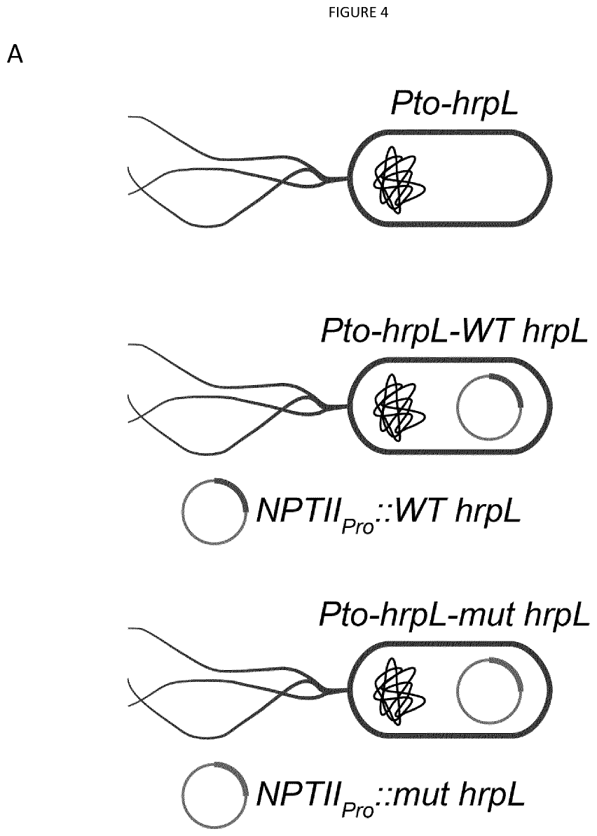

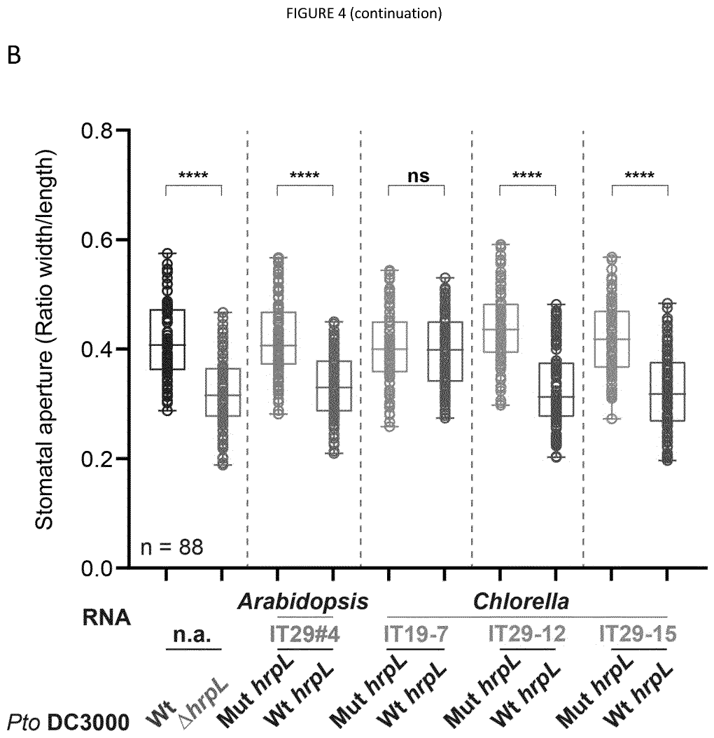

FIG. 4. Chlorella artificial small RNAs directed against Pto DC3000 hrpL transcripts are causal for the suppression of hrpL-mediated stomatal reopening function
A) Schematic representation of the Pto DC3000 ΔhrpL strain along with the complementation strains generated upon transformation with the plasmids encoding WT hrpL or mut hrpL respectively, under the control of the constitutive promoter NPTII.
B) Stomatal reopening assay at 3 hpi on Arabidopsis (Col-0) leaf sections incubated with total RNAs (20 pg) from Arabidopsis thaliana (At) or Chlorella transgenic lines expressing the IR-HRPL construct (Arabidopsis, clone IT29 #4; Chlorella, clones IT29 #12 an IT29 #15) or the control IR-CYP51 construct (Chlorella, IT19 #7). Stomatal reopening response was assessed as described in FIG. 3B.

FIG. 5. Stomatal reopening assay using concentrated media (CM) and P40 fractions from transgenic Chlorella vulgaris lines
A) Stomatal reopening assay at 3 hpi on Arabidopsis (Col-0) leaf sections incubated with water (Mock), total RNA (20 μg) or concentrated media (CM) from Chlorella transgenic lines (IT20 #3 and IT20 #5) and wild type (Wt) lines. Total RNA (20 μg) from the Arabidopsis IR-CFA6/HRPL #4 reference line was also used as a control. Leaf sections were inoculated with Pto DC3000 Wt or mutant strains. N, total number of analyzed stomata. Statistical analyses were performed using 2way ANOVA and compared with the mock condition (p-value: ns>0.05; ***<0.001; ****<0.0001).
B) as in A) except that the P40 fraction from the IT20 #3 line was used for this assay. RNA extracts from the same Chlorella transgenic line were also used as a positive control. N, total number of analyzed stomata. Statistical analyses were performed using 2way ANOVA and compared with the mock condition (p-value: ns>0.05; ***<0.001; ****<0.0001).
C) as in B) but the P40 fractions were subjected to a 30′ incubation at 37° C. in the presence or absence of 300 U/ml of Mnase. The digestion reaction was blocked by adding EGTA, at a final concentration of 20 mM.
D) Coverage of small RNAs reads from the P100 fraction sample computed as the total count of mapped reads across the IR-CFA6/HRPL inverted repeat is depicted and includes both the plus and minus strands of the construct. The reads in black map to the cfa6 sequence region, while the reads in dark grey map to the hrpL sequence region.
E) Stomatal reopening assay performed using the Pto DC3000 ΔhrpL bacteria complemented either with the Wt or the Mut hrpL versions. Both P40 and P100 EVs fractions from the Chlorella IR-HRPL (IT29 #12) and IR-CYP51 (IT19 #7) lines were treated with the Mnase as described, before performing the assay. Stomatal reopening response was assessed as described in A).

FIG. 6. Schematic representation of the reporter systems that will be used to monitor EV-embedded siRNA activity in bacterial cells
A) Schematic representation of the bipartite cassette designed to detect a specific gain of GFP expression upon treatment with a given EV-embedded siRNA population. The system is composed of a first construct that includes a short-lived variant of a transcriptional repressor, such as the depicted lacI-lite, which contains a siRNA target region of interest in its downstream region (or potentially in its upstream region), driven by a constitutive promoter (e.g., the nptII promoter sequence depicted here as an example) and a downstream terminator sequence (e.g., the SoxR terminator sequence); and a second construct that includes a destabilized GFP reporter sequence (e.g., the intermediate stability GFP variant gpf-aav sequence (Campbell-Valois et al., 2014; Elowitz & Leibler, 2000)), whose transcriptional activity is controlled by the lacO operator in a pLac promoter, and is composed of a downstream terminator sequence (e.g., the SoxR terminator sequence). In normal conditions, the GFP expression is repressed by the presence of the LacI-lite repressor (1). Silencing of such a repressor, triggered by EV-contained small RNAs targeting the regulatory region “X”, releases the inhibition allowing GFP expression (2).
B) Kinetics of the fluorescence induction mediated by different concentrations of IPTG on E. coli TOP10 cells containing the R37 construct. This construct has a small RNAs targeted region corresponding to the Pto DC3000 genefusA cloned at the 3′ end of the lac gene. The GFP fluorescence obtained by subtracting the fluorescence values of the −IPTG and the +Chloramphenicol conditions was used as control. The fluorescence and OD₆₀₀ (not shown) were monitored in a Tecan Infinite 200 plate reader system, by means of specific filters performing data acquisition every 5′ for 15 hours at 37° C.
C) Kinetics of the fluorescence induction mediated by the IPTG on Pto DC3000 Wt cells containing the R37 construct. The GFP fluorescence obtained by subtracting the fluorescence values of the −IPTG and the +Chloramphenicol conditions was used as control. The fluorescence and OD₆₀₀ (not shown) were monitored in a Tecan Infinite 200 plate reader system, by means of specific filters performing data acquisition every 5′ for 15 hours at 28° C.
D) Schematic representation of the first dual reporter family cassette designed to detect a specific decrease in GFP expression upon treatment with a given EV-embedded siRNA population. The dual reporter cassette is composed of a first DsRed reporter construct driven by a constitutive promoter (e.g., the Rpsm promoter sequence depicted here as an example) and a downstream terminator sequence (e.g., the TonB terminator sequence depicted here as an example); a second construct composed of a destabilized GFP reporter version (e.g., the GFPsmf2 sequence carrying a degradation tag in its downstream region or the intermediate stability GFP variant gpf-aav sequence (Campbell-Valois et al., 2014; Elowitz & Leibler, 2000) that contains the siRNA target sequence region of interest cloned in its downstream region (or upstream, not shown), driven by a constitutive promoter (e.g., the NPTII promoter sequence depicted here as an example), with a downstream terminator sequence (e.g., the SoxR terminator sequence depicted here as an example).
E) Schematic representation of the second tripartite cassette designed to detect a specific gain of GFP expression upon treatment with a given EV-embedded siRNA population. The tripartite cassette is composed of a first construct that includes a short-lived variant of a transcriptional repressor, such as the depicted TetR-lite, which contains a siRNA target region of interest in its downstream region (or eventually in its upstream region, not shown), driven by a constitutive promoter (e.g., the NPTII promoter sequence depicted here as an example) and a downstream terminator sequence (e.g., the SoxR terminator sequence depicted here as an example); a second construct that includes a destabilized GFP reporter sequence (e.g., the GFPsmf2 sequence carrying a degradation tag in its downstream region or the intermediate stability GFP variant gpf-aav sequence (Campbell-Valois et al., 2014; Elowitz & Leibler, 2000), whose transcriptional activity is controlled by the tetO2 operator, and composed of a downstream terminator sequence (e.g., the soxR or tonB terminator sequences depicted here as an example); a third construct that includes a DsRed reporter sequence driven by a constitutive promoter (e.g., the Rpsm promoter sequence depicted here as an example) and a downstream terminator sequence (e.g., the TonB terminator sequence depicted here as an example).
F) Schematic representation of the third bipartite cassette designed to detect a specific gain of a reporter gene expression upon treatment with a given EV-embedded siRNA population. The bipartite cassette is composed of a first construct that includes a short-lived variant of a transcriptional repressor, such as the depicted TetR-lite, which contains a siRNA target region of interest in its downstream region (or eventually in its upstream region, not shown), driven by a constitutive promoter (e.g., the NPTII promoter sequence depicted here as an example), and a downstream terminator sequence (e.g., the SoxR terminator sequence depicted here as an example); a second construct that includes a destabilized GFP reporter sequence (e.g., the GFPsmf2 sequence carrying a degradation tag in its downstream region or the intermediate stability GFP variant gpf-aav sequence (Campbell-Valois et al., 2014; Elowitz & Leibler, 2000) or a bioluminescence reporter (e.g., the Photorhabdus luminescens operon luxCDABE (Meighen, 1991), whose transcriptional activity is controlled by the tetO2 operator, and composed of a downstream terminator sequence (e.g., the soxR or tonB terminator sequence depicted here as an example).

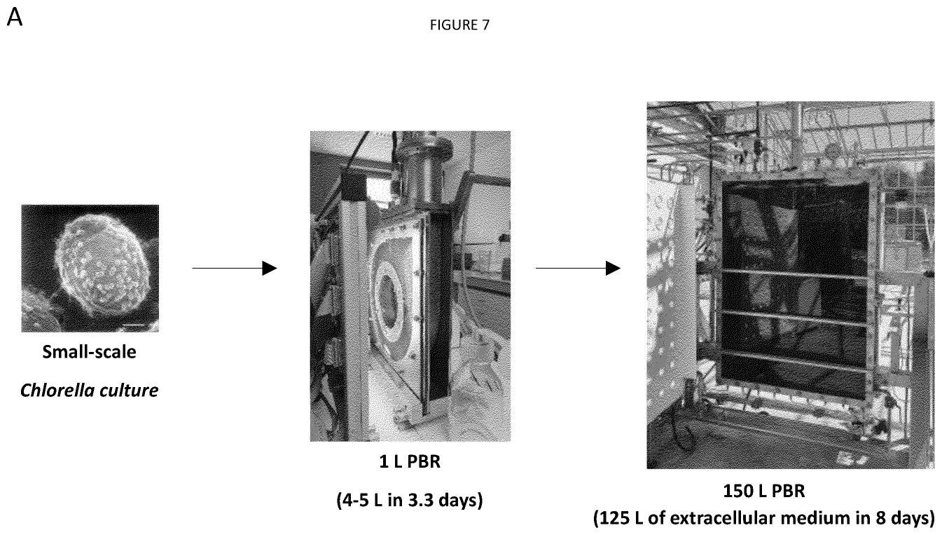

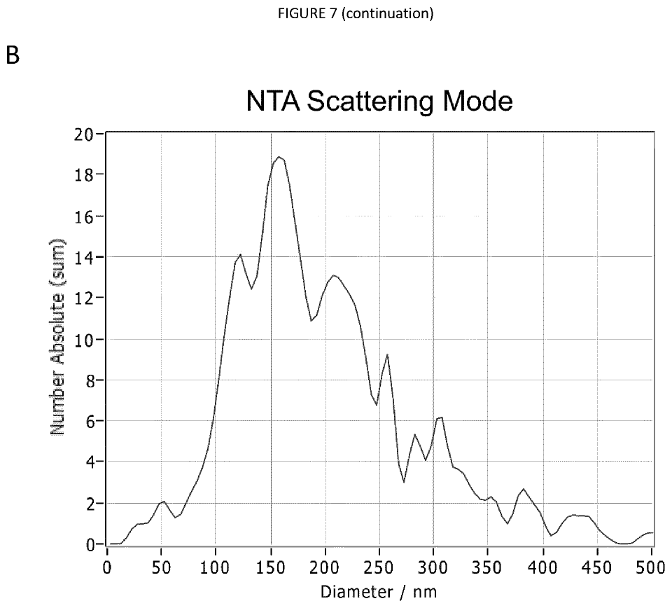

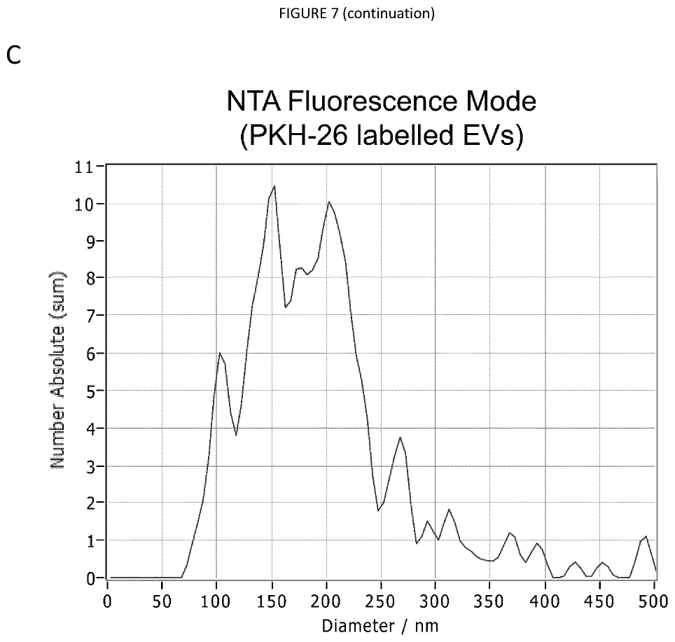

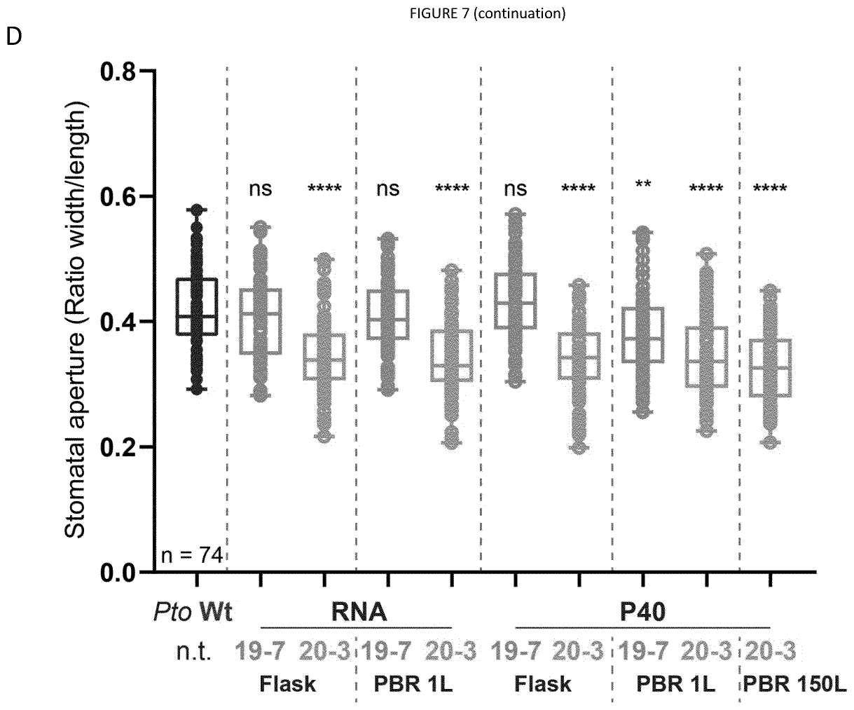

FIG. 7. The cultivation of a reference Chlorella line in photobioreactors does not affect the quality and functionality of the corresponding EVs fractions
A) Transgenic Chlorella, such as the IT20 #3 reference line, can be easily cultivated in photobioreactors (PBRs) of different sizes, from 1 to 150 L PBRs (AlgoSolis, Saint Nazaire, France).
B) Size distribution, in the range 0-500 nm, of Chlorella particles from P40 fractions obtained from a 150 L PBR culture, determined through scattering analysis (using the Particle Metrix ZetaView system).
C) Size distribution, in the range 0-500 nm, of PKH26-labeled Chlorella particles from P40 fractions obtained from a 150 L PBR culture using the Particle Metrix ZetaView system. The measurements were performed using the 488 nm laser.
D) Stomatal reopening assay performed using total RNAs and P40 fractions from the reference IT20 #3 and the control IT19 #7 lines. Samples were prepared using cell biomass and medium from different production systems as depicted. Stomatal reopening response was assessed as previously described.

FIG. 8. Treatment with supernatants of heat-killed bacteria improves Chlorella EVs production and/or secretion
A) Scheme of the treatment to increment EVs production. A freshly diluted Chlorella culture is left to grow to early stationary phase (2 to 4×10⁶ cells/ml). The culture is then treated with the equivalent of 25 mg/ml of supernatants from heat-killed E. coli and Pto DC3000 cells resuspended in water. After two days, the P100 fractions from the different treatments (untreated, +E. coli and +Pto DC3000) are collected and quantified. The total number of Chlorella cells is also determined to check possible effects on the microalgae growth.
(B) Particle concentration in the P100 fractions of the untreated (Ctl), +E. coli and +Pto DC3000 (DC3000) cultures. N=4 independent biological replicates.

### EXAMPLES

### Example 1: Materials and Methods

Chlorella vulgaris Material and Growth Conditions

The wild type C. vulgaris strain UTEX265 was kept in BG11, 1% agar plates and grown in autotrophic conditions in a Sanyo MLR-351 growth chamber. Environmental conditions were kept at 25° C., 14h/10h photoperiod and about 100 μmol/m²/s of light intensity. Transgenic Chlorella lines were kept in the same condition using plates containing 20 μg/ml of Hygromycin. Liquid culture was started by inoculating a single colony in BG11 (pH 7) in aerated 25 cm² plastic flasks with no agitation and then regularly diluted once or twice per week (dilution ratio 1:10) in order to reach the final volume (200-800 ml split in several aerated 75 cm² flasks). Culture density was assessed by using a Malassez chamber. To assess culture axenicity, routine contamination tests were performed by adding 1 ml of culture to BG11 supplemented with peptone. The mixture was kept in the dark for 3 weeks and bacterial growth followed by microscopic observation. Chlorella production in the 150 L PBR was carried out under continuous light cycle regime, with a light intensity increasing from 150 to 400 μmol/m²/s of white light to cope with the growing cell density in the PBR, a mean temperature of 22.9±6° C. and a fixed pH at 8. In those standard growth conditions, the transgenic Chlorella cells reached a maximum culture density of about 1.1 g/L after 8 days. Cell-free medium collection was performed by two successive rounds of centrifugation at 3600 g, for a gross cell precipitation, and 4000 g to remove all the remaining cells.

### Bioinformatics, Sequence Conservation and Phylogenetic Analyses

To identify protein sequences belonging to the vesicle and extracellular vesicle biogenesis or functions, candidate human and plant protein sequences were used as query for BLASTP analyses on the NCBI and JGI (Chlorella variabilis) databases. The first 10 hits were retained and used for local alignments with the query sequence. The best candidates (i.e., the ones with the higher sequence similarity) were also analyzed on the Pfam (http://pfam.xfam.org/) and SMART (http://smart.embl-heidelberg.de/) databases and using the PHMMER search (https://www.ebi.ac.uk/Tools/hmmer/search/phmmer) in order to compare the protein domain composition with the query. The Chlorella proteins showing high sequence similarity and a conserved domain composition were considered as “putative orthologs”.

For C. vulgaris analyses, the transcriptome of the UTEX 395 strain was used (Guarnieri et al., 2018) to perform local blastp and blastn searches. The retrieved sequences were analyzed for similarity and domain architecture as described.

For Chlorella AGO and DCL protein phylogenetic analysis, the protein sequences of AGO and DCL of Homo sapiens and Arabidopsis thaliana were used as queries for BLASTP analyses against the C. variabilis genome (JGI). The protein sequences for plant, animal and fungal AGOs (Murphy et al., 2008) and DCL (Mukherjee et al., 2013; Gao et al., 2014) were obtained from the literature. A total of 111 AGO and 77 DCL proteins were retained after preliminary alignments to eliminate the divergent sequences. Protein domain architecture was analyzed to unambiguously identify AGO and DCL proteins similar to the canonical ones. The sequence alignments were manually trimmed to keep only the most conserved regions for the analysis corresponding to 288 aa for AGO and 436 aa for DCL. MEGA X software was used to perform the NJ phylogenies and the trees edited using FigTree 1.4.

### Generation of Constructs for Small RNAs Production in Chlorella

Inverted repeat constructs designed to produce artificial small RNAs targeting specific regions of virulence and essential genes from various bacterial plant pathogens were generated using the Green Gate assembly strategy. The gene specific or chimeric targeted regions were cloned as “B” (sense) and “D” (antisense) modules and assembled in expression constructs. All the generated hairpins contain a specific intron sequence from the Petunia Chalcone synthase gene CHSA (SEQ ID NO: 149) and were under the control of Cauliflower Mosaic Virus (CaMV) 35S promoter, including a Hygromycin resistance cassette. The chimeric cfa6-hrpL construct (IT20) has been previously described (PCT/EP2019/072169, PCT/EP2019/072170).

All the chimeric constructs were obtained through simultaneous ligations of the different DNA fragment into a “B” Green Gate module and specific oligonucleotides were used to generate and clone the antiparallel strand as a “D” module. All the plasmids were verified by restriction analysis, Sanger sequencing and then introduced into the Agrobacterium tumefaciens strains C58C1 by electroporation.

Generation of C. vulgaris Transgenic Lines

C. vulgaris genetic transformation was performed using a disarmed A. tumefaciens strain. In more detail, 5×10⁸ total cells from an exponentially growing culture were plated on BG11 agar plates and grown under normal light irradiance for 5 days. A. tumefaciens carrying the appropriate inverted repeat construct was pre-inoculated the day before the transformation either from glycerol stock or from a LB plate at 28° C., 180 rpm shaking. The day of the transformation, 5 ml of the A. tumefaciens pre-inoculum was used to seed 50 ml of LB and grown up to OD₆₀₀=0.8-1.2. At the right optical density, the bacteria were collected, washed and resuspended in induction medium (BG11, pH 5.6, acetosyringone 100 μM) at OD₆₀₀=0.5. Chlorella cells were gently scraped form the plates, resuspended in 200 μl of bacteria and co-cultivated for 2 days on induction medium agar plates in the dark at 25° C. After the co-cultivation, the cells were harvested, put in 7 ml of BG11 supplemented with 50 μg/mL of Ticarcillin disodium/clavulanate potassium (TIM, T0190, Duchefa) or Cefotaxime (C7039, Merck) and left in the dark for 2 days at 25° C. Finally, the cells were collected and plated onto BG11 agar plates supplemented with 20 μg/ml of Hygromycin and 50 μg/ml of Ticarcillin disodium/clavulanate potassium (TIM, T0190, Duchefa) or Cefotaxime (C7039, Merck). After 2 days in the dark, the plates were exposed to light. After 2-3 weeks, 20-30 colonies were plated on fresh BG11 agar plates with 20 μg/ml of Hygromycin.

Selection and Identification of C. vulgaris Transgenic Lines

To identify the clones carrying the expression construct, gDNA from the transformant colonies was collected as follows. A few Chlorella cells were scraped with a sterile plastic tip from the colony growing on agar plates and put in 10 μl of HotShot5 lysis buffer (150 mM NaOH, 0.1 mM EDTA, 1% Triton X-100). The mix was incubated for 10′ at RT and boiled for 15′ at 95° C. The lysate was then diluted by adding 100 μl of H₂O and 1-5 μl used as template for a PCR reaction using IT-specific oligonucleotides. The wild type strain was included as negative control and the corresponding IT plasmid (5 ng per reaction) as positive control.

### Total RNA Extraction (Chlorella)

Fifty to 800 ml of liquid Chlorella culture (5×10⁶-1×10⁷ cells/ml) were harvested by centrifugation (Beckman rotor JS5.3, 5000 g, 15′, 18° C.), the pellet washed in 1×PBS and flash frozen in LN. The frozen pellet was ground to a fine powder in liquid nitrogen, using a mortar and pestle. Total RNA extraction was performed using Tri-Reagent (Sigma, St. Louis, MO) according to manufacturer's instructions using about 100 mg of powder.

### Chlorella EVs Fraction Purification

To isolate Chlorella EVs, two cell-free medium concentration/purification strategies were employed: by centrifugal concentration (Pall macrosep 100 kDa devices) or tangential flow filtration (Sartorius VivaFlow 50R 100 kDa device). For the first approach, the BG11 collected after cell separation was further centrifuged (Beckman rotor JS5.3, 5000 g, 10′, 18° C.) to eliminate all residual cells. The supernatant was then filtered using Pall Macrosep 100 kDa devices (MAP100C37) according to manufacturer's instructions. The recovered concentrated medium (CM) was then passed through 0.45 μm filters and stored at 4° C. before performing further purification steps. For the second strategy, the BG11 collected after cell separation was further centrifuged (Beckman rotor JA18, 10000 g, 10′, 4° C.) and vacuum-filtered onto 0.65 μm Whatman paper filters, to eliminate all residual cells. The supernatant was then filtered using the Sartorius VivaFlow 50R 100 kDa system (VF05H4) according to manufacturer's instructions. The recovered concentrated medium (CM) was then passed through 0.45 μm filters and used to purify Chlorella EVs. Starting from the CM, the P40 fraction was obtained by ultracentrifugation at 40,000 g and the P100 fraction at 100000 g, for 1 hour at 4° C., in a Sorvall WX 80 Ultracentrifuge (ThermoFischer). After centrifugation, the supernatant was discarded and the purified EVs pellet, either from P40 or P100 purifications, resuspended in 1 ml of filtered 1×PBS and filtered using a 0.22 μm filter. For sample quality analysis, 1/200 of the EVs sample was processed using a Nanoparticle Tracking system (ParticleMetrix ZetaView). To estimate the amount of exosome-like EVs in the sample, the particles were labeled using the PKH26 dye.

To recover the P40 fraction from the cell-free extracellular medium of 150 L PBR a modified protocol of ultrafiltration and ultracentrifugation was employed. At first, two rounds of vacuum filtration on Millipore Glass Fiber Prefilters AP25 (2 μm) were performed. Then, the sample was centrifuged at 5000 g (10′, 4° C.) followed by a second vacuum filtration on MF-Millipore 0.65 μm filters, required to eliminate the suspended organic matter still present in the cell-free medium. The clarified medium was then processed as described above to purify the P40 fraction by centrifugal filtration and ultracentrifugation.

### Chlorella EVs Production Improvement Using Bacterial Supernatants

A fresh (4 days old max) Wt Chlorella culture was diluted and split in 3 different 75 cm 2 aerated flasks with 50 ml of culture at ˜5×10⁵ cells/ml. The flasks were left to reach the end of the exponential phase, ≈3/4 days in our conditions, at 2/4×10⁶ cells/ml before starting the treatment with the bacterial supernanatant. The bacteria, both E. coli K12, TOP10 and Pto DC3000 Wt, were scraped from plates at confluent growth, the recovered pellet resuspended in 300 μl of H₂O and weigthed before being heat inactivated for 15′ at 95° C. The inactivated bacteria were spun down by centrifugation and the supernantant diluted to a concentration of 10 μg of pellet/100 μl. The Chlorella cultures were treated with the bacterial supernatant to a final concentration of 10 μg/100 ml and then put back in the incubator, in standard conditions (25° C., 14/10 light/dark, no shaking), for 48 hours. At the end of the incubation, the Chlorella cells were counted using a Malassez chamber to verify that the treatment did not affect the cell growth. Then, the P100 fractions were prepared as described (Sartorius Vivaflow 50R 100 kDa) and analyzed by NTA profiling.

Labeling of EVs with PK1H26 or DiR dyes

For EV labeling with the PKH26 dye (Sigma), the P40 fraction and an ultracentrifuge tube containing the same volume of BG11 medium were brought up to 1 ml with diluent C. Then, 6 μl of PKH26 dye were added to both tubes according to the manufacturer's protocol. The samples were mixed continuously for 30″ and incubated 5′. After the incubation at room temperature, 2 ml of 1% BSA in PBS were added and completed up to a volume of 8.5 ml with BG11. Before the precipitation, 1.5 ml of a 0.931M Glucose solution was carefully stratified at the bottom of the ultracentrifugation tube. The sample was ultracentrifuged at 190000 g for 2 hours, 4° C. and all the supernatant carefully discarded. The resulting pellet was washed with 1×PBS at 100000 g for 30′ at 4° C. The labeled EVs were syringe-filtered through a 0.45 μm filter before further processing (NTA analysis or internalization experiments).

For DiR labeling, a working solution at 1 mg/ml in 100% Ethanol of the dye was prepared and 5 μl of this solution added to 1 ml of freshly prepared P40 fraction to a final concentration of 5 μM. The sample was incubated 1 hour at 37° C. and then centrifuged at 100000 g for 30′ at 4° C. The resulting pellet was washed with 1×PBS at 100000 g for 30′, 4° C. to remove the free dye and finally resuspended in 1 ml of 1×PBS. The labeled EVs were passed through a 0.45 m filter before use.

### Stomatal Reopening Assay

Plants (4/5 weeks old, 8h/16h light/dark photoperiod) were kept under light (100 μ/m²/s) for at least 3 hours before subjecting them to any treatment to ensure full expansion of stomata. Intact young leaf sections, at least 6 per condition, were immersed in water or bacterial suspension (at a concentration of 10⁸ cfu/ml, OD₆₀₀=0.2). One hour prior to the bacterial infection, the sections were treated with either the EVs (from ˜10 μM) or total RNAs (20 μg). After 3 hours of infection with the bacteria, the leaf sections were labeled 10′ with Propidium Iodide (10 ng/ml in H₂O) washed 5′ in H₂O and observed under SP5 laser scanning confocal microscope. For each condition, 10-15 pictures were taken from different leaf surface regions. From the pictures, at least 60 stomata per condition were manually measured using ImageJ 1.53c to obtain their width and length. The width/length ratio was calculated using excel and statistical analysis performed using the 2way ANOVA test.

For Mnase protection assay, before incubation with the leaf sections, the samples were treated incubating them for 30′ at 37° C. in presence or absence of 300 U/ml of Mnase. The reaction was stopped by adding EGTA to a final concentration of 20 mM before using the samples for the stomatal reopening assay.

### Generation of Constructs to Detect Small RNAs Activity in Bacterial Cells

To detect small RNAs activity from total RNA extracts or purified EVs samples, gain-of-function lacI-based reporter constructs were generated using the Green gate approach. All the elements of the reporter system were cloned in different Green gate modules using the repressilator plasmids as template for PCR amplification (Elowitz & Leibler, 2000). The strategy aimed in the assembly of two different cassettes in the same construction: one constitutively expressing the LacI repressor fused to a siRNAs target sequence either at its 5′ or 3′ end (cassette C-F), and one expressing a GFP reporter gene only in absence of the LacI repressor (cassette A-B). To this end, the pLac (with RBS) promoter was cloned as A module, the destabilized GFPaav with the tRrnB Ti terminator as B module, the constitutive pNPTII promoter (with RBS) as C module, the LacI-lite destabilized repressor as modules D and E, the small RNAs target region as modules D and E, and the tRrnB Ti terminator as module F. The construct R37, bearing the fusA target region at the 3′ end of the LacI gene, was selected to test the kinetics of GFP induction in E. coli TOP10 cells using the lacI inhibitor IPTG.

| | Green Gate Modules | | | | | |
|---|---|---|---|---|---|---|
| Construct | A | B | C | D/E | | F |
| R37 | pLac (100 bp) | GFPaav::tRrnB T1 (903 bp) | pNPTII (142 bp) | Lacl-lite (1125 bp) | fusA IR (83 bp) | tRrnB T1 (140 bp) |

### Detection of Small RNAs Activity Using Bacterial Reporter Systems

To test the reporter constructs in bacterial cells, E. coli TOP10 cells carrying the R37 construct were inoculated O/N at 37° C., 180 rpm, in LB supplemented with 50 μg/ml of Spectinomycin. The following day, an overday culture was performed for 4-5 hours in the same conditions, by inoculating 1:1000 of the O/N culture in fresh medium. At the end of the preculture, the OD₆₀₀ was measured and the culture serially diluted to reach OD₆₀₀=0.02. A total of 180 μl of diluted culture was put in technical duplicates in a 96-well plate to perform kinetics in a TECAN Infinite 200 Pro plate reader. The cells were treated with 20 μl of LB containing either different IPTG concentrations (from 1 to 0.001 mM) (+IPTG condition), 25 μg/ml of Chloramphenicol (background control) or LB diluted with H₂O (−IPTG condition). The OD₆₀₀ and the GFP fluorescence were simultaneously measured at each time point (5′) over 12-16 hours kinetics by means of specific filters in the plate reader. At the end of the kinetics, the OD₆₀₀ values were analyzed to confirm the correct cell growth over the time course. The GFP fluorescence was normalized as follows: the mean values of the technical replicates from the +IPTG treatments was subtracted from the means of the control Chloramphenicol wells and −IPTG conditions.

### Small RNA Sequencing and Data Mining

Custom libraries for up to 43 nucleotides for small RNAs sequencing of total and EV-derived RNAs from the P100 fraction of the Chlorella reference line IT20-3 (IR cfa6/hrpL) were constructed and sequenced by Fasteris®. Reads adaptors were trimmed using the UMI library v0.2.3 (https://github.com/CGATOxford/UMI-tools). Low quality reads were filtered-out based on a base-call threshold of Q20 (99% base call accuracy). In order to represent the small RNA production from the cfa6/hrpL hairpin, we selected a subset of read size comprised between 10 and 25 nucleotides for further analyses and graphical representation. Reads were mapped to the IR cfa6/hrpL sequence using bowtie (Langmead et al., 2009), allowing zero mismatches. We then used an inhouse R script to load aligned reads from the ‘.bam’ files, and represent reads abundance on both extremities of the cfa6/hrpL haipin using the GenomicAlignments package (https://github.com/Bioconductor/GenomicAlignments).

### Transmission Electron Microscopy Observation of Chlorella EVs

For transmission electron microscopy, a droplet of purified EVs (2 to 10 μl at 10⁹ to 10¹¹ particles/ml) was deposited on formvar/carbon coated grids for 20′. After the incubation, the excess sample was removed and the grids fixed with 2% paraformaldehyde/1% glutaraldehyde in 0.1 M PBS (pH 7.4) for 20′ at RT. After six 1′ washes in H₂O, the samples were contrasted with Uranyl acetate in Methylcellulose (4% Uranyl acetate in H₂O/Methylcellulose, ratio 1:9) for 10′. At the end of the incubation period, the excess contrast was removed and the grid air-dried before visualization. Electron micrographs were acquired on a Tecnai Spirit electron microscope (Thermo Fisher, Eindhoven, The Netherlands) equipped with a 4 k CCD camera.

### Example 2: Chlorella Microalgae Possess Both Highly Conserved EV Biogenesis Factors as Well as Plant-Related EV Factors

To determine whether Chlorella could be exploited as scaffold for EV-embedded and/or-associated small RNA production, we have first investigated the possible presence of core components required for EVs biogenesis and functions in its genome or transcriptome. To this end, we have conducted an in silico comparative analysis using available genomes and transcriptomes of Chlorella variabilis NC64a, Chlorella vulgaris UTEX 395, Saccharomyces cerevisiae, Homo sapiens and Arabidopsis thaliana. Results from this analysis revealed that C. variabilis encodes putative orthologs of the ESCRT-I, ESCRT-II and ESCRT-III complexes and of the plant FREE1/FYVE1-like protein, a plant-specific ESCRT essential for intracellular vesicle biogenesis (Table 1, Kolb et al., 2015).

TABLE 1
Comparison of the factors encoding ESCRT complexes and other microvesicle-related proteins
in Yeast, Human, Plant and Chlorella

| Regulators | Yeast (Sc) | Human (Hs) | Plant (At) | C. variabilis (NC64a) | C. vulgaris (UTEX 395) |
|---|---|---|---|---|---|
| ESCRT-0 and | VPS27 | HRS | — | [symbol] | — |
| Equivalent | Hse1 | STAM1, 2 | — | [symbol] | Transcript_contig_56508 |
| | — | — | FREE1/FYVE1 | XP_005846966 | Transcript_contig_54179 |
| | — | — | PROS | [symbol] | — |
| ESCRT-0 functional | GGA 1, 2 | GGA 1, 2, 3 | - | [symbol] | — |
| analogs | — | TOM1, L1, L2, L3 | TOL1 to TOL9 | [symbol] | — |
| ESCRT-I | VPS23 | TSG101 | VPS23A/ELC | XP_005843934 | Transcript_contig_53739 |
| | — | — | VPS23B | XP_005843933 | — |
| | VPS28 | hVPS28 | VPS28-1/-2 | XP_005846911 | Transcript_contig_61269 |
| | — | — | | — | Transcript_contig_68519 |
| | VPS37 | VPS37 A, B, C, D | VPS37-1/-2 | XP_005846305 | Transcript_contig_56274 |
| | MVB12 | hMVB12 A, B | — | [symbol] | — |
| | UBAP1 | — | — | [symbol] | — |
| ESCRT-II | VPS22 | EAP30 | VPS22 | XP_005846063 | Transcript_contig_57148 |
| | VPS25 | EAP20 | VPS25 | XP_005851443 | Transcript_contig_3694 |
| | VPS36 | EAP45 | VPS36 | XP_005844559 | — |
| ESCRT-III | VPS20 | CHMP6 | VPS20-1 | XP_005848234 | — |
| | SNF7 | CHMP4 A, B, C | | XP_005850888 | Transcript_contig_59252 |
| | (VPS32) | | | | |
| | — | — | SNF7-1/-2 | [symbol] | Transcript_contig_59300 |
| | — | — | | [symbol] | Transcript_contig_55347 |
| | VPS24 | CHMP3 | VPS24-1/-2 | XP_005846960 | Transcript_contig_59620 |
| | VPS2 | CHMP2 A/B | VPS2-1/-2/-3 | XP_005849915 | Transcript_contig_67861 |
| | — | — | | XP_005851546 | Transcript_contig_54164 |
| | Did2 | CHMP1/5/1A/1B | CHMP1A/B | [symbol] | — |
| | VPS60 | CHMP5 | VPS60-1/-2 | XP_005849964 | Transcript_contig_53967 |
| | IST1 | — | ISTL1 | XP_005842806 | Transcript_contig_89181 |
| | Cmp7 | CHMP7 | CHMP7 | [symbol] | — |
| Other ESCRT- | BRO1 | ALIX | BRO1/ALIX | XP_005850236 | Transcript_contig_474* |
| related proteins | — | — | Bro1L1, L2 | | Transcript_contig_55676* |
| | Doa4 | AMSH | AMSH1, 2, 3 | XP_005850797 | Transcript_contig_58892 |
| ESCRT Independent | NMASE2 | — | — | [symbol] | — |
| | Rab27A/B | Rab27A/B | — | ✓ | ✓ |
| | — | — | EXO70E2 | XP_005849494 | Transcript_contig_57551 |
| VPS4 and accessory | VPS4 | VPS4/SKD1 | VPS4/SKD1 | XP_005847253 | Transcript_contig_54972 |
| proteins | VTA1 | LIP5 | LIP5 | XP_005845860 | Transcript_contig_56834 |
| | | | | XP_005852259 | Transcript_contig_59235 |
| Microvesicles- | ARF1/ARF6 | ARF1/ARF6 | — | ✓ | ✓ |
| related | RAC1 | RAC1 | — | ✓ | ✓ |
| | RHOA | RHOA | — | XP_005847786 | — |
| Other EVs markers | — | — | Syn121/PEN1 | XP_005845633 | Transcript_contig_54538 |
| | HSP70 | HSP70 | HSP70 | ✓ | ✓ |

| | | | |
|---|---|---|---|
| — | Tetraspanin(s) | [symbol] | — |

✓ = several potential candidates [symbol]
= not identified by blast search
* = probably part of the same transcript

By analyzing the C. vulgaris UTEX 395 transcriptome, we were also able to identify most of the typical ESCRT factors involved in EVs biogenesis. Surprisingly, we did not identify canonical ESCRT-O-related proteins (e.g., human STAM1/2) in the genome of C. variabilis, although a single transcript encoding such a putative factor was retrieved in the transcriptome of C. vulgaris. However, the low sequence similarity between the human and the C. vulgaris proteins suggests that the Chlorella ESCRT-0 complex is more likely composed of different and yet-unknown factors. Another intriguing observation is the apparent absence of tetraspanin in the Chlorella genome and transcriptome: searches using specific candidates (e.g., human CD63) or the tetraspanin domain (PF00335) against pfam or JGI protein domain databases failed to identify such factors (which are known to be present in both plants and mammals). The absence of tetraspanin factor was observed also in other green algae (Wang et al., 2012) and, whilst this protein family is considered present in all multicellular organisms, in unicellular species data show a more complex scenario of gene gain and loss that needs further investigations. By contrast, potential ESCRT-independent EVs biogenesis factors, like Rab GTPases (e.g., orthologs of human Rab27a and Rab27b, which control different steps of exosome secretion (Ostrowski et al., 2010)), were recovered in Chlorella. Furthermore, we retrieved putative orthologs of the syntaxin PENETRATION1 (PEN1), which has recently been characterized as an exosome marker in both Arabidopsis and Nicotiana benthamiana (Rutter & Innes, 2017; Zhang et al., 2020). In addition to this factor, we were also able to identify homologs of other plant EV markers like the HSP70 and BRO/ALIX (Table 1).

Overall, our data indicate that Chlorella microalgae possess conserved EV-related factors, shared between humans, yeasts and plants, but also EV-related factors that have so far been exclusively recovered from plant genomes. They also suggest that the mechanisms of Chlorella EVs biogenesis and functions are more closely related to the ones from plants than from yeasts or humans.

### Example 3: The Extracellular Medium of Chlorella vulgaris Contains EVs that are in a Size Range Between 50 and 200 nm

To determine whether Chlorella could produce EVs, and to characterize these lipid-based vesicles, we next decided to adapt protocols that have been previously used for the isolation and purification of Arabidopsis leaf apoplastic EVs (Rutter & Innes, 2017; PCT/EP2019/072169, PCT/EP2019/072170). Briefly, cell-free culture medium from Chlorella grown in flasks was first concentrated using 100 kDa MWCO Pall membranes by centrifugation or 100 kDa Sartorius VivaFlow 50R tangential filtration devices, in order to obtain a concentrated medium (30-50×) to be used for EVs purification. The resulting concentrated medium (referred to as “CM”) was further subjected to ultracentrifugation at a centrifugation speed of 40000 g, 4° C., to separate Chlorella EVs from the secreted proteins/polysaccharides, as previously reported in Arabidopsis (Rutter & Innes, 2017). The latter purification step leads to the recovery of a fraction referred to as the “P40 fraction”. Alternatively, a “P100 fraction” was also obtained through a CM ultracentrifugation step at 100000 g, 4° C. Nanoparticle tracking analysis (NTA) of these fractions revealed the presence of particles populations with a size range between 50 to 350 nm, and with a more discrete and abundant particle population centered around ˜120 nm (FIG. 1A, B for P40 and P100, respectively). To further confirm these results and get more insights into the morphology of these Chlorella EVs, the P100 fraction was analyzed by transmission electron microscopy (TEM). The latter analysis unveiled the presence of round shaped particles with an apparent lipidic bilayer, a morphology that resembles mammalian and plant EVs (FIG. 1C, Rutter & Innes, 2017; Zhang et al., 2020; Noble et al., 2020). Size measurement revealed the presence of heterogenous particles with a ˜130 nm mean diameter, suggesting that the particles detected through NTA do correspond to Chlorella EVs (FIG. 1D). Further labeling of the P40 fraction with the lipophilic dye PKH26, which uses aliphatic tails to anchor into lipid bilayer (Fick et al., 1995; Askenasy et al., 2002), and that is classically used to label mammalian exosomes for fluorescence imaging (Chuo et al., 2018), revealed that the vast majority of particles that are above 200 nm in size are not lipid-based particles (FIG. 1E). Based on this fluorescence imaging coupled with TEM and NTA analyses, we conclude that Chlorella EVs, which are in a size range between 50 and 200 nm, can be recovered from the cell-free culture medium of flasks. To date, these results provide the first evidence supporting the presence of Chlorella EVs in a cell-free culture medium.

### Example 4: Chlorella Cells Produce Small RNAs, Suggesting that the RNAi Machinery Present in this Microalga is Functional

In algae and microalgae, the presence of small RNAs and/or of RNAi activity has been demonstrated in a few species from several different lineages, including Rhodophyta, Chlorophyta, Haptophyta, Stramenopiles and Dinoflagellata (Cerutti et al., 2011). Although the Chlorella genome contains a simple RNAi machinery composed of single DCL and AGO proteins (Cerrutti et al. 2011), which we found phylogenetically related to their plant counterparts (FIG. 2A), there is currently no evidence indicating that this green alga could produce small non-coding RNAs. To test this possibility, we performed small RNA-sequencing (sRNA-seq) from total RNAs extracted from C. vulgaris cells. As a control, we also used sRNA-seq datasets that we had previously generated from total RNAs extracted from Arabidopsis adult leaf tissues. Intriguingly, we found that the major small RNA population produced by C. vulgaris was 18 nt in size, which is very different from the two main 21 and 24 nt long small RNA species typically recovered from Arabidopsis and other plant species (FIG. 2B). Furthermore, we noticed that C. vulgaris and A. thaliana can additionally produce minor peaks of 15 and 16 nt long small RNAs, respectively (FIG. 2B). Given that Dicer acts as a molecular ruler and cuts the dsRNA substrate at a precise length that is determined by the distance between the PAZ and the RNAse III domains (Zhang et al., 2004), it is possible that the structure of the Chlorella DCL enzyme exhibits distinct features compared to the one of plant DCL enzymes, which would favour the production of shorter small RNA species. Alternatively, or additionally, other RNAse III enzymes, which were also retrieved from Chlorella genome and transcriptome (Table 2), or other biogenesis factors, could contribute to this process. Altogether, these data provide evidence indicating that Chlorella can produce small RNA species, with distinct size classes compared to the ones previously reported in plants. They also suggest that Chlorella is equipped with a functional machinery for small RNA biogenesis.

TABLE 2
Putative RNAse III and DCL-like factors identified by blast
searches in C. variabilis and C. vulgaris

| Factor | C. variabilis (NC64a) | C. vulgaris (UTEX 395) |
|---|---|---|
| DCL-like | XP_005849661 | Transcript_contig_56994* |
| | — | Transcript_contig_79877* |
| | — | Transcript_contig_64095* |
| RNAse III | XP_005851019 | Transcript_contig_78810 |
| | XP_005848456 | Transcript_contig_60223 |
| | XP_005845370 | Transcript_contig_55978 |
| | XP_005849661 | Transcript_contig_68382 |

*Potentially part of the same transcript

### Example 5: Chlorella can be Engineered to Produce Small RNAs with Antimicrobial Activity

Previous studies demonstrated that the exogenous administration of small RNAs and/or long dsRNAs can be effective against eukaryotic pathogenic/parasitic (micro)organisms including fungi, oomycetes, insects and nematodes (Ivashuta et al., 2015; Wang et al., 2016; Koch et al., 2016; Wang & Jin., 2017; Wang et al., 2017. Environmental RNAi can also occur against pathogenic bacteria and relies on small RNA entities rather than on long dsRNAs (Singla-Rastogi & Navarro, PCT/EP2019/072169, PCT/EP2019/072170). Based on these studies, and on the ability of C. vulgaris to produce small RNA species (EXAMPLE 4), we reasoned that we could make use of this biological system to produce antimicrobial small RNAs. To test this possibility, C. vulgaris was stably transformed with an inverted repeat (IR) transgene carrying sequence homology with two major virulence factors of Pseudomonas syringae pv. tomato strain DC3000 (Pto DC3000), which is a Gram-negative bacterium previously shown to be sensitive to environmental RNAi (Singla-Rastogi & Navarro, PCT/EP2019/072169, PCT/EP2019/072170, FIG. 3A). The first targeted virulence factor is the coronafacic acid polyketide synthase I (cfa6) gene, which encodes a major structural component of the phytotoxin coronatine (COR) (Brooks et al., 2004). The second one is hrpL, which encodes an alternative sigma factor that is known to directly control the expression of type III-secretion system associated genes, and to indirectly regulate the expression of COR biosynthesis genes (Fouts et al., 2002; Sreedharan et al., 2006). Interestingly, when stably expressed in Arabidopsis, the IR-CFA6/HRPL inverted repeat is efficiently processed by endogenous plant DCLs into anti-Cfa6 and anti-HrpL siRNAs, which in turn target the Cfa6 and hrpL genes in Pto DC3000, thereby resulting in the dampening of its pathogenicity (Singla-Rastogi & Navarro, PCT/EP2019/072169, PCT/EP2019/072170). Importantly, some of these phenotypes are fully recapitulated upon exogenous administration of total RNAs from these transgenic plants, which contain effective anti-cfa6 and anti-hrpL siRNAs (Singla-Rastogi & Navarro, PCT/EP2019/072169, PCT/EP2019/072170). In particular, the exogenous application of these RNA extracts suppresses the ability of Pto DC3000 to trigger stomatal opening, a major virulence response employed by this bacterium to enter through stomata and colonize inner leaf tissues (Melotto et al., 2006; Singla-Rastogi & Navarro, PCT/EP2019/072169, PCT/EP2019/072170). By using the same stomatal reopening readout, which is highly sensitive to anti-Cfa6 and anti-HrpL siRNAs, we found that RNA extracts derived from the five independent Chlorella IT20 lines tested, which express the TR-CFA6/HRPL transgene, suppressed stomatal reopening events (FIG. 3B). Importantly, these phenotypes were comparable to the one observed in the presence of RNA extracts derived from the control Arabidopsis IR-CFA6/HRPL #4 plants, and mimicked the impaired stomatal reopening phenotype detected in response to a Pto DC3000 mutant strain unable to produce COR (FIG. 3B). Furthermore, because this phenotype is known to be dependent on anti-cfa6 and anti-hrpL siRNAs, but not on unprocessed IR-CFA6/HRPL transcripts (Singla-Rastogi & Navarro, PCT/EP2019/072169, PCT/EP2019/072170), our data suggest that Chlorella IR-CFA6/HRPL lines are likely competent for producing antibacterial small RNA species. Accordingly, small RNAs mapping to the IR-CFA6/HRPL inverted transgene transcripts were recovered from the Chlorella TR-CFA6/HRPL IT20 #3 reference line (FIG. 3C). It is also noteworthy that these small RNAs were produced from both the cfa6 and hrpL regions of the inverted repeat transcripts, with an enhanced accumulation of small RNAs corresponding to the hrpL region (FIG. 3C). Collectively, these data provide evidence that Chlorella can be engineered to produce small RNAs exhibiting antibacterial activity.

### Example 6: Chlorella Artificial Small RNAs Directed Against the Virulence Factor hrpL are Causal for the Suppression of hrpL-Mediated Stomatal Reopening Function

To determine whether Chlorella artificial small RNAs could be causal for the observed antibacterial activity, we next took advantage of previously described recombinant bacteria expressing a small RNA-resilient version of the hrpL gene (Singla-Rastogi & Navarro, PCT/EP2019/072169, PCT/EP2019/072170). This mutated version of the hrpL gene contains as many silent mutations as possible in the small RNA targeted region, in order to alter the binding of anti-hrpL small RNAs to hrpL mRNAs, whilst producing wild type HrpL proteins. Both the mutant and Wt versions of the hrpL gene were cloned in a plasmid, under the control of the neomycin phosphotransferase II (NPTII) promoter, and further transformed in the Pto DC3000 ΔhrpL strain, which is deleted of the hrpL gene and thus fully impaired in its ability to reopen stomata (Singla-Rastogi & Navarro, PCT/EP2019/072169, PCT/EP2019/072170, FIG. 4A). It is noteworthy that the resulting recombinant bacteria, referred to as Pto DC3000 ΔhrpL WT hrpL and mut hrpL, were previously shown to restore the ability of the Pto DC3000 ΔhrpL strain to reopen stomata, indicating that both transgenes are functional (Singla-Rastogi & Navarro, PCT/EP2019/072169, PCT/EP2019/072170). These recombinant bacteria were subsequently used in a stomatal reopening assay in the presence of total RNA extracts from the Arabidopsis IR-HRPL #4 line or the Chlorella IT29 #12, IT29 #15 lines, which express from the Chlorella genome an IR-HRPL inverted repeat that specifically targets the hrpL gene. As controls, we used the Pto DC3000 Wt and ΔhrpL strains as well as total RNAs from the IT19 #7 reference line, which expresses from the Chlorella genome an IR-CYP51 inverted repeat that does not exhibit sequence homology with the Pto DC3000 genome, but instead targets three cytochrome P450 lanosterol C-14α-demethylase (CYP51) genes of the fungal phytopathogen Fusarium graminearum (Koch et al., 2013). We found that the bacteria complemented with the hrpL Wt gene were sensitive to RNA extracts derived from the two independent Chlorella IT29 #12 and IT29 #15 lines, but also from the control Arabidopsis IR-HRPL #4 line, as manifested by an altered ability of these bacteria to reopen stomata (FIG. 4B). By contrast, these recombinant bacteria reopened stomata in the presence of RNA extracts derived from the Chlorella IT19 #7 reference line (FIG. 4B), supporting a specific effect of anti-hrpL small RNAs in this phenomenon. Importantly, we found that the bacteria complemented with the mutated hrpL gene were fully resistant to RNA extracts derived from the two independent Chlorella IT29 lines and the control Arabidopsis IR-HRPL #4 line, allowing normal stomatal reopening phenotypes (FIG. 4B). The latter data indicate that the suppression of stomatal reopening phenotype is not due to potential off-target effects of these anti-hrpL small RNAs, but rather caused by their targeting effects over the hrpL transcript sequence. They also indicate that anti-hrpL small RNAs, produced from either Chlorella or Arabidopsis transgenic lines, are causal for the suppression of hrpL-mediated stomatal reopening function. The results reported in EXAMPLES 5 and 6 provide thus a proof-of-concept demonstrating that Chlorella can be engineered to produce effective antibacterial small RNAs acting in a sequence-specific manner.

### Example 7: EVs from Chlorella IR-CFA6/HRPL Transgenic Lines Exhibit Antibacterial Activity

Previous studies have reported that plant EVs can deliver biologically active antimicrobial small RNAs in fungal, oomycetal and bacterial cells (Cai et al., 2018; Teng et al., 2018; Hou et al., 2019; PCT/EP2019/072169, PCT/EP2019/072170), thereby reducing their pathogenicity. To investigate whether this phenomenon holds also true for antimicrobial small RNAs embedded in, and/or associated with, Chlorella EVs, we first collected the cell-free medium from two independent Chlorella IT20 lines, which express the IR-CFA6/HRPL transgene, and further used an ultrafiltration method designed to retain particles that are above 30-90 nm. The resulting concentrated medium (CM), corresponding to a 30-50 time concentrate of the original Chlorella medium, was additionally filtered using a 0.45 μm sterilized filter to eliminate possible bacterial contaminants derived from the ultrafiltration process. The antibacterial activities of the CM were further analyzed using a stomatal reopening assay, and RNA extracts from the corresponding Chlorella IT20 cells, and from the Arabidopsis IR-CFA6/HRPL #4 plants were included in the assay as positive controls. Interestingly, the CM from two independent Chlorella IT20 lines suppressed stomatal reopening events, to the same extent as total RNA extracts derived from the same producing cells or from the Arabidopsis IR-CFA6/HRPL #4 plants (FIG. 5A). These results suggest that Chlorella IT20 lines could produce extracellular EVs—bigger than 30-90 nm—containing anti-cfa6 and/or anti-hrpL small RNAs. Consistent with this idea, we found that the P40 fraction from the reference Chlorella IT20 #3 line was fully competent in suppressing Pto DC3000-induced stomatal reopening, to the same extent as total RNAs from Chlorella cells or from the Arabidopsis IR-CFA6/HRPL #4 plants (FIG. 5B). We conclude that EVs from Chlorella IT20 lines are likely loaded and/or associated with anti-cfa6 and anti-hrpL small RNAs, which must be delivered into Pto DC3000 cells to trigger the detected antibacterial effect.

We have previously shown that plant EVs protect antibacterial small RNAs from digestion mediated by the non-specific Micrococcal nuclease (Mnase) (Singla-Rastogi & Navarro, PCT/EP2019/072169, PCT/EP2019/072170). Here, to determine whether Chlorella EVs could share similar features, we treated the P40 fraction from the IT20 #3 reference line with Mnase and further used it in a stomatal reopening assay. In more detail, the samples were incubated for 30′ at 37° C. in the presence or absence of 300 U/ml of Mnase. At the end of this incubation period, EGTA, at a final concentration of 20 mM, was added to inhibit Mnase activity, and the samples were further used for stomatal reopening assay. Significantly, we found that the P40 fraction treated with Mnase remained fully capable of suppressing Pto DC3000-induced stomatal reopening, such as the untreated P40 fraction used as control (FIG. 5C).

These data suggest that anti-cfa6 and anti-hrpL small RNAs are protected from ribonuclease-mediated digestion when embedded into, and/or associated with, Chlorella EVs. Consistent with this hypothesis, we were able to detect through sRNA-seq analysis both anti-cfa6 and anti-hrpL small RNA reads from Mnase-treated EV samples produced by the Chlorella IT20 #3 reference line (FIG. 5D).

Based on these overall results, we propose that EV-associated anti-cfa6 and anti-hrpL small RNAs produced by the Chlorella IT20 #3 line are intravesicular and/or extravesicular but likely associated with ribonucleoprotein complexes, and thus protected from RNAses.

### Example 8: Chlorella EV-Embedded and/or -Associated Small RNAs Directed Against hrpL are Causal for the Suppression of hrpL-Mediated Stomatal Reopening Function

To confirm that antibacterial small RNAs are the bioactive cargoes, and to compare the antibacterial potential of both the P40 and P100 fractions, we performed a stomatal reopening assay with the Pto DC3000 ΔhrpL Wt hrpL and Mut hrpL bacteria described in EXAMPLE 4. The P40 and P100 fractions were collected from the Chlorella IT19 #7 (IR-CYP51) and IT29 #12 (IR-HRPL) reference lines and treated with Mnase, as previously described, using the untreated Pto DC3000 Wt and ΔhrpL strains as controls. As observed using the total RNA extracts in EXAMPLE 4, only the bacteria complemented with the Wt hrpL gene were sensitive to EVs from the Chlorella IT29 #12 reference line, with the P40 and P100 fractions showing similar antibacterial effects (FIG. 5E). By contrast, these recombinant bacteria reopened stomata in the presence of the Mnase-treated P40 and P100 fractions derived from the Chlorella IT19 #7 line (FIG. 5E), supporting a specific effect of EV-embedded and/or-associated anti-hrpL small RNAs. Importantly, the recombinant bacteria expressing the hrpL mutant version were fully refractory to the suppression of stomatal reopening effects mediated by the Mnase-treated P40 and P100 fractions produced by the Chlorella IT29 #12 reference line (FIG. 5B). These data support a causal role for EV-embedded and/or-associated anti-hrpL small RNAs in suppressing the ability of Pto DC3000 to reopen stomata. They also indicate that Chlorella EVs likely deliver anti-hrpL small RNAs in Pto DC3000 cells to target the hrpL gene in a sequence-specific manner, thereby suppressing bacterial-triggered stomatal reopening.

### Example 9: Generation of Stable Chlorella Lines Expressing Inverted Repeat Transgenes Targeting Essential and/or Virulence Factors from Agriculturally Relevant Phytopathogens

To produce Chlorella EV-embedded and/or-associated small RNAs that might be ultimately used as RNA-based biocontrol agents against agriculturally relevant phytopathogens, we are generating inverted repeat constructs targeting essential genes and/or key virulence factors from the bacterial phytopathogens Xylella fastidiosa, Candidatus Liberibacter asiaticus, Pseudomonas syringae pv. actinidiae, Pseudomonas syringae pv. tomato, Erwinia carotovora, Rastonia solanacearum, Xanthomonas campestris pv. campestris, Xanthomonas horturum pv. vitians, Xanthomonas citri pv. fucan, Acidovorax valerianella, Acidovorax citrulli; the fungal phytopathogens Fusarium graminearum, Botrytis cinerea, Colletotrichum species, Zymoseptoria tritici; the oomycetal phytopathogens; Phytophthora infestans, Plasmopara viticola and the viral phytopathogen Plum Pox Virus (PPV).

The following IR constructs target Xylella fastidiosa genes (they contain the intron of SEQ ID NO: 149, apart from the target sequences):
IR-cheA/gaCA/tolC/pglA/engXCA1/engXCA2, SEQ ID NO:1-2;
IR-cheA/GumH/GumD/XpsE/LesA HolC, SEQ ID NO:3-4;
IR-LesA/gumH/gumD, SEQ ID NO:5-6;
IR-XpsE/HolC/LesA, SEQ ID NO: 7-8;
IR-cheA/pglA/LesA, SEQ ID NO: 9-10;
IR-cheA/engXCA1/engXCA2, SEQ ID NO:11-12;
IR-gacA/pglA/engXCA2, SEQ ID NO: 13-14;
IR-cheA/tolC/engXCA1, SEQ ID NO:15-16.

The following IR constructs target Candidatus Liberibacter asiaticus genes (they contain the intron of SEQ ID NO: 149, apart from the target sequences):
IR-wp015452784 WP012778510 wp015452939 act56857 wp012778668, SEQ ID NO:55-56;
IR-wp012778355 wp015452784 WP012778510, SEQ ID NO:57-58;
IR-wp015452939 act56857 wp012778668 NO, SEQ ID NO:59-60.

The following IR constructs target Erwinia carotovora genes (they contain the intron of SEQ ID NO: 149, apart from the target sequences):
IR-GyrB/MreB/rbfA/RsgA/FliA/QseC, SEQ ID NO:47-48;
TR-GyrB/rbfA/QseC, SEQ ID NO:49-50;
IR-fliA/MreB/QseC NO, SEQ ID NO:51-52;
IR-GyrB/MreB/RsgA NO, SEQ ID NO:53-54.

The following IR constructs target Pseudomonas syringae pv. actinidiae genes (they contain the intron of SEQ ID NO: 149, apart from the target sequences):
IR-GyrB/Hfq HrpR HRPS/MreB RpoD, SEQ ID NO:61-62;
IR-GyrB/Hfq/MreB, SEQ ID NO:63-64;
IR-HrpR/HrpS/Hfq, SEQ ID NO:65-66;
IR-HrpR/GyrB/RpoD, SEQ ID NO:67-/148.

The following IR constructs target genes from Pseudomonas syringae pv. tomato strain DC3000 (they contain the intron of SEQ ID NO: 149, apart from the target sequences):
IR-CFA6/HRPL, SEQ ID NO:90-91;
IR-HRPL, SEQ ID NO:92-93;
IR-FusA, SEQ ID NO:136-137;
IR-GyrB, SEQ ID NO:138-139;
IR-FusA/GyrB, SEQ ID NO:140-141;
IR-AvrPto/AvrPtoB, SEQ ID NO:94-95;
IR-AvrPto/AvrPtoB/HopT1-1: SEQ ID NO: 130-131
IR-rpoB/rpoC/FusA, SEQ ID NO:132-133;
IR-secE/rpoA/rplQ, SEQ ID NO:134-135;
IR-secE, SEQ ID NO:142-143;
IR-GyrB/hrpL, SEQ ID NO:144-145;
IR-dnaA/dnaN/gyrB, SEQ ID NO:146-147.

The following IR constructs target genes from Ralstonia solanacearum (they contain the intron of SEQ ID NO: 149, apart from the target sequences):
IR-HRPG HRPB HRCC, SEQ ID NO:98-99;
IR-HRPB/HRCC/TssB/XpsR, SEQ ID NO:100-101.

The following IR constructs target genes from Xanthomonas campestris pv. campestris (they contain the intron of SEQ ID NO: 149, apart from the target sequences):
IR-HRPG/HRPX/RsmA, SEQ ID NO:102-103;
IR-NadHb/NadHd/NadHe, SEQ ID NO: 104-105;
IR-DnaA/DnaE1/DnaE2, SEQ ID NO:106-107.

The following IR constructs target genes from Xanthomonas hortorum pv. vitians (they contain the intron of SEQ ID NO: 149, apart from the target sequences):
IR-GyrB, SEQ ID NO:17-18;
IR-FusA, SEQ ID NO: 19-20;
IR-ZipA, SEQ ID NO:21-22;
IR-GyrB/FusA/ZipA, SEQ ID NO:128-129.

The following IR constructs target genes from Xanthomonas citri pv. fucan (they contain the intron of SEQ ID NO: 149, apart from the target sequences):
IR-GyrB/FusA/MreB/HrpG/PhoP/FhaB, SEQ ID NO:120-121;
IR-GyrB/RbfA/MreB, SEQ ID NO:122-123;
IR-HrpG/PhoP/FtsZ, SEQ ID NO:124-125;
TR-FhaB/FusA/MreB, SEQ ID NO:126-127;
IR-GyrB/FusA/ZipA, SEQ ID NO:128-129.

The following IR constructs target genes from Acidovorax valerianella (they contain the intron of SEQ ID NO: 149, apart from the target sequences):
IR-rimM/rsgA/rbfA/MreB/gyrB/FtsZ, SEQ ID NO:23-24;
IR-rimM/rbfA/FtsZ, SEQ ID NO:25-26;
IR-RsgA/gyrB/MreB, SEQ ID NO:27-28;
IR-RsgA/rbfA/FtsZ, SEQ ID NO:29-30.

The following IR constructs target genes from Acidovorax citrulli (they contain the intron of SEQ ID NO: 149, apart from the target sequences):
IR-MreB/ybeY/rbfA/gyrB/FtsZ/rsgA: SEQ ID NO:31-32;
IR-MreB/ybeY/rbfA: SEQ ID NO:33-34;
IR-rsgA/gyrB/MreB: SEQ ID NO:35-36;
IR-YbeY/RsgA/MreB: SEQ ID NO:37-38.

The following IR constructs target genes from Fusarium graminearum (they contain the intron of SEQ ID NO: 149, apart from the target sequences):
IR-CYP51A/CYP51B/CYP51C. SEQ ID NO:96-97.

The following IR constructs target genes from Botrytis cinerea (they contain the intron of SEQ ID NO: 149, apart from the target sequences):
IR-TOR/CGF1/DCL1/DCL2/LTF1/HBF1, SEQ ID NO:76-77;
IR-TOR/DCL/DCL2, SEQ ID NO:78-79;
IR-CGF1/HBF1/LTF1, SEQ ID NO:80-81;
IR-DCL1/HBF1/TOR, SEQ ID NO:82-83.

The following IR constructs target genes from Colletotrichum species (they contain the intron of SEQ ID NO: 149, apart from the target sequences):
IR-CclA/ACS1/ACS2/ELP1/ELP2/MOB2, SEQ ID NO:84-85;
IR-CclA/ACS1/ACS2, SEQ ID NO:86-87;
IR-ELP1/ELP2/MOB2, SEQ ID NO:88-89.

The following IR constructs target genes from Zymoseptoria tritici (they contain the intron of SEQ ID NO: 149, apart from the target sequences):
IR-Br1A2/StuA/F1bc/PKS1/PPT1, SEQ ID NO:108-109;
IR-Br1A2/StuA/F1bc, SEQ ID NO:110-111;
IR-StuA/PKS/PPT1, SEQ ID NO:112-113.

The following IR constructs target genes from Phytophthora infestans (they contain the intron of SEQ ID NO: 149, apart from the target sequences):
IR-NPP1/INF1/GK4/piacwp1-1/piacwp1-2/piacwp1-3, SEQ ID NO:68-69;
IR-piacwp1-1/piacwp1-2/piacwp1-3, SEQ ID NO:70-71;
IR-GK4/INF1/NPP1, SEQ ID NO:72-73;
IR-GK4/INF1/piacwp1-1, SEQ ID NO:74-75.

The following IR constructs target genes from Plasmopara viticola (they contain the intron of SEQ ID NO: 149, apart from the target sequences):
IR-PITG_17947/PITG_10772/PITG_13671/PITG_16956/PITG_00891, SEQ ID NO: 114-115;
IR-PITG_17947/PITG_10772/PITG_13671, SEQ ID NO:116-117;
IR-PITG_13671/PITG_16956/PITG_00891, SEQ ID NO:118-119.

The following IR constructs target genes from Plum Pox Virus (PPV) (they contain the intron of SEQ ID NO: 149, apart from the target sequences):
IR-P1/HC-Pro/CP, SEQ ID NO:39-40;
IR-P1, SEQ ID NO:41-42;
IR-HC-Pro, SEQ ID NO:43-44;
IR-CP, SEQ ID NO:45-46.

### Example 10: Design and Generation of Bacterial Small RNA Reporters to Validate the Biological Activity of P40 Fraction Batches Produced from the Chlorella Reference Lines

To validate the biological activity of EVs produced from Chlorella transgenic lines expressing antimicrobial small RNAs, we developed engineered bacteria (including the Escherichia coli K12 strain). These reporter systems are expected to exhibit a differential reporter gene expression when EV-embedded and/or associated small RNAs are internalized by the recipient cells. One reporter system family can be based on the plasmid expression of a bipartite cassette composed of a first construct expressing a short-lived variant of the transcriptional repressor, namely LacI-lite, carrying in its 5′ or 3′ ends the antimicrobial siRNA target region of interest, and a second construct composed of an intermediate stability variant of the GFP (Andersen et al., 1998; Elowitz & Leibler., 2000), whose transcriptional activity is directed by the pLac promoter and regulated by the lacO operator (FIG. 6A). In the absence of EV-embedded and/or associated small RNAs, LacI-lite proteins should be constitutively produced in bacteria and in turn shut-down the expression of the GFP, resulting in an absence of GFP fluorescence signal. By contrast, when a given small RNA population is internalized and active in bacterial cells, the silencing of LacI-lite results in the derepression of the GFP expression, leading to the detection of GFP fluorescence signal (FIG. 6A). Of note, other systems than LacI-lacO could also be used for the same purpose, such as the TetR-lite/tetO2 or cI-lite/λPR systems. We assembled the bipartite reporter system in destination plasmids with backbones adapted for expression and replication in both E. coli and Pto DC3000. In both cases, we introduced a small RNA target sequence specific to the Pto DC3000 gene fusA at the 3′ end of the LacI-lite repressor, giving the R37 construct. As a first characterization step, we made use of the Isopropyl β-d-1-thiogalactopyranoside (IPTG) ability to directly inhibit the LacI protein, in order to test the reporter system and its sensitivity once transformed in bacteria. We therefore incubated E. coli TOP10 bacterial cells transformed with the R37 construct with different IPTG concentrations, from 10⁻³ to 1 mM, and continuously measured both the OD₆₀₀ and GFP fluorescence over a 15 hours kinetic experiment. The GFP fluorescence of the +IPTG conditions was normalized using the −IPTG and a chloramphenicol control, in order to determine the correct induction kinetics once the background signal was removed. The analysis revealed that an IPTG concentration of 0.1 mM was sufficient to trigger the GFP induction already after 60′, with a sharp fluorescence increase starting after about 2-3 hours of incubation (FIG. 6B). Interestingly, neither the presence of the reporter nor of the IPTG itself affected the bacterial growth rate over the kinetics, as shown by OD₆₀₀ measurements (data not shown). After generating a Pto DC3000 compatible vector with the R37 construct, we performed the same IPTG test in this bacterial phytopathogen. The normalized GFP fluorescence revealed that the reporter system was active and responded to the IPTG induction also in this Pseudomonas species starting from 2-3 hours of incubation (FIG. 6C). However, we noticed that the overall GFP levels were lower compared to the ones detected in E. coli cells.

We also engineered three other dual reporter systems that exhibit a differential siRNA targeted reporter gene expression when EV-embedded siRNAs are internalized in bacterial cells.

A first reporter system family relies on the plasmid-based expression of a cassette composed of a first construct constitutively expressing a non-targeted DsRed reporter that is used as an internal control for normalization, and a second construct carrying a destabilized GFP reporter, containing in its downstream region the antimicrobial siRNA target region of interest (FIG. 6D). When expressed in bacteria (e.g., E. coli), this system is predicted to result in a specific decrease in GFP expression and fluorescence signal upon internalization of a given EV-embedded siRNA population.

A second dual reporter system family is based on the plasmid expression of a tripartite cassette composed of a first construct expressing a short-lived variant of the transcriptional repressor, namely TetR-lite, carrying in its downstream region the antimicrobial siRNA target region of interest, a second construct composed of an intermediate stability variant of the GFP (Andersen et al., 1998; Elowitz & Leibler, 2000), whose transcriptional activity is controlled by the tetO2 (or tetO1) operator, and a third construct expressing a non-targeted DsRed reporter, which serves as internal control for normalization (FIG. 6E). In the absence of EV-embedded small RNA, TetR-lite proteins should be constitutively produced in bacteria and in turn shut-down the expression of the GFP, resulting in an absence of GFP fluorescence signal (only the fluorescence of the DsRed reporter should be detected). By contrast, when a given siRNA population is internalized and active in bacterial cells, the silencing of TetR-lite results in the derepression of the GFP expression, leading to the detection of GFP fluorescence signal.

A third family of reporter system, relies on the plasmid-based expression of a bipartite cassette composed of a first construct expressing a short-lived variant of the transcriptional repressor, namely TetR-lite, carrying in its downstream region the antimicrobial siRNA target region of interest and a second construct composed of an intermediate stability variant of the GFP (Andersen et al., 1998; Elowitz & Leibler, 2000), or a bioluminescence reporter (e.g., the Photorhabdus luminescens operon luxCDABE (Meighen, 1991), whose transcriptional activity is controlled by the tetO2 (or tetO1) operator (FIG. 6F). When a given siRNA population is internalized and active in bacterial cells, the silencing of TetR-lite results in the derepression of the GFP or luxCDABE operon expression, leading to the detection of GFP fluorescence or bioluminescence signals. Of note, other systems than TetR-tetO2 could also be used for the same purpose, such as the lacI-lite/lacO or cl-lite/λPR systems.

### Example 11: Chlorella IR-CFA6/HRPL EVs Produced in a One Liter Photobioreactor Maintain a Normal Size Distribution and Production Rate

A prerequisite for the development of MIGS-derived applications, is to verify that Chlorella EVs maintain their integrity when produced in photobioreactors (PBRs). To address this issue, the reference Chlorella transgenic line IT20 #3 expressing the IR-CFA6/HRPL transgene was grown under continuous light conditions (270 μmol/m²/s) in a 1 L PBR for 3.3 days (FIG. 7A). It is noteworthy that the growth rate of this line was comparable to the one achieved with a wild type Chlorella vulgaris strain grown in the same PBR conditions, indicating that the expression of the inverted repeat transgene seems not to alter the fitness of this microalgae (data not shown). This is an important distinction from mammalian cell lines that trigger a potent inflammatory response upon sensing of long dsRNAs by RIG-I-like receptors (RLRs) (Fan & Jin, 2019). The extracellular medium from the above Chlorella IT20 #3 culture was further collected and separated from microalgae cells using a low-speed centrifugation method, two rounds of centrifugation at 3000 to 4000 g for 10 to 15 min. Chlorella EVs were further purified using the ultrafiltration and ultracentrifugation methods described in EXAMPLE 3, and the resulting P40 fractions were analyzed by NTA. We found that the size distribution of EVs was similar to the one retrieved from the same Chlorella line grown in flask conditions (data not shown). Furthermore, when comparable volumes of cell-free media recovered from flask and PBR conditions were analyzed, we detected a similar number of PKH26-positive exosome-like particles, ranging from 3.7×10⁷ to 3.8×10⁸ particles per ml from 1 liter of collected extracellular medium. These data indicate that we can maintain a normal size distribution and production rate of Chlorella EVs when produced in small PBRs.

### Example 12. Chlorella EV-Contained Antibacterial Small RNAs can be Relatively Easily Produced and Purified from the Extracellular Medium of a 150 L PBR without Altering their Yield, Integrity and Functionality

We next wanted to verify whether the scaling-up from a few liters of production (laboratory) up to a few m³ (pre-industrial) would not impact the yield nor the integrity of Chlorella EVs. To test this, we grew the reference Chlorella IT20 #3 line in a 150 L PBR, as detailed in EXAMPLE 1 (FIG. 7A). The NTA analysis revealed that, despite the presence in the original sample of suspended organic matter, the size distribution of Chlorella EVs was well centered around 150 nm diameter, with ˜70% of the sample being in a size range between 100 and 200 nm and the rest were above 200 nm (FIG. 7B). Further PKH26-labeling of the P40 fractions recovered from the 150 L PBR, followed by a NTA analysis in a fluorescence mode, exhibited a size distribution similar to the one observed when a NTA analysis was conducted from unlabeled P40 fractions in a scattering mode (FIG. 7B/C). This result suggests that the detected particles from unlabeled P40 fractions are likely lipid-based EVs. Interestingly, the particle concentration obtained from 5 L of cell-free medium was quite high: 3.3×10¹⁰ particles/ml, corresponding to ˜60 μM in ˜2.5 ml. In comparison, the production of EVs obtained from several flasks (˜800 ml of cell-free medium), usually yield ˜0.5 μM (3×10⁸ particles/ml) in 1 ml. Therefore, Chlorella EVs production in a 150 L PBR is ˜20 times more productive than in flasks. Collectively, these data indicate that Chlorella EVs recovered from a 150 L PBR exhibit a normal size distribution. They also show that a ˜20-fold increase in EVs yield can be obtained in such PBRs, even when standard growth conditions were used. It is therefore anticipated that the EVs yield will be relatively easily enhanced by optimizing the growth conditions and subsequent pre-filtration, filtration and purification steps.

Finally, we evaluated the biological activity of the Chlorella EV-embedded and/or-associated small RNAs obtained from the 150 L PBR culture. To this aim, we compared total RNAs and P40 fractions from different production systems, (flask, 1 L and 150 L PBRs) of both the IT20 #3 (IR-CFA6/HRPL) and IT19 #7 (IR-CYP51) lines in a stomatal reopening assay, as described in EXAMPLE 5. This comparative analysis revealed that both the total RNAs and P40 fractions from the Chlorella IT20 #3 line have similar antibacterial effects, inhibiting Pto DC3000-triggered stomatal reopening, independently of the production method employed (FIG. 7D).

Our results therefore provide a proof-of-concept demonstrating that Chlorella EV-contained antibacterial small RNAs can be relatively easily produced and purified from the extracellular medium of a 150 L PBR without altering their yield, integrity and functionality.

### Example 13. Optimization of EVs Production and/or Secretion Through Treatments of Chlorella Cultures with Supernatants from Heat-Killed Bacteria

Our data from Chlorella cultures grown in different conditions (flask vs PBRs) suggest that the growth conditions—as the ones used in the 150 L PBR—can elevate the yield of purified EVs (EXAMPLE 12). Besides growth conditions, there are emerging data indicating that abiotic and biotic stresses can promote EVs production and/or secretion from eukaryotic cells (Collett et al., 2018; Nakase et al., 2021). For example, specific temperature conditions or viral infections were shown to trigger an enhanced EVs release from cells (Bewicke et al., 2017; Schatz et al., 2017). Furthermore, a heightened plant EVs secretion was found in response to Pseudomonas syringae infection, as well as upon treatment with salicylic acid (Rutter & Innes, 2017), which is a phytohormone known to promote disease resistance against (hemi)biotrophic phytopathogens (Durrant & Dong, 2004). In addition, we detected an increased plant EVs secretion and/or biogenesis in response to the bacterial Microbe- or Pathogen-Associated Molecular Pattern (MAMP/PAMP) flagellin peptide flg22, which is sensed by the Pattern Recognition Receptor (PRR) Flagellin Sensing 2 (FLS2) and triggers plant immune signaling (Gomez-Gomez & Boller, 2000; Zipfel et al., 2004; Navarro et al., 2004; data not shown). To determine whether Chlorella EVs secretion and/or biogenesis could be similarly enhanced in response to biotic stresses, we decided to work with cultures in early stationary phase to obtain enough biomass and maximize the eventual positive effect of biotic stresses over EVs production and/or release. The Chlorella cultures were further treated with supernatants from heat-killed E. coli K12 TOP10 or Pto DC3000 Wt cells and then set in standard growth conditions, to avoid the risk of applying multiple stresses at the same time, which could alter Chlorella growth and/or EVs production/secretion. The rationale for using supernatants from heat-killed bacterial cells was that they should contain cocktails of molecules, including MAMPs/PAMPs, which could be sensed by yet-unknown Chlorella PRRs, thereby resulting in enhanced EVs production and/or secretion as found in plants. After 2 days of incubation, we quantified the cells and the purified EVs (FIG. 8A). Interestingly, although the treatments did not significantly affect Chlorella growth over the 2 days of incubation (data not shown), a 5 to 7 times increment in the yield of purified EVs was detected upon treatment with either bacterial supernatant compared to the control condition (FIG. 8B). These biotic stresses can thus be employed to increase Chlorella EVs production and/or secretion. Given that supernatants from heat-killed bacteria can be easily produced and in a cost-effective manner, our findings reveal promising conditions that might be suitable for enhancing the production of Chlorella EVs in large PBRs.

### BIBLIOGRAPHIC REFERENCES

Andersen, J. B., Sternberg, C., Poulsen, L. K., Bjorn, S. P., Givskov, M., & Molin, S. (1998). New unstable variants of green fluorescent protein for studies of transient gene expression in bacteria. Applied and environmental microbiology, 64(6), 2240-2246.
Askenasy, N., & Farkas, D. L. (2002). Optical imaging of PKH-labeled hematopoietic cells in recipient bone marrow in vivo. Stem cells (Dayton, Ohio), 20(6), 501-513.
Bai, L. L., Yin, W. B., Chen, Y. H., Niu, L. L., Sun, Y. R., Zhao, S. M., Yang, F. Q., Wang, R. R., Wu, Q., Zhang, X. Q., & Hu, Z. M. (2013). A new strategy to produce a defensin: stable production of mutated NP-1 in nitrate reductase-deficient Chlorella ellipsoidea. PloS one, 8(1), e54966.
Baulcombe D C. (2015). VIGS, HIGS and FIGS: small RNA silencing in the interactions of viruses or filamentous organisms with their plant hosts. Curr Opin Plant Biol. 26, 141-6.
Bewicke-Copley, F., Mulcahy, L. A., Jacobs, L. A., Samuel, P., Akbar, N., Pink, R. C., & Carter, D. (2017). Extracellular vesicles released following heat stress induce bystander effect in unstressed populations. Journal of extracellular vesicles, 6(1), 1340746. https://doi.org/10.1080/20013078.2017.1340746.
Blanc, G., Duncan, G., Agarkova, I., Borodovsky, M., Gurnon, J., Kuo, A., Lindquist, E., Lucas, S., Pangilinan, J., Polle, J., Salamov, A., Terry, A., Yamada, T., Dunigan, D. D., Grigoriev, I. V., Claverie, J. M., & Van Etten, J. L. (2010). The Chlorella variabilis NC64A genome reveals adaptation to photosymbiosis, coevolution with viruses, and cryptic sex. The Plant cell, 22(9), 2943-2955.
Bolognesi, R., Ramaseshadri, P., Anderson, J., Bachman, P., Clinton, W., Flannagan, R., Ilagan, O., Lawrence, C., Levine, S., Moar, W., Mueller, G., Tan, J., Uffman, J., Wiggins, E., Heck, G., & Segers, G. (2012). Characterizing the mechanism of action of double-stranded RNA activity against western corn rootworm (Diabrotica virgifera virgifera LeConte). PloS one, 7(10), e47534.
Broglie, K. E., Cerf, D. C., Herrmann, R., Lu, A. L., McGonigle, B. and Presnail, J. K. (2011). Composition and Methods for Insecticidial Control of Stinkbugs. CA 2 799 453, WO 2011/153418
Brooks, D. M., Hernández-Guzmán, G., Kloek, A. P., Alarcón-Chaidez, F., Sreedharan, A., Rangaswamy, V., Penaloza-Vizquez, A., Bender, C. L., & Kunkel, B. N. (2004). Identification and characterization of a well-defined series of coronatine biosynthetic mutants of Pseudomonas syringae pv. tomato DC3000. Molecular plant-microbe interactions: MPMI, 17(2), 162-174.
Cai, Q., Qiao, L., Wang, M., He, B., Lin, F. M., Palmquist, J., Huang, S. D., & Jin, H. (2018). Plants send small RNAs in extracellular vesicles to fungal pathogen to silence virulence genes. Science (New York, N.Y.), 360(6393), 1126-1129.
Campbell-Valois, F. X., Schnupf, P., Nigro, G., Sachse, M., Sansonetti, P. J., & Parsot, C. (2014). A fluorescent reporter reveals on/off regulation of the Shigella type III secretion apparatus during entry and cell-to-cell spread. Cell host & microbe, 15(2), 177-189.
Cerutti, H., Ma, X., Msanne, J., & Repas, T. (2011). RNA-mediated silencing in Algae: biological roles and tools for analysis of gene function. Eukaryotic cell, 10(9), 1164-1172.
Cha, T. S., Yee, W., & Aziz, A. (2012). Assessment of factors affecting Agrobacterium-mediated genetic transformation of the unicellular green alga, Chlorella vulgaris. World journal of microbiology & biotechnology, 28(4), 1771-1779.
Chien, L. F., Kuo, T. T., Liu, B. H., Lin, H. D., Feng, T. Y., Huang, C. C. (2012). Solar-to-bio H(2) production enhanced by homologous overexpression of hydrogenase in green alga Chlorella sp. DT, Int. J. Hydrog. Energy 37 (2012) 17738-17748.
Colao, I. L., Corteling, R., Bracewell, D., & Wall, I. (2018). Manufacturing Exosomes: A Promising Therapeutic Platform. Trends in molecular medicine, 24(3), 242-256. Dalakouras A, Jarausch W, Buchholz G, Bassler A, Braun M, Manthey T, Krczal G, Wassenegger M (2018) Delivery of hairpin RNAs and small RNAs into woody and herbaceous plants by trunk injection and petiole absorption. Front Plant Sci 9: 1253.
Collett G. P., Redman C. W., Sargent I. L., Vatish M. Endoplasmic reticulum stress stimulates the release of extracellular vesicles carrying danger-associated molecular pattern (DAMP) molecules. Oncotarget. 2018; 9: 6707-6717
de Andrade, C. J., de Andrade, L. M. (2017). An overview on the application of genus Chlorella in bio-technological processes. J Adv Res Biotech 2(1):1-9.
Durrant W E, Dong X. Systemic acquired resistance. Annu Rev Phytopathol. 2004; 42:185-209. doi: 10.1146/annurev.phyto.42.040803.140421. PMID: 15283665.
Elowitz, M. B., & Leibler, S. (2000). A synthetic oscillatory network of transcriptional regulators. Nature, 403(6767), 335-338.
Escobar, M. A., Civerolo, E. L., Summerfelt, K. R., Dandekar, A. M. (2001). RNAi-mediated oncogene silencing confers resistance to crown gall tumorogenesis. Proc. Natl. Acad. Sci. 98: 13437-13442.
Fan, X., & Jin, T. (2019). Structures of RIG-I-Like Receptors and Insights into Viral RNA Sensing. Advances in experimental medicine and biology, 1172, 157-188.
Fick, J., Barker, F. G., 2nd, Dazin, P., Westphale, E. M., Beyer, E. C., & Israel, M. A. (1995). The extent of heterocellular communication mediated by gap junctions is predictive of bystander tumor cytotoxicity in vitro. Proceedings of the National Academy of Sciences of the United States of America, 92(24), 11071-11075.
Fouts, D. E., Abramovitch, R. B., Alfano, J. R., Baldo, A. M., Buell, C. R., Cartinhour, S., Chatterjee, A. K., D'Ascenzo, M., Gwinn, M. L., Lazarowitz, S. G., Lin, N. C., Martin, G. B., Rehm, A. H., Schneider, D. J., van Dijk, K., Tang, X., & Collmer, A. (2002). Genomewide identification of Pseudomonas syringae pv. tomato DC3000 promoters controlled by the HrpL alternative sigma factor. Proceedings of the National Academy of Sciences of the United States of America, 99(4), 2275-2280.
Gao, Z., Wang, M., Blair, D., Zheng, Y., & Dou, Y. (2014). Phylogenetic analysis of the endoribonuclease Dicer family. PloS one, 9(4), e95350.
Ge Q, Filip L, Bai A, Nguyen T, Eisen H N, Chen J. Inhibition of influenza virus production in virus-infected mice by RNA interference. Proc Natl Acad Sci USA. 2004; 101(23):8676-8681. doi:10.1073/pnas.0402486101.
Gómez-Gómez L, Boller T. FLS2: an LRR receptor-like kinase involved in the perception of the bacterial elicitor flagellin in Arabidopsis. Mol Cell. 2000 June; 5(6):1003-11. doi: 10.1016/s1097-2765(00)80265-8. PMID: 10911994.
Guarnieri, M. T., Levering, J., Henard, C. A., Boore, J. L., Betenbaugh, M. J., Zengler, K., & Knoshaug, E. P. (2018). Genome Sequence of the Oleaginous Green Alga, Chlorella vulgaris UTEX 395. Frontiers in bioengineering and biotechnology, 6, 37. https://doi.org/10.3389/fbioe.2018.00037.
Guo, Z., Li, Y., & Ding, S. W. (2019). Small RNA-based antimicrobial immunity. Nature reviews. Immunology, 19(1), 31-44.
Herrmann, R., Lassner, M., Lu, A. L., Nelson, M., Presnail, J. K. and Rice, J. A. (2012). Compositions and methods for the suppression of target polynucleotides from the family Aphididae. EP 2 405 013 A
Hiruma, K., Gerlach, N., Sacristin, S., Nakano, R. T., Hacquard, S., Kracher, B., Neumann, U., Ramirez, D., Bucher, M., O'Connell, R. J., & Schulze-Lefert, P. (2016). Root Endophyte Colletotrichum tofieldiae Confers Plant Fitness Benefits that Are Phosphate Status Dependent. Cell, 165(2), 464-474.
Hou, Y., Zhai, Y., Feng, L., Karimi, H. Z., Rutter, B. D., Zeng, L., Choi, D. S., Zhang, B., Gu, W., Chen, X., et al. (2019). A Phytophthora Effector SuppressesTrans-Kingdom RNAi to Promote Disease Susceptibility. Cell Host Microbe25, 153-165.e5
Ivashuta, S., Zhang, Y., Wiggins, B. E., Ramaseshadri, P., Segers, G. C., Johnson, S., Meyer, S. E., Kerstetter, R. A., McNulty, B. C., Bolognesi, R., & Heck, G. R. (2015). Environmental RNAi in herbivorous insects. RNA (New York, N.Y.), 21(5), 840-850.
Jones, J D., Vance, R E., Dangl, J L. (2016). Intracellular innate immune surveillance devices in plants and animals. Science. 354, DOI: 10.1126/science.aaf6395.
Kim, D. H., Kim, Y. T., Cho, J. J., Bae, J. H., Hur, S. B., Hwang, I., & Choi, T. J. (2002). Stable integration and functional expression of flounder growth hormone gene in transformed microalga, Chlorella ellipsoidea. Marine biotechnology (New York, N.Y.), 4(1), 63-73. Koch A., Kumar N., Weber L., Keller, H., Imani J., Kogel, K H. (2013) Host-induced gene silencing of cytochrome P450 lanosterol C14 α-demethylase-encoding genes confers strong resistance to Fusarium species. Proc Natl Acad Sci U S A. 110: 19324-9.
Koch, A. et al. Host-induced gene silencing of cytochrome P450 lanosterol C14 alpha-demethylase-encoding genes confers strong resistance to Fusarium species. Proc Natl Acad Sci USA 110, 19324-9 (2013).
Koch, A., Biedenkopf, D., Furch, A., Weber, L., Rossbach, O., Abdellatef, E., Linicus, L., Johannsmeier, J., Jelonek, L., Goesmann, A., Cardoza, V., McMillan, J., Mentzel, T., Kogel, K H. (2016). An RNAi-based control of Fusarium graminearum infections through spraying of long dsRNAs involves a plant passage and is controlled by the fungal silencing machinery. PLoS Pathog. 12: e1005901.
Koch, A., Kogel, K H. (2014). New wind in the sails: improving the agronomic value of crop plants through RNAi-mediated gene silencing. Plant Biotechnol J. 12, 821-31.30
Kolb, C., Nagel, M. K., Kalinowska, K., Hagmann, J., Ichikawa, M., Anzenberger, F., Alkofer, A., Sato, M. H., Braun, P., & Isono, E. (2015). FYVE1 is essential for vacuole biogenesis and intracellular trafficking in Arabidopsis. Plant physiology, 167(4), 1361-1373.
Lacombe, S., Rougon-Cardoso, A., Sherwood, E., Peeters, N., Dahlbeck, D., van Esse, H P., Smoker, M., Rallapalli, G., Thomma, BP., Staskawicz, B., Jones, JD., Zipfel, C. (2010). Interfamily transfer of a plant pattern-recognition receptor confers broad-spectrum bacterial resistance. Nat. Biotechnol. 28:365-9.
Langmead, B., Trapnell, C., Pop, M., Salzberg, S. L., 2009. Ultrafast and memory-efficient alignment of short DNA sequences to the human genome. Genome Biol. 10, R25.
Lin, H. D., Liu, B. H., Kuo, T. T., Tsai, H. C., Feng, T. Y., Huang, C. C., & Chien, L. F. (2013). Knockdown of PsbO leads to induction of HydA and production of photobiological H2 in the green alga Chlorella sp. DT. Bioresource technology, 143, 154-162.
Mansfield, J., Genin, S., Magori, S., Citovsky, V., Sriariyanum, M., Ronald, P., Dow, M., Verdier, V., Beer, S V., Machado, M. A., Toth, I., Salmond, G., Foster, G. D. (2012). Top 10 plant pathogenic bacteria in molecular plant pathology. Mol Plant Pathol. 13:614-29.69
Meighen E. A. (1991). Molecular biology of bacterial bioluminescence. Microbiological reviews, 55(1), 123-142.
Melnyk, C W., Molnar, A., Baulcombe, DC. (2011). Intercellular and systemic movement of RNA silencing signals. EMBO J. 30, 3553-63.
Melotto M, Underwood W, Koczan J, Nomura K, He S Y. Plant stomata function in innate immunity against bacterial invasion. Cell. 2006; 126(5):969-980.
Micali, C O., Neumann, U., Grunewald, D., Panstruga, R., O'Connell. (2011). Biogenesis of a specialized plant-fungal interface during host cell internalization of Golovinomyces orontii haustoria. Cell Microbiol. 13, 210-226.
Michelmore, R. and Govindarajulu, M. (2012). Host-Induced gene silencing in vegetable species to provide durable disease resistance. WO2012/155109
Michelmore, R., Dubcovsky, J., Govindarajulu and M., Cantu, D. (2012). Disease-Resistance in cereals mediated by host-Induced gene silencing. WO2012/155112
Mukherjee, K., Campos, H., & Kolaczkowski, B. (2013). Evolution of animal and plant dicers: early parallel duplications and recurrent adaptation of antiviral RNA binding in plants. Molecular biology and evolution, 30(3), 627-641.
Murphy D., Dancis B., Brown J. R. (2008). The evolution of core proteins involved in microRNA biogenesis. BMC Evol. Biol. 8:92. 10.1186/1471-2148-8-92
Nakase, I., Ueno, N., Matsuzawa, M., Noguchi, K., Hirano, M., Omura, M., Takenaka, T., Sugiyama, A., Bailey Kobayashi, N., Hashimoto, T., Takatani-Nakase, T., Yuba, E., Fujii, I., Futaki, S. and Yoshida, T. (2021), Environmental pH stress influences cellular secretion and uptake of extracellular vesicles. FEBS Open Bio, 11: 753-767. https://doi.org/10.1002/2211-5463.13107.
Navarro L, Zipfel C, Rowland O, Keller I, Robatzek S, Boller T, Jones J D. The transcriptional innate immune response to flg22. Interplay and overlap with Avr gene-dependent defense responses and bacterial pathogenesis. Plant Physiol. 2004 June; 135(2):1113-28. doi: 10.1104/pp. 103.036749. Epub 2004 Jun. 4. PMID: 15181213; PMCID: PMC514144.
Navarro, L. and Singla-Rastogi, M. (2020). RNA-Based Biocontrol Methods to Protect Plants Against Pathogenic Bacteria and/or Promote Beneficial Effects of Symbiotic and Commensal Bacteria. WO/2020/035619, PCT/EP2019/072169
Navarro, L. and Singla-Rastogi, M. (2020). RNA-Based Therapeutic Methods to Protect Animals Against Pathogenic Bacteria and/or Promote Beneficial Effects of Symbiotic and Commensal Bacteria. WO/2020/035620, PCT/EP2019/072170
Niu, Y. F., Zhang, M. H., Xie, W. H., Li, J. N., Gao, Y. F., Yang, W. D., Liu, J. S., & Li, H. Y. (2011). A new inducible expression system in a transformed green alga, Chlorella vulgaris. Genetics and molecular research: GMR, 10(4), 3427-3434.
Noble J M, Roberts L M, Vidavsky N, Chiou A E, Fischbach C, Paszek M J, Estroff L A, Kourkoutis L F. Direct comparison of optical and electron microscopy methods for structural characterization of extracellular vesicles. J Struct Biol. 2020 Apr. 1; 210(1):107474. doi: 10.1016/j.jsb.2020.107474. Epub 2020 Feb. 4. PMID: 32032755; PMCID: PMC7067680.
O'Brien, K., Breyne, K., Ughetto, S., Laurent, L. C., & Breakefield, X. O. (2020). RNA delivery by extracellular vesicles in mammalian cells and its applications. Nature reviews. Molecular cell biology, 1-22. Advance online publication.
Ostrowski, M., Carmo, N. B., Krumeich, S., Fanget, I., Raposo, G., Savina, A., Moita, C. F., Schauer, K., Hume, A. N., Freitas, R. P., Goud, B., Benaroch, P., Hacohen, N., Fukuda, M., Desnos, C., Seabra, M. C., Darchen, F., Amigorena, S., Moita, L. F., & Thery, C. (2010). Rab27a and Rab27b control different steps of the exosome secretion pathway. Nature cell biology, 12(1), 19-13.
Run, C., Fang, L., Fan, J., Fan, C., Luo, Y., Hu, Z., Li, Y. (2016). Stable nuclear transformation of the industrial alga Chlorella pyrenoidosa, Algal Research 17 (2016) 196-201
Rutter, B. D., & Innes, R. W. (2017). Extracellular Vesicles Isolated from the Leaf Apoplast Carry Stress-Response Proteins. Plant physiology, 173(1), 728-741.
Safi, C., Zebib, B., Merah, O., Pontalier, P.-Y., Vaca-Garcia, C. (2014). Morphology, composition, production, processing and applications of Chlorella vulgaris: A review. Renewable and Sustainable Energy Reviews, Volume 35, July 2014, Pages 265-278.
Schatz D, Rosenwasser S, Malitsky S, Wolf S G, Feldmesser E, Vardi A. Communication via extracellular vesicles enhances viral infection of a cosmopolitan alga. Nat Microbiol. 2017 November; 2(11):1485-1492. doi: 10.1038/s41564-017-0024-3. Epub 2017 Sep. 18. PMID: 28924189.
Singla-Rastogi, M., Charvin, M., Thiebeauld, O., Perez-Quintero, A. L., Ravet, A., Fortunato, A. E., Mendu, V., Navarro, L. (2019). Plant Small RNA Species Direct Gene Silencing in Pathogenic Bacteria as well as Disease Protection. bioRxiv 863902;
Sreedharan, A., Penaloza-Vazquez, A., Kunkel, B. N., & Bender, C. L. (2006). CorR regulates multiple components of virulence in Pseudomonas syringae pv. tomato DC3000. Molecular plant-microbe interactions: MPMI, 19(7), 768-779.
Teng, Y., Ren, Y., Sayed, M., Hu, X., Lei, C., Kumar, A., Hutchins, E., Mu, J., Deng, Z., Luo, C., et al. (2018). Plant-Derived Exosomal MicroRNAs Shape the Gut Microbiota. Cell Host Microbe24, 637-652.e8.
Wang, M., Thomas, N., Jin, H. (2017). Cross-kingdom RNA trafficking and environmental RNAi for powerful innovative pre- and post-harvest plant protection. Curr Opin Plant Biol. 38:133-141.
Wang, M., Weiberg, Arne., Lin F M., Thomma, B P H., Huang, H D., Jin, H. (2016). Bidirectional cross-kingdom RNAi and fungal uptake of external RNAs confer plant protection. Nature plants. 16151.
Wang, Q., Zhuang, X., Mu, J., Deng, Z. B., Jiang, H., Zhang, L., Xiang, X., Wang, B., Yan, J., Miller, D., & Zhang, H. G. (2013). Delivery of therapeutic agents by nanoparticles made of grapefruit-derived lipids. Nature communications, 4, 1867.
Wang F, Vandepoele K, Van Lijsebettens M. Tetraspanin genes in plants. Plant Sci. 2012 July; 190:9-15. doi: 10.1016/j.plantsci.2012.03.005. Epub 2012 Mar. 23. PMID: 22608515.
Weiberg, A., Bellinger, M., Jin H. (2015). Conversations between kingdoms: small RNAs. Curr Opin Biotechnol. 32, 207-15.
Weiberg, A., Wang, M., Lin, F M., Zhao, H., Zhang, Z., Kaloshian, I., Huang, H D., Jin, H. 5 (2013). Fungal small RNAs suppress plant immunity by hijacking host RNA interference pathways. Science. 342, 118-23.
Whangbo, J. S., & Hunter, C. P. (2008). Environmental RNA interference. Trends in genetics: TIG, 24(6), 297-305.
Yang, B., Liu, J., Liu, B., Sun, P., Ma, X., Jiang, Y., Wei, D., Chen, F. (2015).
Development of a stable genetic system for Chlorella vulgaris—a promising green alga for CO2 biomitigation, Algal Res. 12 (2015) 134-141.
Zhang, J., Qiu, Y., & Xu, K. (2020). Characterization of GFP-AtPEN1 as a marker protein for extracellular vesicles isolated from Nicotiana benthamiana leaves. Plant signaling & behavior, 15(9), 1791519.
Zhang, M., Viennois, E., Xu, C., & Merlin, D. (2016). Plant derived edible nanoparticles as a new therapeutic approach against diseases. Tissue barriers, 4(2), e1134415.
Zhang, R., Lin, Y. (2009). DEG 5.0, a database of essential genes in both prokaryotes and eukaryotes. Nucleic Acids Res., 37: D455-D458.
Zhang, T., Zhao, Y L., Zhao, J H., Wang, S., Jin, Y., Chen, Z Q., Fang, Y Y., Hua, C L., Ding, S W., Guo, H S. (2016). Cotton plants export microRNAs to inhibit virulence gene expression in a fungal pathogen. Nature Plants.
Zhang H, Kolb F A, Jaskiewicz L, Westhof E, Filipowicz W. Single processing center models for human Dicer and bacterial RNase III. Cell. 2004 Jul. 9; 118(1):57-68. doi: 10.1016/j.cell.2004.06.017. PMID: 15242644.
Zipfel C, Robatzek S, Navarro L, Oakeley E J, Jones J D, Felix G, Boller T. Bacterial disease resistance in Arabidopsis through flagellin perception. Nature. 2004 Apr. 15; 428(6984):764-7. doi: 10.1038/nature02485. PMID: 15085136.
Zuñiga, C., Li, C. T., Huelsman, T., Levering, J., Zielinski, D. C., McConnell, B. O., Long, C. P., Knoshaug, E. P., Guarnieri, M. T., Antoniewicz, M. R., Betenbaugh, M. J., & Zengler, K. (2016). Genome-Scale Metabolic Model for the Green Alga Chlorella vulgaris UTEX 395 Accurately Predicts Phenotypes under Autotrophic, Heterotrophic, and Mixotrophic Growth Conditions. Plant physiology, 172(1), 589-602.

1. A method for producing functional interfering small RNAs, said method comprising at least the steps of:
a) transforming Chlorella cells with a siRNA or miRNA precursor comprising at least one fragment of at least one target gene, and
b) cultivating said Chlorella cells in appropriate conditions so that they express said precursor and release extracellular vesicles (EV)-embedded functional small iRNAs targeting said at least one gene fragment.

2. The method of claim 1, wherein said siRNA or miRNA precursor is a long single- or double-stranded RNA molecule.

3. The method of claim 1, wherein said gene fragment comprises between 50 and 3000 bp.

4. The method of claim 1, further comprising the step of recovering said small iRNAs from said Chlorella cells.

5. The method of claim 1, further comprising the step of recovering the Extracellular Vesicles (EV) released by said Chlorella cells in the extracellular medium.

6. The method of claim 1, wherein said target gene is an oomycete gene, a viral gene, a bacterial gene, or a fungus gene.

7. Chlorella-derived EVs obtained by the method comprising the steps of:
a) transforming Chlorella cells with a siRNA or miRNA precursor comprising at least one fragment of at least one target gene, and
b) cultivating said Chlorella cells in appropriate conditions so that they express said precursor and release extracellular vesicles (EV)-embedded functional small iRNAs targeting said at least one gene fragment,
c) recovering the Extracellular Vesicles (EV) released by said Chlorella cells in the extracellular medium,
said EV containing a population of functional small iRNAs targeting one or several region(s) in said at least one target gene(s).

8. Chlorella-derived EVs of claim 7, wherein said population of functional small iRNAs targets one or several region(s) in at least one viral gene.

9. Chlorella-derived EVs of claim 7, wherein said population of functional small iRNAs targets one or several region(s) in at least one bacterial gene.

10. A method for treating a parasitic infection and/or infectious disease in plants, said method comprising the use of the Chlorella-derived EV as defined in claim 7.

11. A phytotherapeutic composition containing an effective amount of Chlorella-derived EV as defined in claim 7 and an agronomical acceptable vehicle.

12. Recombinant Chlorella cells expressing a siRNA or miRNA precursor comprising at least one fragment of at least one target gene, said Chlorella cells releasing EV-embedded functional small iRNAs targeting said at least one gene fragment.

13. The method of claim 1, or the recombinant Chlorella cells of claim 12, wherein said siRNA precursor has a sequence chosen among SEQ ID NO:1-148.

14. The method of claim 1, comprising the use of the siRNA precursor of SEQ ID NO:1-148.

15. A versatile platform for producing high-throughput amount of EV-embedded functional interfering small RNAs, said platform using the recombinant Chlorella cells as defined in claim 12.

16. A phytotherapeutic composition comprising the recombinant Chlorella cells as defined in claim 12.
